(19) United States
(12) Patent Application Publication
(10) Pub. No.: US 2010/0048678 A1
(43) Pub. Date: Feb. 25, 2010

(54) Nucleic acids and polypeptides that are useful in controlling neuronal regeneration

(76) Inventors: Guus Smit, Amsterdam (NL); Joost Verhaagen, Amsterdam (NL); Ronald Ernst van Kesteren, Almere (NL); Florien Jansje Stam, Amsterdam (NL)

Correspondence Address: MORGAN LEWIS & BOCKIUS LLP, 1111 PENNSYLVANIA AVENUE NW, WASHINGTON, DC, 20004, US

(21) Appl. No.: 11/920,721

(22) PCT Filed: May 18, 2006
(86) PCT No.: PCT/NL2006/050118

§ 371 Date: Nov. 20, 2007

Related U.S. Application Data
(60) Provisional application No. 60/682,798, filed on May 20, 2005.

(51) Int. Cl. A61K 31/7088; C12Q 1/68; C12N 15/63; C07H 21/02

(52) U.S. Cl. 514/44 R; 435/6; 435/320.1; 536/23.1; 536/24.1

(57) ABSTRACT

The present invention relates to methods for promoting regeneration response of peripheral and central nervous systems in mammals in need of such biological effects. The methods comprise altering the activity or steady state level of specific polypeptides that control regeneration of injured or degenerated neuronal cells. Preferably the activity or steady state level of specific polypeptides is altered by introducing nucleic acids to increase or decrease expressing of the polypeptides. These are useful in or suffering from neurodegenerative disorders.

### FIELD OF THE INVENTION

The present invention relates to nucleic acids and polypeptides encoded thereby, whose expression is modulated in cells of the dorsal root ganglia undergoing a regenerative response elicited by crush damage of the sciatic nerve. Such polypeptides are referred to as transcription factors (TFs) herein. These nucleic acids are useful in methods for controlling a regeneration response of peripheral and central nervous systems in mammals in need of such biological effects, including the treatment of humans after neurotraumatic injury, e.g. after lesion, avulsion or contusion of nerve tissue.

### BACKGROUND OF THE INVENTION

Most spinal cord injuries in humans are caused by road traffic, work or sports accidents and involve (i) fractures or dislocations of the vertebrae resulting in contusion of the spinal cord and disruption of the major ascending and descending pathways, including the corticospinal tracts (CST), and/or (ii) avulsion of dorsal and/or ventral spinal roots thereby disconnecting the spinal cord from the peripheral nerves. Both injuries to the long tracts and local nerve root injuries have serious consequences for the patient. About 50% of all spinal cord injured patient are tetraplegic (both arms and legs are affected) and the other half is paraplegic (legs are effected, but arms not). Spinal cord injury affects mostly young, healthy individuals that are part of the workforce and lead productive lives. Most patients surviving the acute phase of spinal cord injury will become wheel chair bound and have a life expectancy of several decades. To date no effective treatments for spinal cord or spinal root injuries are available. In the case of ventral root avulsion some success has been reported with surgical reimplantation of the avulsed roots into the spinal cord. Recovery of arm and shoulder function as a result of this neurosurgical intervention is, however, very limited.

Traumatic injuries to peripheral nerves have a somewhat better prospect then spinal cord or root avulsion lesions. In some cases the proximal and distal stumps of an injured peripheral nerve can be stitched together by the neurosurgeon. In a significant number of patients autologous nerve transplants have to be used to bridge the gap between the proximal and distal stump. Injured axons grow through the transplant and in many instances some of these axons will reconnect to the muscle or skin resulting in some return of function. There is, however, a significant need for improvement as functional recovery after surgical peripheral nerve repair is normally not complete.

It is evident that the injured CNS has a very limited capacity for self-repair. In the CNS, neurons fail to induce the expression of genes required for growth and the glia cells in the neural scar express neurite outgrowth inhibitors. In contrast, in the PNS neurons do initiate a program of gene expression that successfully drives regeneration of injured axons and glia cells in peripheral nerves (the Schwann cells) support regeneration. Cell biological and molecular studies have demonstrated that (1) neuronal (or intrinsic) and (2) glial (or “environmental”) factors play a crucial role in the neuroregeneration process.

So far a small set of neuronal genes has been identified that is upregulated in injured peripheral neurons but not, or to a much lesser extent in injured CNS-neurons. The dichotomy between PNS and CNS neurons to regenerate is thus caused by molecular differences. We and others have overexpressed two of the first “growth-associated” genes (GAP-43 and CAP-23) in neurons in transgenic mice. This results in an enhanced capacity to extent new neurites after injury. These observations have led to the very important notion that injured CNS-neurons can be triggered to regenerate by enhancing the expression of “regeneration-associated” neuronal genes.

CNS and PNS neurons do behave differently in terms of intrinsic regenerative capacity and are exposed to strongly different environments. In contrast to the situation in the CNS, lesioned PNS neurons regenerate axons along Schwann cells present in the distal nerve stump of the injured nerve. Although some neuroma and scar formation occurs in peripheral nerves extensive scar formation as seen in the CNS is much less of a problem. Moreover, regeneration in the PNS occurs by the virtue of the presence of growth promoting Schwann cells. Although from a scientific point of view PNS regeneration has helped to gain more insight into the factors that contribute to successful outgrowth, from a clinical perspective, however, functional recovery in the PNS is in most instances far from complete.

In several recent studies, large scale gene expression changes in dorsal root ganglia (DRG) neurons after injury were characterized. These studies differ in the use of time-point, lesion type, tissue analyzed, and microarray platform, most of them find a marked upregulation of neuropeptides such as galanin and NP-Y, an upregulation of genes related to inflammation and a downregulation of genes associated with neurotransmission (Boeshore et al., 2004 J. Neurobiol. 59(2):216-35; Costigan et al., 2002, BMC Neurosci. 3(1):16; Wang et al., 2002, Neuroscience. 114(3):529-46; Xiao et al., 2002, Neuroreport. 13(15):1903-7). Three studies also report upregulation of cell cycle-related transcripts (Boeshore et al., 2004, supra; Cameron et al., 2003 J Cell Biochem. 88(5):970-85; Wang et al., 2002, supra). Changes in cell-cycle and inflammation related genes possibly reflect proliferation of macrophages in the lesioned tissue (Schreiber et al., 2002, J. Neurobiol. 53(1):68-79), whereas downregulation of genes involved in neurotransmission points to a dedifferentiation of adult neurons to a growth state, during which the normal physiology is altered. The aforementioned studies have not led to novel hypotheses on the molecular processes underlying successful regeneration for several reasons. First, most of these studies have not analyzed gene-expression in a time-course. The study by Xiao et al. is an exception, but due to their approach of analyzing the expression of genes present in a cDNA library of 14d transected and control DRG, detection of genes that are transiently upregulated in the early stages of the process is impossible. Second, most studies have used a transection paradigm (Boeshore et al., 2004, supra; Bonilla et al., 2002, J. Neurosci. 22(4):1303-15; Costigan et al., 2002, supra; Tanabe et al., 2003, J. Neurosci. 23(29):9675-86), whereas it has been shown that nerve crush leads to a more robust regenerative response (Nguyen et al., 2002, Nat. Neurosci. 5(9):861-7; Pan et al., 2003). Third, the number of regulated genes analyzed is small in some studies, either due to the platform used or to (stringent) fold-change cutoffs used when statistics cannot be applied (Bonilla et al., 2002, supra; Cameron et al., 2003, supra; Fan et al., 2001, Cell Mol. Neurobiol. 21(5):497-508; Schmitt et al., 2003, BMC Neurosci. 4(1):8; Tanabe et al., 2003, supra; Xiao et al., 2002, supra). Fourth, injury and regeneration associated genes cannot be distinguished if sciatic nerve (SN) lesion is the only paradigm analyzed.

Thus it is an object of the invention to overcome the short-comings in the above-cited art and to provide for the key proteins and/or encoding nucleic acids in the process of neural repair. It is a further object of the invention to provide for therapies bases on these proteins and/or nucleic acids that promote the repair process leading to return of function in neurotrauma patients.

### DESCRIPTION OF THE INVENTION

Most of the prior art gene expression analyses as discussed above have provided single snapshots of the highly complex biological process of regeneration. Therefore, it is impossible to determine whether regulated genes at a particular timepoint are genes important for the initiation of the outgrowth process, play a role during axon elongation or are involved in target finding or reestablishment of sensory contacts. In order to link genes to a part of this process gene expression analysis should be performed in a time course.

In addition, the biological interpretation of gene expression data is facilitated to a great extent if a second, related but different process is analyzed in parallel. In this respect, the DRG neuron offers the unique opportunity to compare gene expression changes during a robust outgrowth response in the sciatic nerve (SN-crush) and a weak outgrowth response in the dorsal root (DR-crush). This comparison holds the advantages that the tissue samples that will be analyzed are very similar to each other, the only biological difference being the localization of the injury inflicted to the neurite. This is not the case if, for instance, gene expression in the lesioned CNS is compared to gene expression in DRG neurons. Also differential gene expression analysis allows to eliminate stress and injury related gene expression changes which could be similar in both paradigms. If genes that are regulated in a similar fashion by both injuries are excluded from further analysis, chances are that true regeneration-associated genes are enriched. Therefore, a high resolution time-course analysis of gene expression changes after DR and SN crush were used by the present inventors to reveal nucleic acids involved in successful regeneration.

The screens for intrinsic neuronal genes have been performed on primary sensory neurons of the rat DRG (see below). These neurons are uniquely suited to study successful and abortive regeneration. The cell bodies of these neurons are located in the dorsal root ganglia and these neurons possess two branches: one projecting peripherally innervating the skin, and one branch projecting centrally to the spinal cord. The peripheral branch regenerates vigorously while the central branch regenerates virtually not. By comparing changes in gene expression after a peripheral versus a central lesion we identified novel intrinsic, genes that are up-regulated or down regulated after lesion of the peripheral branch, but not after a central branch lesion. The power of this screen was not only the comparison of peripheral versus central regeneration, but also the fact that we specifically examined gene expression during the first 6 to 72 hours (5 time points) of the regenerative response. By doing so and by using used advanced target finding technology developed as a result of the human and rodent genome projects and have discovered a large set of new genes involved in the neuronal response. In particular we were able to discover the key factors that initiate the neuronal gene program that drives successful regeneration.

### DETAILED DESCRIPTION OF THE INVENTION

In one aspect the present invention relates to methods for promoting or controlling generation or regeneration of a neuronal cell. A first method for promoting or controlling generation or regeneration of a neuronal cell comprises the step of altering the activity or the steady state level of a polypeptide in the neuronal cell or in cells in the direct environment of the neuronal cell in need of (re)generation, e.g. the supporting glia cells (see also below). The polypeptide of which the activity or the steady state level is altered preferably is a polypeptide that comprises an amino acid sequence that is encoded by a nucleotide sequence selected from: (a) a nucleotide sequence that has at least 60, 70, 80, 85, 90, 95, 98 or 99% sequence identity with a nucleotide sequence selected from SEQ ID NO.'s 1-146; and, (b) a nucleotide sequence that encodes an amino acid sequence that has at least 60, 70, 80, 85, 90, 95, 98 or 99% amino acid identity with an amino acid sequence that is encoded by a nucleotide sequence selected from SEQ ID NO.'s 1-146. The polypeptides are herein further referred to as polypeptides of the invention, TF polypeptides, or briefly TFs. A TF polypeptide of the invention preferably is a transcription factor or a modulator of gene transcription and preferably its expression levels is altered at least in the early stages (and preferably also in later stages) of regeneration. The TF preferably determines whether neurons successfully regenerate. Changes in the activity or the steady state level of a TF result in an altered gene expression state that is required for robust neurite outgrowth and functional recovery. Preferably a TF of the invention is thus a key switch that determines whether a damaged neuron regenerates successfully or not.

An “alteration of the activity or steady state level of a polypeptide” is herein understood to mean any detectable change in the biological activity exerted by the polypeptide or in the steady state level of the polypeptide as compared the normal activity or steady-state in a healthy individual.

The generation or regeneration of a neuronal cell is understood to mean one or more of the processes including initiation of neuronal outgrowth, neuronal outgrowth, axon elongation, target finding and reestablishment of sensory contacts, up to return of function of the deficient motory or sensory neurons. Suitable assays for generation or regeneration of a neuronal cell are provided in the Examples, e.g. in Example II. The assays may be used to determine if an alteration of the activity or steady state level of a polypeptide of the invention is capable of inducing neurite outgrowth and thereby capable of inducing or promoting neuronal regeneration.

In the method of the invention the activity or steady-state level of the polypeptides of the invention may be altered at the level of the polypeptide itself, e.g. by providing a polypeptide of the invention to the neuronal cells from an exogenous source, or by adding an antagonist or inhibitor of the polypeptide to the neuronal cells, such as e.g. an antibody against the TF polypeptide. For provision of the TF polypeptide from an exogenous source the TF polypeptide may conveniently be produced by expression of a nucleic acid encoding the polypeptide in suitable host cells as described below. An antibody against a polypeptide of the invention may be obtained as described below.

Preferably, however, the activity or steady-state level of a TF polypeptide is altered by regulating the expression level of a nucleotide sequence encoding the polypeptide. Preferably, the expression level of a nucleotide sequence is regulated in the neuronal cells. The expression level of a polypeptide of the invention may be up-regulated by introduction of an expression construct (or vector) into the neuronal cells, whereby the expression vector comprises a nucleotide sequence encoding a TF polypeptide, and whereby the nucleotide sequence is under control of a promoter capable of driving expression of the nucleotide sequence in the neuronal cells. The expression level of a TF polypeptide may also be up-regulated by introduction of an expression construct into the neuronal cells, whereby the construct comprises a nucleotide sequence encoding a factor capable of trans-activation of the endogenous nucleotide sequence encoding the TF polypeptide.

Alternatively, if so required for neuro(re)generation, the expression level of a polypeptide of the invention may be down regulated by providing an antisense molecule to the neuronal cells, whereby the antisense molecule is capable of inhibiting the biosynthesis (usually the translation) of the nucleotide sequence encoding the TF polypeptide. Decreasing gene expression by providing antisense or interfering RNA molecules is described below herein and is e.g. reviewed by Famulok et al. (2002, Trends Biotechnol., 20(11): 462-466). The antisense molecule may be provided to the cells as such or it may be provided by introducing an expression construct into the neuronal cells, whereby the expression construct comprises an antisense nucleotide sequence that is capable of inhibiting the expression of the nucleotide sequence encoding a TF polypeptide, and whereby the antisense nucleotide sequence is under control of a promoter capable of driving transcription of the antisense nucleotide sequence in the neuronal cells. The expression level of a TF polypeptide may also be down-regulated by introducing an expression construct into the neuronal cells, whereby the expression construct comprises a nucleotide sequence encoding a factor capable of trans-repression of the endogenous nucleotide sequence encoding a TF polypeptide.

In the method of the invention the regeneration of the neuronal cell is preferably promoted by increasing the activity or the steady-state level of a polypeptide encoded by a nucleotide sequence selected from: (a) a nucleotide sequence that has at least 80% identity with a sequence selected from SEQ ID NO.'s 23, 35, 45, 65, 14, 32, 43, 72, 11, 12, 28, 8, 51, 4, 3, 42, 48, 52, 61, 5, 6, 10, 17, 49, 55, 58, 64, 70, 73, 27, 40, 96, 108, 118, 138, 87, 105, 116, 145, 84, 85, 101, 81, 124, 77, 76, 115, 121, 125, 134, 78, 79, 83, 90, 122, 128, 131, 137, 143, 146, 100, and 113; and, (b) a nucleotide sequence that encodes an amino acid sequence that has at least 80% amino acid identity with an amino acid sequence encoded by a nucleotide sequence selected from SEQ ID NO.'s 23, 35, 45, 65, 14, 32, 43, 72, 11, 12, 28, 8, 51, 4, 3, 42, 48, 52, 61, 5, 6, 10, 17, 49, 55, 58, 64, 70, 73, 27, 40, 96, 108, 118, 138, 87, 105, 116, 145, 84, 85, 101, 81, 124, 77, 76, 115, 121, 125, 134, 78, 79, 83, 90, 122, 128, 131, 137, 143, 146, 100, and 113. A more preferred selection includes SEQ ID NO.'s 23, 35, 45, 65, 14, 32, 43, 72, 11, 12, 28, 96, 108, 118, 138, 87, 105, 116, 145, 84, 85, and 101; and the most preferred selection includes SEQ ID NO.'s 8, 51, 4, 3, 42, 48, 52, 61, 5, 6, 10, 17, 49, 55, 58, 64, 70, 73, 27, 40, 81, 124, 77, 76, 115, 121, 125, 134, 78, 79, 83, 90, 122, 128, 131, 137, 143, 146, 100, and 113. The activity or the steady-state level of the polypeptide is preferably increased by introducing a nucleic acid construct into the neuronal cell(s), the nucleic acid construct comprising a nucleotide sequence (encoding the polypeptide) under control of a promoter capable of driving expression of the nucleotide sequence in the neuronal cell. Suitable promoters for expression in neuronal cells are further specified herein below.

Alternatively, in the method of the invention the regeneration of the neuronal cell is preferably promoted by decreasing the activity or the steady-state level of a polypeptide encoded by a nucleotide sequence selected from: (a) a nucleotide sequence that has at least 80% identity with a sequence selected from SEQ ID NO.'s 31, 69, 53, 18, 21, 26, 30, 36, 39, 44, 46, 60, 66, 71, 13, 19, 25, 47, 24, 9, 1, 20, 22, 63, 67, 2, 7, 15, 16, 34, 37, 38, 41, 50, 54, 56, 57, 62, 68, 29, 59, 33, 104, 142, 126, 91, 94, 99, 103, 109, 112, 117, 119, 133, 139, 144, 86, 92, 98, 120, 97, 82, 74, 93, 95, 136, 140, 75, 80, 88, 89, 107, 110, 111, 114, 123, 127, 129, 130, 135, 141, 102, 132, and 105; and, (b) a nucleotide sequence that encodes an amino acid sequence that has at least 80% amino acid identity with an amino acid sequence encoded by a nucleotide sequence selected from SEQ ID NO.'s 31, 69, 53, 18, 21, 26, 30, 36, 39, 44, 46, 60, 66, 71, 13, 19, 25, 47, 24, 9, 1, 20, 22, 63, 67, 2, 7, 15, 16, 34, 37, 38, 41, 50, 54, 56, 57, 62, 68, 29, 59, 33, 104, 142, 126, 91, 94, 99, 103, 109, 112, 117, 119, 133, 139, 144, 86, 92, 98, 120, 97, 82, 74, 93, 95, 136, 140, 75, 80, 88, 89, 107, 110, 111, 114, 123, 127, 129, 130, 135, 141, 102, 132, and 105. A more preferred selection includes SEQ ID NO.'s 31, 69, 53, 18, 21, 26, 30, 36, 39, 44, 46, 60, 66, 71, 13, 19, 25, 47, 24, 9, 1, 104, 142, 126, 91, 94, 99, 103, 109, 112, 117, 119, 133, 139, 144, 86, 92, 98, 120, 97, 82, and 74; and the most preferred selection includes SEQ ID NO.'s 20, 22, 63, 67, 2, 7, 15, 16, 34, 37, 38, 41, 50, 54, 56, 57, 62, 68, 29, 59, 33, 93, 95, 136, 140, 75, 80, 88, 89, 107, 110, 111, 114, 123, 127, 129, 130, 135, 141, 102, 132, and 105. The activity or the steady-state level of the polypeptide is preferably decreased by introducing an antisense or interfering nucleic acid molecule into the neuronal cell. The antisense or interfering nucleic acid molecule may be introduced into the cell directly “as such”, optionally in a suitable formulation, or it may be produce in situ in the cell by introducing into the cell an expression construct comprising a (antisense or interfering) nucleotide sequence that is capable of inhibiting the expression of the nucleotide sequence encoding the polypeptide, whereby, optionally, the antisense or interfering nucleotide sequence is under control of a promoter capable of driving expression of the nucleotide sequence in the neuronal cell (see herein below).

In the method of the invention the neuronal cell preferably is a neuronal cell in need of generation or regeneration. Such cells may be found at lesions of the nervous system that have arisen from traumatic contusion, avulsion, compression, and/or transection or other physical injury, or from tissue damage either induced by, or resulting from, a surgical procedure, from vascular pharmacologic or other insults including hemorrhagic or ischemic damage, or from neurodegenerative or other neurological diseases. The neuronal cell in need of generation or regeneration may be neuronal cell of the peripheral nervous system (PNS) but preferably is a cell of the central nervous system (CNS), in particular a neuronal cell of the corticospinal tract (CST). Although the cell in need of generation or regeneration in the methods of the invention will usually be a neuronal cell, other types of cells in the environment (vicinity) of the neuronal cells may influence the ability of the neuronal cell to (re)generate). Therefore the invention expressly includes aspects relating to altering the activity or the steady-state level of a polypeptide of the invention in cells in the environment of the neuronal cell in need of (re)generation. Such environmental cells include e.g. glia cells, Schwann cells, scleptomeningeal fibroblasts, blood borne cells that invade the lesion center, astrocytes and meningeal cells.

In a further aspect, the invention pertains to a method for treating a neurotraumatic injury or a neurodegenerative disease in a subject. The method preferably comprises pharmacologically altering the activity or the steady-state level of a polypeptide of the invention as defined above in an injured or degenerated neuron in the subject. Preferably, the alteration is sufficient to induce (axonal) generation or regeneration of the injured or degenerated neuron. In this method of the invention, the neurotraumatic injury may be as described above, and likewise, the injured or degenerated neurons in the subject may be neurons of the PNS, the CNS and/or the CST.

In the methods of the inventions, the neurodegenerative disease may be a disorder selected from: cerebrovascular accidents (CVA), Alzheimer's disease (AD), vascular-related dementia, Creutzfeldt-Jakob disease (CJD), bovine spongiform encephalopathy (BSE), Parkinson's disease (PD), brain trauma, multiple sclerosis (MS), amyotrophic lateral sclerosis (ALS—Lou Gehrig's disease) and Huntington's chorea.

The methods of the inventions preferably comprise the step of administering to the subject a therapeutically effective amount of a pharmaceutical composition comprising a nucleic acid construct for modulating the activity or steady state level of a TF polypeptide as defined herein. The nucleic acid construct may be an expression construct as further specified herein below. Preferably the expression construct is a viral gene therapy vector selected from gene therapy vectors based on an adenovirus, an adeno-associated virus (AAV), a herpes virus, a pox virus and a retrovirus. A preferred viral gene therapy vector is an AAV or Lentiviral vector. Alternatively the nucleic acid construct may be for inhibiting expression of a TF polypeptide of the invention such as an antisense molecule or an RNA molecule capable of RNA interference (see below). In the method, the pharmaceutical composition comprising the nucleic acid construct is preferably administered at a site of neuronal injury or degeneration.

In a further aspect the invention relates to the use of a nucleic acid construct for modulating the activity or steady state level of a TF polypeptide as defined herein, for the manufacture of a medicament for promoting regeneration of a neuronal cell, preferably in a method of the invention as defined herein above. Preferably, the nucleic acid construct is used for the manufacture of a medicament for the treatment of a neurotraumatic injury or neurodegenerative disease, preferably in a method of the invention as defined herein above.

In yet another aspect, the invention pertains to a method for diagnosing the status of generation or regeneration of a neuron in a subject. The method comprises the steps of: (a) determining the expression level of a nucleotide sequence coding for a polypeptide of the invention in the subject's generating or regenerating neuron; and, (b) comparing the expression level of the nucleotide sequence with a reference value for expression level of the nucleotide sequence, the reference value preferably being the average value for the expression level in a neuron of healthy individuals. Preferably in the method the expression level of the nucleotide sequence is determined indirectly by quantifying the amount of the polypeptide encoded by the nucleotide sequence. More preferably, the expression level is determined ex vivo in a sample obtained from the subject.

A further aspect of the invention relates to nucleic acid constructs. The nucleic acid constructs comprise all or a part of a nucleotide sequence that encodes a polypeptide that comprises an amino acid sequence that is encoded by a nucleotide sequence selected from: (a) a nucleotide sequence that has at least 60, 70, 80, 85, 90, 95, 98 or 99% identity with a nucleotide sequence selected from SEQ ID NO.'s 1-146; and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO.'s 1-146. Preferably, the nucleotide sequence is operably linked to a promoter that is capable of driving expression of the nucleotide sequence in the neuronal cell.

In a preferred nucleic acid construct the nucleotide sequence is selected from: (a) a nucleotide sequence that has at least 60, 70, 80, 85, 90, 95, 98 or 99% identity with a sequence selected from SEQ ID NO.'s 23, 35, 45, 65, 14, 32, 43, 72, 11, 12, 28, 8, 51, 4, 3, 42, 48, 52, 61, 5, 6, 10, 17, 49, 55, 58, 64, 70, 73, 27, 40, 96, 108, 118, 138, 87, 105, 116, 145, 84, 85, 101, 81, 124, 77, 76, 115, 121, 125, 134, 78, 79, 83, 90, 122, 128, 131, 137, 143, 146, 100, and 113; and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO.'s 23, 35, 45, 65, 14, 32, 43, 72, 11, 12, 28, 8, 51, 4, 3, 42, 48, 52, 61, 5, 6, 10, 17, 49, 55, 58, 64, 70, 73, 27, 40, 96, 108, 118, 138, 87, 105, 116, 145, 84, 85, 101, 81, 124, 77, 76, 115, 121, 125, 134, 78, 79, 83, 90, 122, 128, 131, 137, 143, 146, 100, and 113. A more preferred selection includes SEQ ID NO.'s 23, 35, 45, 65, 14, 32, 43, 72, 11, 12, 28, 96, 108, 118, 138, 87, 105, 116, 145, 84, 85, and 101; and the most preferred selection includes SEQ ID NO.'s 8, 51, 4, 3, 42, 48, 52, 61, 5, 6, 10, 17, 49, 55, 58, 64, 70, 73, 27, 40, 81, 124, 77, 76, 115, 121, 125, 134, 78, 79, 83, 90, 122, 128, 131, 137, 143, 146, 100, and 113.

Alternatively, a nucleic acid construct of the invention comprises or consists of a nucleotide sequence that encodes an RNAi agent, i.e. an RNA molecule that is capable of RNA interference or that is part of an RNA molecule that is capable of RNA interference. Such RNA molecules are referred to as siRNA (short interfering RNA, including e.g. a short hairpin RNA). The nucleotide sequence that encodes the RNAi agent preferably has sufficient complementarity with a cellular nucleotide sequence to be capable of inhibiting the expression of a polypeptide that comprises an amino acid sequence that is encoded by a nucleotide sequence selected from: (a) a nucleotide sequence that has at least 60, 70, 80, 85, 90, 95, 98 or 99% identity with a nucleotide sequence selected from SEQ ID NO.'s 1-146; and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO.'s 1-146. In a preferred nucleic acid construct the nucleotide sequence is selected from: (a) a nucleotide sequence that has at least 60, 70, 80, 85, 90, 95, 98 or 99% identity with a sequence selected from SEQ ID NO.'s 31, 69, 53, 18, 21, 26, 30, 36, 39, 44, 46, 60, 66, 71, 13, 19, 25, 47, 24, 9, 1, 20, 22, 63, 67, 2, 7, 15, 16, 34, 37, 38, 41, 50, 54, 56, 57, 62, 68, 29, 59, 33, 104, 142, 126, 91, 94, 99, 103, 109, 112, 117, 119, 133, 139, 144, 86, 92, 98, 120, 97, 82, 74, 93, 95, 136, 140, 75, 80, 88, 89, 107, 110, 111, 114, 123, 127, 129, 130, 135, 141, 102, 132, and 105; and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO.'s 31, 69, 53, 18, 21, 26, 30, 36, 39, 44, 46, 60, 66, 71, 13, 19, 25, 47, 24, 9, 1, 20, 22, 63, 67, 2, 7, 15, 16, 34, 37, 38, 41, 50, 54, 56, 57, 62, 68, 29, 59, 33, 104, 142, 126, 91, 94, 99, 103, 109, 112, 117, 119, 133, 139, 144, 86, 92, 98, 120, 97, 82, 74, 93, 95, 136, 140, 75, 80, 88, 89, 107, 110, 111, 114, 123, 127, 129, 130, 135, 141, 102, 132, and 105. A more preferred selection includes SEQ ID NO.'s 31, 69, 53, 18, 21, 26, 30, 36, 39, 44, 46, 60, 66, 71, 13, 19, 25, 47, 24, 9, 1, 104, 142, 126, 91, 94, 99, 103, 109, 112, 117, 119, 133, 139, 144, 86, 92, 98, 120, 97, 82, and 74; and the most preferred selection includes SEQ ID NO.'s 20, 22, 63, 67, 2, 7, 15, 16, 34, 37, 38, 41, 50, 54, 56, 57, 62, 68, 29, 59, 33, 93, 95, 136, 140, 75, 80, 88, 89, 107, 110, 111, 114, 123, 127, 129, 130, 135, 141, 102, 132, and 105. Optionally, the nucleotide sequence encoding the RNAi agent is operably linked to a promoter that is capable of driving expression of the nucleotide sequence in the neuronal cell.

In the nucleic acid constructs of the invention, the promoter preferably is a promoter that is specific for a neuronal cell. A promoter that is specific for a neuronal cell is a promoter with a transcription rate that is higher in a neuronal cell than in other types of cells. Preferably the promoter's transcription rate in a neuronal cell is at least 1.1, 1.5, 2.0 or 5.0 times higher than in a non-neuronal cell.

A suitable promoter for use in the nucleic acid constructs of the invention and that is capable of driving expression in a neuronal cell includes a promoter of a gene that encodes an mRNA comprising a nucleotide sequence selected from: (a) a nucleotide sequence that has at least 60, 70, 80, 85, 90, 95, 98 or 99% identity with a nucleotide sequence selected from SEQ ID NO.'s 1-146; and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO.'s 1-146. Preferably the nucleotide sequence is selected from SEQ ID NO.'s 31, 69, 53, 18, 21, 26, 30, 36, 39, 44, 46, 60, 66, 71, 13, 19, 25, 47, 24, 9, 1, 20, 22, 63, 67, 2, 7, 15, 16, 34, 37, 38, 41, 50, 54, 56, 57, 62, 68, 29, 59, 33, 104, 142, 126, 91, 94, 99, 103, 109, 112, 117, 119, 133, 139, 144, 86, 92, 98, 120, 97, 82, 74, 93, 95, 136, 140, 75, 80, 88, 89, 107, 110, 111, 114, 123, 127, 129, 130, 135, 141, 102, 132, and 105; more preferably the nucleotide sequence is selected from SEQ ID NO.'s 31, 69, 53, 18, 21, 26, 30, 36, 39, 44, 46, 60, 66, 71, 13, 19, 25, 47, 24, 9, 1, 104, 142, 126, 91, 94, 99, 103, 109, 112, 117, 119, 133, 139, 144, 86, 92, 98, 120, 97, 82, and 74. Other suitable promoters for use in the nucleic acid constructs of the invention and that is capable of driving expression in a neuronal cell include a GAP43 promoter, a FGF receptor promoter and a neuron specific enolase promoter. The promoters for use in the DNA constructs of the invention are preferably of mammalian origin, more preferably of human origin.

In a preferred embodiment the nucleic acid construct is a viral gene therapy vector selected from gene therapy vectors based on an adenovirus, an adeno-associated virus (AAV), a herpes virus, a pox virus and a retrovirus. A preferred viral gene therapy vector is an AAV or Lentiviral vector. Such vectors are further described herein below.

In yet a further aspect, the invention relates to a method for identification of a substance capable of promoting regeneration of a neuronal cell. The method preferably comprising the steps of: (a) providing a test cell population capable of expressing a nucleotide sequence encoding a TF polypeptide of the invention; (b) contacting the test cell population with the substance; (c) determining the expression level of the nucleotide sequence or the activity or steady state level of the polypeptide in the test cell population contacted with the substance; (d) comparing the expression, activity or steady state level determined in (c) with the expression, activity or steady state level of the nucleotide sequence or of the polypeptide in a test cell population that is not contacted with the substance; and, (e) identifying a substance that produces a difference in expression level, activity or steady state level of the nucleotide sequence or the polypeptide, between the test cell population that is contacted with the substance and the test cell population that is not contacted with the substance. Preferably, in the method the expression levels, activities or steady state levels of more than one nucleotide sequence or more than one polypeptide are compared. Preferably, in the method the test cell population comprises primary sensoric neurons (e.g. DRG neuronen), cells of the sensory neuron cell line such as e.g. the F11 cell line and/or other cells or cell lines described in the Examples herein. The test cell population preferably comprises mammalian cells, more preferably human cells. In one aspect the invention also pertains to a substance that is identified in a method the aforementioned methods.

Sequence Identity

“Sequence identity” is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; H is to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

Recombinant Techniques and Methods for Recombinant Production of Polypeptides

Polypeptides for use in the present invention can be prepared using recombinant techniques, in which a nucleotide sequence encoding the polypeptide of interest is expressed in suitable host cells. The present invention thus also concerns the use of a vector comprising a nucleic acid molecule or nucleotide sequence as defined above. Preferably the vector is a replicative vector comprising on origin of replication (or autonomously replication sequence) that ensures multiplication of the vector in a suitable host for the vector. Alternatively the vector is capable of integrating into the host cell's genome, e.g. through homologous recombination or otherwise. A particularly preferred vector is an expression vector wherein a nucleotide sequence encoding a polypeptide as defined above, is operably linked to a promoter capable of directing expression of the coding sequence in a host cell for the vector.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is only active in specific types of differentiated cells/tissues, such as preferably neuronal cells or tissues.

Expression vectors allow the polypeptides of the invention as defined above to be prepared using recombinant techniques in which a nucleotide sequence encoding the polypeptide of interest is expressed in suitable cells, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34: 315 (describing cassette mutagenesis).

Typically, nucleic acids encoding the desired polypeptides are used in expression vectors. The phrase “expression vector” generally refers to nucleotide sequences that are capable of effecting expression of a gene in hosts compatible with such sequences. These expression vectors typically include at least suitable promoter sequences and optionally, transcription termination signals. Additional factors necessary or helpful in effecting expression can also be used as described herein. DNA encoding a polypeptide is incorporated into DNA constructs capable of introduction into and expression in an in vitro cell culture. Specifically, DNA constructs are suitable for replication in a prokaryotic host, such as bacteria, e.g., E. coli, or can be introduced into a cultured mammalian, plant, insect, e.g., Sf9, yeast, fungi or other eukaryotic cell lines.

DNA constructs prepared for introduction into a particular host typically include a replication system recognized by the host, the intended DNA segment encoding the desired polypeptide, and transcriptional and translational initiation and termination regulatory sequences operably linked to the polypeptide-encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of the polypeptide. Generally, DNA sequences that are operably linked are contiguous, and, in the case of a signal sequence, both contiguous and in reading phase. However, enhancers need not be contiguous with the coding sequences whose transcription they control. Linking is accomplished by ligation at convenient restriction sites or at adapters or linkers inserted in lieu thereof.

The selection of an appropriate promoter sequence generally depends upon the host cell selected for the expression of the DNA segment. Examples of suitable promoter sequences include prokaryotic, and eukaryotic promoters well known in the art (see, e.g. Sambrook and Russell, 2001, supra). The transcriptional regulatory sequences typically include a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). Expression vectors include the replication system and transcriptional and translational regulatory sequences together with the insertion site for the polypeptide encoding segment can be employed. Examples of workable combinations of cell lines and expression vectors are described in Sambrook and Russell (2001, supra) and in Metzger et al. (1988) Nature 334: 31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. The host cells may thus be prokaryotic or eukarotic host cells. The host cell may be a host cell that is suitable for culture in liquid or on solid media. The host cells are used in a method for producing a polypeptide of the invention as defined above. The method comprises the step of culturing a host cell under conditions conducive to the expression of the polypeptide. Optionally the method may comprise recovery the polypeptide. The polypeptide may e.g. be recovered from the culture medium by standard protein purification techniques, including a variety of chromatography methods known in the art per se.

Alternatively, the host cell is a cell that is part of a multicellular organism such as a transgenic plant or animal, preferably a non-human animal. A transgenic plant comprises in at least a part of its cells a vector as defined above. Methods for generating transgenic plants are e.g. described in U.S. Pat. No. 6,359,196 and in the references cited therein. Such transgenic plants may be used in a method for producing a polypeptide of the invention as defined above, the method comprising the step of recovering a part of a transgenic plant comprising in its cells the vector or a part of a descendant of such transgenic plant, whereby the plant part contains the polypeptide, and, optionally recovery of the polypeptide from the plant part. Such methods are also described in U.S. Pat. No. 6,359,196 and in the references cited therein. Similarly, the transgenic animal comprises in its somatic and germ cells a vector as defined above. The transgenic animal preferably is a non-human animal. Methods for generating transgenic animals are e.g. described in WO 01/57079 and in the references cited therein. Such transgenic animals may be used in a method for producing a polypeptide of the invention as defined above, the method comprising the step of recovering a body fluid from a transgenic animal comprising the vector or a female descendant thereof, wherein the body fluid contains the polypeptide, and, optionally recovery of the polypeptide from the body fluid. Such methods are also described in WO 01/57079 and in the references cited therein. The body fluid containing the polypeptide preferably is blood or more preferably milk.

Another method for preparing polypeptides is to employ an in vitro transcription/translation system. DNA encoding a polypeptide is cloned into an expression vector as described supra. The expression vector is then transcribed and translated in vitro. The translation product can be used directly or first purified. Polypeptides resulting from in vitro translation typically do not contain the post-translation modifications present on polypeptides synthesised in vivo, although due to the inherent presence of microsomes some post-translational modification may occur. Methods for synthesis of polypeptides by in vitro translation are described by, for example, Berger & Kimmel, Methods in Enzymology, Volume 152, Guide to Molecular Cloning Techniques, Academic Press, Inc., San Diego, Calif., 1987.

Gene Therapy

Some aspects of the invention concern the use of nucleic acid constructs or expression vectors comprising the nucleotide sequences as defined above, wherein the vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol. 10: 448-53; Vigna and Naldini, 2000, J. Gene Med. 2: 308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein.

Particularly suitable gene therapy vectors include Adenoviral and Adeno-associated virus (AAV) vectors. These vectors infect a wide number of dividing and non-dividing cell types including neuronal cells. In addition adenoviral vectors are capable of high levels of transgene expression. However, because of the episomal nature of the adenoviral and AAV vectors after cell entry, these viral vectors are most suited for therapeutic applications requiring only transient expression of the transgene (Russell, 2000, J. Gen. Virol. 81: 2573-2604; Goncalves, 2005, Virol J. 2(1):43) as indicated above. Preferred adenoviral vectors are modified to reduce the host response as reviewed by Russell (2000, supra). Method for neuronal gene therapy using AAV vectors are described by Wang et al., 2005, J Gene Med. March 9 (Epub ahead of print), Mandel et al., 2004, Curr Opin Mol Ther. 6(5):482-90, and Martin et al., 2004, Eye 18(11): 1049-55. For neuronal gene transfer AAV serotype 2 is an effective vector and therefore a preferred AAV serotype.

A preferred retroviral vector for application in the present invention is a lentiviral based expression construct. Lentiviral vectors have the unique ability to infect non-dividing cells (Amado and Chen, 1999 Science 285: 674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2: 308-16).

Generally, gene therapy vectors will be as the expression vectors described above in the sense that they comprise the nucleotide sequence encoding the polypeptide of the invention to be expressed, whereby the nucleotide sequence is operably linked to the appropriate regulatory sequences as indicated above. Such regulatory sequence will at least comprise a promoter sequence. Suitable promoters for expression of the nucleotide sequence encoding the polypeptide from gene therapy vectors include e.g. cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter and the herpes simplex virus thymidine kinase promoter. Suitable neuronal promoters are described above.

Several inducible promoter systems have been described that may be induced by the administration of small organic or inorganic compounds. Such inducible promoters include those controlled by heavy metals, such as the metallothionine promoter (Brinster et al. 1982 Nature 296: 39-42; Mayo et al. 1982 Cell 29: 99-108), R^{U}-486 (a progesterone antagonist) (Wang et al. 1994 Proc. Natl. Acad. Sci. USA 91: 8180-8184), steroids (Mader and White, 1993 Proc. Natl. Acad. Sci. USA 90: 5603-5607), tetracycline (Gossen and Bujard 1992 Proc. Natl. Acad. Sci. USA 89: 5547-5551; U.S. Pat. No. 5,464,758; Furth et al. 1994 Proc. Natl. Acad. Sci. USA 91: 9302-9306; Howe et al. 1995 J. Biol. Chem. 270: 14168-14174; Resnitzky et al. 1994 Mol. Cell. Biol. 14: 1669-1679; Shockett et al. 1995 Proc. Natl. Acad. Sci. USA 92: 6522-6526) and the tTAER system that is based on the multi-chimeric transactivator composed of a tetr polypeptide, as activation domain of VP 16, and a ligand binding domain of an estrogen receptor (Yee et al., 2002, U.S. Pat. No. 6,432,705).

Suitable promoters for nucleotide sequences encoding small RNAs for knock down of specific genes by RNA interference (see below) include, in addition to the above mentioned polymerase II promoters, polymerase III promoters. The RNA polymerase III (pol III) is responsible for the synthesis of a large variety of small nuclear and cytoplasmic non-coding RNAs including 5S, U6, adenovirus VA1, Vault, telomerase RNA, and tRNAs. The promoter structures of a large number of genes encoding these RNAs have been determined and it has been found that RNA pol III promoters fall into three types of structures (for a review see Geiduschek and Tocchini-Valentini, 1988 Annu. Rev. Biochem. 57: 873-914; Willis, 1993 Eur. J. Biochem. 212: 1-11; Hernandez, 2001, J. Biol. Chem. 276: 26733-36). Particularly suitable for expression of siRNAs are the type 3 of the RNA pol III promoters, whereby transcription is driven by cis-acting elements found only in the 5′-flanking region, i.e. upstream of the transcription start site. Upstream sequence elements include a traditional TATA box (Mattaj et al., 1988 Cell 55, 435-442), proximal sequence element and a distal sequence element (DSE; Gupta and Reddy, 1991 Nucleic Acids Res. 19, 2073-2075). Examples of genes under the control of the type 3 pol III promoter are U6 small nuclear RNA (U6 snRNA), 7SK, Y, MRP, HI and telomerase RNA genes (see e.g. Myslinski et al., 2001, Nucl. Acids Res. 21: 2502-09).

The gene therapy vector may optionally comprise a second or one or more further nucleotide sequence coding for a second or further protein. The second or further protein may be a (selectable) marker protein that allows for the identification, selection and/or screening for cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3rd edition), Cold Spring Harbor Laboratory, Cold Spring Harbor Laboratory Press, New York.

Alternatively, the second or further nucleotide sequence may encode a protein that provides for fail-safe mechanism that allows to cure a subject from the transgenic cells, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the IL-10 transgenic cells in the subject (see e.g. Clair et al., 1987, Antimicrob. Agents Chemother. 31: 844-849).

The gene therapy vectors are preferably formulated in a pharmaceutical composition comprising a suitable pharmaceutical carrier as defined below.

RNA Interference

For knock down of expression of specific polypeptides of the invention of the invention, gene therapy vectors or other expression constructs are used for the expression of a desired nucleotide sequence that preferably encodes an RNAi agent, i.e. an RNA molecule that is capable of RNA interference or that is part of an RNA molecule that is capable of RNA interference. Such RNA molecules are referred to as siRNA (short interfering RNA, including e.g. a short hairpin RNA). Alternatively, the siRNA molecules may directly, e.g. in a pharmaceutical composition that is administered at the site of neuronal injury or degeneration.

The desired nucleotide sequence comprises an antisense code DNA coding for the antisense RNA directed against a region of the target gene mRNA, and/or a sense code DNA coding for the sense RNA directed against the same region of the target gene mRNA. In the DNA constructs of the invention, the antisense and sense code DNAs are operably linked to one or more promoters as herein defined above that are capable of expressing the antisense and sense RNAs, respectively. “siRNA” means a small interfering RNA that is a short-length double-stranded RNA that are not toxic in mammalian cells (Elbashir et al., 2001, Nature 411: 494-98; Caplen et al., 2001, Proc. Natl. Acad. Sci. USA 98: 9742-47). The length is not necessarily limited to 21 to 23 nucleotides. There is no particular limitation in the length of siRNA as long as it does not show toxicity. “siRNAs” can be, e.g. at least 15, 18 or 21 nucleotides and up to 25, 30, 35 or 49 nucleotides long. Alternatively, the double-stranded RNA portion of a final transcription product of siRNA to be expressed can be, e.g. at least 15, 18 or 21 nucleotides and up to 25, 30, 35 or 49 nucleotides long.

“Antisense RNA” is an RNA strand having a sequence complementary to a target gene mRNA, and thought to induce RNAi by binding to the target gene mRNA. “Sense RNA” has a sequence complementary to the antisense RNA, and annealed to its complementary antisense RNA to form siRNA. The term “target gene” in this context refers to a gene whose expression is to be silenced due to siRNA to be expressed by the present system, and can be arbitrarily selected. As this target gene, for example, genes whose sequences are known but whose functions remain to be elucidated, and genes whose expressions are thought to be causative of diseases are preferably selected. A target gene may be one whose genome sequence has not been fully elucidated, as long as a partial sequence of mRNA of the gene having at least 15 nucleotides or more, which is a length capable of binding to one of the strands (antisense RNA strand) of siRNA, has been determined. Therefore, genes, expressed sequence tags (ESTs) and portions of mRNA, of which some sequence (preferably at least 15 nucleotides) has been elucidated, may be selected as the “target gene” even if their full length sequences have not been determined.

The double-stranded RNA portions of siRNAs in which two RNA strands pair up are not limited to the completely paired ones, and may contain nonpairing portions due to mismatch (the corresponding nucleotides are not complementary), bulge (lacking in the corresponding complementary nucleotide on one strand), and the like. Nonpairing portions can be contained to the extent that they do not interfere with siRNA formation. The “bulge” used herein preferably comprise 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, preferably 1 to 7, more preferably 1 to 5 in number. Such nonpairing portions (mismatches or bulges, etc.) can suppress the below-described recombination between antisense and sense code DNAs and make the siRNA expression system as described below stable. Furthermore, although it is difficult to sequence stem loop DNA containing no nonpairing portion in the double-stranded RNA region of siRNAs in which two RNA strands pair up, the sequencing is enabled by introducing mismatches or bulges as described above. Moreover, siRNAs containing mismatches or bulges in the pairing double-stranded RNA region have the advantage of being stable in E. coli or animal cells.

The terminal structure of siRNA may be either blunt or cohesive (overhanging) as long as siRNA enables to silence the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 bp double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the “siRNA” is necessarily the cut off structure at both ends as described above, and may have a stem-loop structure in which ends of one side of double-stranded RNA are connected by a linker RNA (a “shRNA”). The length of the double-stranded RNA region (stem-loop portion) can be, e.g. at least 15, 18 or 21 nucleotides and up to 25, 30, 35 or 49 nucleotides long. Alternatively, the length of the double-stranded RNA region that is a final transcription product of siRNAs to be expressed is, e.g. at least 15, 18 or 21 nucleotides and up to 25, 30, 35 or 49 nucleotides long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA, snRNA or viral RNA, or an artificial RNA molecule.

To express antisense and sense RNAs from the antisense and sense code DNAs respectively, the DNA constructs of the present invention comprise a promoter as defined above. The number and the location of the promoter in the construct can in principle be arbitrarily selected as long as it is capable of expressing antisense and sense code DNAs. As a simple example of a DNA construct of the invention, a tandem expression system can be formed, in which a promoter is located upstream of both antisense and sense code DNAs. This tandem expression system is capable of producing siRNAs having the aforementioned cut off structure on both ends. In the stem-loop siRNA expression system (stem expression system), antisense and sense code DNAs are arranged in the opposite direction, and these DNAs are connected via a linker DNA to construct a unit. A promoter is linked to one side of this unit to construct a stem-loop siRNA expression system. Herein, there is no particular limitation in the length and sequence of the linker DNA, which may have any length and sequence as long as its sequence is not the termination sequence, and its length and sequence do not hinder the stem portion pairing during the mature RNA production as described above. As an example, DNA coding for the above-mentioned tRNA and such can be used as a linker DNA.

In both cases of tandem and stem-loop expression systems, the 5′ end may be have a sequence capable of promoting the transcription from the promoter. More specifically, in the case of tandem siRNA, the efficiency of siRNA production may be improved by adding a sequence capable of promoting the transcription from the promoters at the 5′ ends of antisense and sense code DNAs. In the case of stem-loop siRNA, such a sequence can be added at the 5′ end of the above-described unit. A transcript from such a sequence may be used in a state of being attached to siRNA as long as the target gene silencing by siRNA is not hindered. If this state hinders the gene silencing, it is preferable to perform trimming of the transcript using a trimming means (for example, ribozyme as are known in the art). It will be clear to the skilled person that the antisense and sense RNAs may be expressed in the same vector or in different vectors. To avoid the addition of excess sequences downstream of the sense and antisense RNAs, it is preferred to place a terminator of transcription at the 3′ ends of the respective strands (strands coding for antisense and sense RNAs). The terminator may be a sequence of four or more consecutive adenine (A) nucleotides.

Antibodies

Some aspects of the invention concern the use of an antibody or antibody-fragment that specifically binds to a polypeptide of the invention as defined above. Methods for generating antibodies or antibody-fragments that specifically bind to a given polypeptide are described in e.g. Harlow and Lane (1988, Antibodies: A Laboratory Manual, Cold Spring Harbor Laboratory Press, Cold Spring Harbor, N.Y.) and WO 91/19818; WO 91/18989; WO 92/01047; WO 92/06204; WO 92/18619; and U.S. Pat. No. 6,420,113 and references cited therein. The term “specific binding,” as used herein, includes both low and high affinity specific binding. Specific binding can be exhibited, e.g., by a low affinity antibody or antibody-fragment having a Kd of at least about 10⁻⁴ M. Specific binding also can be exhibited by a high affinity antibody or antibody-fragment, for example, an antibody or antibody-fragment having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater.

Peptidomimetics

Peptide-like molecules (referred to as peptidomimetics) or non-peptide molecules that specifically bind to a polypeptide of the invention or to its receptor polypeptide and that may be applied in any of the methods of the invention as defined herein as agonists or antagonists of the polypeptides of the invention and they may be identified using methods known in the art per se, as e.g. described in detail in U.S. Pat. No. 6,180,084 which incorporated herein by reference. Such methods include e.g. screening libraries of peptidomimetics, peptides, DNA or cDNA expression libraries, combinatorial chemistry and, particularly useful, phage display libraries. These libraries may be screened for agonists and antagonsist of TF polypeptides by contacting the libraries with substantially purified polypeptides of the invention, fragments thereof or structural analogues thereof.

Pharmaceutical Compositions

The invention further relates to a pharmaceutical preparation comprising as active ingredient a polypeptide, an antibody or a gene therapy vector as defined above. The composition preferably at least comprises a pharmaceutically acceptable carrier in addition to the active ingredient.

In some methods, the polypeptide or antibody of the invention as purified from mammalian, insect or microbial cell cultures, from milk of transgenic mammals or other source is administered in purified form together with a pharmaceutical carrier as a pharmaceutical composition. Methods of producing pharmaceutical compositions comprising polypeptides are described in U.S. Pat. Nos. 5,789,543 and 6,207,718. The preferred form depends on the intended mode of administration and therapeutic application.

The pharmaceutical carrier can be any compatible, non-toxic substance suitable to deliver the polypeptides, antibodies or gene therapy vectors to the patient. Sterile water, alcohol, fats, waxes, and inert solids may be used as the carrier. Pharmaceutically acceptable adjuvants, buffering agents, dispersing agents, and the like, may also be incorporated into the pharmaceutical compositions.

The concentration of the polypeptides or antibodies of the invention in the pharmaceutical composition can vary widely, i.e., from less than about 0.1% by weight, usually being at least about 1% by weight to as much as 20% by weight or more.

For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable colour, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain colouring and flavouring to increase patient acceptance.

The polypeptides, antibodies or gene therapy vectors are preferably administered parentally. The polypeptide, antibody or vector for preparations for parental administration must be sterile. Sterilisation is readily accomplished by filtration through sterile filtration membranes, prior to or following lyophilisation and reconstitution. The parental route for administration of the polypeptide, antibody or vector is in accord with known methods, e.g. injection or infusion by intravenous, intraperitoneal, intramuscular, intraarterial, intralesional, intracranial, intrathecal, transdermal, nasal, buccal, rectal, or vaginal routes. The polypeptide, antibody or vector is administered continuously by infusion or by bolus injection. A typical composition for intravenous infusion could be made up to contain 10 to 50 ml of sterile 0.9% NaCl or 5% glucose optionally supplemented with a 20% albumin solution and 1 to 50 μg of the polypeptide, antibody or vector. A typical pharmaceutical composition for intramuscular injection would be made up to contain, for example, 1-10 ml of sterile buffered water and 1 to 100 μg of the polypeptide, antibody or vector of the invention. Methods for preparing parenterally administrable compositions are well known in the art and described in more detail in various sources, including, for example, Remington's Pharmaceutical Science (15th ed., Mack Publishing, Easton, Pa., 1980) (incorporated by reference in its entirety for all purposes).

For therapeutic applications, the pharmaceutical compositions are administered to a patient suffering from a neurotraumatic injury or a neurodegenerative disease in an amount sufficient to reduce the severity of symptoms and/or prevent or arrest further development of symptoms. An amount adequate to accomplish this is defined as a “therapeutically-” or “prophylactically-effective dose”. Such effective dosages will depend on the severity of the condition and on the general state of the patient's health. In general, a therapeutically- or prophylactically-effective dose preferably is a dose, which is sufficient to reverse the symptoms, i.e. to restore function of the sensory and/or motory neurons to an acceptable level, preferably (close) to the average levels found in normal unaffected healthy individuals.

In the present methods, the polypeptide or antibody is usually administered at a dosage of about 1 μg/kg patient body weight or more per week to a patient. Often dosages are greater than 10 μg/kg per week. Dosage regimes can range from 10 μg/kg per week to at least 1 mg/kg per week. Typically dosage regimes are 10 μg/kg per week, 20 μg/kg per week, 30 μg/kg per week, 40 μg/kg week, 60 μg/kg week, 80 μg/kg per week and 120 μg/kg per week. In preferred regimes 10 μg/kg, 20 μg/kg or 40 μg/kg is administered once, twice or three times weekly. Treatment is preferably administered by parenteral route.

Microarrays

Another aspect of the invention relates to microarrays (or other high throughput screening devices) comprising the nucleic acids, polypeptides or antibodies as defined above. A microarray is a solid support or carrier containing one or more immobilised nucleic acid or polypeptide fragments for analysing nucleic acid or amino acid sequences or mixtures thereof (see e.g. WO 97/27317, WO 97/22720, WO 97/43450, EP 0 799 897, EP 0 785 280, WO 97/31256, WO 97/27317, WO 98/08083 and Zhu and Snyder, 2001, Curr. Opin. Chem. Biol. 5: 40-45). Microarrays comprising the nucleic acids may be applied e.g. in methods for analysing genotypes or expression patterns as indicated above. Microarrays comprising polypeptides may be used for detection of suitable candidates of substrates, ligands or other molecules interacting with the polypeptides. Microarrays comprising antibodies may be used for in methods for analysing expression patterns of the polypeptides as indicated above.

General

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### 1. Example I

### Identification of Transcription Factors and/or Modulators of Gene Transcription Involved in Neuroregeneration

### 1.1 Surgical Procedures

Adult male Wistar rats (±220 g) (Harlan, The Netherlands) were housed in group cages, maintained on a 12 h light/12 h dark cycle and food and water were available ad libitum. For sciatic nerve crush, rats were anaesthetized using 1.8% isofluorane (in 0.3 l/min O₂; 0.6 l/min N₂O). The sciatic nerve was exposed at mid-thigh level and crushed for 30 s by closing a haemostatic forceps with grooved jaws. For the dorsal root crush, rats were anaesthetized with an intramuscular injection of Hypnorm (fentanyl citrate/fluanisone: 0.06 ml/100 g body weight; Janssen Pharmaceuticals, Beerse, Belgium) and sedated with an intramuscular injection of Dormicum (midazolam: 0.015 ml/100 g body weight; Roche, Almere, The Netherlands). The left lumbar spinal roots were exposed by a laminectomy at the level of L2. The L4, L5 and L6 dorsal roots were crushed by closing a forceps with smooth jaws over the root until an opaque lesion site was visible. The skin and muscle incisions were closed in layers. Animals were sacrificed at 6 hrs, 12 hrs, 24 hrs, 48 hrs, 72 hrs, 7 days and 14 days after surgery by decapitation after sedation in CO₂. Control tissue was taken from uninjured animals. All animals were sacrificed at the same hour of day to prevent the possibility that changes in gene expression can be attributed to the circadian rhythm. Dorsal root ganglia (DRGs) at levels L4, L5 and L6 were dissected, frozen and stored at −80° C. until use.

### 1.2 RNA Isolation and Reverse Transcription

RNA isolation from three DRGs per animal was performed using the method of Chomczynski and Sacchi (1987, Anal Biochem. 162(1):156-9), however omitting sarcosyl in the GTC solution to prevent frothing. Sarcosyl was added after homogenization to a final volume of 0.05%. An additional chloroform extraction was performed to remove traces of phenol. RNA was checked by gel electrophoresis for integrity and quantified by photo spectrometry. In addition, equimolar amounts of RNA of 3 animals were pooled, and then split in one batch for cDNA synthesis, one batch for Cy5 labeling and one batch for Cy3 labeling. cDNA synthesis was performed using oligo dT primers and Superscript II and Cy3 and Cy5 labeling was done using the Agilent Fluorescent Linear Amplification kit (G2554A) according to the instructions provided by the manufacturer.

### 1.3 Microarrays and Hybridization Procedures

Agilent custom made 8.5K 60-mer arrays were used. Arrays were designed to contain all ˜4500 UniGene clusters consisting of at least one mRNA (UG build 101). In addition, ESTs possibly containing protein domains relevant to our biological research question, such as DNA binding domains and LIM domains were identified using blastx (Altschul et al., 1997, Nucleic Acids Res. 25(17):3389-402). ESTs that were highly expressed in, or unique to, neuronal tissues were identified using the NCBI library browser. The remainder of the 8091 genes were selected using search strings “highly similar” and “moderately similar” in UniGene, to obtain clusters containing only ESTs that show similarity to known sequences in rat, mouse or human. Hybridization of arrays was performed according to the manufacturer's instructions. In short, 0.5 μg of a Cy5 labeled and a Cy3 labeled cRNA target were mixed and hydrolyzed for 30 min. Arrays were incubated with these targets in 1× hybridization solution (Agilent technologies) for 18 hours at 60° C. in a rotating hybridization chamber. Arrays were washed in 6×SSC/0.005% triton X-102 for 10 min at RT and 0.1×SSC/0.005% triton X-102 for 5 min at 4° C., placed inside a Falcon 50 mL tube and spun dry in a centrifuge. Arrays were scanned using an Agilent scanner.

### 1.4 Number of Expressed and Regulated Genes on the Microarrays

As a criterium for calling a gene ‘expressed’, we used the p-value that the intensity of the pixels in the spot is equal to the intensity of the pixels in the local background. We used the negative control spots, which consist of 20mers of Arabidopsis thaliana specific sequences, to assess efficiency of our filter. At a cutoff of 1.0E-5, all but one negative control spot were filtered out on all 24 arrays used. This cutoff yields 7107 genes that are present in both DR and SN datasets. Next, we calculated the gene expression differences between all time points and time point 0 (unlesioned control), by fitting a linear model to the normalized log ratios of the signal intensities. A bonferroni corrected t-test was used to filter regulated genes. In total, 1836 genes out of 7107 (26%) were regulated at one or more time points after SN or DR lesion (Table 2).

To narrow down the amount of regulated genes, we made use of the time-aspect of our data. For the time-points that had 24 hrs or less in between, we selected the genes that were regulated at least 2 consecutive time-points. This yielded 1340 genes.

TABLE 2

| Lesion | timepoint | | # | % |
|---|---|---|---|---|
| sn crush | 6 | h | 325 | 4.6 |
| | 12 | h | 288 | 4.1 |
| | 24 | h | 496 | 7.0 |
| | 48 | h | 637 | 9.0 |
| | 72 | h | 761 | 10.7 |
| | 7 | d | 600 | 8.4 |
| | 14 | d | 442 | 6.2 |
| | total | | 1406 | 19.8 |
| dr crush | 6 | h | 210 | 3.0 |
| | 12 | h | 361 | 5.1 |
| | 24 | h | 261 | 3.7 |
| | 48 | h | 184 | 2.6 |
| | 72 | h | 200 | 2.8 |
| | 7 | d | 282 | 4.0 |
| | 14 | d | 178 | 2.5 |

| | | |
|---|---|---|
| total | 697 | 9.8 |

| | | |
|---|---|---|
| Total | 1836 | 25.8 |
| Genes regulated at at least 2 consecutive time points | 1340 | |
| Tfs regulated at at least 2 consecutive time points | 220 | |
| Tfs differentially regulated DR vs SN | 73 | |

### 1.5 Further Selection of Genes by Micro Array Analysis

We performed large scale gene expression profiling with custom-designed Agilent micro arrays of rat dorsal root ganglion (DRG) neurons following damage to the sciatic nerve (SN), which results in successful regeneration, or to the dorsal root (DR), which does not result in successful regeneration. We identified 1,340 genes that were regulated at least 2 consecutive time points following sciatic nerve regeneration or following dorsal root lesion. Of the genes that were regulated in the first 24 hours, an overrepresented group consisted of transcription factors or modulators of gene transcription (TFs). This is in line with the notion that changes in the expression levels of TFs in the early stages (and probably also in later stages) of regeneration determine whether neurons successfully regenerate or not, and that these changes result in an altered gene expression state that is required for robust neurite outgrowth and functional recovery.

Using a combined strategy involving sequence annotation publicly available, Gene Ontology database searching, a protein motif search, and a gene description keyword filtering approach we found the total number of transcription factors analyzed on the array was 548. Of these, 484 (88%) were called present after hybridization of the arrays. Out of 484 present TFs, 220 TFs can be discriminated among the 1340 genes regulated at least 2 consecutive time points following sciatic nerve regeneration or following dorsal root lesion. Of these, 94 TFs were selected that showed significantly regulated expression at any of the 5 time points, 6 h, 12 h, 24 h, 48 h and 72 hours after the crush, compared with time point zero, in either regeneration paradigm. The genes showing early expression are considered to be of prime interest as these might be successfully targeted to initiate successful regeneration. We then eliminated TFs that were not specifically regulated between the SN and DR crush paradigms. Only genes with significantly different average regulation (P<0.16) over the first 5 time points were selected resulting in a group of 73 genes (Table 2).

The regulated expression of all TFs used is confirmed by qPCR analysis for DRG and also for F11 cells (a hybridoma of rat DRG and mouse neuroblastoma cells). For this we repeat the crush paradigm (certainly the sciatic nerve crush, and preferably also the dorsal root crush; n=8), only for the time points that are relevant for our studies. Six RNA samples are isolated for qPCR determination of the candidate TFs selected. RNA is also stored for analysis of other future candidate genes.

Table 3 list the 73 genes selected as described above (Table 2), their SEQ ID No.'s, database accession no.'s and annotations. Table 4 provides the statistical analysis parameters. Table 5 lists the expression values (log 2 values) as measured with respect to timepoint 0 for the sciatic nerve (SN), the nerve that regenerates. Table 6 lists the expression values (log 2 values) as measured with respect to timepoint 0 for the dorsal root (DR), the nerve that does not regenerate. Table 7 list the human orthologues of the 73 rat sequences, including their SEQ ID No.'s, database accession no.'s and annotations. More detailed information of the human sequences is provided in Appendix A.

### 2. Example II

### Functionality-Based Screens of the Regulated Neuronal Genes

### 2.1 Quantification of Neurite Outgrowth

First, we perform large scale quantification of neurite outgrowth in primary DRG neurons and in rat F11 cells treated with RNAi vectors. DRG neurons grow out spontaneously and gene knock down of relevant genes results in impaired outgrowth. F11 cells need a stimulation of the cAMP pathway in order to generate a growth response. For large scale screening we use automated cell imaging equipment (Kineticscan from Cellomics). This instrument is present at the VUA and allows assaying at multiwell format (e.g. 96 wells). Multiple photographs taken from each well are stored in a database. Cell growth is monitored in a time series and thereby also reveals differences in more subtle outgrowth phenotypes. The analysis typically assesses neurite length, growth rate, number of branch points.

The siRNA screen provides a functionality-based filter to the set of neuronal genes identified previously, in particular TFs, and identifies those genes (TFs) that have a role in neurite outgrowth-related aspects of regeneration. The approach is unbiassed, and identifies components of larger neuronal gene (TF) networks involved in the outgrowth response. Reconstruction of this network may facilitate the identification of key genes that control the initiation and promotion of neuronal regeneration.

In addition, using the same type of assays, we score gain of function. In particular F11 cells are very suited for this because they do not show spontaneous outgrowth and the specific genes the activation of which is necessary for growth are readily revealed in this way. Adenoviral vectors encoding full-length neuronal genes (TFs) are generated for this purpose.

### 2.1.1 Neurite Outgrowth Assay 1: Dissociated Adult Dorsal Root Ganglion Neurons

The role of neuronal genes is studied in a neurite outgrowth assay based on cultured dorsal root ganglion neurons from adult Wistar rats. Dissociated DRG neurons are plated in 96 well tissue culture plates and transduced with the appropriate adenoviral vector. The effect of knock down as well overexpression of each of the selected neuronal genes of the invention is measured at short (12, 24, 36 hours) and longer time points (up to 6 days) in culture.

### 2.1.2 Neurite Outgrowth Assay 2: Based Embryonic Dorsal Root Ganglion on Monolayers of OEG or SC

The role of the identified glial genes in neurite outgrowth is studied in co-cultures of embryonic dorsal root ganglia plated on monolayer of olfactory ensheething glia cells (OEG) or SC. 96-Well plates are coated with PLL and seeded with 8.5×10³ OEG or SC. Cells are cultured in medium containing PEX and forskolin. Cells will be transduced with different adenoviral vectors to knock-down or activate the expression of selected neuronal genes of the invention. Each well is used as a single bioassay to analyze one gene and each gene will be analyzed in triplicate. After three days, dorsal root ganglia (DRGs) are removed from embryos of embryonic day 14 (E14) pregnant rats. In each well, one DRG is placed on top of the monolayer of transduced OEG or SC. Co-cultures of OEG or SC with DRGs are grown for 24 hours in 10% FCS/1% PS in DMEM. To visualize neurite outgrowth, cultures are fixed with 4% PFA in PBS, incubated with the antibody 2H3 against rat neurofilament and subsequently with Cy3-conjugated secondary antibody. Neurite outgrowth from each well is quantified in a high throughput fashion with cell screen instruments and software previously described. Control assays from DRGs grown on uninfected OEG or SC are included to compare neurite outgrowth.

### 2.1.3 Neurite Outgrowth Assay 3: Based on F-11 Dorsal Root Ganglion Cell Line

F11 cells is a hybridoma of rat embryonal DRG neurons and the mouse neuroblastoma cell line N18TG2 (Platika et al., PNAS, 1985). F-11 cells are maintained under standard culture conditions; DMEM supplemented with 10% FCS, 100 U/ml penicillin and 100 mg/ml streptomycin at 37° C., 5% CO₂. To induce differentiation, cells are incubated in DMEM with 0.5% FCS and 0.5 mM db-cAMP or 10 μM forskolin. F— 11 cells are being cultured in multiwell format.

### 3. Example III

### Repairing Injured Spinal Cord, Ventral Roots and Sciatic Nerve by Cell and Gene Therapy

We and others have combined cellular implants and gene therapy (ex vivo gene therapy) to modify the non-permissive terrain of the neural scar formed in the spinal cord after injury. The aim of these studies is to promote the regrowth of injured axons through and beyond the inhibitory neural scar.

In addition to ex vivo gene therapy we have demonstrated the effectiveness of direct gene transfer after ventral root avulsion lesion, a model for brachial plexus injury in humans. After ventral root avulsion motor neurons normally die within 2 to 4 weeks. We were able to rescue a significant number of motor neurons by direct adeno-associated viral vector-mediated expression of the growth factor GDNF into the avulsed motor neurons. The scientific literature on these studies is listed in Appendix B. In recent studies we have been able to promote the regeneration of transected rat peripheral nerves by transducing the Schwann cells distal to the lesion with a viral vector expressing the growth factor NGF. Animals that received the NGF-expressing vector display a much faster return of sensory function in their hind paw than animals treated with the control vector. As a next step towards clinical application we have shown that human nerve biopsies can be genetically modified in culture and as a result do secrete high levels of neurotrophic factor.

Taken together these studies provide extensive proof of principle that it is possible to enhance the regenerative response of injured central and peripheral neurons by cell and gene therapy. Using the newly identified sequences of the invention in similar studies we show that the level of regeneration both at the anatomical and at the functional level is significantly improved. The feasibility of gene therapy with newly identified sequences is demonstrated in the following animal models.

### 3.1 Spinal Cord Transection.

Two major descending spinal motor tracts are lesioned by a spinal cord hemisection. This leads to permanent paralysis of the hind paws and is an animal model for spinal cord injury in humans. Male Wistar rats are deeply anaesthetized and animals are placed in a spinal cord fixator. Access to the spinal cord is obtained via dorsal laminectomy at the level of the fourth cervical vertebra after splitting of the neck musculature. After exposing the spinal cord the dura and pia mater are opened by using a small scalpel knife. Subsequently dorsal hemisection of the spinal cord is performed with a pair of microscissors as deep as 1 mm ventral to the spinal surface. This results in the complete transection of two major spinal cord tracts, the corticospinal tract and the rubrospinal tract. The effect of a sequence encoding a TF polypeptide or an RNAi agents to knock down its expression in a neuronal cell is evaluated by stereotactically infusing the AAV vector comprising the relevant sequence near the cell bodies of the corticospinal neurons in the cortex and near the cell bodies of the rubrospinal neurons in the rubrospinal nucleus. The effect on an “environmental” or glial target cell is evualated by expression of the relavant sequence around and distal from the neural scar. Moreover the effect of on “environmental” target cell is determined by an ex vivo approach in which modified OEG (oliphactory ensheeted cells) that overexpress the relevant sequence are implanted.

### 3.2 Ventral and Dorsal Root Avulsion.

The ventral and dorsal spinal roots form the connections between the spinal cord and the large peripheral nerve (the sciatic nerve) that is essential for the functioning of the hind paws. Avulsion of these roots results in permanent paralysis of the hind paws and is a model for brachial plexus injury and root avulsion lesions that occur frequently in humans. Neurosurgical avulsion of the ventral or dorsal roots is achieved by opening the vertebral column at the level of the T13 to L2 vertebra. Following avulsion of the roots by traction with a watchmakers forceps the roots will be reimplanted into the spinal cord by a microsurgical procedure. The effect of a sequence encoding a TF polypeptide or an RNAi agents to knock down its expression in a neuronal target cell is studied by stereotactically injecting the AAV vector in the ventral horn of the spinal cord (transducing the motor neurons of the sciatic nerve) or by injecting the AAV vector in the dorsal root ganglia containing the cell bodies of the sensory neurons of the sciatic nerve. The effect of a sequence encoding a TF polypeptide or an RNAi agents to knock down its expression on an environmental target cell is determined evaluated by expression in the reimplanted ventral or dorsal roots.

### 3.3 Peripheral Nerve Transection.

The sciatic nerve innervates the hindpaw and transection of the sciatic nerve is a model of peripheral nerve injury in humans. The transected sciatic nerve of experimental animals regenerates to some extent as is the case in humans and neurosurgical repair of a transected nerve has a significantly beneficial effect on recovery of function. The sciatic nerve of adult Wistar rats will be exposed and transected at the mid-thigh level. The proximal and distal nerve stumps will be neurosurgically repaired. The effect of a sequence encoding a TF polypeptide or an RNAi agents to knock down its expression in neuronal target cell is studied by stereotactically infusion the AAV vector in the ventral spinal cord (transducing the motor neurons) or by injecting the vector in the DRG transducing the sensory neurons of the spinal cord. The effect on an environmental cell gene is determined by overexpression in the distal nerve stump to assess the effect on neurite outgrowth.

The neuroregeneration process in all three models is studied at the anatomical level and at the functional level. Anatomical studies include immunohistochemical staining of nerve fibers, tracing of nerve fibers using fluorescent dyes, and analysis of the formation of the neural scar and local effects on sparing of spinal tissue at the site of the lesion. The longitudinal functional studies are performed by testing the performance of the animals in the “catwalk” (a computerized analysis of motor performance using video imaging of the animal walking in a corridor over a glass plate) and in the “rope test” (a test that analyses the performance of the animal walking over a 4 cm thick rope stretched between two platforms).

The gene and cell therapy studies in each model are performed in two steps. The first step consists of a pilot study that is required to determine conditions for optimal delivery of the viral vector and to determine the required level of expression of the transgene at the site of delivery. These studies are performed in a small number of animals at a limited number of post lesion time points and form the basis for step 2, a large experiment that includes longitudinal functional testing and anatomical analysis of the gene therapy treatment for each individual target gene.

TABLE 3

| Original | Feature | SEQ ID | | |
|---|---|---|---|---|
| row | # | No. | Accession # | Annotation |
| 1 | 126 | 1 | NM_032616 | Liver-specific bHLH-Zip transcription factor 7 |
| 2 | 142 | 2 | NM_019193 | SRY-box containing gene 10 |
| 3 | 191 | 3 | BI288410 | Transcription elongation factor A2 |
| 6 | 279 | 4 | NM_057144 | Cysteine-rich protein 3 |
| 7 | 326 | 5 | BF416989 | Similar to transcriptional regulator protein |
| 8 | 425 | 6 | BE109271 | Similar to zinc finger DAZ interacting protein 3 |
| 9 | 652 | 7 | NM_053949 | Potassium voltage-gated channel, subfamily H (eag-related), member 2 |
| 10 | 684 | 8 | NM_013220 | Ankyrin repeat domain 1 (cardiac muscle) |
| 11 | 731 | 9 | NM_019328 | Nuclear receptor subfamily 4, group A, member 2 |
| 12 | 784 | 10 | NM_019334 | Paired-like homeodomain transcription factor 2 |
| 13 | 841 | 11 | BG669451 | Small zinc finger-like protein |
| 14 | 947 | 12 | NM_031586 | Immunoglobulin mu binding protein 2 |
| 15 | 961 | 13 | BI300993 | Similar to helicase-like protein NHL isoform 2 |
| 19 | 1261 | 14 | NM_053310 | Homer, neuronal immediate early gene, 3 |
| 20 | 1276 | 15 | NM_013028 | Short stature homeobox 2 |
| 22 | 1621 | 16 | BI296732 | Similar to zinc finger protein 291 |
| 23 | 1698 | 17 | AF009329 | Basic helix-loop-helix domain containing, class B3 |
| 24 | 1726 | 18 | NM_019208 | Multiple endocrine neoplasia 1 |
| 27 | 2094 | 19 | BE111840 | Similar to zinc finger protein 91 isoform 1 |
| 29 | 2306 | 20 | NM_019220 | Amino-terminal enhancer of split |
| 30 | 2366 | 21 | BF397780 | GLI-Kruppel family member GLI |
| 31 | 2399 | 22 | BG664819 | Paired-like homeodomain trancription factor Drg11 |
| 32 | 2422 | 23 | BF398414 | Similar to RNA helicase A |
| 33 | 2436 | 24 | BE120149 | Similar to checkpoint with forkhead and ring finger domains |
| 34 | 2466 | 25 | AW919327 | Similar to Kruppel-like factor 7 (ubiquitous) |
| 35 | 2500 | 26 | AF216807 | Cas-associated zinc finger protein |
| 36 | 2531 | 27 | NM_021835 | V-jun sarcoma virus 17 oncogene homolog (avian) |
| 39 | 2870 | 28 | NM_053499 | Transcription termination factor, mitochondrial |
| 40 | 2886 | 29 | NM_013058 | Inhibitor of DNA binding 3, dominant negative helix-loop-helix protein |
| 41 | 2907 | 30 | BG662969 | Similar to zinc finger RNA binding protein |
| 43 | 3075 | 31 | AF286470 | Sterol regulatory element binding factor 1 |
| 44 | 3123 | 32 | AW919881 | Similar to transcription factor NRF; ITBA4 gene |
| 45 | 3182 | 33 | AF095585 | Enigma (LIM domain protein) |
| 46 | 3186 | 34 | BG662589 | Similar to ATP-dependent chromatin remodeling protein SNF2H |
| 47 | 3302 | 35 | NM_024134 | DNA-damage inducible transcript 3 |
| 48 | 3910 | 36 | AW142350 | Similar to DNA polymerase epsilon p17 subunit (DNA polymerase epsilon subunit 3) |
| | | | | (Chromatin accessibility complex 17) (HuCHRAC17) (CHRAC-17) |
| 49 | 4004 | 37 | AI044335 | ISL1 transcription factor, LIM/homeodomain 1 |
| 50 | 4085 | 38 | NM_013060 | Inhibitor of DNA binding 2, dominant negative helix-loop-helix protein |
| 51 | 4155 | 39 | BF419978 | Damage-specific DNA binding protein 1 |
| 52 | 4461 | 40 | NM_013130 | MAD homolog 1 (Drosophila) |
| 53 | 4498 | 41 | NM_012555 | V-ets erythroblastosis virus E26 oncogene homolog 1 (avian) |
| 56 | 4888 | 42 | NM_052981 | Cyclin H |
| 57 | 4995 | 43 | NM_031788 | Zinc finger transcription factor REST protein |
| 58 | 5081 | 44 | NM_024359 | Hypoxia inducible factor 1, alpha subunit |
| 59 | 5121 | 45 | NM_022593 | Elongation factor SIII p15 subunit |
| 60 | 5152 | 46 | X54549 | Transcription factor E2a |
| 61 | 5204 | 47 | AW914809 | Similar to putative nucleic acid binding protein RY-1; EST AI449063 |
| 62 | 5216 | 48 | BG380993 | POU domain, class 3, transcription factor 2 |
| 63 | 5347 | 49 | BI279828 | Similar to Zinc finger protein 213 (Putative transcription factor CR53) |
| 65 | 5646 | 50 | NM_024383 | Hairy and enhancer of split 5 (Drosophila) |
| 67 | 5823 | 51 | NM_012912 | Activating transcription factor 3 |
| 69 | 5887 | 52 | NM_053563 | Nuclear RNA helicase, DECD variant of DEAD box family |
| 70 | 5910 | 53 | AI101323 | Similar to ets variant gene 5 |
| 71 | 5998 | 54 | NM_012963 | High mobility group box 1 |
| 75 | 6643 | 55 | NM_053400 | Transducin-like enhancer of split 3, homolog of Drosophila |
| 76 | 6772 | 56 | BG668660 | POU domain, class 4, transcription factor 1 |
| 77 | 6799 | 57 | D32249 | Protein carrying the RING-H2 sequence motif |
| 78 | 6848 | 58 | NM_017062 | Reversion induced LIM gene |
| 79 | 6945 | 59 | BE109673 | Similar to ETS-domain protein ELK-3 (ETS-related protein NET) (ETS-related protein |
| | | | | ERP) |
| 80 | 7269 | 60 | BF552916 | Similar to transcription elongation regulator 1; transcription factor CA150; TATA box |
| | | | | binding protein (TBP)-associated factor, RNA polymerase II, S, 150kD; TATA box- |
| | | | | binding protein-associated factor 2S |
| 81 | 7318 | 61 | U51583 | Transcription factor 8 |
| 82 | 7416 | 62 | NM_021771 | Transient receptor protein 3 |
| 83 | 7549 | 63 | NM_020471 | Insulin related protein 2 |
| 84 | 7730 | 64 | NM_053713 | Kruppel-like factor 4 (gut) |
| 85 | 7741 | 65 | AF281635 | Zinc finger protein 22 (KOX 15) |
| 86 | 7863 | 66 | NM_017347 | Protein kinase, mitogen activated 3 (extracellular-signal-regulated kinase 1, ERK1) |
| 87 | 7882 | 67 | NM_031345 | Glucocorticoid-induced leucine zipper |
| 88 | 8037 | 68 | D25233 | Retinoblastoma 1 |
| 89 | 8042 | 69 | NM_017012 | Glutamate receptor, metabotropic 5 |
| 91 | 8154 | 70 | BG375691 | Similar to heavy metal-responsiv etranscription factor |
| 92 | 8341 | 71 | NM_012747 | Signal transducer and activator of transcription 3 |
| 93 | 8353 | 72 | U66470 | Cell growth regulator with EF hand domain 1 |
| 94 | 5738 | 73 | NM_053727 | Nuclear factor, interleukin 3 regulated (Nfil3) |

TABLE 4

| SEQ ID | | | | | Absolute t | Degrees | |
|---|---|---|---|---|---|---|---|
| No. | GroupA mean | GroupA std. dev. | GroupB mean | GroupB std. dev. | value | freedom | p value |
| 1 | 0.191276 | 0.08998135 | 0.29419667 | 0.09255563 | 17.828.244 | 8.0 | 0.07 |
| 2 | −0.29659885 | 0.2808084 | 0.12643646 | 0.12922186 | 3.060.149 | 8.0 | 0.0 |
| 3 | 0.3147115 | 0.06635538 | 0.026922759 | 0.12285895 | 46.086.206 | 8.0 | 0.0 |
| 4 | 2.518.249 | 21.951.451 | −0.034472745 | 0.20320874 | 25.892.398 | 8.0 | 0.02 |
| 5 | 0.25047973 | 0.123529054 | 0.018793244 | 0.03619274 | 4.024.696 | 8.0 | 0.0 |
| 6 | 0.18367681 | 0.08937598 | −0.020447996 | 0.119798265 | 30.538.113 | 8.0 | 0.04 |
| 7 | −0.25099257 | 0.16835056 | −0.050202977 | 0.17502515 | 18.487.984 | 8.0 | 0.11 |
| 8 | 15.808.077 | 12.640.364 | 0.114515185 | 0.07329481 | 25.895.073 | 8.0 | 0.06 |
| 9 | 0.12608035 | 0.1448254 | 0.27401876 | 0.054940086 | 2.135.627 | 8.0 | 0.05 |
| 10 | 0.2652061 | 0.12347023 | 0.08195016 | 0.054686677 | 30.344.772 | 8.0 | 0.01 |
| 11 | 0.26772735 | 0.02931422 | −0.10165246 | 0.18136877 | 44.956.846 | 8.0 | 0.0 |
| 12 | 0.11256783 | 0.1809302 | −0.13704787 | 0.14957842 | 23.776.278 | 8.0 | 0.01 |
| 13 | −0.25252366 | 0.13448788 | 0.47734118 | 0.1266071 | 8.835.806 | 8.0 | 0.0 |
| 14 | 0.028603777 | 0.09328679 | −0.18086287 | 0.07400084 | 39.335.437 | 8.0 | 0.0 |
| 15 | −0.41715294 | 0.22783229 | −0.19586258 | 0.16561553 | 1.756.759 | 8.0 | 0.07 |
| 16 | −0.34696865 | 0.13574399 | 0.12652393 | 0.039355658 | 7.491.202 | 8.0 | 0.0 |
| 17 | 0.39461547 | 0.10670859 | 0.10036655 | 0.075278394 | 5.038.397 | 8.0 | 0.0 |
| 18 | −0.15230462 | 0.11851204 | 0.25972503 | 0.10725563 | 5.764.048 | 8.0 | 0.0 |
| 19 | −0.30426946 | 0.24465492 | 0.29665762 | 0.07125915 | 52.731.614 | 8.0 | 0.0 |
| 20 | −0.36855772 | 0.06607565 | −0.08192224 | 0.12781338 | 44.545.736 | 8.0 | 0.0 |
| 21 | −0.12965891 | 0.15702012 | 0.22366826 | 0.07841842 | 45.014.567 | 8.0 | 0.0 |
| 22 | −0.4119574 | 0.13982894 | −0.006071449 | 0.16206208 | 4.240.131 | 8.0 | 0.01 |
| 23 | 0.51229817 | 0.20145048 | −0.4808197 | 0.14537545 | 8.938.935 | 8.0 | 0.0 |
| 24 | 0.10055641 | 0.20218638 | 0.641132 | 0.09545781 | 5.406.214 | 8.0 | 0.0 |
| 25 | 0.06401521 | 0.12373511 | 0.5095577 | 0.13166857 | 551.382 | 8.0 | 0.0 |
| 26 | −0.03969292 | 0.09091078 | 0.40250492 | 0.15438524 | 55.188.932 | 8.0 | 0.0 |
| 27 | 0.8786588 | 0.24957162 | 0.43626887 | 0.14500387 | 34.271.739 | 8.0 | 0.01 |
| 28 | −0.30927378 | 0.23727387 | 0.017242765 | 0.066337585 | 29.634.485 | 8.0 | 0.01 |
| 29 | −0.37353358 | 0.22566256 | −0.06296162 | 0.12967944 | 26.682.317 | 8.0 | 0.05 |
| 30 | −0.25151688 | 0.15946624 | 0.2875095 | 0.12290419 | 59.866.014 | 8.0 | 0.0 |
| 31 | −0.025436472 | 0.10006186 | 0.061424304 | 0.032329798 | 1.847.049 | 8.0 | 0.14 |
| 32 | −0.12040377 | 0.058398876 | −0.50525177 | 0.06903979 | 9.516.545 | 8.0 | 0.0 |
| 33 | −0.18445475 | 0.02656519 | −0.021483378 | 0.06970635 | 48.851.295 | 8.0 | 0.0 |
| 34 | −0.3011972 | 0.12883674 | −0.011422397 | 0.0534899 | 4.644.868 | 8.0 | 0.0 |
| 35 | 0.24577789 | 0.13545124 | −0.34958506 | 0.07800097 | 8.517.154 | 8.0 | 0.0 |
| 36 | −0.15085049 | 0.09533041 | 0.29347834 | 0.06168665 | 8.750.046 | 8.0 | 0.0 |
| 37 | −0.19971208 | 0.17492615 | −0.021404076 | 0.07006274 | 211.589 | 8.0 | 0.08 |
| 38 | −0.3668775 | 0.15605511 | 0.03247493 | 0.11628215 | 45.884.557 | 8.0 | 0.0 |
| 39 | −0.11933086 | 0.060890943 | 0.17831984 | 0.11124986 | 5.247.974 | 8.0 | 0.0 |
| 40 | 0.51476455 | 0.18589973 | 0.304589 | 0.14521387 | 19.922.832 | 8.0 | 0.08 |
| 41 | −0.326088 | 0.13640533 | −0.051604014 | 0.16174503 | 2.900.807 | 8.0 | 0.01 |
| 42 | 0.24490833 | 0.08820734 | 0.13157728 | 0.053220194 | 2.459.894 | 8.0 | 0.03 |
| 43 | −0.16488808 | 0.11349886 | −0.2932312 | 0.11894246 | 17.455.815 | 8.0 | 0.11 |
| 44 | −0.45315677 | 0.14812614 | 0.7625928 | 0.20267113 | 10.829.302 | 8.0 | 0.0 |
| 45 | −0.01357463 | 0.090939574 | −0.33551374 | 0.054550964 | 67.883.353 | 8.0 | 0.0 |
| 46 | −0.14261559 | 0.13845421 | 0.23903897 | 0.062182453 | 5.622.763 | 8.0 | 0.0 |
| 47 | −0.37772548 | 0.107589774 | 0.2215704 | 0.090646684 | 95.252.695 | 8.0 | 0.0 |
| 48 | 0.1647337 | 0.17172422 | 0.0048121377 | 0.05610436 | 1.979.418 | 8.0 | 0.05 |
| 49 | 0.26632026 | 0.08344636 | −0.05779774 | 0.18947724 | 3.500.557 | 8.0 | 0.0 |
| 50 | −0.20546076 | 0.079123475 | −0.016621966 | 0.076550834 | 3.835.438 | 8.0 | 0.01 |
| 51 | 33.425.648 | 13.553.762 | 17.055.057 | 0.53917086 | 25.095.108 | 8.0 | 0.07 |
| 52 | 0.37559083 | 0.13448776 | 0.22962713 | 0.14686035 | 1.639.009 | 8.0 | 0.15 |
| 53 | −0.096040644 | 0.04775077 | 0.10626793 | 0.01720458 | 8.912.819 | 8.0 | 0.0 |
| 54 | −0.9999626 | 0.21742286 | 0.29818207 | 0.33789507 | 7.224.283 | 8.0 | 0.0 |
| 55 | 0.27150935 | 0.21007533 | −0.05582723 | 0.11544348 | 30.535.223 | 8.0 | 0.02 |
| 56 | −0.34967607 | 0.21199721 | 0.14851996 | 0.12974373 | 4.482.027 | 8.0 | 0.0 |
| 57 | −0.4712569 | 0.20201696 | −0.1871186 | 0.04520126 | 30.691.566 | 8.0 | 0.07 |
| 58 | 0.48246637 | 0.29227796 | 0.14581501 | 0.12336912 | 23.728.285 | 8.0 | 0.11 |
| 59 | −0.26658517 | 0.058810614 | 0.010586207 | 0.029567739 | 9.415.471 | 8.0 | 0.0 |
| 60 | 0.086990654 | 0.092771776 | 0.24751826 | 0.045154605 | 34.789.712 | 8.0 | 0.02 |
| 61 | 0.51558256 | 0.1287256 | −0.035235208 | 0.13657714 | 6.562.603 | 8.0 | 0.0 |
| 62 | −0.42423612 | 0.19547437 | 0.058338486 | 0.13330784 | 45.606.666 | 8.0 | 0.0 |
| 63 | −0.41394395 | 0.097937435 | −0.0447161 | 0.09067719 | 61.858.244 | 8.0 | 0.0 |
| 64 | 0.41700354 | 0.17227173 | 0.06550292 | 0.116308324 | 37.813.184 | 8.0 | 0.01 |
| 65 | −0.038928088 | 0.12589741 | −0.21717148 | 0.15188214 | 2.020.325 | 8.0 | 0.1 |
| 66 | −0.32716554 | 0.18830921 | 0.31073037 | 0.18019658 | 5.472.683 | 8.0 | 0.0 |
| 67 | −0.6056122 | 0.29451895 | −0.25393927 | 0.16444688 | 2.331.219 | 8.0 | 0.03 |
| 68 | −0.45481467 | 0.17811894 | −0.005240664 | 0.18820198 | 38.794.932 | 8.0 | 0.0 |
| 69 | −0.045707893 | 0.14868051 | 0.45712274 | 0.06720846 | 6.890.953 | 8.0 | 0.0 |
| 70 | 0.4051269 | 0.2082053 | 0.24677925 | 0.081244566 | 15.842.669 | 8.0 | 0.13 |
| 71 | 0.02021561 | 0.3245123 | 0.29193884 | 0.15816365 | 168.306 | 8.0 | 0.1 |
| 72 | 0.19998685 | 0.057265796 | −0.26813203 | 0.11891636 | 7.930.694 | 8.0 | 0.0 |
| 73 | 0.4336165 | 0.33120352 | 0.18354729 | 0.03169812 | 16.806.232 | 8.0 | 0.12 |

TABLE 5

| SEQ ID | | | | | | | |
|---|---|---|---|---|---|---|---|
| No. | sn06 | sn12 | sn24 | sn48 | sn72 | sn7d | sn14d |
| 1 | 0.1558361 | 0.09771143 | 0.3246557 | 0.2369242 | 0.1412525 | −0.1432063 | 0.06901464 |
| 2 | −0.7317736 | −0.4287063 | −0.1260795 | −0.1181773 | −0.0782577 | −1.174.161 | −0.2389063 |
| 3 | 0.2067104 | 0.3698325 | 0.29979 | 0.3346767 | 0.3625479 | −0.1298397 | 0.09151845 |
| 4 | 0.5432757 | 0.1412659 | 247.242 | 4.877.961 | 4.556.322 | 2.606.621 | 3.154.104 |
| 5 | 0.1282869 | 0.1188404 | 0.2683012 | 0.3510266 | 0.3859435 | −0.04560748 | −0.0103544 |
| 6 | 0.3203745 | 0.07251105 | 0.1680532 | 0.161187 | 0.1962584 | 0.08184798 | 0.1599001 |
| 7 | −0.08693782 | −0.1011402 | −0.2999101 | −0.2683889 | −0.4985858 | −0.2863907 | −0.1861458 |
| 8 | 0.4568064 | 0.1504916 | 1.654.679 | 2.831.141 | 2.810.921 | 1.574.868 | 1.824.694 |
| 9 | 0.2155158 | 0.2207655 | −0.03378421 | 0.2572749 | −0.02937032 | 0.1117986 | 0.385201 |
| 10 | 0.4604446 | 0.2301594 | 0.1792831 | 0.303876 | 0.1522675 | 0.1939013 | 0.2727779 |
| 11 | 0.2562839 | 0.2848116 | 0.2814723 | 0.2943501 | 0.2217189 | −0.01952131 | −0.1746757 |
| 12 | 0.4151186 | 0.1413324 | −0.004345622 | −0.02566317 | 0.0363969 | 0.1686308 | −0.2756614 |
| 13 | −0.07229844 | −0.2484052 | −0.451717 | −0.2395484 | −0.2506492 | −0.1599629 | 0.2420716 |
| 14 | −0.03600459 | −0.01509115 | −0.01603422 | 0.191787 | 0.01836185 | 0.02904026 | −0.06005953 |
| 15 | −0.2502049 | −0.1027764 | −0.5788009 | −0.6199992 | −0.5339832 | −0.4823366 | −0.360056 |
| 16 | −0.2224019 | −0.2149414 | −0.3881334 | −0.5437289 | −0.3656376 | −0.2254509 | 0.05255105 |
| 17 | 0.378263 | 0.4620893 | 0.2910398 | 0.3012116 | 0.5404735 | 0.4174726 | 0.1452751 |
| 18 | 0.04619979 | −0.1532156 | −0.266115 | −0.2067056 | −0.1816867 | −0.1249904 | −0.1124252 |
| 19 | −0.1097077 | −3.14E+01 | −0.3785719 | −0.4336688 | −0.5990847 | 0.2020479 | 0.04033961 |
| 20 | −0.345684 | −0.3705025 | −0.2787399 | −0.4609265 | −0.3869356 | −0.3295858 | −0.05790312 |
| 21 | 0.1494448 | −0.2138654 | −0.2146659 | −0.1710479 | −0.1981602 | −0.05509081 | −0.04233075 |
| 22 | −0.4748215 | −0.1923171 | −0.375397 | −0.5633458 | −0.4539057 | −0.5945381 | −0.5162931 |
| 23 | 0.3070603 | 0.5254416 | 0.3669248 | 0.5361904 | 0.8258737 | −0.1063013 | 0.4886672 |
| 24 | 0.1878881 | 0.3407511 | −0.1441231 | 0.191226 | −0.07296006 | 0.6950469 | 0.9677822 |
| 25 | −0.06725325 | 0.07345419 | 0.08446003 | 0.2532059 | −0.0237908 | −0.2031508 | 0.3372429 |
| 26 | 0.08937457 | 0.01058607 | −0.1450715 | −0.0807027 | −0.07265103 | 0.05635493 | 0.06964444 |
| 27 | 1.085.842 | 0.7981197 | 0.863625 | 1.133.722 | 0.5119855 | 196.982 | 0.835328 |
| 28 | 0.02987168 | −0.1502678 | −0.4425677 | −0.4748913 | −0.5085138 | 0.140724 | −0.1690773 |
| 29 | −0.05868223 | −0.2301114 | −0.6227213 | −0.4735978 | −0.4825551 | 0.06948337 | 0.0946847 |
| 30 | −0.1048203 | −0.08390493 | −0.3575769 | −0.2577335 | −0.4535488 | 0.02948847 | 0.1732134 |
| 31 | 0.1327854 | −0.1164908 | −0.06555498 | −0.0876905 | 0.009768522 | 0.008128723 | 0.09064383 |
| 32 | −0.08977795 | −0.06756138 | −0.08734803 | −0.1468078 | −0.2105237 | −0.02889196 | 0.006059056 |
| 33 | −0.1793946 | −0.2301167 | −0.1755856 | −0.1605238 | −0.176653 | −0.1612568 | −0.1887727 |
| 34 | −0.2405098 | −0.114286 | −0.3836187 | −0.3231977 | −0.4443738 | −0.2548061 | 0.09023677 |
| 35 | 0.1338123 | 0.161746 | 0.470169 | 0.1926194 | 0.2705428 | −0.1877333 | −0.2015085 |
| 36 | 0.01050078 | −0.1947252 | −0.1401482 | −0.2093325 | −0.2205473 | −0.1320272 | 0.005240137 |
| 37 | −0.1157724 | 0.04999444 | −0.4094232 | −0.2875514 | −0.2358079 | −0.2410946 | −0.0376626 |
| 38 | −0.1582297 | −0.2476876 | −0.5054303 | −0.425733 | −0.4973068 | −0.3732851 | 0.1456612 |
| 39 | −0.1067891 | −0.07854577 | −0.1919344 | −0.04823912 | −0.1711459 | −0.2232564 | 0.1343341 |
| 40 | 0.2510975 | 0.4103342 | 0.5500253 | 0.7054257 | 0.65694 | 0.9431555 | 0.7757996 |
| 41 | −0.183555 | −0.2378237 | −0.3963155 | −0.5257383 | −0.2870075 | −0.06271061 | 0.4335159 |
| 42 | 0.2064566 | 0.1199865 | 0.2744849 | 0.2659126 | 0.357701 | 0.01089832 | 0.2334591 |
| 43 | −0.3355998 | −0.05144871 | −0.10658 | −0.2207079 | −0.110104 | −0.0788228 | −0.2069903 |
| 44 | −0.5874471 | −0.2634761 | −0.4546313 | −0.3530377 | −0.6071917 | 0.02722161 | 0.5163701 |
| 45 | 0.01221227 | 0.1318836 | −0.09888305 | −0.0685196 | −0.04456639 | −0.01921898 | 0.1250597 |
| 46 | 0.08640951 | −0.216589 | −0.2051231 | −0.1162092 | −0.2615661 | −0.06153485 | 0.1669488 |
| 47 | −0.3376728 | −0.2114622 | −0.4027999 | −0.4692307 | −0.4674618 | −0.1154344 | 0.296485 |
| 48 | 0.1090351 | −0.05292181 | 0.1724239 | 0.1708124 | 0.4243189 | 0.1947077 | 0.1300725 |
| 49 | 0.2967858 | 0.2044852 | 0.2106498 | 0.2198006 | 0.3998799 | 0.2773153 | 0.1032894 |
| 50 | −0.1160905 | −0.1249879 | −0.2367333 | −0.2751308 | −0.2743613 | −0.08751065 | −0.15301 |
| 51 | 0.9966935 | 3.366.721 | 4.089.873 | 4.282.469 | 3.977.068 | 3.887.035 | 3.776.157 |
| 52 | 0.257261 | 0.2341419 | 0.3600306 | 0.5230119 | 0.5035087 | 0.3092205 | 0.3863867 |
| 53 | −0.1632029 | −0.06545344 | −0.04673042 | −0.1265081 | −0.07830833 | −0.0261539 | −0.1433064 |
| 54 | −0.9066722 | −0.7027535 | −1.149.601 | −0.9778177 | −1.262.969 | −0.4136318 | 0.1524006 |
| 55 | 0.2164973 | −0.04242942 | 0.2706162 | 0.5136574 | 0.3992053 | 0.730776 | 0.3989139 |
| 56 | −0.291487 | −0.004252673 | −0.5236402 | −0.4497473 | −0.4792531 | −0.1165251 | 0.1769261 |
| 57 | −0.4421018 | −0.1505531 | −0.4906928 | −0.5886399 | −0.6842967 | −0.2562885 | 0.01631659 |
| 58 | 0.1086326 | 0.2303509 | 0.6567723 | 0.7631437 | 0.6534324 | 0.1253972 | 0.3945464 |
| 59 | −0.2683249 | −0.3611744 | −0.2008907 | −0.2442108 | −0.2583251 | −0.05346133 | −0.1886977 |
| 60 | −0.008266453 | 0.1033885 | 0.09539293 | 0.228358 | 0.0160803 | −0.008583952 | 0.2962678 |
| 61 | 0.4178232 | 0.3990861 | 0.4713366 | 0.5824495 | 0.7072174 | 0.4162195 | 0.4638666 |
| 62 | −0.369357 | −0.1140727 | −0.4675957 | −0.5870589 | −0.5830963 | −0.1277637 | 0.04571087 |
| 63 | −0.3439742 | −0.2776483 | −0.4608991 | −0.4937087 | −0.4934895 | −0.2703238 | −0.1343503 |
| 64 | 0.1926589 | 0.3195887 | 0.6448339 | 0.499829 | 0.4281072 | 0.1017064 | 0.187578 |
| 65 | −0.2407009 | 0.0633041 | −0.0803096 | 0.01428494 | 0.04878102 | −0.03194587 | 0.2565298 |
| 66 | −0.6558098 | −0.2588241 | −0.1917353 | −0.2258327 | −0.3036257 | −0.517639 | −0.01484309 |
| 67 | −0.8217477 | −0.189226 | −0.4098531 | −0.7324132 | −0.8748212 | −1.054.455 | −0.4327303 |
| 68 | −0.6958813 | −0.2326875 | −0.5553017 | −0.3617634 | −0.4284396 | −0.3976313 | 0.1629406 |
| 69 | −0.263703 | 0.1015806 | −0.1192767 | 0.06758651 | −0.01472687 | 0.01613022 | −0.1287328 |
| 70 | 0.1899845 | 0.2228689 | 0.3967296 | 0.6838121 | 0.5322395 | 0.2607976 | 0.3476054 |
| 71 | −0.3106052 | −0.2476076 | 0.1909605 | 0.4784587 | −0.01012833 | −0.1196586 | 0.2724296 |
| 72 | 0.2016529 | 0.1156054 | 0.1902213 | 0.2177426 | 0.274712 | 0.1742032 | 0.1199804 |
| 73 | −0.0110776 | 0.1872076 | 0.673576 | 0.7615774 | 0.556799 | 0.3371665 | 0.3368869 |

TABLE 6

| SEQ ID | | | | | | | |
|---|---|---|---|---|---|---|---|
| No. | dr06 | dr12 | dr24 | dr48 | dr72 | dr7d | dr14d |
| 1 | 0.1973283 | 0.3130837 | 0.3817478 | 0.3811041 | 0.1977194 | 0.1941048 | 0.01383735 |
| 2 | 0.339123 | 0.1323143 | 0.007922126 | 0.111571 | 0.04125187 | 0.3093349 | 0.4389845 |
| 3 | 0.16911 | 0.1127011 | −0.06748518 | 0.04666118 | −0.1263733 | 0.002977038 | 0.2089391 |
| 4 | −0.1768374 | −0.2868607 | −0.01736711 | 0.08504497 | 0.2236565 | −0.08455901 | −0.04471036 |
| 5 | 0.05942727 | 0.04301547 | 0.0167006 | 0.01008084 | −0.03525796 | −0.04721277 | 0.052539 |
| 6 | −0.1300111 | −0.1354279 | 0.1255511 | −0.04355285 | 0.08120077 | 0.03270977 | −0.04904264 |
| 7 | 0.1671769 | 0.06688949 | −0.05056747 | −0.163608 | −0.2709058 | −0.1312973 | 0.3174949 |
| 8 | 0.04648873 | 0.02564318 | 0.1825079 | 0.1450475 | 0.1728886 | 0.09373517 | 0.1840112 |
| 9 | 0.2887958 | 0.1970901 | 0.3501625 | 0.2624667 | 0.2715787 | 0.1879753 | 0.1933874 |
| 10 | 0.04521316 | 0.01385129 | 0.1216451 | 0.1482705 | 0.08077077 | 0.06074692 | 0.05992033 |
| 11 | 0.0763087 | 7.56E+01 | −0.06353723 | −0.1244187 | −0.3973714 | −0.4131432 | 0.03402906 |
| 12 | 0.1272038 | −0.1683711 | −0.2270507 | −0.2221186 | −0.1949028 | −0.2705385 | −0.1958942 |
| 13 | 0.6352176 | 0.5672631 | 0.4232376 | 0.4491251 | 0.3118626 | 0.327043 | 0.3726922 |
| 14 | −0.05661885 | −0.1680281 | −0.2314453 | −0.2254891 | −0.222733 | −0.1240921 | −0.04726921 |
| 15 | 0.01214463 | −0.1446584 | −0.1267396 | −0.3093078 | −0.4107517 | −0.4575409 | −0.2919099 |
| 16 | 0.07500994 | 0.1223775 | 0.1050582 | 0.1583941 | 0.1717799 | 0.09542194 | −0.01735669 |
| 17 | 0.1049946 | 0.02572789 | 0.06224912 | 0.2243456 | 0.0845155 | 0.2475339 | 0.01766135 |
| 18 | 0.3581816 | 0.3736712 | 0.2483219 | 0.1980018 | 0.1204488 | 0.1369549 | 0.2437013 |
| 19 | 0.3725278 | 0.3321066 | 0.3376625 | 0.2233114 | 0.2176799 | 0.1381619 | 0.2949439 |
| 20 | −0.0158671 | 0.1074211 | −0.1553513 | −0.1330158 | −0.2127981 | −0.1705037 | −0.1607512 |
| 21 | 0.3253706 | 0.1494467 | 0.1973042 | 0.2865524 | 0.1596674 | 0.1897787 | 0.2462874 |
| 22 | 0.1843009 | 0.0355324 | −0.2602938 | 0.03827436 | −0.02817109 | −0.1347184 | 0.1220883 |
| 23 | −0.2339508 | −0.482065 | −0.6075991 | −0.5251544 | −0.5553294 | −0.5158129 | −0.5724622 |
| 24 | 0.5186466 | 0.5811743 | 0.6362222 | 0.7230513 | 0.7465658 | 0.6545382 | 0.5683355 |
| 25 | 0.579884 | 0.698916 | 0.486355 | 0.396002 | 0.3866316 | 0.5456986 | 0.6063886 |
| 26 | 0.2387611 | 0.2793844 | 0.4867993 | 0.3903312 | 0.6172484 | 0.2794189 | −0.08279405 |
| 27 | 0.4169806 | 0.5513372 | 0.2468202 | 0.3601926 | 0.6060138 | 0.03835345 | 0.1201191 |
| 28 | −0.04504331 | −0.06163245 | 0.08755855 | 0.04776689 | 0.05756414 | 0.02526058 | 0.1675307 |
| 29 | 0.1059745 | 0.03646894 | −0.1340731 | −0.1150623 | −0.2081162 | 0.1641866 | 0.1490791 |
| 30 | 0.3169668 | 0.483706 | 0.2422345 | 0.2343532 | 0.1602871 | 0.2168385 | 0.2017017 |
| 31 | 0.03268796 | 0.03064528 | 0.1023292 | 0.05397614 | 0.08748292 | 0.02350884 | 0.1337322 |
| 32 | −0.5688593 | −0.5889388 | −0.4571512 | −0.475443 | −0.4358665 | −0.3757831 | −0.3438554 |
| 33 | −0.1169383 | 0.02302856 | −0.008717444 | 0.05864297 | −0.06343267 | 0.0856768 | 0.1047072 |
| 34 | 0.01726067 | −0.009388895 | −0.08562127 | −0.0353266 | 0.05596411 | 0.01449971 | 0.02263383 |
| 35 | −0.2179217 | −0.3435856 | −0.385734 | −0.4162495 | −0.3844344 | −0.2425506 | −0.1396149 |
| 36 | 0.2818771 | 0.2385894 | 0.3706768 | 0.233094 | 0.3431545 | 0.1290513 | 0.2045317 |
| 37 | 0.0774812 | −0.1179101 | −0.003562745 | −0.0282981 | −0.03473063 | 0.1033266 | −0.04810641 |
| 38 | 0.05360406 | −0.134962 | 0.01394541 | 0.03848819 | 0.191299 | 0.2420216 | 0.05520156 |
| 39 | 0.2952613 | 0.2402279 | 0.1072127 | 0.2272537 | 0.02164353 | 0.006201866 | 0.2212615 |
| 40 | 0.162745 | 0.2972065 | 0.5140217 | 0.3706618 | 0.17831 | 0.06610773 | 0.3507646 |
| 41 | −0.2893791 | −0.1463984 | 0.09556912 | 0.02932505 | 0.05286324 | 0.1176323 | −0.1679851 |
| 42 | 0.1840403 | 0.1452603 | 0.07841027 | 0.177428 | 0.0727475 | 0.02342524 | −0.1080731 |
| 43 | −0.2551091 | −0.1218556 | −0.4478359 | −0.3073151 | −0.3340402 | −0.3051284 | −0.3409655 |
| 44 | 0.458244 | 0.7069098 | 1.010.945 | 0.8002864 | 0.8365789 | 0.596075 | 0.652618 |
| 45 | −0.3344343 | −0.3366241 | −0.3120624 | −0.2728041 | −0.4216437 | −0.5473869 | −0.2561142 |
| 46 | 0.2018315 | 0.2203151 | 0.2102207 | 0.2131865 | 0.349641 | 0.297656 | 0.2829125 |
| 47 | 0.1535599 | 0.3617828 | 0.2643609 | 0.1724312 | 0.1557171 | 0.1373513 | 0.1813142 |
| 48 | −0.03321126 | 0.01256621 | 0.0243397 | 0.08314148 | −0.06277544 | −0.007596061 | 0.1032248 |
| 49 | −0.1096246 | −0.3122666 | 0.05445588 | −0.1103411 | 0.1887877 | −0.0942788 | −0.1788934 |
| 50 | 0.04410363 | 0.0733694 | −0.0663999 | −0.02191465 | −0.1122683 | −0.08194525 | 0.05703912 |
| 51 | 1.019.113 | 2.025.991 | 2.428.693 | 1.494.072 | 155.966 | 1.121.835 | 0.4917749 |
| 52 | 0.1221304 | 0.1229114 | 0.3199252 | 0.137479 | 0.4456897 | 0.07182215 | −0.03496951 |
| 53 | 0.09642483 | 0.1334007 | 0.09869235 | 0.09031255 | 0.1125092 | 0.06997328 | 0.03882476 |
| 54 | −0.2645099 | 0.2387706 | 0.5476069 | 0.5339329 | 0.4351098 | 0.5914686 | 0.3475495 |
| 55 | −0.03683314 | 0.131334 | −0.1418236 | −0.1565529 | −0.07526052 | 0.04747103 | −0.2149953 |
| 56 | 0.1226165 | 0.07721383 | 0.3671463 | 0.1444229 | 0.03120021 | 0.1047943 | 0.2809938 |
| 57 | −0.2534786 | −0.206769 | −0.1560744 | −0.180948 | −0.138323 | −0.1669703 | −0.2644261 |
| 58 | −0.05026714 | 0.1320359 | 0.1467168 | 0.2645922 | 0.2359973 | −0.1044135 | 4.83E+01 |
| 59 | 0.03132684 | 0.01598264 | 0.0344023 | −0.03911732 | 0.01033657 | −0.07825692 | −0.1510012 |
| 60 | 0.3151016 | 0.2568678 | 0.195989 | 0.2186471 | 0.2509857 | 0.3383734 | 0.2410399 |
| 61 | −0.2627268 | −0.058442 | 0.05008607 | 0.02130223 | 0.07360446 | −0.09612732 | −0.1868379 |
| 62 | 0.253318 | −0.03776859 | −0.09168954 | 0.08131995 | 0.08651261 | 0.06665391 | 0.204232 |
| 63 | 0.09107962 | 0.004654835 | −0.09307675 | −0.1256639 | −0.1005743 | −0.03899443 | 0.2239193 |
| 64 | −0.004486644 | −0.03020328 | 0.2030966 | −0.02224341 | 0.1813513 | −0.1448908 | −0.3284973 |
| 65 | −0.2348853 | −0.4002066 | −0.312244 | −0.1224733 | −0.01604818 | −0.1434013 | 0.002946273 |
| 66 | 0.03656826 | 0.4465074 | 0.4635356 | 0.2234279 | 0.3836126 | 0.4112017 | 0.1227124 |
| 67 | −0.01823391 | −0.3795384 | −0.4285532 | −0.2678823 | −0.1754886 | −0.5498711 | −0.4208958 |
| 68 | −0.2813799 | 0.008948142 | 0.05483907 | −0.04790244 | 0.2392918 | −0.1170033 | −0.3862413 |
| 69 | 0.4647072 | 0.4007395 | 0.391959 | 0.5593327 | 0.4688752 | 0.4163386 | 0.4892034 |
| 70 | 0.1829931 | 0.1365568 | 0.300214 | 0.3015548 | 0.3125776 | 0.06650015 | 0.1067704 |
| 71 | 0.06652456 | 0.3168936 | 0.4563009 | 0.2076103 | 0.4123649 | 0.2072641 | 0.1462762 |
| 72 | −0.3491965 | −0.384726 | −0.2409813 | −0.2856528 | −0.08010367 | −0.2723119 | −0.1457383 |
| 73 | 0.1720967 | 0.1582755 | 0.2331718 | 0.1583415 | 0.1958509 | −0.0815734 | 0.004344626 |

TABLE 7

| SEQ ID | SEQ ID | Accession # | |
|---|---|---|---|
| No. Rat | No. Human | Human | |
| sequence | orthologue | orthologue | Annotation human orthologue |
| 1 | 74 | NM_205834 | Liver-specific bHLH-Zip transcription factor LISCH7 |
| 2 | 75 | NM_006941 | SRY (sex determining region Y)-box 10 SOX10 |
| 3 | 76 | NM_198723 | Transcription elongation factor A (SII), 2 TCEA2 |
| 4 | 77 | NM_003476 | Cysteine and glycine-rich protein 3 (cardiac LIM protein) CSRP3 |
| 5 | 78 | NM_013260 | Transcriptional regulator protein HCNGP |
| 6 | 79 | NM_014648 | Zinc finger DAZ interacting protein 3 DZIP3 |
| 7 | 80 | NM_000238 | Potassium voltage-gated channel, subfamily H (eag-related), member 2 KCNH2 |
| 8 | 81 | NM_014391 | Ankyrin repeat domain 1 (cardiac muscle) ANKRD1 |
| 9 | 82 | NM_006186 | Nuclear receptor subfamily 4, group A, member 2 NR4A2 |
| 10 | 83 | NM_153427 | Paired-like homeodomain transcription factor 2 PITX2 |
| 11 | 84 | NM_012456 | Translocase of inner mitochondrial membrane 10 homolog (yeast) TIMM10 |
| 12 | 85 | NM_002180 | Immunoglobulin mu binding protein 2 IGHMBP2 |
| 13 | 86 | NM_032957 | Homo sapiens regulator of telomere elongation helicase 1 (RTEL1), transcript variant 2, mRNA |
| 14 | 87 | NM_004838 | Homer homolog 3 (Drosophila) HOMER3 |
| 15 | 88 | NM_006884 | Short stature homeobox 2 SHOX2 |
| 16 | 89 | NM_020843 | Zinc finger protein 291 ZNF291 |
| 17 | 90 | NM_030762 | Basic helix-loop-helix domain containing, class B, 3 BHLHB3 |
| 18 | 91 | NM_130799 | Multiple endocrine neoplasia I MEN1 |
| 19 | 92 | NM_053023 | Zinc finger protein 91 homolog (mouse) ZFP91 |
| 20 | 93 | NM_001130 | Amino-terminal enhancer of split AES |
| 21 | 94 | NM_005269 | Glioma-associated oncogene homolog 1 (zinc finger protein) GLI1 |
| 22 | 95 | NM_021926 | Aristaless-like homeobox 4 ALX4 |
| 23 | 96 | NM_030588 | DEAH (Asp-Glu-Ala-His) box polypeptide 9 DHX9 |
| 24 | 97 | NM_018223 | Checkpoint with forkhead and ring finger domains CHFR |
| 25 | 98 | NM_003709 | Kruppel-like factor 7 (ubiquitous) KLF7 |
| 26 | 99 | NM_133476 | Zinc finger protein 384 ZNF384 |
| 27 | 100 | NM_002228 | V-jun sarcoma virus 17 oncogene homolog (avian) JUN |
| 28 | 101 | NM_006980 | Transcription termination factor, mitochondrial MTERF |
| 29 | 102 | NM_002167 | Inhibitor of DNA binding 3, dominant negative helix-loop-helix protein ID3 |
| 30 | 103 | NM_016107 | Zinc finger RNA binding protein ZFR |
| 31 | 104 | NM_001005291 | Smith-Magenis syndrome chromosome region, candidate 6 SREBF1 |
| 32 | 105 | NM_015129 | Septin 6 SEPT6 |
| 33 | 106 | NM_203353 | PDZ and LIM domain 7 (enigma) PDLIM7 |
| 34 | 107 | NM_003601 | SWI/SNF related, matrix associated, actin dependent regulator of chromatin, subfamily a, member |
| | | | 5 SMARCA5 |
| 35 | 108 | NM_004083 | DNA-damage-inducible transcript 3 DDIT3 |
| 36 | 109 | NM_017443 | Polymerase (DNA directed), epsilon 3 (p17 subunit) POLE3 |
| 37 | 110 | NM_002202 | ISL1 transcription factor, LIM/homeodomain, (islet-1) ISL1 |
| 38 | 111 | NM_002166 | Inhibitor of DNA binding 2, dominant negative helix-loop-helix protein ID2 |
| 39 | 112 | NM_001923 | Damage-specific DNA binding protein 1, 127 kDa DDB1 |
| 40 | 113 | NM_001003688 | SMAD, mothers against DPP homolog 1 (Drosophila) SMAD1 |
| 41 | 114 | NM_005238 | V-ets erythroblastosis virus E26 oncogene homolog 1 (avian) ETS1 |
| 42 | 115 | NM_001239 | Cyclin H CCNH |
| 43 | 116 | NM_005612 | RE1-silencing transcription factor REST |
| 44 | 117 | NM_181054 | Hypoxia-inducible factor 1, alpha subunit (basic helix-loop-helix transcription factor) HIF1A |
| 45 | 118 | NM_005648 | Transcription elongation factor B (SIii), polypeptide 1 (15 kDa, elongin C) TCEB1 |
| 46 | 119 | NM_003200 | Transcription factor 3 (E2A immunoglobulin enhancer binding factors E12/E47) TCF3 |
| 47 | 120 | NM_178439 | Germ cell-less homolog 1 (Drosophila) GCL |
| 48 | 121 | NM_005604 | POU domain, class 3, transcription factor 2 POU3F2 |
| 49 | 122 | NM_004220 | Zinc finger protein 213 ZNF213 |
| 50 | 123 | NM_001010926 | Hairy and enhancer of split 5 (Drosophila) HES5 |
| 51 | 124 | NM_001674 | Activating transcription factor 3 ATF3 |
| 52 | 125 | NM_005804 | DEAD (Asp-Glu-Ala-Asp) box polypeptide 39 DDX39 |
| 53 | 126 | NM_004454 | Ets variant gene 5 (ets-related molecule) ETV5 |
| 54 | 127 | NM_002128 | High-mobility group box 1 HMGB1 |
| 55 | 128 | NM_005078 | Transducin-like enhancer of split 3 (E(sp1) homolog, Drosophila) TLE3 |
| 56 | 129 | NM_006237 | POU domain, class 4, transcription factor 1 POU4F1 |
| 57 | 130 | NM_014819 | Praja 2, RING-H2 motif containing PJA2 |
| 58 | 131 | NM_003687 | PDZ and LIM domain 4 PDLIM4 |
| 59 | 132 | NM_005230 | ELK3, ETS-domain protein (SRF accessory protein 2) ELK3 |
| 60 | 133 | NM_006706 | Transcription elongation regulator 1 TCERG1 |
| 61 | 134 | NM_030751 | Transcription factor 8 (represses interleukin 2 expression) TCF8 |
| 62 | 135 | NM_003305 | Transient receptor potential cation channel, subfamily C, member 3 TRPC3 |
| 63 | 136 | NM_145805 | ISL2 transcription factor, LIM/homeodomain, (islet-2) ISL2 |
| 64 | 137 | NM_004235 | Kruppel-like factor 4 (gut) KLF4 |
| 65 | 138 | NM_006963 | Zinc finger protein 22 (KOX 15) ZNF22 |
| 66 | 139 | NM_002746 | Mitogen-activated protein kinase 3 MAPK3 |
| 67 | 140 | NM_004089 | TSC22 domain family 3 DSIPI |
| 68 | 141 | NM_000321 | Retinoblastoma 1 (including osteosarcoma) RB1 |
| 69 | 142 | NM_000842 | Glutamate receptor, metabotropic 5 GRM5 |
| 70 | 143 | NM_005955 | Metal-regulatory transcription factor 1 MTF1 |
| 71 | 144 | NM_139276 | Signal transducer and activator of transcription 3 (acute-phase response factor) STAT3 |
| 72 | 145 | NM_006569 | Cell growth regulator with EF hand domain 1 CGREF1 |
| 73 | 146 | NM_005384 | Nuclear factor, interleukin 3 regulated NFIL3 |

### APPENDIX B

Hu, Y., Leaver, S. G., Plant, G. W., Hendriks, W. T. J., Niclou, S. P., Verhaagen, J., Harvey, A. R., Ciu, Q., (2005) Lentiviral vector-mediated transfer of ciliary neurotrophic factor to Schwann cells within reconstructed peripheral nerve grafts enhances survival and axonal regeneration of adult retinal ganglion cells, Molec. Therapy. In press.
Ruitenberg, M. J., Levinson, D. B., Voon Lee, S., Verhaagen, J., Harvey, A. R., Plant, G. W., (2005) NT-3 expression from engineered olfactory ensheathing glia promotes spinal sparing and regeneration. Brain, In press.
Willis D, Li K W, Zheng J Q, Chang J H, Smit A B, Kelly T, Merianda T T, Sylvester J, van Minnen J, Twiss J L. (2005) Differential transport and local translation of cytoskeletal, injury-response, and neurodegeneration protein mRNAs in axons. J. Neurosci. 25(4):778-91.
Jimenez C R, Stam F J, Li K W, Gouwenberg Y, Homshaw M P, De Winter F, Verhaagen J, Smit A B. (2005) Proteomics of the injured rat sciatic nerve reveals protein expression dynamics during regeneration. Mol Cell Proteomics. 4: 120-32.
Spijker S, Houtzager S W, De Gunst M C, De Boer W P, Schoffelmeer A N, Smit A B. (2004) Morphine exposure and abstinence define specific stages of gene expression in the rat nucleus accumbens. FASEB J. 18: 848-50.
Hendriks, W. T. J., Ruitenberg, M. J., Boer, G. J., Verhaagen, J. (2004) Viral vector-mediated gene transfer of neurotrophins to promote regeneration of the injured spinal cord. Progr. Brain Res. 146:451-76.
Ruitenberg, M. J., Blits, B., Dijkhuizen, P. A., te Beek, E. T., Bakker, A., van Heerikhuize, J. J., Pool, C. W., Hermens, W. T. J., Boer, G. J., Verhaagen, J. (2004) Adeno-associated viral vector-mediated gene transfer of brain-derived neurotrophic factor reverses atrophy of rubrospinal neurons following both acute and chronic spinal cord injury. Neurobiol. of Disease, 15: 394-406.
Ahmed, B., Chakravarthy, S., Eggers, R., Hermens, W. T. J. M. C., Zhang, J. Y., Niclou, S. P., Lévelt, C., Sablitzky, F., Anderson, P. N., Lieberman, A. R., Verhaagen, J. (2004) Efficient delivery of Cre-recombinase to neurons in vivo and stable transduction of neurons using adeno-associated and lentiviral vectors. BMC Neuroscience 5: 4.
Blits, B., Carlstedt, T. P., Ruitenberg, M. J., de Winter, F., Hermens, W. T. J. M. C., Dijkhuizen, P. A., Claasens, J. W. C., Eggers, R., van der Sluis, R., Tenenbaum, L., Boer, G. J., and Verhaagen, J., Rescue and sprouting of motoneurons following ventral root avulsion and reimplantation combined with intraspinal adeno-associated viral vector-mediated expression of glial cell line-derived neurotrophic factor or brain-derived neurotrophic factor., Exp. Neurol. 189 (2004) 303-16.
Blits, B., Oudega, M., Boer, G. J., Bunge, M., Verhaagen, J. (2003) Adeno-associated viral vector-mediated neurotrophin gene transfer improves hindlimb function in the injured adults rat spinal cord. Neuroscience 118: 271-81.
Ruitenberg, M. J., Plant, G. W., Hamers, F. P. T., Wortel, J., Blits, B., Dijkhuizen, P. A., Gispen, W. H., Boer, G. J., Verhaagen, J. (2003) Ex vivo adenoviral vector-mediated neurotrophin gene transfer to olfactory ensheathing glia: effects on rubrospinal tract regeneration, lesion size and functional recovery following implantation in the injured rat spinal cord. J. Neurosci. 23: 7045-58.
Harvey, A. R., Kamphuis, W., Eggers, R., Symons, N. A., Blits, B., Niclou, S., Boer, G. J., Verhaagen, J. (2002) Intravitreal injection of adeno-associated viral vectors results in the transduction of different types of retinal neurons in neonatal and adult rats: a comparison with lentiviral vectors. Mol. Cell. Neurosci. 21: 141-57.
Blits, B., Boer, G. J., Verhaagen, J. (2002) Pharmacological, cell, and gene therapy strategies to promote spinal cord regeneration. Cell Transplantation 11: 593-613.
Eaton, M., Blits, B., Ruitenberg, M. J., Verhaagen, J., Oudega, M. (2002). Amelioration of chronic neuropathic pain after partial nerve injury by adeno-associated viral (AAV) vector-mediated over-expression of BDNF in the rat spinal cord. Gene Therapy 9: 1387-95.
Ruitenberg, M. J., Boer, G. J., Verhaagen, J. (2002). Adeno-associated viral vectors as agents for gene delivery: towards application in disorders and trauma of the central nervous system. Methods 28: 182-94.
Ruitenberg, M. J. Plant, G. W., Christensen, C. L., Blits, B., Niclou, S., Harvey, A. R., Boer, G. J., Verhaagen, J. (2002) Viral vector-mediated expression in olfactory ensheathing glia implants in the lesioned rat spinal cord. Gene Therapy 9: 135-46.
Blits, B., Dijkhuizen, P. A., Hermens, W. T. J. M. C. Hermens, Esseveldt, L. E., Boer, G. J., Verhaagen, J. (2000). Use of adenoviral vectors and ex vivo transduced neurotransplants: towards promotion of neuroregeneration. Cell Transplantation 9: 169-78.
Blits, B., Dijkhuizen, P. A., Boer, G. J., Verhaagen, J. (2000) Intercostal nerve implants transduced with an adenoviral vector encoding neurotrophin-3 promote regrowth of injured rat corticospinal tract fibers and improve hindlimb function. Exp. Neurol. 164: 25-37.
Dijkhuizen, P. A. and Verhaagen, J. (1999) The use of neurotrophic factors to treat spinal cord injury: advantages and disadvantages of different delivery methods. Neurosci. Res. Comm. 24: 1-10.
Hermens, W. T. J. M. C., Ter Brake, O., Dijkhuizen, P. A., Sonnemans, M. A. F., Grimm, D., Kleinschmidt, A. and Verhaagen, J. (1999) Purification of recombinant adeno-associated virus by iodixanol gradient ultracentrifugation allows rapid and reproducible preparation of vector stocks for gene transfer in the nervous system. Human Gene Therapy 10: 1885-91.
Blits, B., Dijkhuizen, P. A., Carlstedt, T. P., Poldervaart, H., Schiemanck, S., Boer, G. J. and Verhaagen, J. (1999) Adenoviral vector-mediated expression of a foreign gene in peripheral nerve tissue bridges implanted in the injured peripheral and central nervous system. Exp. Neurol. 160: 256-67.
Hermens, W. T. J. M. C. and Verhaagen, J. (1998) Viral vectors, tools for gene transfer in the nervous system. Prog. in Neurobiol. 55: 399-432.
Dijkhuizen, P. A., Pasterkamp, R. J., Hermens, W. T. J. M. C., De Winter, F., Giger, R. J., Verhaagen, J. (1998) Adenoviral vector mediated gene transfer to injured rat peripheral nerve. J. Neurotrauma 15: 387-97.
Zhang, Y., Dijkhuizen, P. A., Anderson, P. N., Liebermann, A. R., Verhaagen, J. (1998) Neurotrophin-3 delivered via an adenoviral vector promotes the regeneration of dorsal root afferents into spinal cord of adult rats. J. Neurosci. Res. 54: 554-62.
Hermens, W. T. J. M. C., Giger, R. J., Holtmaat, A. J. G. D., Dijkhuizen, P. A., Houweling, D., Verhaagen, J. (1997) Transient gene transfer to neurons and glia: analysis of adenoviral vector performance in the CNS and PNS. J. Neurosci. Meth. 71: 85-98.
Holtmaat, A. J. G. D., Hermens, W. T. J. M. C., Sonnemans, M. A. F., Giger, R. J., Van Leeuwen, F. W., Kaplitt, M. G., Oestreicher, A. B., Gispen, W. H., Verhaagen, J. (1997) Adenoviral vector-mediated expression of B-50/GAP-43 induces alterations in the membrane organization of olfactory axon terminals in vivo. J. Neurosci. 17: 6575-86.
Dijkhuizen, P. A., Hermens, W. T. J. M. C., Teunis, M., Verhaagen, J. (1997) Adenoviral vector directed expression of neurotrophin-3 in rat dorsal ganglion explants results in a robust neurite outgrowth response. J. Neurobiol. 33: 172-84.
Buffo, A., Holtmaat, A. J. G. D., Savio, T., Verbeek, S., Oberdick, J. O., Oestreicher, A. B., Gispen, W. H., Verhaagen, J., Rossi, F., Strata, P. (1997) Targeted overexpression of the neurite growth-associated protein B-50/GAP-43 in cerebellar Purkinje cells induces sprouting in response to axotomy, but does not allow axon regeneration into growth permissive transplants. J. Neurosci. 17: 8778-91.
Holtmaat, A. J. G. D., Dijkhuizen, P. A., Oestreicher, A. B., Romijn, H. J., Van der Lugt, N. M. T., Berns, A., Margolis, F. L., Gispen, W. H. and Verhaagen, J. (1995) Directed expression of the growth-associated protein B-50/GAP-43 to olfactory neurons in transgenic mice results in changes in axon morphology and extraglomerular fiber growth. J. Neurosci. 15: 7953-65.

1. A method for promoting generation or regeneration of a neuronal cell, the method comprising administering a nucleic acid comprising (i) a nucleotide sequence of any of SEQ ID NO: 1-146 or a fragment thereof or (ii) a nucleotide sequence antisense to any of SEQ ID NO: 1-146 or a fragment thereof to a subject in an amount effective to alter the activity or the steady state level of a polypeptide in the neuronal cell, wherein the polypeptide comprises an amino acid sequence that is encoded by a nucleotide sequence selected from:
(a) a nucleotide sequence that has at least 80% identity with a sequence selected from SEQ ID NO: 1-146; and,
(b) a nucleotide sequence that encodes an amino acid sequence that has at least 80% amino acid identity with an amino acid sequence encoded by a nucleotide sequence selected from SEQ ID NO: 1-146.

2. The method according to claim 1, wherein the activity or steady-state level of the polypeptide is altered by regulating the expression level of a nucleotide sequence encoding the polypeptide in the neuronal cell.

3. The method according to claim 1, wherein regeneration of the neuronal cell is promoted by increasing the activity or the steady-state level of a polypeptide encoded by a nucleotide sequence selected from:
(a) a nucleotide sequence that has at least 80% identity with a sequence selected from SEQ ID NO: 23, 35, 45, 65, 14, 32, 43, 72, 11, 12, 28, 8, 51, 4, 3, 42, 48, 52, 61, 5, 6, 10, 17, 49, 55, 58, 64, 70, 73, 27, 40, 96, 108, 118, 138, 87, 105, 116, 145, 84, 85, 101, 81, 124, 77, 76, 115, 121, 125, 134, 78, 79, 83, 90, 122, 128, 131, 137, 143, 146, 100, and 113; and,
(b) a nucleotide sequence that encodes an amino acid sequence that has at least 80% amino acid identity with an amino acid sequence encoded by a nucleotide sequence selected from SEQ ID NO: 23, 35, 45, 65, 14, 32, 43, 72, 11, 12, 28, 8, 51, 4, 3, 42, 48, 52, 61, 5, 6, 10, 17, 49, 55, 58, 64, 70, 73, 27, 40, 96, 108, 118, 138, 87, 105, 116, 145, 84, 85, 101, 81, 124, 77, 76, 115, 121, 125, 134, 78, 79, 83, 90, 122, 128, 131, 137, 143, 146, 100, and 113.

4. The method according to claim 3, wherein the activity or the steady-state level of the polypeptide is increased by introducing a nucleic acid into the neuronal cell, wherein the nucleic acid comprises a nucleotide sequence encoding the polypeptide, and wherein the nucleotide sequence is under control of a promoter capable of driving expression of the nucleotide sequence in the neuronal cell.

5. The method according to claim 1, wherein regeneration of the neuronal cell is promoted by decreasing the activity or the steady-state level of a polypeptide encoded by a nucleotide sequence selected from:
(a) a nucleotide sequence that has at least 80% identity with a sequence selected from SEQ ID NO: 31, 69, 53, 18, 21, 26, 30, 36, 39, 44, 46, 60, 66, 71, 13, 19, 25, 47, 24, 9, 1, 20, 22, 63, 67, 2, 7, 15, 16, 34, 37, 38, 41, 50, 54, 56, 57, 62, 68, 29, 59, 33, 104, 142, 126, 91, 94, 99, 103, 109, 112, 117, 119, 133, 139, 144, 86, 92, 98, 120, 97, 82, 74, 93, 95, 136, 140, 75, 80, 88, 89, 107, 110, 111, 114, 123, 127, 129, 130, 135, 141, 102, 132, and 105; and,
(b) a nucleotide sequence that encodes an amino acid sequence that has at least 80% amino acid identity with an amino acid sequence encoded by a nucleotide sequence selected from SEQ ID NO: 31, 69, 53, 18, 21, 26, 30, 36, 39, 44, 46, 60, 66, 71, 13, 19, 25, 47, 24, 9, 1, 20, 22, 63, 67, 2, 7, 15, 16, 34, 37, 38, 41, 50, 54, 56, 57, 62, 68, 29, 59, 33, 104, 142, 126, 91, 94, 99, 103, 109, 112, 117, 119, 133, 139, 144, 86, 92, 98, 120, 97, 82, 74, 93, 95, 136, 140, 75, 80, 88, 89, 107, 110, 111, 114, 123, 127, 129, 130, 135, 141, 102, 132, and 105.

6. The method according to claim 5, wherein the activity or the steady-state level of the polypeptide is decreased by introducing a nucleic acid into the neuronal cell, wherein the nucleic acid comprises an antisense nucleotide sequence that is capable of inhibiting the expression of the nucleotide sequence encoding the polypeptide, and wherein, optionally, the antisense nucleotide sequence is under control of a promoter capable of driving expression of the antisense nucleotide sequence in the neuronal cell.

7. The method according to claim 1, wherein the neuronal cell is a neuronal cell in need of regeneration.

8. The method according to claim 7, wherein the neuronal cell is a cell of the central nervous system, preferably a neuronal cell of the corticospinal tract.

9. The method according to claims 4 or 6, wherein the promoter is neuronal cell specific promoter.

10. The method according to claims 4 or 6, wherein the promoter is a promoter of a gene that encodes an mRNA comprising a sequence selected from SEQ ID NO: 31, 69, 53, 18, 21, 26, 30, 36, 39, 44, 46, 60, 66, 71, 13, 19, 25, 47, 24, 9, 1, 104, 142, 126, 91, 94, 99, 103, 109, 112, 117, 119, 133, 139, 144, 86, 92, 98, 120, 97, 82, and 74.

11. The method of claim 1, wherein the subject is suffering from a neurotraumatic injury or a neurodegenerative disease.

12. The method according to claim 11, wherein the neurotraumatic injury comprises a lesion, avulsion and/or contusion of nerve tissue.

13. The method according to claim 11, wherein the neurotraumatic injury comprises neurons of the central nervous system, preferably a neurons of the corticospinal tract.

14. The method according to claim 11, wherein the neurodegenerative disease is selected from: cerebrovascular accidents (CVA), Alzheimer's disease (AD), vascular-related dementia, Creutzfeldt-Jakob disease (CJD), bovine spongiform encephalopathy (BSE), Parkinson's disease (PD), brain trauma, multiple sclerosis (MS), amyotrophic lateral sclerosis (ALS-Lou Gehrig's disease) and Huntington's chorea.

15. The method according to claim 11, wherein the method comprises the step of administering to the subject a therapeutically effective amount of a pharmaceutical composition comprising a nucleic acid construct as defined in claims 4 or 6.

16. The method according to claims 15, wherein the nucleic acid further comprises a viral gene therapy vector selected from gene therapy vectors based on an adenovirus, an adeno-associated virus (AAV), a herpes virus, a pox virus and a retrovirus.

17. The method according to claim 15, wherein the pharmaceutical composition is administered at a site of neuronal injury or degeneration.

18. A method of promoting regeneration of a neuronal cell in a subject in need thereof comprising administering a composition comprising an effective amount of the nucleic acid of claim 1.

19. A method of treating neurotraumatic injury or neurodegenerative disease in a subject in need thereof comprising administering a composition comprising an effective amount of the nucleic acid of claim 1.

20. A method for diagnosing the status of generation or regeneration of a neuron in a subject, the method comprising the steps of:
(a) determining the expression level of the nucleotide sequence of the polypeptide as defined in claim 1 in the subject's generating or regenerating neuron; and,
(b) comparing the expression level of the nucleotide sequence with a reference value for expression level of the nucleotide sequence, the reference value preferably being the average value for the expression level in a neuron of healthy individuals.

21. The method according to claim 20, wherein the expression level of the nucleotide sequence is determined indirectly by quantifying the amount of the polypeptide.

22. The method according to claim 20, wherein the expression level is determined ex vivo in a sample obtained from the subject.

23. A nucleic acid comprising a nucleotide sequence encoding a polypeptide that comprises an amino acid sequence that is encoded by a nucleotide sequence selected from:
(a) a nucleotide sequence that has at least 80% identity with a sequence selected from SEQ ID NO: 1-146; and,
(b) a nucleotide sequence that encodes an amino acid sequence that has at least 80% amino acid identity with an amino acid sequence encoded by a nucleotide sequence selected from SEQ ID NO: 1-146,
wherein the nucleotide sequence is operably linked to a promoter that is capable of driving expression of the nucleotide sequence in the neuronal cell.

24. The nucleic acid according to claim 23, wherein the nucleotide sequence is selected from:
(a) a nucleotide sequence that has at least 80% identity with a sequence selected from SEQ ID NO: 23, 35, 45, 65, 14, 32, 43, 72, 11, 12, 28, 8, 51, 4, 3, 42, 48, 52, 61, 5, 6, 10, 17, 49, 55, 58, 64, 70, 73, 27, 40, 96, 108, 118, 138, 87, 105, 116, 145, 84, 85, 101, 81, 124, 77, 76, 115, 121, 125, 134, 78, 79, 83, 90, 122, 128, 131, 137, 143, 146, 100, and 113; and,
(b) a nucleotide sequence that encodes an amino acid sequence that has at least 80% amino acid identity with an amino acid sequence encoded by a nucleotide sequence selected from SEQ ID NO: 23, 35, 45, 65, 14, 32, 43, 72, 11, 12, 28, 8, 51, 4, 3, 42, 48, 52, 61, 5, 6, 10, 17, 49, 55, 58, 64, 70, 73, 27, 40, 96, 108, 118, 138, 87, 105, 116, 145, 84, 85, 101, 81, 124, 77, 76, 115, 121, 125, 134, 78, 79, 83, 90, 122, 128, 131, 137, 143, 146, 100, and 113.

25. A nucleic acid comprising a nucleotide sequence encoding an RNAi agent that is capable of inhibiting the expression of a polypeptide that comprises an amino acid sequence that is encoded by a nucleotide sequence selected from:
(a) a nucleotide sequence that has at least 80% identity with a sequence selected from SEQ ID NO: 1-146; and,
(b) a nucleotide sequence that encodes an amino acid sequence that has at least 80% amino acid identity with an amino acid sequence encoded by a nucleotide sequence selected from SEQ ID NO: 1-146,
wherein optionally the nucleotide sequence encoding the RNAi agent is operably linked to a promoter that is capable of driving expression of the nucleotide sequence in the neuronal cell.

26. The nucleic acid according to claim 25, wherein the nucleotide sequence is selected from:
(a) a nucleotide sequence that has at least 80% identity with a sequence selected from SEQ ID NO: 31, 69, 53, 18, 21, 26, 30, 36, 39, 44, 46, 60, 66, 71, 13, 19, 25, 47, 24, 9, 1, 20, 22, 63, 67, 2, 7, 15, 16, 34, 37, 38, 41, 50, 54, 56, 57, 62, 68, 29, 59, 33, 104, 142, 126, 91, 94, 99, 103, 109, 112, 117, 119, 133, 139, 144, 86, 92, 98, 120, 97, 82, 74, 93, 95, 136, 140, 75, 80, 88, 89, 107, 110, 111, 114, 123, 127, 129, 130, 135, 141, 102, 132, and 105; and,
(b) a nucleotide sequence that encodes an amino acid sequence that has at least 80% amino acid identity with an amino acid sequence encoded by a nucleotide sequence selected from SEQ ID NO: 31, 69, 53, 18, 21, 26, 30, 36, 39, 44, 46, 60, 66, 71, 13, 19, 25, 47, 24, 9, 1, 20, 22, 63, 67, 2, 7, 15, 16, 34, 37, 38, 41, 50, 54, 56, 57, 62, 68, 29, 59, 33, 104, 142, 126, 91, 94, 99, 103, 109, 112, 117, 119, 133, 139, 144, 86, 92, 98, 120, 97, 82, 74, 93, 95, 136, 140, 75, 80, 88, 89, 107, 110, 111, 114, 123, 127, 129, 130, 135, 141, 102, 132, and 105.

27. The nucleic acid according to claims 23 or 25, wherein the promoter is a promoter that is specific for a neuronal cell.

28. The nucleic acid according to claim 27, wherein the promoter is selected from a GAP43 promoter, a FGF receptor promoter and a neuron specific enolase promoter.

29. The nucleic acid according to claims 23 or 25, wherein the promoter is a promoter of a gene that encodes an mRNA comprising a sequence selected from SEQ ID NO: 1-146.

30. The nucleic acid according to claim 29, wherein the promoter is a promoter of a gene that encodes an mRNA comprising a sequence selected from SEQ ID NO: 31, 69, 53, 18, 21, 26, 30, 36, 39, 44, 46, 60, 66, 71, 13, 19, 25, 47, 24, 9, 1, 104, 142, 126, 91, 94, 99, 103, 109, 112, 117, 119, 133, 139, 144, 86, 92, 98, 120, 97, 82, and 74.

31. The nucleic acid according to claims 23 or 25, wherein the nucleic acid construct is a viral gene therapy vector selected from gene therapy vectors based on an adenovirus, an adeno-associated virus (AAV), a herpes virus, a pox virus and a retrovirus.

32. A method for identification of a substance capable of promoting regeneration of a neuronal cell comprising the steps of:
(a) providing a test cell population capable of expressing a nucleotide sequence encoding a polypeptide as defined in claim 1;
(b) contacting the test cell population with a substance;
(c) determining the expression level of the nucleotide sequence or the activity or steady state level of the polypeptide in the test cell population contacted with the substance;
(d) comparing the expression, activity or steady state level determined in (c) with the expression, activity or steady state level of the nucleotide sequence or of the polypeptide in a test cell population that is not contacted with the substance; and,
(e) identifying if the substance produces a difference in expression level, activity or steady state level of the nucleotide sequence or the polypeptide, between the test cell population that is contacted with the substance and the test cell population that is not contacted with the substance.

33. The method according to claim 32, whereby the expression levels, activities or steady state levels of more than one nucleotide sequence or more than one polypeptide are compared.

34. The method according to claim 32, whereby the test cell population comprises primary sensoric neurons (e.g. DRG neuronen) or cells of a sensory neuron cell line, preferably primary sensoric neurons (e.g. DRG neuronen), cells of the sensory neuron cell line such as e.g. the F11 cell line.

35. The method according to claim 32, whereby the test cell population comprises mammalian cells, preferably human cells.

36. A substance identified in a method according to claim 32.


SEQUENCE LISTING (146 sequences)

SEQ ID NO: 1 - DNA, 2097 bp, Rattus norvegicus
  misc_feature: >gi|14249139|ref|NM_032616.1| Rattus norvegicus liver-specific bHLH-Zip transcription factor 7 (Lisch7), mRNA
accgctcaccaggtcagttgtccccggaaagccgaaggcatgagcttcgcccaagttctttttatgggttagaactcctccagagcgggggaaaaaggacttggaataggggcgggacggagcacgcacccttctccgccttggttctcgccgcgccccctactctcgggatacttgggaggggacgcgcgggcaccgtcgctgctagacggccgcgatggcgccggcggccggcgcgtgtgctggggcgcctgactcccacccagctaccgtggtcttcgtgtgtctctttctcatcattttctgcccagaccctgccagtgccatccaggtgactgtgtctgacccctaccacgtagtgatcctgttccagccagtgaccctgccctgcacctatcagatgagcaacactctcacagtccccatcgtgatctggaagtacaagtcattctgccgggaccgtattgccgatgccttctctcctgccagtgtggacaaccagctaaatgcccagttggcagctggcaaccccggctacaacccctatgtggagtgccaggacagtgtacgcactgtcagggtggtggccaccaaacagggcaatgcggtgaccctgggagactactaccaaggcaggaggatcaccataacaggaaatgctgacctgaccttcgagcagacagcctggggagacagtggagtgtattactgctctgtggtctcggcccaagatctggatggaaacaacgaggcgtacgcagagctcatcgtccttggcaggacctcagaggcccctgagctcctacctggttttcgggcggggcccttggaagattggctctttgtggtcgtggtctgcctggcgagcctcctcctcttcctcctcctgggcatctgctggtgccagtgctgtcctcacacctgctgctgctatgtccgatgtccctgctgcccagacaagtgctgttgccctgaggctctttatgctgctggcaaagcagccacctcaggtgtcccgagcatctatgcccccagcatctatacccacctctcacctgccaagaccccaccacctccgcctgccatgattcccatgggccctccctatgggtaccctggagactttgacagacatagctcagttggtggccacagctcccaagtacccctgctgcgtgacgtggatggcagtgtatcttcagaagtacgaagtggctacaggatccaggctaaccagcaagatgactccatgagggtcctatactatatggagaaagagctagccaactttgacccttcccgacctggccctcccaatggcagagtggaacgggccatgagtgaagtaacctccctccatgaagatgactggcgatcgaggccttccagggctcctgccctcacccccatcagggatgaggagtggaatcgccactccccacagagtcccagaacatgggagcaggaaccccttcaagaacaaccaaggggtggttgggggtctggacgccctcgggcccgctctgtggatgctctagatgatatcaaccggcctggctccactgaatcaggacggtcttctcccccaagtagtggacggagaggacgggcctatgcacctccaagaagtcgcagccgggatgacctctatgacccggacgatcctagggacttgccacattcccgagatccccactattatgacgacatcaggtctagagatccacgtgctgaccccagatcccgtcagcgatcccgagatcctcgggatgctggcttcaggtcaagggaccctcagtatgatgggcgactattagaagaggctttaaagaaaaaggggtcgggcgagagaaggagggtttacagggaggaagaagaggaagaggagggccaataccccccagcacctccaccttactcagagactgactcgcaggcctcacgggagaggaggctgaaaaagaatttggccctgagtcgggaaagtttagtcgtctgatccacgttttgtatgtagcttttgtactttttttttaattggaatcaatattgatgaaacttcaagcctaataaaatgtctaatcacaaaaaaaaaaa

SEQ ID NO: 2 - DNA, 3030 bp, Rattus norvegicus
  misc_feature: >gi|9507130|ref|NM_019193.1| Rattus norvegicus SRY-box containing gene 10 (Sox10), mRNA
cggacactagactgtagaggtgagaccaaggataggagccctcgggcaacgggaggaagaacatagaatagccttgaagggcagaaaagggggagggggtcttttctcagtggggaagagtgactgagggacttcactacatccagggctgagaccctttcccaggagaggaagagctcagcatggaagtgagacccctccccccaacccaatgtcctcctcacccttcttgaagagtacccactcccctccccctccttccttccccagccgtctgcagcggctcagtcgctcagtcagtctcgggctgtccagccagggtgtttggtggtgaggattcaggctccgtccagacaaggcagtggcctgaggctcagggccccccagcccctccctcccagtccatcagcgtcactccccagccccgagctggaccgcacaccttgggacacggttttccacttcctcaggacgagccccagactggaggagaggtcggaggaggtgggcgttgggctcttcacgaggaccccggcggcgggcccgggggaggcggccgaagcggcggcggccgggagcgacatggccgaggagcaagacctatcagaggtggagctgagccccgtgggctcggaggaacctcgctgcctgtccccaagcagcgcgccgtcgctgggacccgacggcggcggcggtggctcaggcctgcgagccagcccagggcccggcgaattgggcaaggtcaagaaggaacagcaggacggcgaggcagacgatgacaagttcccggtgtgcatccgcgaggcggtcagccaggtgctcagcggctacgactggacgctggtgcccatgcccgtgcgggtcaacggtgccagcaagagcaagccgcacgtcaaacggcccatgaacgcctttatggtgtgggcacaggcggcacgcagaaagttagccgaccagtacccgcacctccacaatgctgagctcagcaagacgctgggcaagctctggaggttgctgaacgagagtgacaagcgccccttcattgaggaggccgagcggctccggatgcagcacaaaaaagaccatccggactacaagtaccaacctcggcggcggaagaatgggaaggcagcccagggggaggcagagtgcccaggcggggagaccgaccaaggaggggctgctgctatccaggctcactacaagagtgcccacctggaccaccggcacccagaagaaggctcccccatgtcagatgggaacccagagcacccctcaggccagagccatggccctcctacccctccaaccaccccaaagacagagctgcagtccggcaaggcagatcccaaaagggatgggcgctccttgggggagggcgggaagccccacatcgacttcggcaatgtggacatcggggagatcagccatgaggtaatgtccaacatggagacctttgatgtgactgagctggaccaatacctgccgcccaatgggcacccaggccacgtgggcagctactcggcagctggctacgggctgagcagtgccctggctgtggccagtggacactctgcctggatctccaagccaccaggtgtggctctgcccacggtctcgccccctgctgtggatgccaaagcccaggtgaagacagagaccacagggccccagggacccccacactacaccgaccagccgtccacttcccagatcgcctacacctccctcagtctgccccactacggctccgccttcccctccatctcacgcccccagtttgactactctgaccatcagccctcaggaccctattatggccacgcaggccaggcctctggcctctactcagctttttcctacatgggcccctctcagcggcccctctacacggccatctctgaccccagcccctcagggccccagtcccacagtcccacacactgggaacagccagtatatacgactctatcccgaccttagaggggccctgtcaccattggtgcccacgtggcccagggccccttcccccagcctgtgtgccctgctcctcatcagcctcatgcccagcagtggcagtggaggaggctgcagaggctgacagcagcaggcaggcttctgccagctcctgccctgatgacctccacccaccctggctcgggaggccaagccctgactgagctggcaaaggaggagtttggtggccatcccagactgagggtgaggggccacccatccccagaaataaccctctatcccaggacctgagaagtcgttctcaagtctttcagggaggcagaaaagggtatggtgggaaacctcactgctcctgttctctcctgggagagcaaggggctcaagggcctgtgtgccactgtgtcactgtgcctaaaagctaggctgcctagggacctgcttcaaagcctggagctggctctgtcctgtcactaaggactcactgaggacagctttgaacagctttgtgaagagcagggtgggacactcaatggacactctgatcctttcacccccctcctcacctgaatgggaactggccgaggtccccagatccagttctgcattagtaacctcatccctcacctaactgaggagagtcctcatgttcttccccacgacctccactcctcaaggcccaccccaggtgaggaggcaggggctccaggaaagaatcagaggtgtccagccttcctctcgccttctgtcagcctcagttagcactgcacgggccctagccccagagcccagtacctgggttgctggcagcaggctggacactaaaccccttccatgtacagtctacctttgcctcgcaccctttgctccagcgatatcttaataaagtacgtagctctgtctccccatctcctgtctgcagcccagtccacacgtaacacatcgctgcccctttatctccacagtcaccatgttcccatccctctagctactccagccccaattaacttgcattaagcactccaagtaataaaatcaaagcttctggttgcctgc

SEQ ID NO: 3 - DNA, 310 bp, Rattus norvegicus
  misc_feature: >gi|14944972|gb|BI288410.1|BI288410 UI-R-DK0- cda-e-11-0-UI.s1UI-R-DK0 Rattus norvegicus cDNA clone UI-R-DK0- cda-e-11-0-UI 3′,mRNA sequence
ttttttttttttttttttgaaggcaaaaaagaaacatttaatgatgaggggcaaagggcaggcaggtgcacaccaggcaggctggaacggggcatgccacctgcagggtatctccagagaagctgtgtccaccaagagcgttgcaggaggttcagggttagccatggccagggctacatcagtacaaggactcagcagaacttccatcgattcccacactcattgcagacaacataggtggtcatgggctcatcagagctacgggtctgcacctgcgtgtangtgcagttcttcttcctgcacttgttgc

SEQ ID NO: 4 - DNA, 853 bp, Rattus norvegicus
  misc_feature: >gi|16924003|ref|NM_057144.1| Rattus norvegicus cysteine-rich protein 3 (Csrp3), mRNA
tcatctcggcagattgactttggcccctgagtcttcaccatgccgaactggggtggaggtgcaaaatgtggagcctgcgacaagacggtttaccatgcagaagaaatccagtgcaatgggaggagcttccataagacctgtttccactgcatggcctgcaggaaggctctggacagcaccacagtggcagctcatgagtcagagatctactgtaaggtctgctatgggcgcaagtatggccccaaggggatcgggtttggacaaggcgctggctgcctcagcacagacaccggcgagcaccttggcctgcagttccaacaatccccaaagccagcacgtgcagccaccacgagcaacccttccaagttctctgcgaagtttggagaatcagagaagtgcccacgatgcggaaagtcggtatatgctgctgagaaggtcatgggaggtggcaagccctggcataagacctgctttccgtgcgccatctgtgggaagagcctggagtccacaaatgtcactgacaaggatggggagctttattgcaaagtttgctatgccaaaaattttggccccacaggcattgggtttggagggcttacacaccaagtggaaaagaaggagtgaaacggcgtgctttctcacagccctgctagtcaacagcagggcttagtgaccccatagatgtaactttggccccgcagcctcctttgtagctggactgcatttctcttcagaagcagtcataggctgcactgaccgtaaaaagagaccaccggaaatgaatctgcaaaagatgcttcattatttactatttttggggtgggagagacaataaacgttttaatggtaaaaa

SEQ ID NO: 5 - DNA, 511 bp, Rattus norvegicus
  misc_feature: >gi|11404978|gb|BF416989.1|BF416989 UI-R-CN0- bkw-e-04-0-UI.s1UI-R-CN0 Rattus norvegicus cDNA clone UI-R-CN0- bkw-e-04-0-UI 3′,mRNA sequence
ttttttttttttttttttgccaggacccaagaagcagtacttttaattgggtgagacccaagaggtgtgcgcacagaccctgtccaatgcacacactgcctctgctgtgacgtccttccggctggaatgccgaggctcctcttggaaggcatgtggggagtgcgtatcagtcccaaacaaaggcacctcccagtgggtggaagttactgggttgcacagtcctgccaagtcctggggtgtcctctggtcactgcttggccttcttcacgatggtacccacggcagaaatgacagtggtcttggagccgctggcactggtggtgacagtgaccactgctggcagggtggctgtggtggtgaggatggtgggctgcgctattgtcgtcacggggatggccgagtcccatttgctcttcctcttctgggcatctgcgacagctgtgctggcagtgctggtggtggtggccgtggcattggttgtggtgcctttcttggtgcctgttacaaactc

SEQ ID NO: 6 - DNA, 678 bp, Rattus norvegicus
  misc_feature: >gi|8501376|gb|BE109271.1|BE109271 UI-R-BS1- azd-h-04-0-UI.s1UI-R-BS1 Rattus norvegicus cDNA clone UI-R-BS1- azd-h-04-0-UI 3′,mRNA sequence
tttttttttttttttttagcatttgattttcctgccaccgaaatgtcactgttttcagttgccttttgtaatcttcacattcatactgcagtttataaagctgatgttgatgagcgagccgctccctttcagtctccttcaagtctgcactgtccttcccagatgagcccggctctaccatgtccaggatatgcagttcgtccagactacagaacttgtcctcggctgcagagtcgtcttgtgcatcacttgtatctgttttcacatatgcactgagtgtgctcatgtctacttgttctgtctccagctggtcgtccttctctaccatatatgaggcctgctcgtcttctgagtccttgtcctttctattactcttagttcttttctttttctttgttttacttttgacctcctgaagatctttaacagattcggttgaagattcactcggtattgaggctacatctttatttatttctgctaactgtatttcagtgtttgatttcacatctatattgtattccatcacaggtggactcaattcttcttcatcattctgtaagtcttctacttctatacgctgactgcctgttattgactgtagagtaattccttgttgatagcaggatacatcctctggatgatttctggtactggtatgcctagataaatggacag

SEQ ID NO: 7 - DNA, 3889 bp, Rattus norvegicus
  misc_feature: >gi|16758827|ref|NM_053949.1| Rattus norvegicus potassium voltage-gated channel, subfamily H (eag-related), member 2 (Kcnh2), mRNA
atgccggtgcggaggggccacgtcgcgccgcagaacaccttcctcgacaccatcatccgcaagtttgagggccagagccgcaagttcatcatcgctaacgctcgtgtagagaactgcgccgtcatctactgcaacgacggcttctgcgaactgtgcggctactctcgggccgaggtgatgcagcggccctgcacctgcgatttcctgcatgggccgcgcacgcagcgccgtgccgccgcgcagatcgcgcaggccctgctgggcgcggaggagcgcaaagtggagatcgccttctaccggaaagatgggagctgcttcctgtgtttggtggatgtggtgcccgtgaagaatgaggatggggctgtcatcatgtttatcctcaactttgaagtggtgatggagaaggacatggtagggtcgccagctcatgacaccaatcacagggggccctctaccagctggctagcttctggccgggccaagaccttccgcctgaagctgcctgccttgttggcactgactgccagggaatcgcccatgcggacaggaagcacgggcagtcctggagcccccggggcagtggtggtggatgtggacctgacgccggcagcacccagcagtgagtccctggccttggatgaggtttctgccatggacaaccatgtggctggacttgggcctgcagaagagaggcgagcactggtgggcccagcgtctgcttcaccggtagccagcatcccaggccctcacccgtcccctcgagctcagagccttaaccctgacgcctctggttccagctgcagtctggcccgaacacgctcacgggagagctgcgctagtgtgcgccgggcgtcttccgcagatgacatcgaggcaatgcgggctggggcgctgcccctcccgccccgacatgctagtacaggggccatgcaccccctgcgcagtggcctgcttaactccacctcagactctgacctcgtgcgctaccgaaccattagcaagataccccaaatcaccctcaactttgtggacctcaaaggcgaccctttcctggcttcacccaccagtgaccgggaaatcatagcacccaagataaaagagcggacccacaatgtcaccgagaaggtcacccaggtcctgtccttgggagccgatgtgcttcctgaatataagctgcaggccccaagaatacaccgctggaccatcctacactacagccccttcaaggcagtgtgggactggctcattctgctgctggtcatctacacagccgtcttcacaccgtactcggctgccttcctgctgaaggagactgaagatgggtcccaagcccctgactgcggctatgcctgccagcctctggctgtagtggacctcctcgtggacatcatgttcattgtggacatccttatcaatttccgcaccacctacgttaacgccaatgaagaggtggtcagccatcctggccgcatcgctgtgcactacttcaagggctggttcctcatcgacatggtggctgccatccccttcgacctgctcatctttggctctggctctgaggagctcatcggactgctgaaaactgccaggctgctgcggctggtgcgagtggctcggaagctggaccgctactcggagtatggggcagcagtgctcttcctgctcatgtgcaccttcgcgctcattgcacactggttggcctgcatctggtacgccatcggcaacatggaacagcctcacatggactcacacattggctggctgcacaacttgggcgaccagatcggcaagccctacaacagcagcggcctgggaggcccctccatcaaggacaagtacgtcacagcgctctacttcaccttcagcagcctcaccagcgtgggctttggcaatgtctctcccaacaccaactcagaaaagatcttctccatctgtgtcatgctcattggctccctcatgtatgccagcatcttcggcaacgtgtccgccatcatccagcggctgtactcgggcacggcccgctaccacacacagatgctccgggtgcgggagtttatccgcttccatcagattcccaacccgctacgccagcgcctcgaggagtacttccagcacgcttggtcctacaccaacggcatcgacatgaatgcggtgctgaagggtttccccgagtgcctgcaggctgacatctgcctacatctaaaccgctcactgctgcagcactgcaaaccattccgaggggccactaaaggctgcctgagggccctggccatgaaattcaagaccacacatgcgccaccaggggacacgctggtgcacgctggggacctgcttactgccctctacttcatctccaggggctccatcgagatcctgcggggtgatgtcgtggtggccatcttggggaagaacgacatctttggagagcctctgaacctgtatgcacgtcctgggaagtccaatggggatgtgagggccctcacttactgtgacctgcacaagatccatcgagacgatctgctggaggtgttggacatgtaccccgagttctcagaccacttttggtctagcctggagatcaccttcaaccttcgagataccaacatgattcctggctcccccagcagtgcggaactagagagcggcttcaacaggcagcgaaagcgcaaactgtccttccgcaggcgtactgacaaggacacagagcagccaggggaggtgtcagccctggggcaaggccctgcccgagttgggccagggccgagttgccggggacagccgggagggccatggggggagagcccatccagtggcccttccagcccagagagcagtgaggatgagggcccaggtcgcagctccagccccctccgcctggtgcccttctccagccccaggcctcccggagactccccaggtggggagcctttgacagaggatggagagaaaagcagtgacacttgtaatcccctgtcaggtgccttctctggggtgtccaatattttcagcttctggggggacagtcgggggcgccagtaccaggagctgcctcgatgccctgccccggcccctagcctcctcaacatccccttgtctagccctggccggcgttcccggggtgacgtggagagcaggctggatgcactccagagacagctgaacaggctggaaacccggctaagtgcagacatggccactgtcctacagctgctacagaggcaaatgaccctggtccctcctgcctacagtgctgtgaccacccctgggcctggccccacttccacgtcccctttgttgcctgtcggccccgtccccactctcaccctggactcgctttctcaggtttcccagttcgtggcgtttgaggagcttcctgcaggagccccagagctcccccaagatggccccactcgacgcctctccctgccgggccagctgggggccctcacctcccagcccctgcacagacatggctcagatccaggcagttagtgacgctgcccggtgtggacacgtggctcaccctggggtcaggggattacctgaccgctctccttggagaccctggtttgaaggggagaacagtgcaggagcacctcctcccccgcccttgggaccagcatctcctcctgcaataccctgggccccactgggaggggcaggggcagagctgggcagtggacagggggtctgtggtccccccactgccctgggggcattagctggtctaactgcccggaggcacccggccctgggccttaggcacctcaaggacttttctgctatttactgctcttattgttaaggataataattaaggatcatatgaataattaatgaagatgctgatgactatgaataataaataattatcctgaggaaaaaaaaa

SEQ ID NO: 8 - DNA, 1749 bp, Rattus norvegicus
  misc_feature: >gi|7106247|ref|NM_013220.1| Rattus norvegicus ankyrin repeat domain 1 (cardiac muscle) (Ankrd1), mRNA
cccgggcaggtccaggggttcagccacaagagggaaaaacatagactaacggctgccaacatgatggtttttcgagtagaggagctggtaacgggcaaaaagaacagcaatgggtcctcaggggagttccttcctggcgagttcagaaatggagagtatgaagctgctgttgccttggagaaacaagaggacttgaagacacttccagccaacagtgtgaacctgggggaagaacaacggaaaagtgagaaagttcgagaggcagagctcaaaaagaaaaaactagaacaaagatcaaagcttgaaaacttagaagaccttgaaataattgttcaactgaagaaaagaaaaaaatacaagaaaactaaagttccagttgtgaaggaaccggagcctgaaattattactgaacctgtggatgtgccaaggtttctgaaagctgcactggagaacaaactgccagttgtagagaaattcctgtcagacaagaacagccccgacgtctgcgatgagtataaacggaccgctctccatagagcatgcttagaaggacacttggcgatcgtggagaagttaatggaggctggagcccagattgaattccgagatatgctggaatccacagccatccactgggcatgtcgtggaggaaacctggatgtcctgaaactgttgctaaacaaaggtgccaaaatcagtgcccgagacaagctgctcagcacagcgctgcacgtggcggtgaggaccggtcactacgagtgtgctgagcacctcatcgcctgcgaggcggatctcaatgccaaggacagagaaggagacacccctctgcatgatgcggtgaggttgaatcgctacaagatgatccggctcttgatgaccttcggtgcggacctcaatgtcaagaactgtgctgggaagatccctatggatctggtgttgcactggcagaatggaaccaaagcgatattcgacagcctcaaggagaatgcctacaaaaactcgcgcatagctacgttctgaggaaagagactcgacaggagctgttctcagccgttttcaaaacatttcccagcagcggagcaaccagctataacacccagcttttgtttgccagccaaatagggagataagctttaaagacgcttcatggaaggaccctgggttagagatgctaacatgtcctctagcgatgctcaccatttttatctgctcctatttatttatttatttatttcatagcactggtagtcaggttttcatgagcatcattttgggggagcaagacttaatctgtgtgtaacattcggagtcttcccgtggctctaaaaccgcgtgggtgaatggtgtctaatctcccttttgtgtcctcacttgcattaaagctactcaaggtatttttgaaagccaagggttggtcggtcgctgcagcccttctctggattctcactgctgatatccgaatgaagcagcagaaggtgtacaggaatgcagaaggggatgggagcagttctgcaggacctaagccgtggctcgccaggcacccacaggtgtggtttgccggatcacacagtgccgaggtgaaggccacgtcaggagcgtgtgtggagggggatggagggagtaggcaatggggcctcactaacctgaggtcgatgagaggaaaaagtccaaaaggacatgccctgtaaatgtgtaaatggtattgtgttgtatgcatattttatattcctaataaatgcaaactaattc

SEQ ID NO: 9 - DNA, 2154 bp, Rattus norvegicus
  misc_feature: >gi|31543339|ref|NM_019328.2| Rattus norvegicus nuclear receptor subfamily 4, group A, member 2 (Nr4a2), mRNA
cggcacgagccagggccagtccgccccgcgctccgcacggctctgcgtccctctctcctgcccagccggctgcctccctctcctgcggccgggctggctgcgtgtggctctccgcgccccgctcccgcagcgctccagcggaccccggctcctctgctcccggaggaactgcacttcggcggagttgaatgaatgaagagagcggagaaggagatctgacgggctggatccccaaattgctttttaaaaaatatcttggaaactttgtcctttgctgaattacgacactgtccacctttaatttcctcgaaaactccaatcactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcgggtcggtttactacaagccctcttcgcccccgacacccagcaccccgggcttccaggtacagcatagcccgatgtgggacgatccgggctcccttcacaacttccaccagaactacgtggccactacgcatatgatcgagcagaggaagacacctgtctcccgcctttcactcttctcctttaagcagtcgcccccgggcactcctgtgtctagctgccagatgcgctttgacgggcctctgcacgtccccatgaacccggagcccgcgggcagccaccacgtagtggatgggcagaccttcgccgtgcccaatcccattcgcaagccggcatccatgggcttcccgggcctgcagatcggccacgcgtcgcagttgcttgacacgcaggtgccctcgccgccgtcccggggctctccctccaatgagggtctgtgcgctgtttgcggtgacaacgcggcctgtcagcattacggtgttcgcacttgtgagggctgcaaaggtttctttaagcgcacggtgcaaaaaaacgcgaaatatgtgtgtttagcaaataaaaattgcccagtggataagcgccgccgaaatcgttgtcagtactgtcggtttcagaagtgcctggctgttgggatggttaaagaagtggttcgcacggacagtttaaaaggccggagaggtcgtctaccctcaaaaccgaagagcccacaggatccctctcccccctcacctccggtgagtctgatcagtgccctcgtcagagcccacgtcgactccaatccggcaatgaccagcctggactattccaggttccaggcaaaccctgactatcagatgagtggagatgatactcaacatatccagcagttctacgatctcctgactggctctatggagatcatcagagggtgggcagagaagattcctggctttgctgacctgcccaaagccgatcaggacctgctttttgaatcagctttcttagaattatttgttctacgcttagcatacaggtccaacccagtggagggtaaactcatcttttgcaatggggtggtcctgcacaggttgcaatgcgtgcgtggctttggggaatggattgattccattgttgaattctcctccaacttgcagaatatgaacatcgacatttctgccttctcctgcattgctgccctggctatggtcacagagagacacgggctcaaggaacccaagagagtggaagagctacaaaacaaaattgtaaattgtcttaaagaccatgtgactttcaataatgggggattgaaccgacccaactacctgtccaaactgttggggaagctcccagaacttcgcaccctttgcacacaggggctccagcgcattttctacctgaaattggaagacttggtaccaccaccagcaataattgacaaacttttcctggacaccttacctttctaagactttctcccatgcacgtcaa

SEQ ID NO: 10 - DNA, 1789 bp, Rattus norvegicus
  misc_feature: >gi|9506974|ref|NM_019334.1| Rattus norvegicus paired-like homeodomain transcription factor 2 (Pitx2), mRNA
ggaattcggcacgagagagagagtgcgagaccgagagagaaagacggagagcagcagacagaaactgcccgcacccgctagctttagcagccctcagcgtggaccctctggcaacttggcaccctcaagatccctgcggttccaaccagacccgctccacggcgctggctgtgccagcccgagcctcggcggcctggcagtcaccctgggaagcggtgggtcggggagacagccgttctctctggtagccgataacggggaatggagaccaattgtcgcaaactagtgtcggcctgtgtgcaattagagaaagataagggccagcagggaaagaatgaggatgtgggcgcagaggacccgtccaagaagaagcggcaacgccggcagaggactcactttactagtcagcagctccaggagctggaagccactttccagagaaaccgctacccagacatgtccactcgcgaagaaatcgccgtgtggaccaaccttacggaagcccgagtccgggtttggttcaagaatcgccgggccaaatggagaaagcgggagcgcaaccagcaggccgagctgtgtaagaatggcttcgggccgcagttcaatgggctcatgcagccgtacgatgacatgtaccccggctactcgtacaacaattgggctgccaagggcctcacgtcagcgtctctgtccaccaagagcttccccttcttcaactctatgaacgtcaatcccctatcctcccagagcatgttttccccgcccaactccatctcatccatgagtatgtcgtccagcatggtgccgtccgcggtgaccggcgtcccgggctccagcctcaatagcctgaataacttgaacaacctgagcagcccgtcgctgaattccgcggtgcccacgcccgcctgtccttacgcgccgccgactcctccgtacgtttatagggacacatgtaactcgagcctggccagtctgagactaaaagcaaagcaacactccagcttcggctacgccagcgtgcagaacccggcctccaacctgagcgcttgccagtatgcggtcgaccggccggtgtgaagcccagggcgcgggattccgaggactgtcggagtgggcaactctgcccgagaaagactgagaattgtgctagaaggtcgtgcgcactacggggagaaagagggggggacaaaagagatcagaggaaaagaaaccactgaattcaaagagagagagcgcctttgatttcaaaggaatgcccccaagtgtctgccatcttccgctgaaagtattcccaacagttggaggacgcgtgcaccaacaaatgtttgactggatatgacattttaacattactataagcttgttattttttaagtttagcattgttaacattaaaatgactgaaaggatgtatatatatcgaaatgtcaaattaattttataaaagcagttgttagtactctcacgacagtgtttttaaaggctaggctttaaaataaagcatgttatccagaatcagttaggatttttcgcttgcagcaaaggaatgtatatactaaatgccacactgtatgtttctaacatattattattataaaaaatgtgtgaatataagttttagagtagtttctctggtggatgccttgtttctgaaactgctacgtacaacctatcctgtgtataacatttcgtacgatattgttttacttttcagcaaatatgaaaaaatgtgttttatttcttgggagtaaaatataccgcatacaaaaaaaaaaaaaaaaaactcgag

SEQ ID NO: 11 - DNA, 425 bp, Rattus norvegicus
  misc_feature: >gi|13891373|gb|BG669451.1|BG669451 DRNAAFE08 Rat DRG Library Rattus norvegicus cDNA clone DRNAAFE08 5′, mRNA sequence
gtttccagctccgagccccatgacatcttgagtgaccttgaactggacactgtcccctttaagggcctcatttccacttctggactaaagaccaggaggctattttctccacggtgctaagttgacatggatccgctcagagcccagcaactggcagcggagctggaggtggagatgatggccgacatgtacaacagaatgaccagtgcctgccaccggaagtgcgtgccttcccactacaaggaggcagagctgtccaaaggcgagtctgtgtgtctggaccgatgcgtatccaagtacttggacatccatgagaggatgggaaaaaagttgacagagttgtctatgcaggatgaagagctgatgaagcgggtacagcagagctctgggcctgcatgaggccccgggccatgtacaccccgggt

SEQ ID NO: 12 - DNA, 3166 bp, Rattus norvegicus
  misc_feature: >gi|13928793|ref|NM_031586.1| Rattus norvegicus immunoglobulin mu binding protein 2 (Ighmbp2), mRNA
atggcctcgtacaccgtggagagttttgtggcccagcagctacagctgttggagctagagcgggacgccgaggtggaggagcgcaggtcctggcaggaacacagttctctgaaagagcttcagagccgaggggtgtgtttgctgaagcttcaggtatcgggccagcgcaccgggttgtatggacagcggctggtcacctttgagcccaggaagtttgggcctgcagtggtgcttcccagcaacagcttcacctctggtgatatcgtgggtctgtatgatactaatgaaagcagccaactggccactggggtcctgacccgcatcacccaaaaatcggtcatagtggcctttgatgagtcccatgatttccagttgaacctggaccgagaaaatacctacagactgctgaagcttgccaatgacgtcacctacaaacgcctgaaaaaagctctgctgacactgaagaagtaccattctgggccagcatcctcgctcattgatgtcctgttgggtggctccacccccagtcctgccactgaaatacccccgctcactttctacaacacgaccctggacccttcccagaaagaagctgtgtcttttgcgctggcgcagaaagaagttgccatcatccatgggcctcctggcactgggaaaaccacaactgtggtggaaataatccttcaagctgtgaagcaaggcttaaaggttctatgctgtgctccctccaacatcgctgtggacaacctggtggagcgtctggctctgtgcaagaagcagattcttcgcctgggtcaccccgcccgcctcctggagtctgttcagcagcactcactggacgcagtgctagcacgcagtgacaatgcccagattgttgctgacatcagaagggacattgaccaggtctttggcaagaacaaaaagacccaagataagagagaaaaaagtaattttcgaaatgaaattaagctgctaaggaaggaactgaaggaaagggaagaagcagccatagttcagagcctcagtgcagcagatgtggttctagccaccaacacaggtgcatctactgatggccccctgaagctgctgcctgaggactactttgatgtggtggtggtggacgagtgcgcccaggccctagaagccagctgctggattcccctgctgaaggcccctaagtgcatcctagctggagaccacaaacagctgccacccaccactgtctctcacaaggcagcactggctgggctgtcccgcagcctgatggagcgtctggcagagaagcatggtgctgctgtggtaaggatgctggcggtccagtaccgaatgcaccaggccatcacgcgctgggcctcggaagccatgtaccacggacagctcactgcccatccctctgtggcaggacaccttctgaaggacctcccaggtgtggctgacacagaggagacgagtgtccctctgctgctcatagacacagctggctgcgggctgctggagctggaggaggaagacagccagtccaagggaaaccccggtgaagttcgcctcgtcactttgcacatccaggctctggtggatgctggggtccaggctggtgacattgccgtcatcgcaccctacaaccttcaggtggatctgctcagacagagcctctctaacaagcaccctgagctggagatcaagtctgttgacggctttcaaggccgagagaaggaggctgtgatcctgacctttgtcaggtccaacaggaaaggtgaagttggttttctggctgaggacaggcggatcaatgttgctgtcacccgtgctaggcggcatgtggcggtcatctgtgattcccacactgtcaacaaccacgcctttttgaagaccttggtggattatttcacagagcatggggaggtacgcacagcctttgagtacctggatgacatcgtccctgagaactatacccatgagggctcccggagccacagttgtgcccccaaacccaagtgccccaccacctcagtcagaaagcctgccagtgctcaggagagtagacaagaggccagagcagccactggacacagccgcaggaagccaagtgagaagcccctaggctctcaagtccagccccagcacagctccaaagcaaatggctctgaccgaactggaggcacagaccggacagagcactttagggctatgattgaggagttcgtggctagcaaggaggcccagttggagtttcccacatccctgagctcccatgacagacttcgagtccaccaattagctgaggagttcgggctgaagcatgacagcaccggggaggggaaggcacggcacatcacagtgagcaggaggagccctgctggttctggcagtgcaaccccacaacctccctcaccgcccagccctgcacaggctgagcctgagcctcaggtagagcagcctgtaggacagccacatggctccacacagctggatctgaaggcacttcacctggagaggctgcagcgacagcaaggctgccaggcccagtctcagctgggcgggggttcgaggccacagaaggctccacagaagaaaaagaaaaaagaaccgaaaggcccagccatggctctgccctctgaggaggacttcgatgccctagtgtcagctgtggtgaaggctgacaacacctgtagcttcaccaagtgctcggccagcaccaccacgctgggccagttctgcatgcactgtagccgccgctattgcctcagccaccatctgcccgagatccatggctgtggtgaaaaggctcgtgcccatgcccggcagaggattagccgggaaggagtactctacgcaggcagtgggaccaaggacagggccctggacccagccaagagggcccagctgcagaggaaactggacaagaagctgggcgagctcagcagccagaggacaagcaagaagaaggagaaggagagggggacgtgaggccacaccctcggggagggctgtgtgggcgagagtcagcaggtgggacacagtcctacactggctatttctctactcgagctagtgtgtccctaactctctcatggcaaggagacaagcctagttaccatcagagtaactgccttgaccggccaggtctcattaacgctgtgagtgccttgcatggccagctagccat

SEQ ID NO: 13 - DNA, 623 bp, Rattus norvegicus
  misc_feature: >gi|14977273|gb|BI300993.1|BI300993 UI-R-DK0- cfk-f-11-0-UI.s2UI-R-DK0 Rattus norvegicus cDNA clone UI-R-DK0- cfk-f-11-0-UI 3′,mRNA sequence
ttttttttttttttttttaatgttggtaattcctactaagccttgaaaattgaaaacagaccccacttccgggtgaagcgctcagacaaaaccccacgaggacactatttgtctccaacgacagacagcacttgggatcatgaggttagcacctatccctcaccttccaggtttggtgactcctctgtcatccccaggcagctgaacagcatcaatagcctcctccaggtgcagcaggatcatcttcaactttgcgatgtcttccagctccatgttgagacctgagctggacgtgtctataatgaactcctgttggagttcaccatgctgagccacccgggcttgctcctccaaaacctggttgataacctccagtcctgaagccacatctcggggagtcaagtcaaaggaggctgactcctcacatatcttttccacattgtgagcttcatcaaagattacaaccgttcccttcaggtcaatgttgtgtgccttacgactctgtaagtgtgcagagagaaatcgtgacatccgcctccatcttttctaggctgctcttcccctcttgctccggtctcctctcctctctaaaacttttctcctgcccatccttccttctcgtt

SEQ ID NO: 14 - DNA, 1207 bp, Rattus norvegicus
  misc_feature: >gi|16758963|ref|NM_053310.1| Rattus norvegicus homer homolog 3 (Drosophila) (Homer3), mRNA
tgactcctgtcacccctctgggtgtcccttagaggtcatccagcccagcctgaccaatgtccacagccagggaacagccaatcttcagcaccagggcgcacgtgttccagatcgaccccgctacgaagaggaactggattcccgctggcaagcatgcacttaccgtgtcctatttctatgatgcaacccgaaatgtgtaccgcatcatcagcatcggaggcgccaaggccatcatcaacagcacagtcactcccaacatgaccttcaccaaaacctctcagaagttcgggcaatgggcagacagtcgagccaacactgtgtatggcctaggctttggctcggaacagcagctgacccagtttgctgagaagtttcaggaggtcaaagaagctgccagactggctcgggagaaatctcaagatggtggagaactcactagtactgccctggccatggcctcccaccaggttcctccaagccccttggtcagcaccaatggtccagaagagaagctgttccgcagccagagcgcggacgcccctggccccactgagcgggagcggttgaagaagatgctgtcagaaggctccgtaggggaggtccagtgggaggctgagttctttgcacttcaggacagcaaccagaggctggcaggggcccttcgagaggccaacgcggccgccactcagtggaggcagcagctggaggtccagcgtgcagaggctgaacacttgcggcagcgggtggctgagctggaggcccaggtggctgcagagccggtccgggcaggagagaaagaagcaaccagccagtcggtggagcagctggaggctcgggtgcagaccaaggaccaggagatccagactttgaagaatcagagcactggcccacgagaggctcctgacaccgccgagcgggaggagacacagcagcaggttcaggacctggagacccggaatgcagaactggagcagcagctgcgggcgatggaaagcaacctggaggaggcgcgggccgagcgggagcgcgcacgagcagaggtgggccgggctgcgcagctgttggacgtgcggctgttcgagctgagcgagctgcgcgagggcctggctcgcctggcagaggcagcaccctagtttgctctggaggcctgcggccccaaggcaccctggcagggaccagggggccccagctgtctctgagctttgca

SEQ ID NO: 15 - DNA, 1588 bp, Rattus norvegicus
  misc_feature: >gi|6981533|ref|NM_013028.1| Rattus norvegicus short stature homeobox 2 (Shox2), mRNA
ggaattcggcacgagccgggagctggacatgggagccgcggagcggagcagggagcccggcagcccgcggctgacggaggtgtcccctgaactgaaggatcgcaaagaggatgcgaaagggatggaggacgaaggccagaccaaaatcaagcagcggcgaagtcggaccaattttaccctggaacaactcaacgagctggagaggcttttcgatgagacccactatccagacgcttttatgcgcgaggaactgagccagcgactggggctatctgaggcccgagtacaggtttggtttcaaaatcgaagagctaagtgtagaaaacaggaaaatcaacttcataaaggtgtccttataggggctgctagccagtttgaagcttgtagagttgcaccctatgtcaacgtaggtgctttaaggatgccatttcagcaggatagtcattgcaacgtgacgcccttgtcctttcaggttcaggcgcacgtgcagctggacagcgccgtgcgagccgcgcaccaccacctgcatccgcacctggccgcgcacggcccttacatgatgttcccggcaccgcccttcggactgccgctggccacgctggcggcggactcggcctccgccgcctcggtggtggctgccgccgccgccgccaagaccaccagcaagaactccagcatcgcggatctcagactgaaagctaaaaagcacgcggccgccctgggtctgtgacgccggcgccacggccacggtcggtggagccttctatgcggcgcgaccccgcacgctctccgcgaccggctgcttctcccgcacccgcttcggaccgactaagcctgtcccttccccgctgactgccgccttttctttctgcaccctggatccctagggcgggactctgcgctggacccgggatcccacgaggaccaccagcccgggctcctgccacccgcccttgggactaaaattcggctttgtaggagcgggtttggggaagtctggatagagactggacaaaggagtgtggcaacagtgaccacagctgcggcgacggactcgttcataggaataaaatcttgctaacaacaaatgagcaaaaacaaaaaaggatagagaacaaactaaaaaaagaaaatgagaaagggggcttgtttcttattgctatttggctggaattagagagagaacgatgaagcaaaacaattcttaaaattattttgtacataaaaatacggaaaaataacctggatgaatcttgagagaaaggggggagggatttaccaactaaaatgtatgtttttttttaaatggtccaaggaaacaaaacagaaggaagaggtatactatttaagaattaagatgaaaaaaaaaagcgcagctgggaagttcacaggttttgaagctgacctttttctgcgaagttcactttaatacgagacatttgataagagaggcgggcctccttttacgttgaatctgatgctttgagttttaaaacaccatgtttggaagagcaagaagagggaagacagtgtggaaacgaggagggaaaaaaggacgttcatacaaaaaaaaaaaaaaaaactcgag

SEQ ID NO: 16 - DNA, 644 bp, Rattus norvegicus
  misc_feature: >gi|14961453|gb|BI296732.1|BI296732 UI-R-CV2- cgv-f-08-0-UI.s1UI-R-CV2 Rattus norvegicus cDNA clone UI-R-CV2- cgv-f-08-0-UI 3′,mRNA sequence
ttttttttttttttttttcaaattcgtactgctttaatgtgaaaagtcaacactgaaaactgtgttacttcattacaagaatgatatttgtctaatcttactcatattacaattgcaacttacacagagtggaagtagacaaacgccaaaataagtgcataaatgagacgaactcaatctgcaaagttaggaggacagcccggtctgtacagtctctataaacactgccaagaggagcacatctacagcggagggaagaggatggacagaccacaagttcccaaaggaacacagaaagcactaacaaaggtactcaaatacagaattggctaaaaaaaaaaaattattttttttcttttttcaagaaaaattgccgggcttcttcccaggcctgctgaggaaatctgttagccagctccaggtagtccccagggtttccgcatttccctttgggattcgaagactggttatccacatgacccggagtctgtgcaaagtcctgaatgaatgtggccagtaaaacacagctcatctcttgctccagaatgagcttgttctgatggttgttatgacacgcagcgataagtgaagggaacagactttgatgagccttgggtcactgaaatactggaaaggcagctggc

SEQ ID NO: 17 - DNA, 3101 bp, Rattus norvegicus
  misc_feature: >gi|2267586|gb|AF009329.1|AF009329 Rattus norvegicus enhancer-of-split and hairy-related protein 1 (SHARP-1) mRNA, complete cds
tgtctacaccataattcctttgtctttgagccagctcacaaatgtcactgtggttctgagtgtgggggtcttggtgcagtccctcccctcccagtcccttccgtcgaggagcatggtgctagtgctgccacagcctggagacgcacacaaccccccaaaatctctccagacgaccgtcccacgatcacaggacagaaccctccaaatcgaaacggaggaaacggacagccattgaacatggacgaaggaatccctcatttgcaagagagacagttactggaacatagggattttataggactggactattcctctttgtatatgtgtaaacccaaaaggagcttgaagcgagacgacaccaaggatacctacaaattaccgcacagattaatagaaaagaagagacgagaccgaattaatgaatgtattgctcagctgaaagacttactgcctgaacatctgaaattgacaacactggggcatctggagaaagcagtagtcttggaattaactttgaagcacttaaaagctttaacagccttaacggagcagcagcatcagaagataattgctttacagaatggggagcgctctctgaaatcgccggtccaggccgacttggatgcgttccactcggggtttcaaacctgcgccaaagaagtcttgcaatacctcgcgcgctttgagagctggacgcccagggaaccgcgctgcgcacagctcgtcagccacctgcacgccgtggctacccagcttctgacgccacaggtgaccccaggcaggggccctgggcgcgcgccctgcagcgctggggctgcagccgcctccggttccgagcgcgtcgcccgctgcgtgccggtcatccagcggactcagcccggcacggagcccgagcacgacacggacaccgacagcggctatggaggcgaggcggagcagggccgcgccgccgtcaagcaggagccacccggggacccgtcgctgcgcccaagaggctgaagctggaggcgcgcggcgcgctcctgggcccggagcccgcgctgctcggctctctcgtggcgttgggcgggggtgcgcccttcgcgcagcccgccgccgcgcccttctgcctgcccttctacctgctgtcgccgtccgccgccgcctacgtacagccctggctagacaagagtggcctggacaagtatctgtaccccgcggcggccgcgcccttcccgctgctgtatcccggcatccccgcagcagccgccgctgccgccgccgccgctttcccttgcttgtcgtccgtgctatcgccacccccggagaaggcaggttcggccgctggtgccccattcctggcgcacgaggtggcgcccccggggtcgctgcgcccccagcacgcgcatagccgcacccacctgccgcacgccgtgaacccagagagctctcaggaagatgccacgcagccggccaaggacgccccctgaacccagcattccttccagaacagggcagggggctcctgaggagtcgccaggtttccaagttcaaacatcccctaaggcgtaccagggaggaagagtaagagatgctctgctcgacaggcttaggacaaaaacaggtgttttgtgtatgtttggagttcctgttttgcccctttctcacccttctgccaccccaccctctaccctttgacactcccttccccatccctgctgtcacagagcctccctgagaaatactggttatcttaaattaccctcccttacatttagttcacgtcctctgtttccaaacatagaccctggttcaggagtctgttgggtgggagagccacacggaaccagttagagtgcctggtatcagggctccttgacccaggcctggaacagtagctgtgtcccctgtctgtccccttaggaggtgacccataactgagggtctctgaaagttacattgacgtgtcagtattttgtattcttcagctttttggaaggtacctctttttcaaagaagtgaggatgccattgccctgttgtgaggtggctggagtggtgtctttataccttgcacctgttgggagaaactgagagttggggccatcttcaggcactgtgtcagtgtgggagctggaagagggagtttggagcccgtggcgcctttctcgcactttattgacaaattgacctcaacccctttgtcccatgtctcaactcacagatatatgtcataggttatatatttgtgtttctgatccctcgttattttatccatcatggtcccaaatttttgtaatgttactggggtttggggtggggtggggtgttaaagtgctctgggctggaaaaagacaagcccaaacctattgattgtcgaattcttagatgacagaagtggagagaggggcttgtggtcccttgtgatgggaagtgctgtgaacatgtagaaggccctgccagcctcgctctctcaagtctgtatgtatttttcgggagaccaaaccagacaccagataatcaggaagaaagctttttaaaataaggcaaaaaccgagaccttgtctagatatttttagtttgttgccaaggtagcactgagaaatctcacttgaatgttacataaggagtgattcacaatagtctagagtgaagaaagttatctgggtctgtgagtgttcgggtccgtttgctgctgctgttgctactgtttgcctcaaacgctgtgtttaaacaacgttaaacttcttagcctaccaaggcggccgtatgtacatagctgttaatacccccaactaatgtctgacatgctatttttgtagggagaagatacctgctagtgatattttgagttaaaatatcttttggggcggacttggtgaaatgtttgcactttggtcacaatgcttctactgcttggtgcaacgttacgctgtcttaaattattaaacaaataaaaaatactatctgcaagaaaaaccagctggtttagacaagtttagtatgtaaagataagctagaaactatctttatattctagtattttcagcactccatattacctaaatattgccacactattttgtgatttaaaagttcttactaaggaataaaatctttata

SEQ ID NO: 18 - DNA, 3067 bp, Rattus norvegicus
  misc_feature: >gi|9506894|ref|NM_019208.1| Rattus norvegicus multiple endocrine neoplasia 1 (Men1), mRNA
cggcaggcagtgctatcagaggaggtgcaggacttcatttcccagcaggcttcaggcggcgggcgccgcggtgccttgtgtgggatgtaagcgcggaggtgggctcgggggaccgaagccaggactctccttgggacttgcgggaggctgctttaggttcgccttttggacaggctccagagggctaggtagagattgcagggaccgggaggagtggcctttgagggtccccaagaggtctttgggacgcagcctcatcggggctgggggtcccgggattgtagggaactagaaggcccttggttgaaaggtctcagaaaccgagccgccgcccgtgtgcgcaggacgaagcggctggcgaagcagaattgtggcggagggtagagccgtagccgagcctgggagggggctctctcgccgagcccgaccctcccttcctcggcttgcccgcaggccaccgcccgccgccatggggctgaaggccgcccagaagacgctgttccctctgcgctctatcgacgacgtggtgcgcctgttcgctgcggagctgggccgagaggagcctgacctggtgctcctctccttggttctgggcttcgtagagcatttcctggctgtcaaccgtgtcatccccaccaacgtgccggagctcaccttccagcccagcccggcacccgaccctcctggtggcctcacctacttcccagtggccgacctatccatcattgctgccctctatgcccgattcaccgctcagatccgcggtgctgtggacctctccctctatcctcgagaggggggcgtttccagtcgcgaactggtaaaaaaggtctcggatgtcatatggaacagcctcagccgctcctacttcaaggaccgggcccacatccagtctctcttcagcttcatcacaggcactaaactggacagttcgggcgtggcctttgcagtggtaggggcctgccaggctctaggtctcagagatgtccatctggccctgtccgaggatcatgcctgggtggtgttcgggtccaatggggaacagacggctgaggtgacctggcatggcaaaggcaacgaggaccgcagaggccagacagtcaacgctggtgtggctgagcggagctggctgtacctgaaaggatcgtacatgcgctgtgaccgcaagatggaggtggcgttcatggtgtgtgccatcaacccttccattgatcttcacactgactctttggaactgttgcaactacagcagaagctgctctggctgctgtatgacctaggacatctggaaaggtaccccatggcgctagggaacttggcagacctggaggagctggaacccacccctggtcggccagacccgctcaccctctatcacaaggggattgcctcagctaagacctactaccaggatgaacacatctacccctacatgtacctggctggataccattgtcgcaaccgcaatgtgcgtgaagccctgcaggcctgggccgacactgccactgttatccaagactacaactactgtcgggaggatgaagagatctacaaggaattctttgaagtggccaatgacgtcatccccaacctgctgaaggaggcagccagcctgctagaagcaggcgaggagcggccaggagagcaagcccagggcacccaaggccaaggttctgccctccaggacccagagtgctttgcccacctgctgcgattctatgatggcatctgcaaatgggaggagggtagccccacaccagtactgcacgtgggctgggcaactttccttgtgcagtccctaggacgctttgagggacaggtgcggcagaaggtgcacatagttagccgggaagccgaggcagcagaggcggaagaaccatggggggacgaagcccgagaaggtcgtcggcgcggtccccgccgagagtccaagcctgaggagccaccaccacccaagaagcctgcgttggacaagggcccaggctcagggcaaagtgcagggtcgggacctcctaggaaaacgtcagggactgtctcaggtactgcccgtggcacagaagtcagcagtgctgctcaggctccagcacctgcagcatcgccacccccagagggcccagtgctcactttccagagtgagaaaatgaaaggaatgaaggagctactggtggccaccaagatcaactctagtgccatcaagctgcaactcacggcacagtcccaagtgcagatgaagaaacagaaggtgtccacacccagcgactacacactctctttcctaaagcggcagcgcaagggcctctgaactcttcgggattctgtgacaaggcccccggcctgcccacactcccatcaggcttcactctagtcctctaactcaagtctagactctgcctacagtcctaggtggcccagaactgcccagcagtgactgggagccttcatttagccccttttcgggccccagagccctggctcggttcttagaatttaggtttcatctcccaccagccagccaattggaacattgtaggtccatgcaaccggtctttacaggtccaaccccgtcccccgaaattcagtggaccatgacgggttccctagtcgagcctgaacagtccggcattcctcccacttgggggactagattgaccttagcccacaagcacagttgctacctggaaacccaggaaccaaggaccattcagtccctaggggtagatttccgcactttatagttgcatcctttggcaatccaagtcccctaccccaaagaaattaacatattgttgcctgaatctggaaatcttcctcatctccctagccccggagtccagggcatgaaggtcggactcttatctgtgaatactgagcctcgcccccttcctgaccaaactggccggagaacagagcaggacctcttccagccctctgggaacctccttgaggtcccaggaggaaatctgcacggggtgggggagggccagtcttttcctgttttgtacatagatttatttttcagttccaaggaagataaatacattttgttaagaaaaaaaaaaaaaaaaaa

SEQ ID NO: 19 - DNA, 710 bp, Rattus norvegicus
  misc_feature: >gi|8503945|gb|BE111840.1|BE111840 UI-R-BJ1- avz-d-03-0-UI.s1UI-R-BJ1 Rattus norvegicus cDNA clone UI-R-BJ1- avz-d-03-0-UI 3′,mRNA sequence
tttttttttttttttttccaagaagataacttttatttttcttctttacatacttattttgaatatgattacctagattaaaaggaaacaagattttaaaagcaaagtatcccttttattataatttttttcttttacataactttaaacctagatcaagtaaccacctatctctcatcacaggaaaagggccctgcgcattttaacaggcaaaaagttttaagagatataattgccccattacaattttcctattttcttgagtatagtggaaaattgatagaaagacaggagggaaagtagtcctgcagcaaggctgtaagacctcacttaggttggtggtagttcatgttcgtcctccaaacaggtgcaagggaggtccaagtgctctatgctggtgttttctttgctcattcccagaactcccagccagtgtctgactaaaccctgtaaaatgtgttagggcctaattatgcaaaatacattagcgatccaagcaaggaggccacataactcccagaaataccgagtaattatcacaactatatctaaaaatttacaattgccaattaaaaatggaagtatggctacttaaacggtaacaaaatgagtatgaaatatcgattttcaagattaaaaggaccccttagaaggacaattaaatgagtggcatgggagaaaaggaaatggtatgcagcttagtttgctta

SEQ ID NO: 20 - DNA, 1356 bp, Rattus norvegicus
  misc_feature: >gi|9506376|ref|NM_019220.1| Rattus norvegicus amino-terminal enhancer of split (Aes), mRNA
cccggagctgtcgatcggggacaaagcgcggccgcccgcgccccccgagccgcccgagccggggccagccccccgcgactgacatgatgtttccgcaaagccggcactcgggctcctcccacctcccccagcagctcaagttcaccacctccgactcctgcgaccgcatcaaggatgagttccagctgctacaagcgcagtaccacagcctgaagctggagtgtgacaagctggccagcgagaagtcagagatgcaaaggcattacgtcatgtactatgagatgtcttacggattgaacatcgagatgcacaaacaggctgaaattgtgaagaggctgaatgggatttgcgcccaggttctaccctatttgtcccaggagcatcagcagcaggtcctgggagctatcgagagagccaagcaggtcacggctcccgagctgaactccatcatccgacagcagctccaggctcaccagctgtcccagctccaggcgctggccctgcccctgacaccgctgcctgtcgggctccagccgccatccctccctgcggtcagcgcaggcacaggcctgctgtcactctctgctctgggctctcagacccacctctccaaggaggacaagaacggacacgatggggacacccaccaggaggatgatggagagaagtccgattagctcctggggagagggagggggaggggggataccaggggagacaggcacagagagcacagtgttctgagggccacactgcacacagctcggtccctacccaagcttccacctccagtcctggttccagctgtgcaccctgggcctaggagacgccagagggacagcccaggctacagggaaaattccccaccttcaacatttccccgggctacagccaccaatctcggcccagaatgtacacaacgctagccgcccctttccaccctgtgcaccccgaatttgccccctttccccttctctgcttcctgcactgcacacctgctaggcccagacccctccccccagctcacccagctctggttccctcaaccgcggaaagaaagcattggctcgggactgcagtgggggtgggcaccccacaaacttccctctccccttccccaaataaacgtgagggttaaaagtcggtttgtttattatttctttttcgtcccagtactctatagctaccatctaagagagaaaaagaaggaagccctggagcgagcgagggtctgtctgtgcatatagttagatttttctggtcgcctcgctttgcaccgagaggtggagggccgctgtactgagggtggggtctagctgtgccttcaaaataaaaacatgaaaaggtcaaaaaaaa

SEQ ID NO: 21 - DNA, 511 bp, Rattus norvegicus
  misc_feature: >gi|11382764|gb|BF397780.1|BF397780 UI-R-BS2- bee-e-11-0-UI.s1UI-R-BS2 Rattus norvegicus cDNA clone UI-R-BS2- bee-e-11-0-UI 3′,mRNA sequence
ttttttttttttttgttctcagcgttcctttattgtcaggaaactgtgtcatcattaaagtttttttttcccctggaacatacatagactaaatacagcccccaacccaaacctcccatccctgggtaccgcgtgtgtgtagccatttaggagacgtggtccagttggggttccttatccttttaggcactagagttgaggaactgcgtctctccaggcagagaccccagtaagacactcatgttaccgactgccatgttgggggggcccggagggaggtggggtgtttttagagctgtccccttgctcgtgggaaggaggagggctcagcccttgagcttcatctaggatagccacaaagtccagctgagtgttgtccaggtcaagagagtccagagggttacacacctgcccttcagggggagggtacaaggcagggcctgctcctaacctgcccacttcaggatgcccacagctggggttggcatctcccaccttggaggggcccata

SEQ ID NO: 22 - DNA, 588 bp, Rattus norvegicus
  misc_feature: >gi|13886741|gb|BG664819.1|BG664819 DRABLF11 Rat DRG Library Rattus norvegicus cDNA clone DRABLF11 5′, mRNA sequence
cctaagtgtatcgtgctttactttttgttactgcaacacatttgatggaacacactgtctcctttaaaaaagaaaaaaagaaaaaaaaaaaagccagacccccgttcttgctgtattttgcttgttgagtacaaagatgaagcgatttgccaaaaagcggccaagttgctcccatttccctgtaattacgaaggtttggttgggcgatgtacgctccagccctcatgcacactaactcttcaatctaattgtttgattaaactcgtatttcctccagaaaggtacataaataagtgaactgttgggcaaattaagaatacttaacagcattgcatccgaaaagtagttatgctgattaaattttgtgtccttgaggactttcaggaaattacttttgatcgtcaataacacaattaagtaaactacacacacattagcatgaataattgataaagaaattatgtattaccacgaagcactgatttataaatcatgacgcaacactccaaggactgtgcaaaactggataacattgaaaaagctggcttgcgttctggaagaaaagaaaaaaaaaaaaaaaaaaaaaaa

SEQ ID NO: 23 - DNA, 517 bp, Rattus norvegicus
  misc_feature: >gi|11383403|gb|BF398414.1|BF398414 UI-R-BS2- beo-f-01-0-UI.s1UI-R-BS2 Rattus norvegicus cDNA clone UI-R-BS2- beo-f-01-0-UI 3′,mRNA sequence
tttttttttttttttttcaattttcagtatactttattttctattaaatgaaaaccaagaaatgtcaagttaagttaaaaggctttaccctttcaagaaaaaaatacaaactgtatggtgtgatacttgtataacaggtcatattttacattacacaatataaacaaaatcactaatctacaattatttgaaactacacatgaactacagattaagtgggaaatgggtttacttttttggtgcaaataaacatcttggaaaaacatacagcatgcattttttaactgtatgcataagctctgacagtccattcttaatagccgccaccaccaccaccaccaccacctcctcttccttgtccaaaataaccacctccataaccccttcgctggaatcctccagatcctctatagcccccaccagatccccggtagtctccactagatcctctgtagtctccaccgaagttgtttctgtagcctccttgggaacctcctctataacctccctcgtgccg

SEQ ID NO: 24 - DNA, 595 bp, Rattus norvegicus
  misc_feature: >gi|8512254|gb|BE120149.1|BE120149 UI-R-CA0- bap-b-07-0-UI.s1UI-R-CA0 Rattus norvegicus cDNA clone UI-R-CA0- bap-b-07-0-UI 3′,mRNA sequence
tttttttttttttttttgagaaacaaacacagctcatttattagaattaaaaaaagagaaccatctcccacaaacacagagtactgcctctcagtctatatcaagagcaagacaacagttgaaaattgtattcctgagaagcaaaaagttaccagtttgcaaacaaatataacaggtgatttcaataaaagataaaaagtttaaaaaaaactaagaacaaaatttcaagtattattccagatgacatggcaaacctaataatgtaccagatcagggctggagaagcaaggccctgtcagtctggagccatctggagcttcagcagggagttgctctcagggactggagggctaagggcttggcctgctggacaagaggcattggccagccctgtgtcaaggacttggatactcaagacatcctccccaggattagcagttttggacatttgataagtttttaaataaacaaaaacatatgctcattcttccaaatgtcaggctattcctaattttagtcaatttgtaaattataaattatagtatttgtgctacagtttttaatatttaaatcttattagaagataagcaccacc

SEQ ID NO: 25 - DNA, 615 bp, Rattus norvegicus
  misc_feature: >gi|8085120|gb|AW919327.1|AW919327 EST350631 Rat gene index, normalized rat, norvegicus, Bento Soares Rattus norvegicus cDNA clone RGIFP82 5′ end, mRNA sequence
gcttaaaggcccaccacaggactcttacaggtgagattccttacaagtgctcatgggaaggatgtgagtggcgttttgcccggagcgatgagctcacaaggcactaccggaaacacacaggtgcaaagccctttaagtgtaaccactgcgacaggtgtttctccaggtctgatcatcttgctctccatatgaagagacatatctaaacaaaacagaacaaaatttaaaaaaaacaaacaaacaaaagtaaacaacaacaagagcaaaacaaccgcaaaggccagagttgccatggcagcagccaatgtccgaaggaaatgccgtgaccagaggactggacttcaggcgggagcactgcctcgcagaagaaagttctcacttacaaacctctgtgtacatacatgtacacgcacacgtacacacaccctctcacacacagacccacacacatacccactgtcatgcactcaactatatttaaagtatatacatctatttcttatgccttgccctagccagatgatagaagacaaggaaggaggaagccaggtgaactgggcgagcactggctgctgactccagcactactggatctcttcctgctgttgccaaagg

SEQ ID NO: 26 - DNA, 3615 bp, Rattus norvegicus
  misc_feature: >gi|11870008|gb|AF216807.1|AF216807 Rattus norvegicus clone NP/NMP4 28H nuclear matrix transcription factor mRNA, complete cds
gccgcagcaccacttccggaaagttcaggagccctggaaaggagaaggaataagacgacaggaggaagagagagagaggggagaatggaagagtctcacttcaattctaacccgtacttctggccttctatccccacggtctcagggcagattgaaaacactatgttcatcaacaagatgaaggatcagctgttgccagagaagggctgtggactggcaccacctcactatcccactttgctgacagtgcctgcctcagtgtccctgtcctctggcatcagcatggacacagagtccaagtcagaacagctgaccccacacagtcaggcatctgttacccagaacatcacagtggtccctgtaccatctacaggactcatgactgcgggagtctcctgttctcagaggtggagaagagaagggagtcaatcaaggggtcctggtttggtaatcacatccccttcaggctctcttgtgaccacagcctcatcagctcagactttccctatctcgactcccatgattgtctcagctcttccccctggctcacaagccctacaggtggtccctgacctctccaagaaggtagcatcaaccctaactgaggaaggaggcggaggtggtggtggaggtggcactgtggctcctcctaaacctcctcggggccgcaagaagaagcggatgctggaatcagggcttcctgagatgaatgacccttacgtccttgcccctggggatgacgatgaccatcagaaagatggcaagacctacaggagcgaagggaactgcggcacaggaaatggacagagccttgggctcatggattcagttcccggctccaccacgaacttgctgtgtgaccctgggtgccggatgtgctcactgaccttctactcaaagtcggagatgcagatccactccaagtcacacaccgagaccaagccccacaagtgcccacactgctccaagaccttcgccaacagctcctacctggcccagcacatccgtatccattcaggggccaagccctatagttgtaacttttgtgagaaatccttccgtcagctctcacacctgcagcagcacacccggattcactccaagatgcacacggagaccatcaagccccacaagtgcccgcactgctccaagaccttcgccaacacctcctacctggcccagcacctccgtatccactcgggggccaagccctacaactgttcctactgccagaaggccttccgccagctctcccacctccagcagcacacacggtaagggagaggggcgggctattgatcccgccccgctggcatgccctccccacccccgccccggagacacacaaccacccgcatgcgggggggcgggggagagacctggctggaggtgagacctggctggaggagaatatgactggcaacagagaaaagaagtggaagccaagagtctttctggagcacaaactgtattgaaaagacacagtgattgtgtggtactttcctgattgattaaagacagaggcaaacttggagcaaagaggactctttccttcgctttcctccctggaaataccaagcacacgtcttaccagatttctgctctggtgcctcagggcacagttagaatcgggagacgtaatcagatgaagctggcaccagcctggatacactcacagcaagaagcaaggttgatttcaagggatgccaagagaagagcacaggaggagacatgaactgaacattcacagaggagtggaaacaaacgaaaagctcttacttcataagcaggatgcttgtttgcatttcatctcaatgtggagttactagggaaaaggcagtctgacagcttgggaccaaagagctgatggtttccttttcctttggtgatctatcaagatgaagtccagacttggcaattggccatctcaccctgcttcctgtggatgtgatgttctcacctgaaaatggtcagacgtggagtttgctgtcttcgtgtatttgaggccttgatcacagagatggtctgaagccctaaaaaggagcaggtaatccacactggcgatagaccatacaaatgtgcacacccgggctgcgagaaggctttcacacaactctccaacctgcagtctcacagacggcagcacaacaaagataaacccttcaagtgccacaactgtcaccgagcatacacagacgcagctcactagaggctcatttatctacacacacagtgaagcacgccaaggtttatacctgcacaatctgtagtcgggcatatacatcagaaacatatcttatgaaacatatgcgcaaacacaaccctcctgatctccaacaacaagtgcaagcggcggcagcggcagcggcagtggcccaggctcaggcccaggctcaggctcaggcccaggcccaggctcaggcccaggcccaggctcaggctcaggcccaggcccaagcttcacaggcatcacagcagcagcaacagcagcagccaccacctccacaaccaccacacttccagtctcctggggcagctccccagggagggggtggtggggacagcaaccagaaccctccaccccagtgttcctttgacctgaccccctataagccggcagagcatcataaagacatctgcctcactgtcaccaccagcaccatccaggtggaacacctggccagctcttagagatccgtactgccatccattggaaaggggagaagtcctggtccctcctttgtcaactcttcttggtgggaaagttctcttcttccttgacaagtcctgtctccagctccttgggtctcaggcatggcttttcttcgcaagataaccatccttaatctcagctgctcctgattggccaaggaatcctgagctgatagtgttatccaacagcccccatccaagcagagcttttaaaactggggcttggtgcttgagagaaggatccgctgtaacctcacatcttgtccagtgtgtgggcgattacctttctctccatcctcaaagttggggctgatagaagactagtggctggccctcccagataacagagaaggaagctctaaatgtcgccagccttccgttctatgcttacctgtaaaagaacagattttgcacgtggcctgatccctgtgagccattttcttccccttacatggtctcacttcatattgggtggtgactataccttttttatttgtaaggggggcaatagctgatacatggtcaccaaggtgccagagattccccaggtgaccaacggtggcttaagagtgtgagttaccatttttccttacaacctccttccttctctgccaaccaagggcctatatactcagtctcactaccccagtccaaggagctctgggagctgtgtaagctgtgttctctgcgatgccatctgggctttgcagagaagggagaattcagggcacacacaggactggacttagcctccctaggtgccacggtcagatgccggacatggatttatatataaatatatatataaatatatatatatacctgctcatcacggccatctttgttgtaaccatttctgtgtttataaatgcattatctctgagaaaaaaaaaaaaaaaaaaa

SEQ ID NO: 27 - DNA, 2573 bp, Rattus norvegicus
  misc_feature: >gi|19923684|ref|NM_021835.2| Rattus norvegicus v-jun sarcoma virus 17 oncogene homolog (avian) (Jun), mRNA
gacgactggtctgcgccgccggagaacctctgtcgctggggctggtccgcgggctccgaggaaccgctgcttcctgagagcgctccgtgagtgaccgcgacttttcaaagctccggatcgcgcgggaccaaccaacgtgagtgcaagcggtgtcttaaccctgcgctccctggagcgaactggggaggagggcgcaggggggagcactgcggtctggagcgcacgctcctaaacaaactttgttactgaagcggggacgcgcgggtatcccccgcttcccggcgcgctgttgcggccccgaaacttctgcgcacagcccaggctaaccccgcgtgaagtgaccgactgttctatgactgcaaagatggaaacgaccttctacgacgatgccctcaacgcctcgttcctccagtccgagagtggcgcctacggctacagtaaccctaagattctgaagcagagcatgaccttgaacctggccgacccggtgggcaatctgaagccgcacctccgagccaagaactcggaccttctcacgtcgcccgacgtcgggctgctcaagctggcgtcgccggagctggagcgcctgatcatccagtccagcaatgggcacatcaccactacaccgacccccactcagttcttgtgccccaagaacgtgaccgacgagcaggagggcttcgccgaaggcttcgtgcgcgccctagctgaactgcatagccagaatacgctgcccagtgtcacctccgcggcacaacctgtcagtggggcgggcatggtcgctcccgctgtggcctcagtagctggcgctggcggcggcggcggctacagcgccagcctgcacagtgagcctccggtctacgccaacctcagcaacttcaacccgggtgcgctgagcagcggcggtggggcgccctcctatggcgcgaccgggctggcctttccatcgcagccccagcagcagcagcagccgcctcagccgccgcaccacttgccccaacagatcccggtgcagcacccgcggctgcaggcgctgaaggaagagccgcagacggtgccggagatgccgggagagacgccgcccttgtcccccatcgacatggagtctcaggagcggatcaaggcggagaggaagcgcatgagaaaccgcatcgctgcctccaagtgccggaaaaggaagctggagcggatcgcccggctagaggaaaaagtgaaaaccttgaaagcgcaaaactccgagctggcgtccacggccaacatgctcagggaacaggtggcacagcttaaacagaaagtcatgaaccacgttaacagtgggtgccaactcatgctaacgcagcagttgcaaacgttttgaggacagactgtcagggctgaggggcagtggaagaaaaaaataacagagataaacttgagaacttgactggttgcgacagagaaaaaaaagtgtccgagtactgaagccaagggtacacaagatggactgggttgcgacctgacggcgcccccaatgtgctggagtgggaaggacgtggcgcgcctggctttggcgaggagccagagagcagcggcctgttcgcgacgctttgcgaacgggctgtgccgcgaccagaacgatggacttttcgttaacattgaccaagaactgcatggacctaacattcgatctcattcagtattaaggggggtgggtgggggcttacaaactgcaatagagactgtagattgcttctctagtgctccgtaagaacacaaagcagggagggctgggaaggggagggaggcttgtgagtaccaggctagattgcggatgaactcccctggcctgcctctctcaactgtgtatgtacatatatttttttttaatttgatgaaagctgattactgtctataataaacagcttcctgcctttgtaagttatttcatgtttgtttgtttgggtgtcctacccagtgtttgtaaataagagatttgaagcattctgagtttaccatttgtaataaagtatataatttttttatgttttgtttctgaaaatttccagaaaggatatttaagaaaacacaataaaccattgagaagtagcccccaacctctctgctgcattatcgatagataatgagagctaggtgaaatgacagctgagtgtctgtatgctggggtgagagcctgtagtccctcccgtctggttgtaggaatagacaccctgcatgctatcattgactcatactctctccccagcaacccacaagtccagactgttgaccagaagatggtgcagtgtttcttaaggctggaagaagagggtgttgcaaggggagagggacagcccactgggaaagcagatgcttgagttgagagccatatcaagtagcatgcgctgtgatcgtttataataataataaagacttcagcaattagatgtttatctcaaagcagggacccatggaaggttttacaaaaggtgtctccttccaacttggattctgacgactcctagaaaaagatgacctttgcttgtgcatatttataatagttcgtatcacaataaatgtattcaaataatggtttt

SEQ ID NO: 28 - DNA, 1506 bp, Rattus norvegicus
  misc_feature: >gi|16758257|ref|NM_053499.1| Rattus norvegicus mitochondrial transcription termination factor 1 (Mterf), mRNA
gtaaaaggtgtctaccgcgagctttggtaccggacgcgaatacaaagttgagttcaggctggggttggagagacgctgtaccgttggatttctcttgaatgttctgtggatgttcatctgtctaccatgatgctaagcatctgaagagaacgcactgtatagggtagtaatctttaaccctaaaggcttcgtatgtgtatggcttttatgtatggacttgaaaagctttggactatcctgtgataatggcatcaagaaacatctggcgtgtgagaaggaacttcctctttgatttaagaggttgggtgccccaatattcagcagaagttttcctcaagtcgattccattcaggccttttagtgtggaatgtaacagtaaagacggggagaatggggacctactcaacaacttactcaccatgggagttgatgtcgacatggcaaggagacgacagcctggagtttttaacaaggctgtcacgaatgagcaggagctgaagatgttccttctgtccaaaggagcaagtgacaaagtgattggtagcatcatatcaagatatccacgagccataacacgcactcccgaaagtctttcaaaacggtgggatctgtggagagagattatggcatcagaccttgaaattgtaaatattttggagcgttctcctgaatccttctttcggtctaataacaacctaaacttagagaataacataaagttcctctgctctgttgggttgactcataagtgcctctgccgactgttgaccagtgctcccaggaccttctccaacagtctgaatttgaataaacaaatggttgaatttttgcaggagaccggtatatccttaggtcacaataatcccacagattttgtcaggaagataatttctaaaaatccttccatcttaattcagagcaccaaacgtgtgaaaactaacattgaatttttacagtcaactttcaacttagacaaagaggacctgctgcttctgatatgtggtccaggagctagaatcttagacctttccaacgactgtaccaaaagaaactacacaaacatcaaaaagaggctgctctctctcggatgcactgaggaggaggtacagaagtttgtcctaagctatctgaatatgatcttcttgtcagagaaaaagttcaatgataaaatagactgcctcctagaagagaaaatcagtacttcacaaatacttgaaaatccaagggtattagactcgagcatacatactttaaaaactcgcatcagagagttggcacatgctgggtatgacgtgagcacctcaagcattgctctgctgtcctggagtcagagacgatatgaagctaaactgaagagattaagtgggtaggccagtgcaggcggagtcaccaggaatcttgcaagtagagtgctggttccagtttggacttttcaaacaggagagatttttttaaataatttttttacttaaatcatttaaataaatattttaaaatatttaaaaa

SEQ ID NO: 29 - DNA, 983 bp, Rattus norvegicus
  misc_feature: >gi|42734494|ref|NM_013058.2| Rattus norvegicus Inhibitor of DNA binding 3, dominant negative helix-loop-helix protein (Id3), mRNA
tttttctgttcttaaactttgctctccaacctccaacatgaaggcgctgagcccggtgcgcggctgctacgaggcggtgtgctgcctgtcggaacgtagcctagccattgcgcgaggccggggtaagagcccgtcggcggaggagccgcttagcctcttggacgacatgaaccactgctactcgcgcctgcgggaactggtgccgggagtcccgcggggcactcagcttagccaggtggaaatcctgcagcgtgtcatagactacatcctcgaccttcaagtggttctggcagagcccgcgcctggacccccggatggtccgcatctcccgatccagacagctgagctcactccggaacttgtgatctccaaggacaagaggagtttttgccactgacccggtcgtcctggcacctcccgaacgcaggtgctggcgcccgttccgcttgggaccctgggactctgggaccctctctccagccggaagcctgaggggcatggatgagcttcgatcttgccacagccctcttcatttaccctgaactcatgcctcgaggctggacctggagcccgagagcgaaggactgaacttgggtggcctgaagagctagctagcactcgctggtcagcagctgggcaacgtcactctgtccccaccctgactcaagtcttaaaaagactggcttttttccgaaaatggggtgtcgagaagggtgtggggggtacacgagtggctgccctgcacacactgccaaggcagcataagagctggtcttctggtttccttggagaaaagctctgttgccctgattatgaactctataatagagtatatagcttttgtaccttttttttacaggaaggtgactttctgtaaccatgtgatgtatattaaactttttataaaagttaacattttgcataataaacaatttttgaacaaaaaaaaaaaaaaaaaaaaaaaaaaaaaaaaaaaaaaaaaa

SEQ ID NO: 30 - DNA, 549 bp, Rattus norvegicus
  misc_feature: >gi|13884891|gb|BG662969.1|BG662969 DRA05H06 Rat DRG Library Rattus norvegicus cDNA clone DRA05H06 5′, mRNA sequence
gaaaaaccttccaagtcattattaagccgtattgcagaaaacctacccaaacagcttgctgttataagtcctgagaagtatgacataaaatgtgcggagtctgaggcagcgatcattttgaactcgtgtgtggaacccaaaatgcaagtcactatcaccctgacatctccaattattcgagaagagaacatgagggaaggagatgtaacctcgggtatggtgaaagacccaccggacgtcttggacaggcaaaaatgccttgacgctctggctgctctacgccacgctaagtggttccaggctagagctaatggcctacagtcctgtgtgattatcatacgtattctccgagacctttgtcagcgagttccaacatggtctgagtttatccctttttaactgctagagtactgtagaaaagcaatagcagtgcttctagtcctcagagcccttgagatgccctgagaagagtatttgagtgcatttcttcaagggattattctccaaggcagccatggacttctggaaccttgtgaaaaggatcccttt

SEQ ID NO: 31 - DNA, 2972 bp, Rattus norvegicus
  misc_feature: >gi|12249194|gb|AF286470.2|AF286470 Rattus norvegicus strain SHR/Ola ADD1/SREBP-1c (Srebf1) mRNA, partial cds
ggagccatggattgcacatttgaagacatgcttcagctcatcaacaaccaagacagtgacttccctggcctatttgatgccccctatgctgggggtgagacaggagacacaggccccagcagccctggtgccagctctcctgagagcttctcttctcctgcttctctgggctcctctctggaagccttcctgggaggacccaaggtgacacctgcacccttgtcccctccaccatcggcacccactgctgtaaagatgtacccgtccgtgccccccttctcccctgggcctggaatcaaagaggagccagtgccactcaccatcctgcagcccccagcaccacagccatcgccagggaccctgttgcctccgagcttccctcctccacctgtgcagctcagccctgctcctgtgctggggtactcaagcctgccttccggcttctcaggaacccttcctgggaacacccagcagacgccatctagcctgccactgggctccacgccaggaatctcgcccacccccttacacacccaggtccagagctcggccgcccagcagccgccgccagcctcagcagcccctagaatgagcactgtggcctcacagatccagcaggtccccgttgtactgcagccacacttcatcaaggcagactcgctgctgctgacagctgtaaagacagacacaggagccacaatgaagaccgcaggcatcaacaccctggctcctggcacagccgtgcaggcaggccccttgcagaccctggtgagtggagggaccatcctggccacagtaccactggttgtggacacagacaaactgcccatccaccgactagcagctggtggcaaggccctgggctcagctcagagccgtggtgagaagcgcacagcccacaatgccattgagaagcgctaccgttcctctatcaatgacaagattgtggagctcaaggacctggtggtgggcactgaggcaaagctgaataaatctgctgtcttgcgcaaggccatcgactacatccgcttcttacagcacagcaaccagaaactcaagcaggagaacctgaccctgcgaagtgctcacaaaagcaaatcactgaaagacctggtgtcagcttgtggcagtggaggaggcacagatgtgtctatggagggcatgaaacctgaagtggtagaaacgctgacccctccaccctcagacgccggctcaccctcccagagtagccccttgtccttgggcagcagaggcagcagcagtggtggcagtgactctgagcccgacagcccagcctttgaggataaccaggtgaaagcccagcggctgccttcacatagccgaggcatgctggaccgctcccgcctggccctgtgtgtactggtcttcctgtgtctgacctgcaacccattggcctcactgtttggctggggcatcctcactccctctgatgcttcgggtgtgcaccgtagttctgggcgcagcatgctggaggccgagagcagagatggctctaattggacccagtggttgctgccacccctagtctggctggccaatggactactagtgttggcctgcttggctcttctctttgtctacggggaacctgtgaccaggccacactccggcccggctgtacacttctggagacatcgcaaacaagctgacctggatttggcccggggagattttgcccaggccgctcaacagctgtggctggccttgcaagccctgggccggcccctgcccacctcaaacctggatctggcctgcagcctgctttggaacctcgtccgccacctgctgcagcgtctttgggtgggccgctggctggcaggccaggctgggggcctgcagagggactacaggctgagaaaggatgctcgtgccagtgcccgagatgcggctgtcgtctaccataagctgcaccagctgcatgccatgggcaagtacacaggaggccatcttgttgcttctaacctggcactgagtgcccttaacctggctgagtgtgcaggagatgctatatccatggcaacactggcagagatctacgtggcagctgccctaagggtcaaaaccagcctccccagagccttgcacttcttgacacgtttcttcctgagtagtgcccgccaggcctgcctggcacagagtggtgcagtgcctcttgccatgcagtggctctgccaccctgtaggtcaccgtttcttcgtggatggggactgggctgtacacggtgccccccaggagagtctgtacagcatggctgggaacccagtggatccactggcccaggtgacccgactattctgtgaacatctcctggagcgagcattgaactgtatcgctcagcccagcccaggggcagctgatggagacagggagttctcagatgctcttggatatctacagttgctaaatagctgttctgacgctgtcggagctcctgcgtgcagcttctctgtcagttccagcatggctaccaccactggcacagacccagtggccaagtggtgggcctcactgacagccgtggtgatccactggctgaggcgggatgaggaggcagctgaacgcttatacccactggtagagcacattccccaagtgctgcaggaaactgagagaccccttcccagggcagctctgtactccttcaaggctgcccgggctctgctggaccacagaaaggtggaatccagcccagccagcctggccatctgtgagaaggccagtgggtacctgcgggacagcttagcctctacatcaactgccagttccattgacaaggccatgcagctgctcctgtgtgatctacttcttgtggcccgcaccagcctatggcggcgccaacagtcagcagcttcagcccagggagctcacggtaccagcaatggaccccaggcctctgctctggagctgcgtggtttccaacatgacctgagcagcctgaggcgcttggcaca

SEQ ID NO: 32 - DNA, 658 bp, Rattus norvegicus
  misc_feature: >gi|8085685|gb|AW919881.1|AW919881 EST351185 Rat gene index, normalized rat, norvegicus, Bento Soares Rattus norvegicus cDNA clone RGIGO01 5′ end, mRNA sequence
ggacccatgcccggcactacgaattgtaccgccgctgtaagctggaggagatgggcttcaaggacaccgaccctgacagcaaaccattcagtttacaggagacatatgaggcgaaaagaaacgagttcctgggggagctccagaagaaagaagaggagatgagacagatgtttgtccagagagtcaaatgacgatgtctggcccctgaccaagaagccaacaatcacgtggcttggagatacattccatacctcagtccctctcctagtacccttagccatatgcacccccacttccccaatctatcttcgccagactgtcatgtgtttacgggttgctgtttgccacattttatgtactccatttgaattccactgatacttgtttccttgaagtatttccagccactgactggataatactcctgcatccttgtcactgggattgttctcccactcctaatgccaggagcatagcccctcatcacaggttctaaaagaattgctggagatgcctatttcttctactatcttagcccagccctttctcgattccatttcctcggaatcactttgcacaagctatgttaaaagtcagccacacatttatttacaccaagcccttcctatacaaaatacaactgctggggcagacagtc

SEQ ID NO: 33 - DNA, 1696 bp, Rattus norvegicus
  misc_feature: >gi|4235388|gb|AF095585.1|AF095585 Rattus norvegicus LIM-domain protein LMP-1 mRNA, complete cds
gcacgaggatcccagcgcggctcctggaggccgccaggcagccgcccagccgggcattcaggagcaggtaccatggattccttcaaggtagtgctggagggacctgccccttggggcttccgtctgcaagggggcaaggacttcaacgtgcccctctccatctctcggctcactcctggaggcaaggccgcacaggccggtgtggccgtgggagactgggtactgagtatcgacggtgagaacgccggaagcctcacacacattgaagcccagaacaagatccgtgcctgtggggagcgcctcagcctgggtcttagcagagcccagcctgctcagagcaaaccacagaaggccctgacccctcccgccgaccccccgaggtacacttttgcaccaagcgcctccctcaacaagacggcccggcccttcggggcacccccacctactgacagcgccctgtcgcagaatggacagctgctcagacagctggtccctgatgccagcaagcagcggctgatggagaatactgaagactggcgcccgcggccagggacaggccagtcccgttccttccgcatccttgctcacctcacgggcacagagttcatgcaagacccggatgaggaattcatgaagaagtcaagccaggtgcccaggacagaagccccagccccagcctcaaccataccccaggaatcctggcctggccccaccacccccagccccaccagccgcccaccctgggccgtagatcctgcatttgctgagcgctatgccccagacaaaaccagcacagtgctgacccgacacagccagccagccacacctacgcctctgcagaaccgcacctccatagttcaggctgcagctggagggggcacaggaggaggcagcaacaatggcaagacgcctgtatgccaccagtgccacaagatcatccgcggccgatacctggtagcactgggccacgcgtaccatcctgaggaatttgtgtgcagccagtgtgggaaggtcctggaagagggtggcttcttcgaggagaagggagctatcttttgcccctcctgctatgatgtgcgctatgcacccagctgtgccaaatgcaagaagaagatcactggagagatcatgcatgcgctgaagatgacctggcatgttccctgcttcacctgtgcagcctgcaaaacccctatccgcaacagggctttctacatggaggagggggctccctactgcgagcgagattacgagaagatgtttggcacaaagtgtcgcggctgtgacttcaagatcgatgccggggaccgtttcctggaagccctgggtttcagctggcatgatacgtgttttgtttgcgcaatatgtcaaatcaacttggaaggaaagaccttctactccaagaaggacaagcccctgtgcaagagccatgccttttcccacgtatgagcacctcctcacactactgccaccctactctgccagaagggtgataaaatgagagagctctctctccctcgacctttctgggtggggctggcagccattgtcctagccttggctcctggccagatcctggggctccctcctcacagtcccctttcccacacttcctccaccaccaccaccgtcactcacaggtgctagcctcctagccccagttcactctggtgtcacaataaacctgtatgtagctgtg

SEQ ID NO: 34 - DNA, 781 bp, Rattus norvegicus
  misc_feature: >gi|13884511|gb|BG662589.1|BG662589 DRAAJB09 Rat DRG Library Rattus norvegicus cDNA clone DRAAJB09 5′, mRNA sequence
atggaactaggagaagaggagaaggcagagaaaaagaaaagaggaccaaagccttccacacagaaacgtaagatggatggtgcccctgatggccgaggaagaaaaaagaagctgaaactatgaatgcgttttgtcttataatcactaactagtagttctttaatttacgggtcttcataagatgttctgtacagtgctcaactgttactgtcattcagagacatgaggtcatctgcttactgagctagaaacatagtatgtagtttcccttttttaaatgcaacagctgtgctgaaattttttttatcattaacaattgaagtaataaaacaggcttcatttattaataaatgtttcatttgatttatttttctaatagtttcttttgtgaggattgtggctttttatcagctactttaacacttgtatggcttanaaacaagcaaacaagtaaaaaaaaaaaatgcaaataacatttgattgggcccttctancctggtttgtaattacttcacattctttgctgattttttttttttttttttttttggtnagactattgagtctcatctggaaagattggggaaattgcattcttgcttattatagaatggggggaaggcaagacatattatttttctgggcatatttctgtacactttctgttctatggttctgangngaatgggcctattttgttggatgatggccttttttagttgatagccaagactttagattttttggtctttaaggaaggattatttggaaaggaattttt

SEQ ID NO: 35 - DNA, 806 bp, Rattus norvegicus
  misc_feature: >gi|13162307|ref|NM_024134.1| Rattus norvegicus DNA-damage inducible transcript 3 (Ddit3), mRNA
gcttgaatctaatacgtcgatcataccatgttgaagatgagcgggtggcagcgacagagccaaaataacagccggaacctgaggagagagaaaccggtccaattacagtcatggcagctgagtctctgcctttcgcctttgagacagtgtccagctgggagctggaagcctggtatgaggatctgcaggaggtcctgtcctcagatgaaattgggggcacctatatctcatccccaggaaacgaagaggaagaatcaaaaaccttcactactcttgaccctgcatccctagcttggctgactgaggagccagggccagcagaggtcacaagcacctcccaaagccctcgctctccagattccagtcagagttctatggctcaggaagaagaagaggaagatcaaggaagaactaggaaacggaaacagagtggtcagtgcgcagcccgggctgggaaacagcgactgaaggagaaggagcaggagaatgagaggaaagtggcacagcttgctgaagagaacgagcggctcaacgaggaaatcgagcgcctgaccagggaggtagagaccacacggcgggctctgatcgaccgcatggtcagtctgcaccaagcatgaactgttggcatcacctcctgtctgtctctcccggagtgtacccagcaccatcacgccagtgccaagcatgtaatctccagtgcacatgctgaggaggggactgagggtagaccaaaggagaggggcttgtacactgtacattctttattcattccatacccagtaaagtgactttgtgtgaaaaaaaaa

SEQ ID NO: 36 - DNA, 566 bp, Rattus norvegicus
  misc_feature: >gi|6162249|gb|AW142350.1|AW142350 EST292600 Normalized rat kidney, Bento Soares Rattus sp. cDNA clone RGIAH46 5′ end, mRNA sequence
aggtagacaactgactggagagagtgggcagactctgacacggtggaagggaccctcagatactgagatgtgtttatgtgaagcctctccctgcaaggcagagccatcttccgcaacggtgcctgagatcaaaataaaaactgaccagaaagatcaaagctagctagctcttcccaaactcagagcgtctgagttacagaaaccagctttgcttcatcagtctgcaggttatgccttattagtgtaagggattccgaatgactacatagtttggtattgcttgtttacttaaagtgtatagctagctagtttttgatcctatttccctgctcctccaaaaaagtaataacatgtttcttgctgcgttccaagttgcagagttgctaaccggtcagggggaaatggggcttggaccatggtcaccctgtttgactgtacagtaattgacaatagatattaatgctcagaatggttgctctttaggaaatgactaaaaagctgtgagagaataaggaaatcttctcactcgatggtagcattgcttggcttctggctttctacactta

SEQ ID NO: 37 - DNA, 501 bp, Rattus norvegicus
  misc_feature: >gi|3291238|gb|AI044335.1|AI044335 UI-R-C1-jx- h-03-0-UI.s1UI-R-C1 Rattus norvegicus cDNA clone UI-R-C1-jx-h-03- 0-UI 3′,mRNA sequence
cggccgcacgtccacaaacagcccgagaagaccacccgagtgcggactgtgctcaacgaaaagcagctgcacaccttgcggacctgctacgcagccaaccctcggccagatgcgctcatgaaggagcaactagtggagatgaccggcctcagtccccgagtcatccgggtctggtttcaaaacaagaggtgcaaggacaagaaacgcagcatcatgatgaagcagctccagcagcagcaacccaacgacaaaactaatatccaggggatgacaggaactcccatggtggctgctagtccggagagacatgatggtggtttacaggctaacccagttgaggtgcaaagttaccagccgccctggaaagtactgagtgacttcgccttgcaaagtgacatagatcagcctgcttttcagcaactggtcaatttttcagaaggaggaccaggctctaattccactggcagtgaagtagcatcgatgtcctctcagctcccagat

SEQ ID NO: 38 - DNA, 1290 bp, Rattus norvegicus
  misc_feature: >gi|55926201|ref|NM_013060.2| Rattus norvegicus Inhibitor of DNA binding 2, dominant negative helix-loop-helix protein (Id2), mRNA
cgcagccacctcagccccctgcggcggctccctcccggcctttcctcctacgagcagcatgaaagccttcagtccggtgaggtccgttaggaaaaacagcctgtcggaccacagcttgggcatctcccggagcaaaaccccggtggacgacccgatgagtctgctctacaacatgaacgactgctactccaagctcaaggaactggtgcccagcatcccccagaacaagaaggtgaccaagatggaaatcctgcagcacgtcatcgattatatcttggacctgcagatcgccctggactcgcaccccactatcgtcagcctgcaccaccagagacctggacagaaccaaacgtccaggacgccgctgaccaccctgaacacggacatcagcatcctgtccttgcaggcgtctgaattcccttctgagcttatgtcgaatgacagcaaagtactctgtggctaaataaatggcatttggggactttttcctttttttttttttttttttatttttactttctctttttcttttgcacaacaagaagtctacaggatcttttaagactgttgttttgttctgctttgttatcaaccatttcaccaggagaacaagttgaatggacctttttaaaaagaaaacggaaggaaaactaagagtgatcatctcccccaggtgttctccggcctggactgtgataaccgttatttatgagagactttcaatgccctttctacagtcggaaggttttctttatatacttattcccaccatggggagcgaaaatgttaaaaaaaaaaaaaaatcacaaggaattgcccaatctaagcagactttgccttttttcaaaggtggagcgtgaataccagaaggacccagtattcggttacttaaatgaagtctttggtcagaaatggcctttttgacacgagcctactgaatgctgtgtatatatttatatataaatatatatatatatattgagtgaaaccttgtggactctttaattagagttttcttgtacagtggcagagataactttctgcattgaaatgtaatgacgtacttatgctaaactttttataaaagtttggttgtaaacttaacccttttatacaaaataaatcaagtgtgtttgtcgattgctcgctttatttcagacaaccagtgctttgatttttttttatgctatgttataactgaacccaaataaataccagttcaaatttatgtagactgtattaagattataataaaatgtgtctgacatcaaaaaaaaaaaaaaaaaaaaa

SEQ ID NO: 39 - DNA, 279 bp, Rattus norvegicus
  misc_feature: >gi|11407967|gb|BF419978.1|BF419978 UI-R-BJ2- bpi-e-05-0-UI.s1UI-R-BJ2 Rattus norvegicus cDNA clone UI-R-BJ2- bpi-e-05-0-UI 3′,mRNA sequence
gcggccgcttaattaagacggatctttactctcccccttgggtctgaagagctccataactgcgatcttcccatacatccccacctctttgactggccgtaggccttctgcggtgaccacatagatctctaatcttgtgtttttagcaatcaacaggttcaagtcttccgctgaagtaaagtgtccggtcacacagccgttcactgcggtaggcttctgcgccgttacgacgtanttgtacgacatattgaggtccttgggcccctcgtgccgaattct

SEQ ID NO: 40 - DNA, 3010 bp, Rattus norvegicus
  misc_feature: >gi|40254782|ref|NM_013130.2| Rattus norvegicus SMAD, mothers against DPP homolog 1 (Drosophila) (Smad1), mRNA
gagcgagcgggcgcaccaaggcgaggccagggccgaggcgcggggacggcggccgagagctaagcgaagcgctgggacggtgactgggaacggatcggagcgcgggacccggcgccgggtctcgtgcgtccccgccgatgggcgcgccgccgagccggtgctaactgggtcttccctgaagcagaaggaacagtatttcctacctttccgaaccgaagaccaagaagctagagaatctatgtaaataaactgaaatctctgttggctttgcacccccagcccggagccgtcacttttcctggtctgaggagcgtgtagacctcggccagccgctatgaatgtgaccagcttgttttcattcacaagcccagctgtgaagagactccttgggtggaaacagggcgacgaagaagagaaatgggcggagaaggctgtggacgctttggtgaagaaactgaagaagaagaaaggggccatggaagagctggagaaggccctgagctgccccgggcagcccagtaactgtgtcaccattcctcgctccctggatggcaggttgcaggtgtcccaccggaagggactgcctcatgtcatttattgccgcgtgtggcgctggcccgacctccagagccatcatgaactaaagcctctggaatgctgcgagttcccatttggttccaagcagaaggaagtctgcatcaatccctaccactataagcgagtggagagccccgttctcccaccggtgctggttccgaggcacagcgagtacaaccctcagcacagccttctggctcagttccgaaacctgggacaaaatgagccccacatgccactgaatgccacgttccccgactccttccagcagccacacagccacccgtttccccagtcccccaacagcagctaccccaactctcctggcagcagcagcagcacctaccctcactccccgaccagctcagacccgggcagcccttttcagatgccagccgacacacccccgcctgcttacctgcctcctgaagaccccatggcccaggatggctctcagcctatggacacgaacatgacgaacatgacggcaccgacactgcccgcggagatcaatagaggagatgttcaagcagttgcttacgaggaaccaaaacactggtgctctattgtgtactatgagctcaacaaccgtgtgggcgaagcgttccatgcctcctccaccagcgtgttggtggatggtttcaccgatccttccaacaataagaaccgattctgccttgggctgctctccaatgttaaccggaactccactattgaaaataccaggcgacacatcgggaaaggagtccacctttattacgtcggaggagaggtgtatgcggagtgcctcagtgacagcagcatcttcgtgcagagtcggaactgcaactaccaccatggctttcatcccaccacggtctgcaagatccccagcgggtgcagcttgaaaatcttcaacaaccaagagttcgctcagctactggcgcagtctgtgaaccatgggttcgagacagtgtatgaactcaccaaaatgtgcactattcgaatgagcttcgtgaagggctggggagcggagtaccaccggcaggatgtcactagcaccccctgctggattgagatacacccgcatggccctctccagtggctggataaagtccttacccagatgggttcgccccacaaccctatttcgtcggtgtcttaaagggccctcggcttctgtctcttgcaaactattgggccttgcatgtacttgaaggacggagaagtcagacaggatggggagctgtaaaggagccgtgatacttgacctctgtgaccaaccgttggatggagaagctgacaggccttggtaacaagctgttgatatcaagaacccgcttagtttacattgtgacattctgttgtaaaatcaactaaaatgctgacttttagcaggactttgtgaatagttagaaaagagatggccaagccaggggcaaattatctattaggaaaaaagcaaaaaaaaaggccctaagcaatccttttgtgtggggtgttggcagaaggttggcgccgatcatctttatggagtgggctctcatcaggctcggagcccacgctgatcatcctctcatgggttttcttaatattttaaaactatttgtttagaaatgaatgggtttttttgtttgttttaaagtaaaggttaattgttatgccatgcgtagtaacctttctgaagctgtatgctgactgtattgctgtcaggatgacgtcaggcatatgccctttgctaaatatgtatatacagaatacttggaggttatgaatagtccaaatggctagtgggtctacacagtatctgaggggcggggtcggggaggaaaaacgacagctgcaaatgtagacattgccgtaaagcccagcctgttgccttaaacggacaagctggtgtcggactttgctgtgtttcaggttttttagagttttacctggcttttcttcctttcttttctgctgtctcatccgctccacaatgcagttaagcagctggttaattcctctaactgtaagggcaaatgagtaattccttctgttcgcaaacggactggctctgcgtcccagttcccggaacatgaaaagcgtggatttaacgagcagttcttatggcgtttacaatacagacataggctttgatttttcaaataaattgtttgccaaacctggtaactctgttcattattcgcaggtttgaagaagatctctatcagaatccatttcaaagttttttttttttaattttgtttcatttttttttgtactatttggttttcttctgttgaagttttctattctccttttctcttatacagcgagttcttttattccaacactagcagggtttttctctactggaaatttttaaataaaacctgtcattattgcttaaaaaaaaaaaaaaaaaaaaaaaaaaaaaaaaaaaaaaaaa

SEQ ID NO: 41 - DNA, 4991 bp, Rattus norvegicus
  misc_feature: >gi|6978818|ref|NM_012555.1| Rattus norvegicus v-ets erythroblastosis virus E26 oncogene homolog 1 (avian) (Ets1), mRNA
gaattcggcacgaggcgccggccagccgagagagtgagcgagcgcccgggacagaggagcgagcgggcgaggcgcgggcgaggctgggacccaagctcgctctctgctccgccaagtgccaacttcgcgccgactggctgggcgcgcacctttctgccgggcgatcagcgggaatttgagattttagggctgcgctctgtccccttcccctgagttcaaaatccctattaaaaagcaaaacaacagttacagcaaacttgctctcatcccgccggccccctcaactccggcaccatgaaggcggccgtcgatctcaagccgactctcaccatcatcaagacagaaaaagtggatttggagcttttcccatccccggatatggaatgcgcagatgtcccactgttaactccgagcagcaaagaaatgatgtcccaggcactgaaagctaccttcagtggtttcacaaaagagcagcagcgactgggaatccccaaagacccccggcagtggacagaaacccatgtccgggattgggtgatgtgggctgtgaatgagttcagcctgaagggtgtggacttccagaagttctgtatgaatggagcagcactgtgcgccctgggtaaagaatgcttcctcgagctggctccagactttgtgggggatatcctgtgggagcatctagagatcctgcagaaagaggatgtgaaaccgtatcaggttaatggagtcaaccctacctacccagaatcccgttacacctcggattacttcattagctatggtatcgagcatgctcagtgtgtccctccctcagagttctcagagcccagcttcatcacagagtcctatcagacgctgcatcccatcagctcggaagaactcctgtccctcaagtatgagaacgactacccttccgtcattctccgggaccctctccagacagacaccttgcagacagactactttgccatcaagcaagaagtgttaactccagacaacatgtgcatggggagagccagtcgtggtaaactcgggggccaggactcttttgagagcatagagagctacgatagttgtgaccgtctcacccagtcctggagcagccagtcatctttcaacagcctgcagcgtgtcccctcctatgacagcttcgactccgaggactatcccgctgccctgcccaaccacaagcccaagggcaccttcaaggactatgtgcgtgaccgtgctgacctcaacaaggacaagcctgtcattcctgctgctgccctggctggctacacaggaagtgggccgatccagctgtggcagtttcttctggaattactcactgataagtcttgtcagtcttttatcagctggacaggagacggctgggaattcaagctttccgacccagatgaggtggccaggagatggggaaagaggaaaaacaagcctaagatgaattatgagaaactgagccgcggccttcgctactattacgacaaaaatatcatccacaagacggcgggcaagcgctacgtgtaccgctttgtctgcgacctacagagcctgctgggatacaccccagaggagctgcatgccatgctggatgtgaagccggatgctgatgagtaacggacacggaaggggctgggggggaccgctgagacctttcaaagaacaacccgtgttggttggactcttaatttttaattgttattctatgttttattttccagaactcatttttcacattcaggggtgggagctaagggagttgcagccacattccattggccattggcaaggccggaaagggaaagtcaggacctgtggggtgggtggggcaagaagttcctgaggagatttttcaggagcgagagggtcttctcagaagccgagcgacttgacttacagaggaaaaggtttatgtgtccagtattctgtttgcaatcaaaggaaagagttcatcttgagttgtggatcgactagcaggaggaattgtcacgttaagatgaaagtcagtcagcgttggtgggtgggaggaaaacagtttcttcaggggatgactaaacgcaagaatttattaacctttctactttttttgaaacaaagatggactttaatggaagggatccaaaactgtttttattgttgaagtttattttattaaattctgtgccagtattttttttcttaaaaaaaatcgtcttaagctctaaggtggtctcagtattgcagtattgtgagttcgttgtaatttgctggctgaggagtctcccacaatgaaaggcagctgtttatatagaccccatggaaaaatccaagtctgtactgagaacagagacccccgaactcttacagctaaaggacgtgaatgctgatttggggaccagggaactgtgtgaattccacctgcggtttgtaaaacaacatttccagtccttttacttactgtcaggctgcgggcttagtgttagttaaggggcattaagtctttgcactgaatgtatttttgcagctctgctttggaactgttgtcagcataggtgcactgtttaagtcttggaaaagaaatcgaaggaggaagttgaacctggtttttggttaaatcgctacccgaaacatagaagactcaaactaaatgctgtttgcatgggcattacccctcggatcttaaggcctaaatgcatgccgttccaaactaacattgtacaatttctcctttatggcttatttccccagttcaccacattgctgcccctccccaccttactaatcagtagagtggaatgtaatgtttcctgataggtgaatgaaagtgtaatttcagacatcacagcacaaatcaaagagtacagactttctagggttcagagagcatggaaacgaggaggctatttcaggagcatgggacatggcctcctttgggaagaggctttcctttgaagaattggagcttgtcaggtaggatttcaggtgaggctgtttgcagatgaagtggtagagagcactgggacttcgagcttgacctgttgctacctcggcagagttaagcgaaagtcagctgcggatgagaggaaaacttgatggagggaatacaacggcaggagctaagaacggagaataaggtggcccgtgacagtgaggtatagttttactttcctcaaagtttaaattcaaagacttaatcgaggcagccatcttcctgccagttgagagaggagactggaaagtaaggcattgtgggtaaaaacaaagtaaggaaacatgcctggtatcattagaatccctcccactgtttaccacgcctagttgagacagcgactgtggacttccatgagagaaaagaggcagttggttgaggtggacctctccaggctcacccttcggggattgtaactacctggtatcatcttcatgagagttgatggctgactcccagattcccttgaagactctgaattcctgcgaacataacttgtccatgggtgttctgtactgaccgattggagcatccccaccggaagcaaaggcaaaacatcccagctctatgttttgatcttaacaaatgcaggtgccttaatgaagctctcaaagtatttaggagctgctcagggagtgttaggtgggatcatttggattatgttgtttttctcttatattatgtgatctttgttgggcactggcagtgtgtgtgtgtgtgtgtgggtgtgtgtgtgtacatttgtataagcctgcagctgaaatagtactgacttttctacttaagtctgtccacatttccataacggcgaaagagtacatacagccagggctgatttcatttctcgctctttgcaacagggcttaaataaaaaaaaaatcacttccccaagtcgtcgtccttgcccctattcagttcatgctgaaaacgcccctctcgagagcctaccgcctgcagccagtagcagataagacgtaagaaagtgcctcccacacggctcagcttgctgacttgcttctccccaggtccaccactttcaggatttgtagataggatattagtcagcttggtggtccatctggccagacgctctttcccatgctgtccaaaggccagagaccatcccaggaagagtggcgggtggtttatacactggaaatgtagcggcattgctgcattgatgctctacaaaaacacgttcacttcagaggaaggatgagcacatctgatccagctgggtggctccattgttttcccagagagatgctttaacctgtgcggttggcttttggctcagagctcaggggaataaggatgctccctgcatagagctctgaccgttgcgccatggaaaccagtggaagcactctagatcagagacactcttcctcactttggagatcaacactctgggttttaaagcattaaccttcatggtgaaatcacaccttctctcttctagccatgctgtgcatgccgctttctctgtcggggggtctctataaatttgttgaactcttacgtacattccaaagaagtttcaaggaaccacaagtatatgtatacaaatacatatatgaagtatatatgttaaaatgaaattatctctatcaggaatactgcctcagttaattgaattttttttaaggatacttttttttaagcggagaattattggggtgtaaaagatgttatattgtgtttgactattttccagcttgtattttcacataatttatattttttaaatgctgaaaatttaaaagcgagatttaaaaaggaaaagcaggtgctttttttttaaaaaaaagaatcagaactgaggtagcttagagatgtagccatgtaagtgtcttaaatgttttgtttttaaaaaaaaaacaaatgcaaaaaaattcttatgggggagttttttgtttgtttcttttagtagctgatgctggcacattttgctggaaagttttttatatactgtagcctgatttcatattgtattttaaactgtgctcgtgccgaattc

SEQ ID NO: 42 - DNA, 1400 bp, Rattus norvegicus
  misc_feature: >gi|31542349|ref|NM_052981.2| Rattus norvegicus cyclin H (Ccnh), mRNA
cgcatccagcacttccattttccaatcaagtccctctctccacaggtcctgaagtccctagtgctcacgagtcctcgggaaccaggatcactgcagccgcacccacttctgaccgctaccaggccacgtccgtcagactcctcccaagcctgggagccccgggcagacgcaggcacgtgaccgtgacgtcacgcgcgcgcctgcggtctgggccgagggttctcgcgttggcagtcgctcgtggctgctgtctcccggagtgtcgctgtttctgtctcggccctgcggcccgtgagggtacagcatgtaccacaacagcagccagaagcggcactggaccttcgctagcgaggagcaactggcgcgtctgcgggccgacgccaaccgcaaattcaagtgcaaagctgtggctaacgggaaggttcttccaaatgatccgttgtttcttgagcctcatgaagaaatgacactttgcaaatactatgaaaaaagattgttggaattttgttcagtgtttaaaccagctatgccacggtctgttgtgggtacagcttgtatgtatttcaagcgtttttatcttaataactcagtaatggaatatcaccctcggataataatgcttacttgtgcatttttggcctgcaaagtagatgaattcaatgtgtctagtcctcagaaagaagggaaccttcgagagagtcctcttggacaggagaaggcactggaacagattttggaatatgaactactacttatacaacaacttaattttcacctcattgtccacaatccatatagaccatttgaaggcttcctcattgatataaagactcgataccccatgttggagaatccagagattttgaggaaaacggctgatgattttcttagtagaattgcattgacagatgcttatcttttgtacacaccttcacaaattgccctgactgccattttatcaagagcctctagggctggaattactatggaaagctatttatcagagagtctaatgctgaaagagaacagaacttgcctgtcacagctactggatataatgaaaagcatgagaaatctagtaaaaaagtatgagccacccagatctgaagaagttgctattctgaaacagaagttggagcgatgtcattcttctgaccttgcacttaatatggttacaaagaagaggaaaggctacgaagacgatgactatgtatcaaagaaacccaaacaggaagaggaggagtggacggacgatgacctggtagatgctctctaaccagtgtaaatgactgcagcagtacttactgaccgacggaagtaggaagcatgtctgacattttacttttgtcaaaatagtgcaatgtgaaaacatcaaaatatattaaacttttctaattctt

SEQ ID NO: 43 - DNA, 3251 bp, Rattus norvegicus
  misc_feature: >gi|13929115|ref|NM_031788.1| Rattus norvegicus RE1-silencing transcription factor (Rest), mRNA
atggccacccaggtgatggggcagtcttctggaggaggaagtctctttaacaacagtggcaacatgggcatggccttacccaacgacatgtatgacttgcacgacctctcgaaagctgaactggcggcacctcagctcattatgttagccaacgtggccctgactggggaagtgaatggcagctgctgtgattacctggttggtgaagagagacagatggccgagttgatgcctgttggagacaaccacttttcagatagcgaaggagaaggccttgaggagtcggctgaactaaaaggtgaccccagtgggctggacaacatggaactgagaagtttggagctaagcgttgtagagccccagcccgtatttgaagcatcagctgccccagaagtgtacagctcgaataaagatcccgcccctgaagcacccgtggcggaggacaaatgcaagaatttgaaggccaaacccttccgttgtaagccatgccagtatgaagcggagtctgaagaacagttcgtacatcacatccgggttcacagtgctaagaagttttttgtggaagagagtgcagagaagcaagccaaagccagggaatctggggcttccccgtctgaggagggcgagttctccaagggtcccatccgctgtgatcgctgtggctacaataccaaccggtatgatcactacacggcacacctgaagcaccacctgagagccggggataacgagcgtgtctacaagtgtatcatttgcacgtacacgacagtcagcgaataccactggcggaaacacctgaggaaccattttcccaggaaagtctacacgtgtagcaagtgcaactatttttcgacagaaaaaaataattatgttcaacacgttcgaactcacacaggagaacgcccttataaatgtgaactgtgtccttactcaagttctcagaagactcatctaactcgacacatgcgtactcactcaggtgagaagccatttaaatgtgatcagtgcaattatgtggcctctaatcagcacgaagtgacccgacacgcaagacaggttcacaacgggcctaaacctcttaattgccctcactgtgactacaaaacagccgataggagcaacttcaagaagcacgtcgagctgcacgtcaaccctcggcagttcaactgccccgtgtgtgactacgcggcctccaagaagtgtaacctgcagtaccatttcaagtccaagcaccccacctgccccagcaagacgatggacgtgtccaaagtgaagctgaagaaaaccaagaggagggaggctgacctgcaccgtgacgccgccgccgccgccactgagcagacggacacagagcaagcgaaaaccaagggggtggacgcgtctgcgaggagaagtgagaggcctgtaaaaggcgttggaaaagatgttccaaaagagaagaagccctgtagcaatgcctctgtggtgcaggtaactacccgaactcggaaatcagcggtggagactaaagcagcggagggaaaacacacagatggacagacaggaaacaacgcagaaaagtcctctaaagctaagaagagcaaaaggaagatggacgccgaggcccatccctcggtcgagcctgtgactgagggacccgtgacaaagaagaaaaagacggagagcaaacccaagaccagcggcgaagtgccgaagggcagcagagtggaggacaggaaggcggacaaacagcaaagtgcttccattaagaaaggcgggaagaagacggctctcaagactaagacagctaaaaaaggcagcaaacttgctccgaagtgggtggggcacacagaaccttcctcggagatggctcaaggaggggagtctccagttcctgctctcactcaggcggtggtcaccccatcaggatctactcagacagagctctcttctcccatggatattgctcagacagagcctgcccagatggacgtttcccagacagggccgcctcaggtgcagcggcctcttcctgtggagcctgctcaattggagccgtctcctcctcaggagcctccccaggtagagccacctgcctgtgtggagcctccccctcccgtggagcctccatgtcccatggagcctgctgagatggaaccgtcccctcccatggagccttcccaggtggagccacctcctcatttggagcctccgcttcccatggagctgcctcaggtggagctgcctcctgtggaggattgtcagaaggagctgcctcctgtggagcatgctcagactaaggttgctcagacaggtcctactcaggtgggagctgttcaggaggagccccttttctgtctccgagccacctcaagtcaagctaaccagaaggtcatctccccgaaagaccgtgccaaggagaagttgagcgtgctgagtgagatggcgaggcaggagcaggttcttattgaggttggcttagtgcctgtcagagatagccagcttctgaaggccagcaagagcgcaccggacctcccagccccaccgtcaccactgccaaagggacacttgagaagagaagagacacccaaggaccaagaaatgttctctgacggggaaggaaataaagtatcccctctcgagaaaggaggaacagaggaagctggtgagagtcgagctgagctggctgctcccatggaatctaccagtgctttatcctctgaacaaagctcaaatgcaccagatggtgaaacattacacagcgagtgtcaggctgactccactgcggtttgtgaaatggaagtggacactgagcagaagacagaccgtgtccctctgaaagactcagcagtagaaccagtgtcacctcttaacccaagagtggaccctgaagcagcggcaccagctgtggtggcctcccctcctatcactttggccgagtctcaggaaattgatgaggatgaaggcattcacagccatgatggaagtgacctgagcgacaacatgtctgaggggagtgatgactcaggactgcatggggctcggccagcaccacaggaagctacgtcaaaaagtggaaaggaagggttggctgtcaaagtaactgagggagagtttgtttgtattttttgtgatcgttcttttagaaaggaaaaagactatagcaaacacctcaatcgccatttggttaatgtgtacttccttgaagaagcagctgaggagcaggagtagagtagctgatcctcgaggagaagcgcaatgcgactttgtaa

SEQ ID NO: 44 - DNA, 3718 bp, Rattus norvegicus
  misc_feature: >gi|13242248|ref|NM_024359.1| Rattus norvegicus hypoxia inducible factor 1, alpha subunit (Hif1a), mRNA
gacaccgcgggcaccgattcgccatggagggcgccggcggcgagaacgagaagaaaaataggatgagttccgaacgtcgaaaagaaaagtctagggatgcagcacgatctcggcgaagcaaagagtctgaagttttttatgagcttgctcatcagttgccacttccccacaacgtgagctcccatcttgataaagcttctgttatgaggctcaccatcagttacttacgtgtgaggaaacttctaggtgctggtgatcttgacattgaagatgaaatgaaagcacagatgaactgcttttatctgaaagccctggatggctttgttatggtgctaacagatgatggtgacatgatttacatttctgataacgtgaacaaatacatggggttgactcagtttgaactaactggacacagtgtgtttgattttacccatccatgtgaccatgaggaaatgagagaaatgcttacacacagaaatggcccagtgagaaaggggaaagaacaaaacacgcagcgaagcttttttctcagaatgaaatgtaccctaacaagccgggggaggacgatgaacatcaagtcagcaacgtggaaggtgctgcactgcacaggccacattcatgtgtatgataccagcagtaaccagccgcagtgtggctacaagaaaccgcctatgacgtgcttggtgctgatttgtgaacccattcctcatccatcaaacattgaaattcctttagacagcaagacatttctcagtcgacacagcctcgatatgaaattttcttactgtgatgaaaggattactgagttgatgggttatgagccagaagaacttttgggccgttcaatttatgaatattatcatgctttggactctgatcatctgaccaaaactcatcatgacatgtttactaaaggacaagtcaccacaggacagtacaggatgcttgcaaaaagaggtggatatgtctgggttgagactcaagcaactgttatatataatacgaagaactctcagccacagtgcattgtgtgtgtgaattatgttgtaagtggtattattcagcacgacttgattttctcccttcaacaaacagaatctgtcctcaaaccagttgaatcttcagatatgaaaatgacccagctgttcactaaagtggaatctgaggacacgagctgcctcttcgacaagcttaagaaagagcccgatgccctgactctgctagctccagcggctggggacacgatcatatcactggacttcggcagcgatgacacggaaactgaagaccaacaacttgaagatgtcccgttgtacaatgatgtaatgttcccctcttctaatgagaaattaaatataaatctggcaatgtctccattacctgcctctgaaactccaaagccacttcgaagtagtgctgatcctgcactgaatcaagaggttgcattgaagttagagtcaagcccagagtcactgggactttcttttaccatgccccagattcaagatcagccagcaagtccttctgatggaagcactagacaaagctcacctgagcctaacagtcccagtgagtactgctttgatgtggacagcgatatggtcaatgtattcaagttggaactggtggaaaaactgtttgctgaagacacagaagcgaagaatccattttcagctcaggacactgatttagacttggaaatgctggctccctatatcccaatggatgatgatttccagttacgttcctttgatcagttgtcaccattagagagcaattctccaagccctccgagtgtgagcacagttacaggattccagcagacccagttacagaaacctaccatcactgtcactgccaccgcaactgccaccactgatgaatcaaaagcagtgacgaaggacaatatagaagacattaaaatactgattgcatctccaccttctacccaagtacctcaagaaatgaccactgctaaggcatcagcatacagtggtactcacagtcggacagcctcaccagacagagcaggaaagagagtcatagaaaaaacagacaaagctcatccaaggagccttaacctatctgtcactttgaatcaaagaaatactgttcctgaagaagaattaaacccaaagacaatagctttgcagaatgctcagaggaagcgaaaaatggaacatgatggctccctttttcaagcagcaggaattggaacgttactgcagcaaccaggtgaccgtgcccctactatgtcgctttcttggaaacgagtgaaaggatacatatctagtgaacaggatggaatggagcagaagacaatttttttaataccctctgatttagcatgtagactgctggggcagtcaatggatgagagtggattaccacagctgaccagttacgattgtgaagttaatgctcccatacaaggcagcagaaacctactgcagggtgaagaattactcagagctttggatcaagttaactgagcttttcctaatctcattcctttgattgttaatttttgtgttcagttgttgttgttgtctgtggggtttcgtttctgttggttgttttggacactggtggctcagcagtctatttatattttctatatctcatttagaggcctggctacagtactgcaccaactcagatagtttagtttgggccccttcctccttcattttcactgatgctctttttaccatgtccttcgaatgccagatcacagcacattcacagctccccagcatttcaccaatgcattgctgtagtgtcgtttaaaatgcacctttttatttatttatttttggtgagggagtttgtcccttattgaattatttttaatgaaatgccaatataattttttaagaaggcagtaaatcttcatcatgatgataggcagttgaaaattttttactcatttttttcatgttttacatgaaaataatgctttgccagcagtacatggtagccacaattgcacaatatattttcttaaaaataccagcagttactcatgcatatattctgcatttataaaactagtttttaagaagaaactttttttggcctatggaattgttaagcctggatcatgatgctgttgatcttataatgattcttaaactgtatggtttctttatatgggtaaagccatttacatgatatagagagatatgcttatatctggaaggtatatggcatttatttggataaaattctcaattgagaagttatctggtgtttctttactttaccggctcaaaagaaaacagtccctatgtagttgtggaagcttatgctaatattgtgtaattgatattaaacattaaatgttctgcctatcctgttggtataaagacattttgagcatactgtaaacaaaaaaatcatgcattgttagtaaaattgcctagtatgttaatttgttgaaaatacgatgtttggttttatgcactttgtcgctattaacatcctttttttcatatagatttcaataattgagtaattttagaagcattattttagaaatatagagttgtcatagtaaacatcttgtttttttttctttttttctatgtacattgtataaatttttcattcccttgctctttgtagttgggtctaacactaactgtactgttttgttatatcaaataaacatcttctgtggaccaggaaaaaaaaaaaaaaaaaaaaaaaaaaa

SEQ ID NO: 45 - DNA, 996 bp, Rattus norvegicus
  misc_feature: >gi|54262170|ref|NM_022593.2| Rattus norvegicus transcription elongation factor B (SIII), polypeptide 1 (Tceb1), mRNA
gggggcacttccttcggacgcaagttattagcttactgcttctcggcctcgccaggaaactagtaacttcctctgggatcaaatagaattttataagaacacaatggatggagaggagaaaacctatggtggctgtgaaggccctgatgccatgtatgtgaaattaatatcttctgatggtcatgaatttattgtaaaaagagaacatgcattaacatcaggaacaataaaggccatgttgagtggtccaggtcagtttgcggagaatgaaaccaatgaggtcaactttagagagatcccttcacatgtgctatcgaaagtgtgcatgtattttacctacaaggtccgctatactaacagctccactgaaattcctgaattcccaattgcacctgaaattgcactggaactgctgatggccgcgaacttcctagattgttaaataaaataaattataataaactgttaatttttttcagtatttaatacctgtagttcagttagtaatttttcacatgtagcatgttgcctgtatgaagctgaactctaaagtgaatcgctgagcagatttcttctgtcatttgcatagcagagttgaaatttgtttgctacatcaacaaattaaggaagtttcacaagccaaaaaaataaataaataatgccagtttataggtggtttgctttctcctttggatgtgacctttaaagttatacctgatatagcaaatcctggaatgtaagcgtaaatgaatatattctacaggtaaatacaggggctaaggatttttatgtttttagtgtttttttaagaaccagttctgatcttaagccactgagcattattagaattatgcaaataattacatgaaaaacatgtttataacttagccgaaccttgatttttgtaactcaaagaataagagttgaaatttcttatgtgttttttgataaactgcagtattgtctaaaaaaaaaaaaaaaaaaaaaaaaaaaaaa

SEQ ID NO: 46 - DNA, 2106 bp, Rattus norvegicus
  misc_feature: >gi|35278|emb|X54549.1|HSPAN2 R.norvegicus Pan-2 mRNA
tggtgtcactacacagggctgcctctaggcctggggaacgcaccccagttccgacacctgctgccctggctggatgatgaaccagtctcagagaatggcacctgttggctcggacaaagaactgagtgacctcctggacttcagtatgatgttcccgctacctgtggccaatggcaagggccggcccgcctccctagctggaacccagttcgcaggctcaggactggaggaccgacccagctcagagtcctggggcaacagtgaacagaacagttcttcctttgaccctagccgggcatacagcgaaggtgcccacttcagtgactcccacagcagcctgccgccttccacgttcctaggagctgggcttggaggcaagggcagtgaacggaatgcctatgccaccttcgggagagacaccagtgttggcaccctgagtcaggctggcttcctaccaggtgagctgggcctcagcagtcccgggccactgtccccatcaggcgtcaagagcagctcccagtattacacctcattccccagcaaccctcggcggagagctgcagatggtggcctggatactcagcccaagaaggtccggaaggttccgcctggtcttccttcctcggtatatccatccagctcaggtgataactacagcagggacgccacagcctacccctccgccaagacccccagcagcgcttacccctcccccttctacgtggcagacggcagcctgcacccctcagcggagctctggagtccccctggccaggtgggctttgggcccatgctaggtgatggctcggcccccctgccgcttgcaccgggcagcagttccgtgagcagtggtgcctttgggggcctccagcagcaggatcgcatgggctaccagctgcatgggtctgaggtcaatggtacgctcccagctgtatccagcttctcagctgcccctggcacttacagtgggacttctggccacacgccacccgtgagcggggccgacagcctcttaggcacccgagggactacagccagcagctcaggggatgcccttgggaaggcactggcctccatctactccccggatcactccagcaataatttctcgcccagcccctcaacgcctgtgggttcaccccagggcctgccagggacatcacagtggccccgggcaggagcgcccagtgccttatcccccaactacgatgcaggtctccatggcctgagtaaaatggaagatcgcttggacgaggccatccacgtcctccgcagtcacgctgtcggcactgcaagcgagctccatggactgctgcctggccacagcacgttgaccacaagctttgcgggccccatgtcactgggcgggcggcatgcaggcctggtcagcggaagccaccctgaggatggcctcacaagtggtgccagtcttttgcataaccatgccagccttcccagccagcccagctccctccctgacctctcacagaggccacccgactcctttagtggactcgggagggcaggcgtgactgcaggcgccagtgagatcaagcgggaggagaaagaggacgaggaggtcacatcagtggcagacgctgaggaagacaagaaggacctgaaggtcccacgcacacgtaccagcccagacgaggacgaggacgaccttctccccccagagcagaaggccgagcgggagaaggagcgccgggtggccaataacgcccgagagcgcctgcgtgtccgcgacatcaatgaggcctttaaggagctcggccgcatgtgccagctgcacctcagcactgagaagccgcagaccaaactgctcatcctgcaccaggccgtggccgtcatcctcagcctggagcagcaggtgcgagaacgcaacctgaaccccaaagcagcctgcttgaagcggcgggaggaggagaaggtatctggcgtggtcggggatccgcagctggcgctgtcagccgcccacccgggcctgggtgaggcccacaatccagctgggcacctgtgagccgtcacagggtcttcgttggaccagggaccaccacatctctgcccggggtgcacccaggatggtcctggatgagacatctc

SEQ ID NO: 47 - DNA, 674 bp, Rattus norvegicus
  misc_feature: >gi|8080489|gb|AW914809.1|AW914809 EST346113 Normalized rat ovary, Bento Soares Rattus sp. cDNA clone RGIBH57 5′ end, mRNA sequence
gcgcttgagaaagaccaggagcaagtggtgatgaacttggacagcagactgctgatcttccctctatacatctgctgtaacttcttgtatatatcaccagaaaaaagaactgagagtaatcgtcacccagaaaacccaggacactgaggcacgcatcggtggccagctttaacttaatgacctactgcgttcacgtccaaggtgactaacagtgaccggccttatgaactgtgtggacgatgcagatgtcctcaccctcattacatttctatgcacatatgaaaaagtatgttaaaaccaagaaagcatctgtcaaaccatatatgttaaaaggatatctatcagctctagctttcatttagtagcagtagaaaattgctgtaggtaaatttctcacttctttgcaacaaaatatagaattgattttgtgcttgaattttgatcctatctaatgttagtgaacttattcatctgtaaatgtgttcctgttttgttaagagaatgctaaggatgtagtttgagtggccaatcgtaaatgctctttcaatcggtgcctttaaagctgtcctttcatttttcagtctcccggttaagtcctctgtttagtttaaccctttccagtctctgcttctttttaaccattatttcctttntcatggtttgggagactaagc

SEQ ID NO: 48 - DNA, 318 bp, Rattus norvegicus
  misc_feature: >gi|13305465|gb|BG380993.1|BG380993 UI-R-CT0- bul-h-06-0-UI.s1UI-R-CT0 Rattus norvegicus cDNA clone UI-R-CT0- bul-h-06-0-UI 3′,mRNA sequence
ttttttttttttttttttggcggatcaaactaggatttactcaagcagacgtggggctggcgcttggcaccctgtacggcaacgtcttctcgcagaccaccatctgcaggtttgaggccctgcagctgagcttcaagaacatgtgcaagctgaagcctttgttgaacaagtggttggaagaggcagactcatcctcgggcagccccaccagcatagacaagatcgctgcgcaagggcgcaaacggaaaaagcggacctccatcgaggtgagcgtcaagggggctctggagagccatttcctcaaatgccctaagccct

SEQ ID NO: 49 - DNA, 483 bp, Rattus norvegicus
  misc_feature: >gi|14928015|gb|BI279828.1|BI279828 UI-R-CU0s- cbr-d-08-0-UI.s1UI-R-CU0s Rattus norvegicus cDNA clone UI-R-CU0s- cbr-d-08-0-UI3′, mRNA sequence
ttttttttttttttttttatttcaaaagtgtttttaatgggaagatgacaggtcaacaaacgagcagttctgagggagcctcggggacatcagctgagctgatccttctgagaggtgcgtgccactataaagacctctgcctgttccctgtaccagcaccccgctttccacaggtgaagccaccaccacacgccccgcccactgagcacaggtggcttaactcgagggcagcatcccgggagttttctctcttgatatcccagaagaggtttctctgagcagggtccagggagctccattcttctcgggaaaaatagaaagggatgtctcccaatgccacagatttttgaaatggggagatatgacccagctcaggtctcccaggtagatcaacttcagaggcaaagcagacatccaacatctctcctgtccgtctctctttaagaacagggggctgggcgtggctgtgctgctcctcgtgcc

SEQ ID NO: 50 - DNA, 592 bp, Rattus norvegicus
  misc_feature: >gi|13242286|ref|NM_024383.1| Rattus norvegicus hairy and enhancer of split 5 (Drosophila) (Hes5), mRNA
cctggctctgctcgctccgctccgctcgccaatcgcctccagagctccaggcatggccccaagtaccgtggcggtggagatgctcagtcccaaggagaaaaatcgactgcggaagccggtggtggagaagatgcgtcgggaccgcatcaacagcagcattgagcagctgaaactgctgctggagcaggagttcgcgcggcaccagcccaactccaaactggagaaggccgacatcctggagatggccgtcagctacctgaagcacagcaaagccttcgccgcggccgccggccccaagagcctgcaccaggactacagcgagggttactcgtggtgcctgcaggaggcggtgcagttcctgaccctgcatgcagccagcgacacgcagatgaagctgctttaccacttccagcggcccccagctcccgccgcgcctgtcaaggagaccccgacgcccggcgctgcgccccagccggcgcgctcctccactaaggccgccgcctccgtctccacctcgcgccaatctgcctgcggcctctggcggccctggtgacccagcggccgacgggtgcctggagctgaccagaggacc

SEQ ID NO: 51 - DNA, 1893 bp, Rattus norvegicus
  misc_feature: >gi|6978540|ref|NM_012912.1| Rattus norvegicus activating transcription factor 3 (Atf3), mRNA
tgtctcactcagcgagacgcgcggtgcacggtgcttccccggcggacgaccgaccaacccgcgctccggcagagtccttggcgctcgcctgccggcgggacagacagcccgcctctagccgctctctggaccctggccgccccgagcgaagactggagcaaaatgatgcttcaacatccaggccaggtctctgcctcagaagtcagcgcgaccgccatcgtcccctgcctctcacctcctgggtcactggtgtttgaggattttgctaacctgacaccttttgtcaaggaagagctgagattcgccatccagaacaagcacctttgccatcggatgtcctctgcgctggagtcagtcaccatcaacaacagacctctggagatgtcagtcaccaagtctgaggtggcccctgaagaagatgagagaaaaaggaggcggcgggaaagaaacaaaattgctgctgccaagtgtcgaaacaagaaaaaagagaagacagagtgcctgcagaaggagtcagagaaactggagagtgtgaatgccgaactgaaggcccagatcgaggagctgaagaatgagaagcagcatctgatttacatgctcaacctgcaccggcccacgtgtatcgtccgggctcagaacgggcggacgccggaagacgagaggaacctttttatccaacagataaaagaaggaacattgcagagctaagcagaggtggcatgggggcaattggggagtccttactgaatcctccttttccaccccaaaccctgaagccattggaaaactggcttcctgtgcacttctagaatctcagcagccacgagctgttgggtcaggagggcctgcggtgactactgcgttgtcccactctgtccccgagtgaaccgtggagcaggcaggagcatcctttgtctcaccggctccaggatttaggccttaccatcccggccattctcagatgacctagctggccccaggctggggtcccatgcaaagcaggatcgcactaatgggatgcaggcagaagtgtctaccttgacaggtggggtggaccacgtcctccactgcggctgacaacatccctcctagggaagatggagtgagaacattcatcattgaagttgtccaatggccagggtatgctttctagaaactatgctgttctgtcctagactgactgtgcatagggcattcatttctgagcctggtgttgtgctatttagatgtttgtcttgcacaacattggcgtgatttttttccgggagtttcatcagacctgatttccgagagtttgggggtctgccactgtggacaatatcccccaaaagtgtttgggtggccatgtaaactggctgatgaccagctgtgctactctgtgctgaccgaggactgatgcctccttcccctgtacccactgctgaggaagaacccgggcacagcagctgtccttggctacaaactgttacaatgtcacagaacgaaggcacaaagtcccgctttcaaagggcgtaggactccacactcagtgacagggcaggaagagccaaggattctccgttttcccttccttcccaccaaaaaccacagcccgtggagactggtatttgaagccaggagtggggcaaggaaggtgtctgcactgtgggatgttaactgcgcttttgtcttgaagctattttgagatgcggtccagagtatttcagctgggaggtccctcccactggccaccagggctctggctactgttaaaattctgatgtttctgtgaaatcctcagtgttcaatccagactcagtagtatattacagttttctgtaagagagaacgttacttatttatcccagtattcctagcctgtcaacgtaataaaatatcagaatgagacctggt

SEQ ID NO: 52 - DNA, 1740 bp, Rattus norvegicus
  misc_feature: >gi|55926218|ref|NM_053563.2| Rattus norvegicus nuclear RNA helicase, DECD variant of DEAD box family (Ddxl), mRNA
gtgtcatggcggctctcggaggcccgcatgtgtactgtggtgggtttgaggcacggggcggctccaggctgcgttttcggtcgcggggagaggacccaggctttctgggcagatgccccgagtctgcactcacctgcttcccgtcttgctccgccgctctcccaggagattcacctccagactccgaagcggaaagccggtgtggcggcccttaggccattcaccgggtgtcgttttcccggtgtgaggccggttcagccagttgcatctcgtcttcctgggcctgtttgaaactttagtgacatggcagaacaggatgtggaaaatgagcttttggattatgatgaagatgaagagccccaggtaccccaggagagcactccagctcccccgaagaaagatgtcaaaggatcttatgtctccatccacagttctggcttccgggactttctgctgaagccggagctcctgagagctatagttgactgtggctttgaacatccttcagaggtccagcatgaatgtattccccaggccattctgggtatggatgtcctgtgccaagccaagtctgggatgggcaagacagctgtgtttgtgctggccaccctgcagcagattgaacccatcaatggccaggtatcagtactggtcatgtgccacacaagggagctggccttccagatcagcaaggagtatgagcgcttctcgaagtacatgcccagtgtcaaggtatctgtgttctttggaggcctctccattaagaaagatgaagatgtgttaaagaagaactgtccccatgttgtggtggggacaccaggccggatcctggccctcgtgcggagcaggagcctcaacctgaggaatgtgaagcactttgtgctagatgaatgtgacaagatgctggaacagctggacatgcgccgggatgtacaggagatctttcgtctgacaccccatgagaagcaatgtatgatgttcagcgccaccctgagcaaggagatccggccagtctgcaggaagttcatgcaggatcctatggaggtgtttgtggacgacgagaccaagctcacactgcatgggctgcagcagtattacgtcaaactcaaggacagtgagaagaatcgtaaactcttcgacctccttgacgtgctagagtttaaccaggtggtgatctttgtcaagtctgtgcagcgctgcatggccctggcccagctcctagtggaacagaattttccggctatcgctattcacagaggcatggcccaggaggagcgcctgtcccgataccagcagttcaaggacttccagcgtcgcatcctagtggctactaatctgtttggcagaggcatggacattgagagagtcaacatcgtcttcaactatgacatgccagaggactcggatacctacctgcaccgagtggctcgtgctggtcgctttggtaccaagggtctggcagtcacttttgtgtcagatgagaatgatgccaaaatccttaatgacgttcaggaccggtttgaagtgaatgtggctgagcttccagaagaaatagatatctccacatacattgagcagagccggtaaccatgtgtgtagccaggcacatggctttctctcctgctgcttcagatcctcctcctaggtggcaatcggcggcctctctttttattgttccaaagctttagctatgttaagaataaacttttattgtgaaaaaaaaaaaaaaaaaaaaaaaaa

SEQ ID NO: 53 - DNA, 564 bp, Rattus norvegicus
  misc_feature: >gi|3706241|gb|AI101323.1|AI101323 EST210612 Normalized rat brain, Bento Soares Rattus sp. cDNA clone RBRBL42 3′ end, mRNA sequence
ctgtttttttccccaaaacaatcaccaaaagcaagatgatccccaccccccaatcaaagcgaaacaaaatactgtcatgttaatcatctctctaaacaagccttctgcgctcagctgtgtgactgggaggagggggactcggtttaacttatggtgacctctgggctggaaggcccaagaggagcagcggtctgagaaccatggagtgagtaacagctctctctctcctggcctccccggtttcagaatgagctacaagagttttgcaaatttcaagcctcaatggtctgagcctccccaacccagccttatcaacttccctatgggtgtgcagtttctttccaaagaattgtgcaagtctaagcctcctgggtgaaggcatttagtagtccatgatcaacgtagattgtcataactcggtcccctctcatgggcgctaacctgaacacgctgctatactgctctgcacagactggccacggtggcaaggaggatccacttctgcatcttcccaggaacggattcagaatcaccagccatgtggtgggtttctgtctccccttg

SEQ ID NO: 54 - DNA, 2256 bp, Rattus norvegicus
  misc_feature: >gi|52138598|ref|NM_012963.2| Rattus norvegicus high mobility group box 1 (Hmgb1), mRNA
cgagagccggacgggcactgggcgactctgtgcctcgcggaggaaaatcaactaaacatgggcaaaggagatcctaagaagccgagaggcaaaatgtcctcatatgcattctttgtgcaaacctgccgggaggagcacaagaagaagcacccggatgcttctgtcaacttctcagagttctccaagaagtgctcagagaggtggaagaccatgtctgctaaagaaaaggggaaatttgaagatatggcaaaggctgacaaggctcgttatgaaagagaaatgaaaacctacatcccccccaaaggggagaccaaaaagaagttcaaggaccccaatgcccccaagaggcctccttcggccttcttcttgttctgttctgagtaccgcccaaaaatcaaaggcgagcatcctggcttatccattggtgatgttgcgaagaaactaggagagatgtggaacaacactgctgcggatgacaagcagccctatgaaaagaaggccgccaagctgaaggagaagtatgagaaggatattgctgcctacagagctaaaggaaaacctgatgcagcgaaaaagggggtggtcaaggctgagaagagcaagaaaaagaaggaagaggaagacgacgaggaggatgaagaggatgaggaagaggaggaagaggaggaagacgaagatgaagaagaagatgatgatgatgaataagttggttctagcgcagtttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagctagccctgtcctggtggtattttcaatagccactaaccttgcctggtacagtctgggggttgtaaattggcatggaaattaaagcaggttcttgttggtgcacagcacaaattagttatatatggggacagtagtttggtttttggtttctttttttttttttttttggttttgttttttttccttttgttttttttttccatcttcagttgtctctgatgcagcttatacgaaggtaattgttgttctgttaactgaataccactctgtaattgcaaaaaaatggcggctgttttgttgacattctgaatgcttctaagtaaatacaattttttttattagtactgttgtccttttcataggtctgaaagttttcttctcaaggggaagctagtcttttgcttttgcccattttgggtcacatggattattagtgtgtatcttcatatagttggaaaaatcttttgtccacacaccctgcatattgtggtaggggtaacattttcatctacagttgaagaattctccaaaattgtgatcagttggataagagatactctacttagcaaagcaaggagtgatcaattctgtcacaccatgggattattagatcaaacagtctgaaagtctgtccttgaaggactaatagaaaagtatgttctgctccttacctgaggactcctttaactgccattactgtgtaatgacagttatattttgcagtttcccctactaaagacctgagaatgtatccccaaaagcgtgagcttaaaatacaagattgctgtactatttgttgaccttagtcccagcgaaggctatcatgagaagctggctgtaatgcctttgcccttctatctaaatacggattgctcaggaaacttgactgtttaaaggtatatttaattagttgagccagcttttaaaattatgccacatttaaaatgaagggtatattttcctatattgtggtttgtccctttataaatcagatacaaggaaagcggataaactttgcatattagtaccagttgtccaagccatttgctttttctttataaagcccgaactcattcattaatcatgtttaatctgcttagtttagggaacaatttggcaattttgtgggttttctttttttctttttcttttttttttttttttgagattatcattctcttaaagtgccagtgttttaaaatagcgttcttgtaattttccgcgcttttgtgatggagtgctgttatatcattttgacttgggttctttacagttgcctttgttctgtaatttgaggaggaatactgaacatgagtcctggatgatactaataaactaataattgcagaggttttaaaaaaaaaaaaaaaaaaaaaaaaa

SEQ ID NO: 55 - DNA, 4776 bp, Rattus norvegicus
  misc_feature: >gi|16758125|ref|NM_053400.1| Rattus norvegicus transducin-like enhancer of split 3, E(spl) homolog (Drosophila) (Tle3), mRNA
gcacgagccgaggcttcctatgccggctggagttaggatgccacggcccattatttgcaccgactaacccggagttcctcctcggccctgggaaggactggcggcggcggctggagcagacagagcagcaggctctcttctccgaggggaggggggggcgtcgaagcgccctcgctgggctctagccctcacccacggtcctcaagccgtcacctcggtggcctgcttcctttttcttctccccgcgactccgggcccgaccgcctctgggatttgacccgcgcgccccggatgctccgagctgcccagacgcgcccccccccctccagcctcatcgtgctgattgacccgtgaggggacaagagaaacccaccccccaacctccgggcctcgggatcctgctctggagctgccagggagccgatgatgtccacgcagacccggcttctgacactgtccacgcagccgggggaaggtggccggccgcagccggccgagccttaggcgcacaaagcgcgagcccgccgacgactttgccgctcgctccgccgctctttgtctgcacttggggcgcacgcccgactctgggatttcgctgcgagagcgggctgggggggccgggggcgagctctcggttccccggccgccccccgctcgggctcgcctcccccctcccccgcacgtccccggctcgggctctcagcgcttccaagctctcggaatcgcgacccctccctgccatgtatccgcaaggcagacatccggcaccccatcaacccgggcagccaggatttaaattcactgtggccgagtcctgtgacaggatcaaagacgaattccagttcctgcaagctcagtatcacagcctcaaagtggagtacgacaagctggcgaacgagaagacagagatgcagcgccattatgtgatgtactatgagatgtcctacggcttgaatatcgaaatgcacaagcagacagagatcgcgaagagactgaacacgattctagcccagatcatgccttttttgtcacaggagcatcagcagcaagtggcgcaggctgtggaacgcgccaagcaggtcaccatgacggagctgaacgccatcatcgggcagcagcaactccaggcacagcacctctcccatgccacacatggtcccccggtccagctgccaccccacccgtcaggcctccaacctcctgggattcccccagtgacaggaagcagctctgggttgctggcacttggtgccctgggaagccaagcccacttggcagtgaaggatgagaagaaccatcatgaactggatcacagagagagagagtccagcacgaacaattctgtgtcaccctcagaaagcctccgggccagtgagaagcaccggggctctgcagactacagcatggaagccaagaagcggaaagcggaagagaaagacagcctgagccgatacgacagcgatggggacaagagtgatgacctggtagtggatgtctccaatgaggacccagcgacaccccgggtgagcccagcacactcccctcctgaaaatgggctggacaaagcccgaggtctgaagaaagacgcccccaccagcccagcctctgtggcttcctccagcagcacgccttcctccaagaccaaagaccttggtcataatgacaaatcttccacacctgggctcaagtccaacacaccaacgccaaggaacgatgccccaactccaggcaccagcaccaccccggggctccggtcaatgccgggcaaacctccaggcatggacccgatagcctcggccctgcgcacacccatctccctcaccagctcctatgcagcaccctttgccatgatgagccaccacgagatgaatggctccctcaccagcccaagcgcctatgctggcctacacaacatcccatcccagatgagcgccgccgcagcagctgcagccgccgcctatggccgatcgccaatggttggttttgaccctcatcccccgatgcgggccacaggcctgccctccagtcttgcctccattcctggagggaaaccggcgtactccttccatgtgagtgccgacgggcagatgcaacctgtgcccttcccccatgatgcgctagcaggccccggcattcccaggcatgcccggcagatcaacacactcagccatggggaggtggtatgtgccgtgaccatcagcaaccccacgaggcacgtctacacaggcggcaagggctgtgtgaagatatgggacatcagccagccgggcagcaagagtcccatctcccagctggactgcctgaacagggacaactacatccgctcatgcaagcttctccctgatgggcgcacgctcattgtgggtggtgaggccagcacactcaccatctgggacctggcctcgcccacgccccgcatcaaggctgagctgacgtcctcggctccagcctgttacgccctggccatcagtcctgatgccaaagtctgtttctcctgctgcagtgatgggaacattgcagtttgggatctgcacaaccagaccctggtcaggcagttccagggccacacggatggggccagctgtatagacatctctcatgatggcactaaactgtggaccgggggcctggacaacactgtgcgctcctgggacctgcgtgaagggcggcagctacagcaacacgacttcacctcccagatcttctccctgggttactgccccactggggagtggctcgccgtgggcatggagagcagcaacgtagaggttctgcaccacaccaagccggacaagtaccaactgcatctgcacgagagctgcgtgctgtccctcaagtttgcctactgtggcaagtggtttgtgagcactgggaaagacaaccttctcaatgcctggaggacgccttacggagccagcatcttccagtctaaagaatcctcgtccgtcttgagctgtgatatttcagcggatgacaaatatattgtaacgggctctggtgacaagaaggccacagtttacgaggtcatctactgaacaaggactctaagaggcctgtcaaactctgggagacacccacgcggccctgacagggagatgctggccaggccccgaggatggcagaagagtgagggacagcattgtggggctgccctccggaaggtggagaggacgcacgcccctttgtggattgctgtgtctgacagacttgaaggactttctcctctccccctcaccagtgctggcagatgcttcaattagatttctctggaggcttctgctttcgtttcccacacagacaccctcatgagtctctgtctcttcctcccctgtctgatctgtcccctccctactccgggttgggggcatgctggagtagacattgttgtttctggggaggggcgcctcgttctgattgtgcagtgcacaaggtttgtgaacaatgtaaataagagactgtggttgctgactggccagtgagggaggaggccccttcccaccagtgctcgccgtgtattttgcctgctgtatttgtaacccactcgcggtggtggcttttctttctttcttttcttaagattttttttaaacagatgttctgtggggaaagtaggcagagatagagagtcggggagtggaatgagaagcttcctggggatgggcaaaccagcctggcccactgggactgaagtatatctccacacgggttttagcaccatcttcagatggaatgataggcagatggacagacaagctgatggacagacagtactgggatgggccagcttgggctattatccacaggaacagcgaggatttcctgtgggtttctctgctccatcttctcttgccttgccctcacacctcctgacctccatctctttctgcccccctccccccgctcgcctctcccactggaccccatctttctctttggtgcacactcaatcattgtgatgaggaatggaagttcaagggaactccctctctgactctcctccgccagcctagaaaggattccatatgggggtgggagggagacaagcgactcagctcagggagctaccagagaaaaacagcaactggtgtgggtgctttttttttttaatttgaataaaaataattagaagtgatatcctttgataagatgccgtctcccctttcctgctctcagccctttctgctccctgaagccggggatatggtgacccccagggtcgtaagtctaagaagatgggcgggggagcgggctgggggctggggcagacggaatgtcatctgcagatatcacccctagggtaccaatgtttgtggtattaggtttgctttgttttgttttggtcttttgttgttttattaaagaaaagctaaagaccgtgtgagccctgctggtgggctttccatggatgtagcatatggaagagagtttttatattgcatttttatggattattttataatgtgggactcagtctgggaagaaagatgagggtgtctaaaacctgcccatctccagtgctccaactgagcggctgccccgaacaggatgtatgttgggacatggaaagaccgtggcaccccgcaccttcctgagaggcatgtcgacatgcctgtctggagtctgtccatctgtctctgggtttcagatgatcactggccaccacttgttaaggctctagccagaagggagggttagggtggaagagagttattcctgaagaaaaaagaaaaattgaaacatcattgtaatgaagctttttatatttttaaaagttac

SEQ ID NO: 56 - DNA, 479 bp, Rattus norvegicus
  misc_feature: >gi|13890582|gb|BG668660.1|BG668660 DRNBDB11 Rat DRG Library Rattus norvegicus cDNA clone DRNBDB11 5′, mRNA sequence
tgcaggttcgagtcgctcacgttgtcgcacaacaacatgatcgcgctcaagcccatcctgcaggcgtggctggaggaggccgagggcgcgcagcgtgagaaaatgaacaagccggagctcttcaacggcggcgagaagaagcgcaagcggacttccatcgccgcgcccgagaagcgctccctcgaggcctattttgccgtacaaccccggccctcctcggagaagatcgccgccatcgccgagaaactggacctcaaaaagaacgtggtgcgggtgtggttttgcaaccagagacagaagcagaagcggatgaaattctctgccacttactgaggagggtccctgtctccctcccttggagaacatgaagttgttgaaaagctgagaagtttctgctttcatttgggggcaggagtcccatgtaagaaaaatcacctaaaatcccagaagaaaggaagagatttaaagatttaaagccn

SEQ ID NO: 57 - DNA, 4758 bp, Rattus norvegicus
  misc_feature: >gi|971274|dbj|D32249.1|RATNDAP1 Rattus norvegicus mRNA for neurodegeneration associated protein 1, complete cds
tggcgggtggaggatcgagcgctgttctcgctctggagccgctgcacatctcggaatcatttgcagcttattcactgtgtaaggaaaaaccaaattacatccataattctattgaactggaagcatagacgctgccatatatgtcacagtatacagaaaaagaaccatcagtaatggatcaagactccagcaaggctgcgtggcctagagcagcaggaggataccagacgattacaggcaggaggtacggaaggagacatgcgtatgtcagttttaagccatgtatgaccaggcatgaacggagcttgggtcgggctggcgatgactatgaagttctggaactggatgacgttgccaaggaaaacaccgcaggttccagttcattggatcaagtccatccttctttacccagtgaaactacagttgaaaaaagtgaaacagaaattcctacttgtggtccagcactgaatcaaagcacggagagcaacccatccgttgccacagtgtgtcatagtgaggaagtcagggagaccttagacagcagtacgaatcttcagaatcacgctgagagagagtgtacgccagcagtttgtaatgcctcaagtgtccagaatggaattgtgttggttcatactgactcttatgatccagacagcaagcatgatgagaatgactctcttcaactttgtgcccaagctgtggaaggtggtagacgtcagaaggtattaggcaatgcagtctttgagctggaaaatggagaggtagagagatatgctgatctgtgtccctcagttccctctctcagtggtgaaataagggaggagtctgaagagctaggttcagcactcttagagaaaaattctgctggcgatgcggaggctgtccatcaggatgggcaggaatttcagaggtcttctgaagacggcattgttagaaagaggcgacaagatgataccgatcagggaagacagacagaaaattcaactgaagatgcagactgtgttccaggccatgttgaacaaaatactagtgagagagccaatcaccatggaagttctcctgaacaggtagtgaggcccaaagttagaaaagtgatcagttcaagccaggtggaccaagagagcggttttaataggcacgaggctaagcaaagaagtgttcagaggtggagagaggctctggaagttgaggaatgcagttcagatgaccctataatcaagtgtgacgattatgatggagaccatgactgcatgttcctaaccccatcctactcaagagttacgccaagggaagcagaacgtcaccgtgcgacagcagaaaatggagccacagcttcaggaaggcaagaggctcgggaaaatgccttttggaatgcctgtggagagtattaccagctctttgacaaagacgaagacagttcagagtgcagtgatggggaatggtctgcttctctgcctcaccgattttctggcacagaaaaagaccagtcctcaagcgatgaaagctgggaaactctgccaggaaaagatgagaatgaacctgagctacagagtgatagtagtggccctgaggaagaaaaccaagaattgtctcttcaggagggagaacagacgtccttggaggagggggagattccctggttacagtacaatgaggtcaatgagagcagcagcgatgaagggaacgagcctgccaatgagtttgcacaaccagaagctttcatgttggatgggaacaacaacctggaggacgactcgagcgtgagtgaagacctggatgtggactggagcctatttgatggttttgcggatggacttggtgttgctgaagctatttcctacgtggatcctcagttcctcacctacatggcactagaagaacgcttagcccaggctatggagactgcactggcacacttagagtctcttgctgtagacgttgaggtggctaacccacctgccagtaaggaaagcatcgacggacttccagaaacccttgtcctagaagatcacaccgctattggtcaggagcagtgctgtcccatctgctgcagtgagtacattaaggatgacatcgcaacagagctgccctgccatcacttctttcataagccttgtgtctccatttggctacagaagtccggaacgtgccctgtgtgccgccgccacttcccacctgcagtgattgacgcatctgcagctgcttcctctgaaccagaccttgatgcctcgcctgcaaacgacaatgctgaggaagccccgtaagccgtcacggggaaatgagatcgaaattctatcacagtaaatctgcaaattccttctaaatctgatgtgcaaataattatatataaatatatttaaaatgctctatatagtatatgccgtagtttagaaagagaatattaacctttctaaactgaatttaggtttgcagaagatactaaacattttcaagctgaatgttgaagcagtgcatccattttctttagttgaacatggtgtccattgtaacgtcagtagtcatctctgtggcatacgtttctgttttgtctgaagtgctgccactaagagatcagaattaagctctcctttccacatcagatgtgaaaggagagcttaattctgatctctgtggcatacgtttctgttttgtctgaagtgctgccactaagagatcagaattaagctctcctttccacatcagatgtgaaaactgtgatcacaacagtagtacagtttggtttcattgaaaataaactgaattctaaagcatgctttttcactggtccctttgcttttgctacttcgagacctcttggtttatataacactgaggttaagattaaagcttttcagaatgccaggcaaagactagagttttgacccgaacacacacacaaaataaaaaaataaaaaataaattacatcttagatgtaaacttcctgaaactgtcatgtaataaactattcagtgaacgttggatgtttaaaggctagtaaagcatgtgaattatctgatgtgcttcatgctcggcacagtctcatgagcttggcctatgaaacactgtctaagccctcatgcagtctgcttctacttgttaaacactgaaggtccagaccagtcttggcatctgattaagaaattggatgtagttagctaaggtagtttgggttctactttaggagatctgaagatttgcacatggccatgtctgttacagcatcaccccagattagaaaggcacagcttttaggtggtcacacgagtaggcaagtctgtgcagacgctggtgtgataggtagcttacaggtgagccctgacatcagcacagggacaacggctcagcaggcccagcactggagcccatccgcgtgcacatctacctgccctgctcatccagttcgggaatatttctatagcttcccaaatcagcttcatctgatttgtgtaatatatctgagtatttatagtctttcttttttctccagtcttaaaaataaatgaattttcactgttggcacatttgaggcttaaatataagaaacctaacacttgcattctgatttttgcatatattgtaaatgtggctggtatttacagcgaatcctgtgtgtccttttatgggtaaaacaaaagtgaacattgcatgcaagtaatgtggtgaatttgtaatttaggggttttctggggcttctgtgactgtggatgcttaaattcaggccttaatgttgctctagctagcatgtttccttctaatgtacatagtcagcctgtatccactaatctctgcttgtcttctcctccgtggtcttcacaccgtatccactgatggtcagttagtgtcagaggtcacccaggttagcatcagttcccatgtgtgccgtgcggattgtgagccgtgtggttgcttggttggttttcttttatgtactttgattagctgtggcacttactgagtaaacttacgttgctgcagactgctgtgtaacaagccacgttttgtgtttagtcagtattacacacgggaccttgttttattcttctagatagcagctgtcccaaagaaaatatttcttctttgcctattaagatttagctattatctgccagttgttaaggggtttgattccaaactcaaccagagttgaacgtaagaaagctgctgtactcttgctgtccatctgttacgtcctccgttgttggctcctgactacgtacccacggctgctgaaggagacttgttttaatttgttttaaaaaggaaaatgaagttttaggactttgatggtggggaagctgacaagttcagaccccagtctgtcatttgctcttagcttggatccttttgaaaagttaagaatatatgaaggtaaattagaaaaagtaaaaatattgataaaatgtcatttatcttatgtctattttatgttgttatttggcctattaattttaattttttgaacattttcttatttcttgtaatatgaatgccgatatttaaagtttgactcacttgggtttcttttgtgtttcttagttgttatctctaagcctaactaacttttaagattatttgtgatatggatttatataagctgttaaatatatatgagctgttaaaatggaatgcaaggttttccaaagaccaggtctaactgtaatgattggtttattgttctacaatcccagcccggcattttccgtgtaaatcataaacaataaacaggatatactcagtgttcatttctaaaaaaaaaa

SEQ ID NO: 58 - DNA, 1191 bp, Rattus norvegicus
  misc_feature: >gi|8394188|ref|NM_017062.1| Rattus norvegicus reversion induced LIM gene (Ril), mRNA
aaggcggcggcaacggcggcggcgactcctccgtgtccgcctccagctccggctgcagctccggcccgcgatgacccacgcggtgaccctgcgcggcccttcaccctggggcttccgcctggtgggcggccgtgacttcagtgcccccctcaccatctcgcgggttcatgctgggagcaaagcggccctggctgccctgtgcccaggtgactcgatccaagccatcaacggtgagagcactgagctcatgacacacctggaggcccagaaccgcattaaaggctgccacgatcaccttacactctcagtaagcaggcctgagaacaagaactggcccagttcccctaatgacaaggcccaagcgcataggatccacattgaccctgaggcccaggatggcagcccagccaccagcaggcggtcctcaatctctggaattagtctggaggacaacagatcgggcctgggatctccgtatggccagccacctcgccttccagttcctcacaatggcagcagcaatgaggtcaccctgccatcccagatgagcgctctgcatgtgtctccaccccccagtgctgacacacccaggattctccctcggaaccgggactgcagggtggacctgggttcagaggtatacaggatgttaagggagccagcagagccagcagcctcagagcccaaacagtctggatccttccgctacttgcaaggcatgctagaggctggcgaaggcggggatcggcctgggtctggtggttcccggaacctcaagccagcagccagcaagctgggtgctccactgagtggcctgcaggggttaccagagtgcacacgctgtggccacgggatcgtgggaaccatcgtcaaggcgagggacaagctctaccatcccgagtgcttcatgtgtagcgactgtggcctgaatctcaagcagcgtgggtacttcttcctagacgaacggctgtactgcgagaaccatgccaaggctcgagtcaagccaccggagggatatgacgtggtggctgtatatcccaatgccaaggtggaacttgtctgagctgggaaatcttgtcttgcctctgcttcatttaatgtcccccctccccacccccgtggcctgtgtaaatatatgtcctcctggcctttctaataaagtccctctgcttgtcctccaaaaaaaaaaaa

SEQ ID NO: 59 - DNA, 660 bp, Rattus norvegicus
  misc_feature: >gi|8501778|gb|BE109673.1|BE109673 UI-R-BJ1- avq-g-12-0-UI.s1UI-R-BJ1 Rattus norvegicus cDNA clone UI-R-BJ1- avq-g-12-0-UI 3′,mRNA sequence
ttttttttttttttgctggactctggtctgcagaccccacggactatttattgtcacaacaagagtggccgggtcttctccccaagggatggcagagacttttttagcttcagagtggatgcaaagtgggtccaaactcattcttcctacttccggctttggatccaggtcaaggtacttcacctctgaactccagtcacctcagccaggcagttgtaatggcaaaggtatccacaccaaagggcgggggtggggctgccgaaacacaggccgcaaggtgtgtgatgttgtggcaggtgtctggcacacgcaaagagtttggtgtctactgttggggctgacggggacagctatgaaaaggccggaggcagggctgggaattccaggccccttgcaacataaccccgattagagcctcaaagctgcaggaggataacgcctgccagttacttggagctggctgggtagggtancatgtccatcatcggaatggattgttaccgtacagcatcacgcagagagcctactgccccacacctcggcagctgggaaggcagaagcagactctagcccaaggctgctaacttctaccaccaacatgcatctgcttggatctgggacacctaacagtctgccctgcagctgaacagacacacctgg

SEQ ID NO: 60 - DNA, 468 bp, Rattus norvegicus
  misc_feature: >gi|11662646|gb|BF552916.1|BF552916 UI-R-C2- mz-g-09-0-UI.r1UI-R-C2 Rattus norvegicus cDNA clone UI-R-C2-mz-g- 09-0-UI 5′,mRNA sequence
cacgaggccacacaagaccagaccccaagttctgctgtttcagttgccacgcctacagttagtgtttcagctcctgctcctacagccacacctgtgcaaactgtaccccagccgcacccacagacgttacctcctgctgttcctcactcagtccctcagccggcagcagcaatacctgctttcccaccagtaatggtgcctccgttccgtgtgccccttcctggcatgccaattccacttccaggtgtagcaatgatgcaaatagtcagctgcccgtatgtaaagacagtcgctaccaccaagaccggtgtattaccaggaatggcccctcctatagtacccatgatacatccccaggttgctattgcagcttcacctgctaccttagctggagccaccgcagtttctgagtggactgaatataaaacagcagatgggaagacgttctattataataatagaacactt

SEQ ID NO: 61 - DNA, 3403 bp, Rattus norvegicus
  misc_feature: >gi|1272333|gb|U51583.1|RNU51583 Rattus norvegicus zinc finger homeodomain enhancer-binding protein-1 (Zfhep-1) mRNA, partial cds
ccaggtgtaagcgcagaaagcaggcgaacccgcggcgcaataacgttacaaattataatactgtggtagaagcaaattcagattcggatgatgaagacaaactccatattgtggaagaagaaagtgtcacagatgcagctgactgtgaaggtggtgtgccagatgatgagctgccaacagaccagacagtgttaccaggaggcagtgacagggcgggcagtgccaagaactgctggcaagacgacgtaaaagatgacgaatgcgactcagatgcagaaaacgagcaaaaccatgatcccaacgtggaagagttcctgcagcaacaggacaccgcggtcatctaccctgaggcacccgaggaggaccagcggcagggcacgccagaagccagcggtcatgatgacaatggaacaccagatgcattttcccagttgctcacctgcccgtattgtgatagaggctacaagcgctttacctctttgaaagaacacattaagtaccgccatgagaagaatgaagacaacttcagctgctccctgtgcagttacacctttgcatacagaacccagcttgaacgtcatatgacatcacataagtcaggaagagagcaaagacatgtgacacagtctggaggtaaccgtaagttcaagtgcaccgagtgtgggaaagcgttcaagtacaaacaccatctgaaggagcacttacggatccacagtggagagaagccatatgaatgcccaaactgcaagaaacgtttttcccattctggctcttatagctcacacataagcagtaagaagtgtattagcttgatgcctgtgaatggtaggcccagatcaggactcaagacatctcagtgttcctcgccatctctttcaacatcaccaggcagtcccacacgcccacagatacggcagaagatagagaataaaccccttcaagaaccgctttctgtaaaccaaatcaaaactgaacctgtggattatgagttcaaacccatagtggttgcttcaggaatcaactgttcaacccctttacaaaatggggtttttagcggtggtggccaattgcaggcaaccagttctcctcagggtgtggtgcaagctgttgttctgccaacagttggtttggtatctcccataagtatcaacttaagtgacattcagaatgtacttaaagtggctgtagatggtaacataatacgacaggttttggagaataatcaagccagtcttgcatccaaagagcaagaagccgtgagtgcttcatccatccagcagggcggccattctgtcatttctgccatcagtcttcctttagttgatcaggatggcacaaccaaaatcatcatcaactacagtcttgagcagcccggccaacttcaggttgttccccagaatctaaagaaagaaaacccagcccctccaaagagctgcaaaagtgagaagtcaccagaagatcttactgtcaagtcagagaaggacaaaagctttgatggggctgcggatgagagcacttgccttttgtgtgaggactgcccaggagacctcaatgcacttccagaactaaagcactatgacccagagcaccctgctcaacctccaccccctgccccagccaccgagaagccagagtcctctgcttcatcagccggcaatggagatttgtctcccagtcagccacctttaaagaaccttctgtcgctcttaaaagcctactatgctctgaatgcacagccaagcacagaagagctcaccaagattgccgattctgtgaacctgccgctggatgtagttaaaaagtggtttgaaaagatgcaagctggacagattcccgggcagtctcttgaaccaccttctccaggaccggggtcaggaaacatccctgcaaaaactgaggagcagcctcaacctgtggatggaaatgagccccaggaagacagcacacgtggacagagtcctctcaagatgacaagctctccagttttaccagtaggatcagccattaacggttccagaagttgcacatcatccccatcccctctaaacctttcctcagccaggaacccgcagggttactcttgtgtgtcagagggtacccaggaggagccccaagtagaacctcttgatctctcactaccaaagcaacagggagagttactggaaaggtcgacagtcagtagcgtttaccagaacagtgtttattctgtccaggaagaacccttgaacttgtcttgtgcaaaaaaggagccacaaaaggacagttgtgttacagactcagaaccagttgtaaatgtagtcccaccaagtgccaaccccataaacattgctattcctacagtcactgcccagttacccacaatcgtggccatcgctgaccagaacagtgttccgtgcttacgggcactcgctgccaacaagcagactattctgattccccaagtggcatatacatactccgctacagtcagcccagccatgcaggagccaccagtgaaggtgatccagccaaatggaaaccaggatgaaagacaagacactagctcagaaggagtctccgtggaggaccagaatgactctgactgcacgccacccaaaaagaaaactcggaaggcagagaatgggatgtacgcatgtgacctgtgtgacaagatatttcagaagagcagctccctgctgagacacaaatatgagcatacaggtaagaggccccacgagtgcggaatctgtagaaaggcatttaaacacaagcatcatttgattgagcacatgcgactgcactctggggaaaagccctaccaatgtgacaagtgtggcaagcgcttctcacactcggggtcttactctcaacacatgaatcaccgctactcctactgcaagagaggagctgaagacagagatgccatggagcaggaagacactgggccggaagccctgccggaagtcctgcccactgagcttgttggtgcccgggcatctccctcacaggctgactcggacgagagagaaagtttgacaagggaagaggatgaagacagtgaaaaggaggaggaggaggaggataaagagatggaagaattgcaggaagacaaggaatgtgagaacccacaggaggaggaggaggaggaagaggaggaagaagaagaagaggaggaggaagaggaagaggaggcagaggaagctgagcatgaggcggcagcagccaagactgggggtgcagtggaggaggaagcggctcagcaggcaggcagcttccagcagaaggccagcgggagcgagagcaagcgactgtctgaagagaagacaaatgaagcctaggagctcttccaaaaggaagtcctgcttggtaatgaaatttgctctatattacccacgcttttcatggaaacatggctcctgtgcta

SEQ ID NO: 62 - DNA, 2855 bp, Rattus norvegicus
  misc_feature: >gi|11120727|ref|NM_021771.1| Rattus norvegicus transient receptor potential cation channel, subfamily C, member 3 (Trpc3), mRNA
gacaaaggggcaaagacagatgtcagaagggagccccttcctctgtcaggcgggcagtccacatgatgctttaactagatgcttatcactaaaggcgtggctgtcctgtttcggtgtctctctatgaccatctgccattctcttgtcctaaccctaaagtcaacccgggcaggtgacgacttctatgcttacgacgaggatggcacacgcttctcacctgacatcacgcccatcatcctggctgcgcattgccataaatacgaggtggtgcacctgctgctactcaagggtgcacgcatcgagcggccacacgactacttctgtcgctgcgccgactgtgctgagaagcaaaggcttgatgccttcagccactcaaggtctaggatcaatgcctacaagggactggccagcccagcatacttgtcgctgtccagcgaggaccctgtgctcacagcgctggaactcagcaatgagctggccaagctggccaacatagagaaggagttcaagaatgactacaggaagctctccatgcagtgcaaagacttcgtagtaggtgtgctggacctgtgccgggactcagaagaggtggaagccattctgaatggagatctggaatcggtggaacccctggagagacacgggcacaaggcgtcgctgagtcgggtcaaacttgccattaaatatgaagtcaaaaagtttgtggctcaccccaactgccaacagcagcttttgaccatctggtacgagaacctctcgggccttcgggagcagaccatctctatcaagtgtctggtcgtgttggtcgtggccttgggccttccattcctcgccatcggctactggattgcaccttgtaccaggctggggaaaattcttcgaagccccttcatgaagttcgtggctcacgctgcctccttcatcatcttcctgggtctgcttgtgttcaacgcctcacaccggtttgaaggcatcaccacgctgcccaacatcaccgttattgactaccccaagcaaatcttcagggtgaagaccacccagttcacatggacagaaatgctaattatggtctgggttctcgggatgatgtggtctgagtgcaaggagctgtggctggaggggccccgggagtacatcgtgcagctgtggaacgtgcttgacttcgggatgctctccatcttcattgctgccttcaccgccaggttcctagcatttctgcaagccaccaaagcgcaccagtatgtggacagccacgtgcaggagagcgacctgagcgaagtcacactcccaccgaaggttcagtatttcacctatgctagagataaatggcttccttctgaccctcagatcatatcggaaggcctctatgccatagctgtggtgctcagcttctcccggatcgcgtacattctccctgcaaacgagagctttgggcccttgcagatctctctggggaggactgtcgaagacatattccagttcatggttctcttcatcatggtgttcctggctttcatgattggcatgttcatactttactcctactaccttggggccaaagtaaaccctgcttttaccacggttgaagaaagtttcaagactttgttttggtccatatttggactctctgaagtgacttctgttgtgctcaaatatgaccacaaattcatagagaacattggctatgtcctttatggaatatacaatgtaactatggtggtcgttctgctcaacatgctgattgctatgattaacagctcataccaagaaatcgaggatgacagtgatgtagagtggaagtttgctcgttccaaactctggctatcctacttcgatgatggaaaaacattacctccacccttcagtctggtccctagtccaaaatcgtttgtttatttcatcatgaggatcactaacttttccaaatgcaggaggagaagacttcagaaggatctggaactgggcatgggtaactcaaagtccaggttaaacctcttcactcagtctaactcgagagtttttgaatcacacagttttaacagcattctcaatcagccaacacgatatcagcagataatgaaaagactcataaaacggtatgttttgaaagcacaagtagacaaagaaaatgatgaggtgaacgaaggtgaactgaaagaaatcaagcaggatatctccagccttcgttacgaacttttggaagataagagccaagcgacggaggaactggccatcttgattcataaactcagtgagaaactgaaccccagtgcgctgaggtgtgaatgagggagccacatgcagtgagactttgactctggcacaaatgtgggcaataatattcctaagtatgaaatacttgaaaaaccgttgtgtaaaaactctagtgttaataccttcaccatgcggagctttaaaattaacctagtgggccacaattcccattacgtgaaaatgatcccaaggttggcctcagcactccaagcagtgacatgctgttgtacttagaagcccagtattaatgtcactgatttttttttttttttgtcatttttgtctttatgtcacctgttggcctccaagagttccttgggggacagggaccacatcaatctcatctttatgtctccaacatttagtgcagtgcctgatacacagtaggtgcttggtaaatgttgaacccagctggcctggacagccagtagcatccaagaacacgaaagaccattgctatgtagcctaccttctcatccgaacgcttgaagcgatttttttaaaaatagaaactgaaggtgttttacgaccagccatgacgtgtggagatctttctcagacctttagacatgcactgacag

SEQ ID NO: 63 - DNA, 1298 bp, Rattus norvegicus
  misc_feature: >gi|9994170|ref|NM_020471.1| Rattus norvegicus insulin related protein 2 (Isl2), mRNA
gaattccgcggaggctgggcgcgcgaccctggtccttctgcccccggccgtacgctttgctcacgtctctctctgcatggtggatattatttttcattatccttttctgggtgctatgggggatcattccaagaagaagcccgggacggccatgtgcgtgggctgcgggagtcagatccacgaccagtttatccttcgggtgtcgcccgacctcgagtggcacgccgcctgcctcaagtgcgctgagtgcagccagtatctggatgagacgtgcacgtgcttcgtgagagacgggaaaacgtactgcaagcgggactacgtcaggctgttcggcatcaagtgtgcccagtgtcaggtgggcttcagcagcagtgacctggtgatgcgggcgcgggacagcgtgtaccacatcgagtgcttccgctgctccgtgtgcagccgccagctgctccctggagacgagttctcgctgagggagcacgagctgctctgccgagccgaccacggcctcctgctggagcgcgctgcggccggcagcccgcgcagccctggcccgctccccggcaccccaccaggcctgcatctgccagacgccgggtccggacaacaggtctcactgcgcacgcacgtgcacaagcaggcggagaagacaacccgggtgcggactgtgctcaacgagaagcaactgcacaccctgcggacatgctacgccgccaacccgcggcccgacgcgctcatgaaggagcagctggtagagatgaccggcttgagcccgcgggtcatccgcgtgtggtttcagaacaagcgttgcaaggacaagaagaagtccattctcatgaagcagctacagcagcagcaacacagcgacaaggcgagtctccagggactaactgggacgctcctggtggcgggcagcccatccgcccatgagaacgccgtgcagggcagcgcagtcgaggtgcagacgtaccagccgccctggaaagctctcagcgagttcgcactccagagcgacctggatcaacccgcctttcaacagctggtctccttctccgagtcaggctccctaggcaactcctctggtagcgacgtgacctctctgtcttcgcagctcccggacacccccaacagtatggtacctagtccggtggagacgtgagacaggacccttccctgccagcccgcggacctcgcatgcctccctgcatgagactcacccatgctcaggccattccagctctgaaaaatctccggcccttgtaattattgttgttatttaaaggcgagcgggcagagga

SEQ ID NO: 64 - DNA, 2393 bp, Rattus norvegicus
  misc_feature: >gi|16758529|ref|NM_053713.1| Rattus norvegicus Kruppel-like factor 4 (gut) (Klf4), mRNA
atcttcgttgacttcggggtttgggtacccctctctcttcttcggactccggaggaccttctgggcccccacattaatgaggcagccacctggcgagtctgacatggctgtcagcgacgctctgctcccgtccttctccacgttcgcgtccggcccggcgggaagggagaagacactgcgtccagcaggtgccccgactaaccgttggcgagaggaactctctcacatgaagcgacttcccccacttcccggccgcccctacgacctggcggcgacggtggccacagacctggaaagtggtggagctggtgcagcttgcagcagtaacaacccggccctaccccggagggagaccgaggagttcaacgatctcctggacctagactttatcctttccaactcgctatcccaccaggaatcggtggccgccaccgtgaccacctcggcgtcagcttcatcctcgtcttccccagctagcagcggccctgccagcgcgccctccacctgcagcttcagctatccgatccgggccgggggtgacccgggcgtggctgcgggcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgagcacccctggcagcgagtacaccagcccttcggtcatcagtgttagcaaaggaagcccagacggcagccaccctgtggtagtggcgccctacagcggtggcccgccgcgtatgtgccccaagattaagcaagaggccgtcccgtcctgcacggtcagccggtccctagaggcccacttgagcgctggaccccagctcagcaacggccacaggcccaacacacacgacttccccctggggcggcagctccccaccaggactacccctacactgagtcccgaggaactgctgaacagcagggactgtcaccctggcctgcctcttcccccaggattccatccccatccggggcccagctaccctcctttcctgccagaccagatgcagtcgcaagtcccctctctccattatcaagagctcatgccaccgggatcctgcctgccagaggagcccaagccaaagaggggaagaaggtcttggccccggaaaagaacagccacccacacttgtgactatgcaggctgtggcaaaacctatacgaagagttctcatctcaaggcacacctgcgaactcacacaggcgagaaaccttaccactgtgactgggacggctgtgggtggaaattcgcccgctcagatgaactgaccaggcactaccgcaaacacaccgggcaccggccctttcagtgccagaagtgcgacagggccttttccaggtcggaccaccttgccttacacatgaagaggcacttttaaattccacatcgtggacatgacccacactgccaggagagagttcagtatttttttttaacctttcacactgtcttcccacgaggggaggagcccagctggcaagcgctacaatcatggtcaagttcccagcaagtcagcttgtgaatggataatcaggagaaaggaagagtccaagggacaaaagaaaagaaaagaaaaaaatactaaaaaacaaacaaacaaaaaaaaaaaacaaaagaaaaaaatcacagaacagatggggtctgagactggatcttctatcattccaataccaaatccgacttgaacaagactggacttacaaaatgccaaggggtgactggaagtttgtggatatcagggtatacattaaatcagtgacctggggggagggaagaccagagttcccttgaattgtgcttcaatgatgcaatatacatggaaagaccaccttgtatgctctttgccttctaaaaagccattatgacgtcagaggaagaggaagcaattcaggtacagaacgtgttctaatagcctaaacgatggtgcttggtgagtcgtggttctaaaggtaccaaacgggggagccaaagttctccaactgctgcatactttgacaaggaaaatctatttttgtcttccgatctacatttatgacctaagtcaggtaaataagcctggtttatttctgtaacattttttatgcagacagtctgttatgcactgtggtttcagatgtgcaataatttgtacaatggtttattcccaagtatgcctttaagcagaacaaatgtgtttttctatatagttccttgccttaataaatatgtaatataaatttaaaaaaaaaaaaaaaaaaaaaaaaaaa

SEQ ID NO: 65 - DNA, 2124 bp, Rattus norvegicus
  misc_feature: >gi|10086310|gb|AF281635.1| Rattus norvegicus Kruppel-type zinc finger protein KROX-25 mRNA, complete cds
atgaggttaggaaagccccaaaaggggggtatttctcgaagtgccacccaaggaaaaacctatgagagcaggcgcaagacagcccgccagcgacagaaatggggggtggccattcgcttcgactcagggttaagtagacgtagaagaaacgtggatgagaagccttacaagtgtactaaatgttctaagagtttcagtcagagctccactctttttcagcacaagaagatccacactgggaagaaatcccataaatgtgccgattgtgggaaaagtttctttcagagttctaacctcattcagcatcggcggatccacacgggggaaaagccctataagtgcgacgagtgtggagagagatttaaacagagctccaaccttatccagcaccagaggattcacaccggggagaagccctattactgcgatgagtgtggccggtgctttagccagagttcccacctcattcagcaccagagaacccacaccggagaaaagccctaccagtgcgaggaatgtgacaagtgcttcagccagaactcccacctgaggcagcacatgaaggtgcaccaagaaaagaagtcgcataaaaggggtaaaaatgccagggcgaaaactcacccggtgtcctggaaacgtggtaaaggaaggaaggcagtggctgggctccgcaggtaaagggagccgcttcgggccttttcaaaaagaagaagtgaatctcgtagcggtgaaagcctctcccaagagggaggacactttacagtagatagagcacttagatactggtcctccctagcatggagatgtcaggagtttcatgacaatgggctgaaactctgagtctagccatcctcatttcctttcagaataaataagtaaatttatgtgacatggagcagaaaactgaaaaaaaagtttggtaagaatttaacatccttggcctcagttgaaatttcttccctacgttttgataaaaagcgtcatgttttcatgattcactggtgtgccaatgattactcttggttttaggaatttctgtatacaaaatcattaaagagaaggaatgagttttcaagttctaggaccgactatatttttaatgttttgtagttttttttaaccattagtatcagcattgattcagtatttgacataataaaatgcattcaagttcatagatttttgtattttttattagcgtatattgtatgaagtaatgagttcgttttccttaatgtgtacgatgtatctgtatgatgtgctttgattatatccattgctcttaccctcacctccacctcctggttctcttcttcctcccagagagttcccttcccacttgaattgttgaccctgggattctgtgaatgacagaaattgtctttggttatttttccccatctgctttactgtgtttactgtaatgttctccagttctatccatttttcgccaaacaacaggattttggcttctttttttttttttttttaactaattccaactaataagttcgtcatagtgtctgatgccttaatgaaggggtgaaaaggtctccatgtggcttttctcctgttcgtttccttgtttaccatattccttagagatttcaaagaatccaaagtgagaaattgcagacctcctcctaagaccattacttgcgatgctgatgccctgcctcttgctttcttgaaatgggaatggtttatgttgaatcatgccagtgccttctgggcgttcctttgttctcttgcaaacagggcctgcatgaataaactatgttaccaagaaagcctgacttacttctggctaaagaactacagtgtcatagactgagctttgactgccgctgactgagacagatgtgtgtctacaagttcctggagccctgttagaacttaactgttctttactttttagcagttgtttaagaagactagcggacatcctttttcgaatgctaagcctgagttcctagttaacccttagttcaagtttttgtttattgaatgctaatgttttctgatttttataatctcacttcttcctgtattaatgacaattatcctcaaaataaagattgagatatttttgcgtaaaaaaaaaaaaaaaaaaaaaaaaaaaaa

SEQ ID NO: 66 - DNA, 1238 bp, Rattus norvegicus
  misc_feature: >gi|8393330|ref|NM_017347.1| Rattus norvegicus mitogen activated protein kinase 3 (Mapk3), mRNA
cggggggaggcggcagcagtggagatggcggcggcggcggcggctccggggggcgggggcggggagcccaggggaactgctggggtcgtcccggtggtccccggggaggtggaggtggtgaaggggcagccattcgacgtgggcccacgctacacgcagctgcagtacatcggcgagggcgcgtacggcatggtcagctcagcatatgaccacgtgcgcaagaccagagtggctatcaagaagatcagccccttcgagcatcaaacctactgtcagcgcacgctgagagaaatccagatcttgctcggattccgccatgagaatgtcataggcatccgagacatcctcagagcacccaccctggaagccatgagagatgtttacattgttcaggacctcatggagacggacctgtacaagctgctaaagagccagcagctgagcaatgaccacatctgctacttcctctaccagatcctccggggcctcaagtacatacactcggccaatgtgctgcaccgggacctgaagccctccaatctgcttatcaacaccacctgcgaccttaagatctgtgattttggccttgcccggattgctgaccctgagcacgaccacactggctttctgaccgagtatgtggccacacgctggtaccgagccccagagatcatgcttaactccaagggctacaccaaatccattgacatctggtctgtgggctgcattctggctgagatgctctccaaccggcctatcttccccggcaagcactacctggaccagctcaaccacattctaggtatactgggttccccatcccaagaggacctaaattgtatcattaacatgaaggcccgaaactacctacagtctctgccctctaaaaccaaggtggcttgggccaagctttttcccaaatctgactccaaagctcttgacctgctggaccggatgttaacctttaacccaaacaagcgcatcacagtagaggaagcactggctcacccttacctggaacagtactatgatccgacagatgaaccagtggctgaggagccattcacctttgacatggagctggatgatctccccaaggagcggctgaaggagctgatcttccaagagacagcccgcttccagccaggggcaccagaggccccctaacaagaacagacacccctgtccttttggacctggtctgctctacctgctccttctctgcagattgttagaaa

SEQ ID NO: 67 - DNA, 1969 bp, Rattus norvegicus
  misc_feature: >gi|13786183|ref|NM_031345.1| Rattus norvegicus delta sleep inducing peptide, immunoreactor (Dsipi), mRNA
ctggcggctgtggacttgtgacatactaggtgacacccctggagccacttcctttcaactccagcttgaaagtgcctgcctggctcagggtctgcactgcagcctactcctagctccagggcttgactgcggcgtcagagcctatcctgtagcagcgcgccagcagccactcaaaccaaccacagctccccggcaacccgaatcatgaacaccgaaatgtatcagacgcccatggaggtggcggtctatcaactgcacaatttctccatctccttcttctcttctctgcttggaggggatgtggtttccgttaaactggataacagtgcctctggagccagcgtggtggccctagacaacaagattgagcaggccatggatctagtgaagaatcatttgatgtatgctgtgagagaggaggtggaggtcctaaaggagcagattcgtgagctggttgagaagaactcgcagctggagcgtgagaataccctcctgaagacgctggcgagcccagagcagctggagaagttccagtcccggctgagccccgaagagccagctcctgaagccccagaaaccccagaagcccctggcggttctgcggtgtaagtggctctgtccttagggtgggcagagccacaacttgttctacctagttctttccagtttgttttttggctccccaagcgtcatctcatgtggagaactttacacctaacatagctggtgccaagagatgtcccaaggacatgcccatctgggtccactccagtgacagacccctgacaaagagcaggtttggagactgagttgcacggggcctagtaaccccaagccagtgagcttgtcgtgccaccgggccctgggggctcccagggtctgggcaacttagttccagctggccaaggagaaagtagttttgagatggaatgccaatatgctccagaaagtataaggggtctgttttcatttccatggacatcttccacagcttcacctgacaacgactgttcctatgaagaagccacttgtgttttaagcagaagcaacctctctcttctcctctgtcttccagacggggacagagatgggagagattgagccaaatcagccttctgttggttaatattgtataatgcatggctttgtgcacagcccagtgtgggattacagctttgggatgactgcttataaagttctgtttggttagtattggcatagtttttctatatagccataaatgcgtatatatatatacccatagggctagaactatatattagtgtagtgatgtatacatatacacatacacatacatgttgaagggcctaaccagctttgggagtactgactggtctcttatctcttaaagctaagtttttgactgtgctaatttaccaaattgatccagtttgtcctttagattaaataagactcgagatataagggagggagagggagaccagcctcaccatgcggccacagatgccttgctgctgcagccctccccaatctgtccactgaagacatgaagtccttttgaatgccaaacccaccattcattggtgctgactacatagaatggggttgagagaagaccagtttggacttcacatttttgtttaagctttaggtcggttttttttttttttttgttttgttttcgtttgtttttgtttgttttttcttttcttttttttttaagttcttgtgggaaactttgggattagtcaaaggatggagtcctgtggtagacaagaagagtaaatagttttgatcccttcggggcctggaaaatgcacccaggaagcttgtgaaagaaggttctgtgacagtgaacactttccactttctgacaccttatcctgctgtatgtctccaggatttggattttgatttttcagatgtagcttgaaatttcaataaactttgctctttttttcaaaaaatcaaaaaaaaaaaaaaaaaa

SEQ ID NO: 68 - DNA, 4432 bp, Rattus norvegicus
  misc_feature: >gi|603961|dbj|D25233.1|RATRP Rattus norvegicus mRNA for retinoblastoma protein, partial sequence
ccggaggacgaccccgcgcaggacagtgaccccgaagagctgcccctgatcaggcttgagtttgaaaaaattgaagaacctgaatttattgcattatgtcagaagttaaaggtgcccgatcatgtcagagagagagcttggctaacgtgggagaaagtttcatctgtggatggaatcctggaaggatatattcagaagaagaaggaactctggggcatctgcatcttcattgcagcagttgacctggatgagatgccattcacttttactgagcttcagaaaagcatagaaaccagtgtctataaattctttgacttattaaaagaaattgataccagtaccaaagttgataatgctgtgtcaaggctattgaagaagtacaatgtgttatgtgcactctacagcaaattagaacggacatgtggacttatatatttgacacagcccagcagtgggttatctactgaaataaattctatgttggtgctaaaagtttcttggatcacctttttactagctaaaggagaagtagtacagatggaagatgatctggtgatctcatttcagttaatgttgtgtgtacttgactattttatcaagctctcgcctcctgcgctgctcagagagccgtacaaaacagctgcaacacccattaatggttcacctcggacacccagaagaggtcagaacaggagtgctcggatagcaaaacaactagaaagtgatacaagaactattgaagttctctgtaaggagcacgagtgtaatgtagatgaggtgaaaaatgtttatttcaaaaattttatcccttttataagttcacttggaattgtatcatctaatggacttccagagcttgaaagtctttctaaacgctacgaagaagtttatcttaaaagcaaagatttagatgcaagactatttttggatcatgacaaaacacttcagactgatactatagacagttttgaaacagagagaactccacgaaaaagcaaccctgatgaagaggcaaacatggttactccacacactccagttaggactgttatgaatactattcaacagttaatggtgattttaaattctgcaagtgatcagccatcagaaaatctgatttcctacttcagtaattgcacagtgaatccaaaagaaaatatcctaaaaagagtaaaggatgttggacacatctttaaagagaagtttgctagcgctgtggggcagggatgtattgacattggagcacagcgatataaacttggggttcgattgtattatcgtgtgatggaatcgatgcttaaatcagaagaagaacgtttgtccattcagaattttagcaaactcctaaatgacaacatctttcatatgtctttattggcctgtgctcttgaagttgtaatggctacatatagcaggagtatgttacagaatcttgattctggaacagatttgtccttcccgtggattctgaatgtacttaatttaaaagcctttgatttttacaaagtgattgaaagttttatcaaagtggaagccaacttgacaagagaaatgataaaacatttagaaagatgtgagcatcgaatcatggaatcccttgcatggctttctgattcacctttgtttgatctcataaagcagtccaaggatggggaaggacctgatcaccttgagtctgcttgttctctcagcctccctctccagagtaaccacactgcagcagatatgtatctttctcctataagatctccaaagaaaagaacttctactacacgtgtaaattctgcggcaaatacagagacgcaagcagcctcagccttccatactcagaagccattgaaatctacctccctttccctgttttacaaaaaagtgtaccgtctagcatatctccgactaaatacactgtgtgcgcgccttctgtctgaccacccagagccagaacacatcatctggactctgtttcagcatacattggaaaatgagtatgagctcatgaaagaccgacatttggaccagattatgatgtgttctatgtatggcatctgcaaggtgaaaaacattgaccttaaattcaaaatcatcgtaactgcgtataaggatcttcctcacgctgcccaggagacctttaaacgtgttttgatcagagaagaggagtttgattccattatagtgttctacaactcagttttcatgcagagactaaaaacaaatattttacagtatgcctccaccaggcctcctaccttgtcaccaatacctcacattcctcgaagcccttacaagttctcgagttcacccttgcggattcctggaggtaacatctatatgtcacccctaaagagtccttataaaatttcagaaggtctgccaacacccacaaaaatgactccaagatcaagaatcttggtctcaatcggtgaatcatttgggacatccgagaagttccagaaaataaaccagatggtgtgtaacagtgacagagtgctcaaaagaagtgctgaaggcggcaatccccccaaacccctgaagaagctgcgctttgacatcgaaggatccgatgaagcagacgggagtaaacatctcccagcggagtccaaattccaacagaaactggcagaaatgacttctactcgaacacgaatgcaaaagcagaaactgaatgatagcatggaaatctcaaacaaggaggaaaagtgatgatctcaggaccctaacactgtagaccctcgggacacctgactcgtgtctcatggatgtgactatagttcccaggttctgctcatgttatagatataaaatgtgcaggtacagctgaatatttatgtgggtgatttgtaagccacttcaatgttgtaatcaatggtatagaattgacagtacacaggattgagaatcttgtgcagatcatgccatttaaaatatgaaagcagattgttggcatttccaacaggagcctgctgccctcagaagtgaggctgcctctggagggagtcctagcaactcccttacatctggctgactactttgcctttccttgtggcacatgtgtgatgtttgctcttggttttattaatttatatgtattttttaatttaacgtgaatatccttagaaaatgtgtcttgtcttccaaatgcaagttgattgattgtccacattcaaccaaattatcttgaatcctctactgaaacagattgttacaaactggggaaaagtactaatttgtacacattggacttgttttaatactagaatctaatgtgccatatgtttgtttcacacattttacctttgaaatagaagtaaaaaaagtataaacatatgatactgtcttactactgacacagatttcatacctcagaccctctaagaaccaattcttttattcatccaactcatactttaaactgaagactattgataatactccaggtttgttttttcttttaatctttcagatcactgaatctttaagtacccatgtagtacttgaaaatcaagtctggccacaactgtacttcagaggaccctagtacagtacaacccaagtgcactttctaatgtctctgggtcctgaggaatcaagatacaaattaactatgttctgtaagcagaccgttaactataggagccttcggtttaactatgttctgtaagcagaccgttaactataggagccttcggttttccctcatacaagtgtctaaccacatctcaacagttcactctgctcttctgcatccagggctgatgtgttccctctggaatggtacatgtcttccagggtcttttgaactggcaggtatctatttttttaagccaatctggtctaataacactggcttcttcaaagccacagcatttctaatccagctgtgccgaagcttcagactaaaacagctgcattgaaagaagaggcactcccttcaccccccccccccacctaaaggtgtttacattatcttgtgggattaacttatttagagatgctataatttaaaataggcggtatttaaggtagcgtcagctagcatttaagaaaatcattttttctaaactccatactttttgaaaagaaatctggtcttgttagaaaacaaatttctactttgtcctcaatttagtttcagttttactaggttgatagttatctcgatgacaaaggccgtagacagctccccccatttcttcattgagttttgcatggccatcacacaggttagttgggtttaggtcaagggctgaccatacttctaggtcttttgctagtaagttcaagttagaattagtgacagaatcataggaattttcagagatcctctattaagatttcttaaaactgcagaccctgcttttgtttttgtactggttaaaactatacattcagattgctatgttcctattttctataatagtttgtctattttaca

SEQ ID NO: 69 - DNA, 3918 bp, Rattus norvegicus
  misc_feature: >gi|8393489|ref|NM_017012.1| Rattus norvegicus glutamate receptor, metabotropic 5 (Grm5), mRNA
tcagacatctcttgagaaactcagcctagtttatccagctgttttgtccacatatgttcataaaatctagttatgaatattctgatcgtatggatgcacctaactcgacagtttctggggactttgctatcttttagagaatatatctaaattgctgactaattcctcaatgcgccactcaacgcagggcatcatttggctgtacctattggagctctgagtttcctccatcatgatatctttattagcttgaattcctttcctaaaatggtccttctgttgatcctgtcagtcctacttctgaaagaagatgtacgagggagtgcacagtccagtgagaggagggtggtggctcacatgccaggtgacatcattattggagctctcttctctgtccaccaccaaccaactgtggacaaagttcatgagaggaagtgtggtgcagttcgtgagcaatatgggattcagagagtggaggccatgctgcataccttggaaaggatcaactcagatcccacactcttgcccaacatcacacttggctgtgagatcagagattcctgctggcattccgctgtggccctagaacaaagcattgagtttataagggattccctcatctcttcagaagaggaggaaggtttggtacgctgtgtagatggatcttcttccttccgctccaagaaacccatagtgggagtcattgggcctggctcgagttctgtggccattcaagtccagaacttactccagcttttcaacataccacagattgcttactctgcaaccagcatggatttgagtgacaagactctattcaagtacttcatgagggttgtaccttccgatgcccagcaagctcgagccatggtagacatagtgaagagatacaactggacttatgtctcagctgtgcacacagaaggcaactatggagaaagtgggatggaggctttcaaagatatgtcagcgaaggaagggatttgcatcgcccactcttacaaaatttacagcaatgctggggaacagagctttgacaagctgttgaaaaaactcagaagtcatttacctaaagcccgggtggtagcttgcttctgtgaaggcatgacagttcgaggtctgctcatggccatgagacgcttaggtctagcaggggagtttctacttctgggcagtgatggctgggccgacaggtatgacgtgacagatggatatcagcgagaagctgtcggtggaattacaatcaagcttcagtctcctgatgtcaagtggtttgatgattattatctgaaactccggccagaaacaaacctcagaaacccttggtttcaagaattttggcagcatcgttttcagtgccggctggaagggtttgcacaggagaacagcaagtacaacaagacttgcaacagttctctgactctgagaacacatcatgttcaagattccaaaatgggatttgtgataaatgcaatctattctatggcttatggactccacaatatgcagatgtccctgtgtccaggctatgcaggcctctgtgatgcgatgaagccaattgatggacggaaacttttggattccctgatgaaaaccaattttactggagtttctggagatatgatcctgtttgatgaaaatggagactctccaggaaggtatgaaataatgaatttcaaggaaatgggaaaagattattttgattacatcaatgttggaagttgggacaatggggaattaaaaatggatgatgacgaagtgtggtccaagaaaaataacatcatcagatctgtgtgcagtgaaccatgtgagaaaggccaaataaaggtgatccggaagggagaagtcagctgttgttggacctgcacaccttgtaaagagaatgagtatgtgtttgatgagtacacctgcaaggcatgccaactgggctcctggcccactgacgacctgacaggctgtgacttgatcccagtccagtatcttcgctggggtgaccctgagccgattgcagctgtggtgtttgcctgcctcggtctgctagccaccttattcgttactgtaatcttcatcatttatcgggacactccggtggtcaagtcctccagcagggagctctgctacattatccttgctggcatctgcctgggatacttatgtaccttttgcctcattgcaaagcccaagcagatttactgctatcttcagagaattggcattggactctctccagccatgagctactcagcccttgtaaccaagaccaaccgtattgcaaggattctagctggaagcaagaagaaaatctgtaccaagaagcccagattcatgagcgcctgtgctcagttagtgatcgctttcattctcatctgtattcagctgggcattatcgtggccctctttatcatggagcctccggatataatgcatgactatccaagcatccgagaagtctacttgatttgtaacaccaccaacctaggggttgttactcctcttggatacaatggattattgattttgagttgcacgttctatgcgtttaagaccagaaatgttccagccaactttaatgaggccaaatatattgctttcaccatgtacacaacctgcatcatatggctggccttcgtgcctatctactttggcagcaactacaaaatcatcaccatgtgtttttcagtcagcctcagtgccacagtggccctgggttgcatgtttgtcccgaaggtgtacatcatcctagccaaaccggagagaaatgtgcgcagcgccttcacaacctctacagtggtacgcatgcatgtaggagacggcaaatcatcgtccgctgccagcagatccagcagcctagtcaacctgtggaagaggaggggctcctctggggaaaccctaagctccaacggaaaatctgtgacttgggcccagaatgagaagagtacccgggggcaacatttgtggcagcgactgtctgtccacatcaacaagaaggagaaccccaaccaaacggcggtcatcaaaccctttcccaagagcacagagaaccgggggcctggtgcagcggcaggtggtggctcgggtcccggtgtagctggtgctggtaacgcaggatgcacagcaacaggtggcccggagccaccggacgccggtcccaaagcgctttatgatgtggcggaggcagaggagagcttccccgcggctgccaggccgcgctcgccatcgcccatcagcacgttgagccacctcgcaggctcggcgggccgcacagatgatgatgcgccgtcgctgcactctgagacagctgctcgcagcagctcgtcccagggctcgctcatggagcagatcagcagcgtagtgacgcgcttcacagccaacatctccgagctcaactccatgatgttgtccaccgcggccacgccggggccccctggtactccaatctgctcctcctacctgatccccaaagagattcagctgcccacgaccatgacgaccttcgcagagatccagccgctgccggccatcgaggtgaccggaggagctcagggggcgacaggcgtatcacctgcccaggagacgcccacaggagctgaatccgccccgggcaaaccagatctggaggagcttgtggccctcactccaccatcgcccttcagggactcggtggactcggggagcaccaccccaaactctccagtctcagaatcggccctctgcatcccatcctctcccaaatatgacactctcatcatcagagattacacgcagagttcttcatcgttgtgagccactggaaacttccctgagataggcctgtgcagcagacctcggtgttccagatgggcgcagcgacacccatagtcaccccaacagagaacccgaggtgaaaaatgccaaggacacacacggtggagacatgag

SEQ ID NO: 70 - DNA, 479 bp, Rattus norvegicus
  misc_feature: >gi|13300163|gb|BG375691.1|BG375691 UI-R-CS0- bte-c-05-0-UI.s1UI-R-CS0 Rattus norvegicus cDNA clone UI-R-CS0- bte-c-05-0-UI 3′,mRNA sequence
ttttttttttttttttttggtgaaatttctactttatttatgatttggagaggaacatctgagatattacaatgcagtattcctcaattacaatgtggccatacaatcaaaagctctagaaagcagtcacaacaaaacaggaagcggatgtgctgttgagcggcatgactacagaaggctcttagcccaggagctcctttaacccccagttaaaactgccagcgcgctgcagaaggagctgccccaaagccacaccgggccctcatttgcggctttacttacttctttccgtttcgtttcaggacattccgactgcagagtaagagtcgcgccttcaatgtttcttggcatgggcgtggaaccagggtttatagtcaaatgaatctgatctggtgagataatgtgttgtacgtaaccctgggacattgcttcctgatctattangaagccctcttcaccttccaccagaaaaggcaagt

SEQ ID NO: 71 - DNA, 4457 bp, Rattus norvegicus
  misc_feature: >gi|56799388|ref|NM_012747.2| Rattus norvegicus signal transducer and activator of transcription 3 (Stat3), mRNA
gggagcttggggttccgacgtcgcggcggagggaacgagccctaaccggatcgctgaggtacaatcccgctcggtgtcgcctgaccgcgtcggctaggagaggccaggcggcccgcgggagcccagcagttcgcgcctggagccagcatagggcggccagtcgggcttcagccccagagacagtggtcacccctgattgcggcaggatggctcagtggaaccagctccagcagctggacacgcgctacctggagcagcttcatcagctgtacagcgatagcttccccatggagctgcggcagttcctggcgccttggattgagagccaagattgggcatatgcagccagcaaagagtcacacgccactctggtgtttcataacctcttgggcgagatcgaccagcagtatagccgattcctgcaggagtccaatgtcctctatcagcacaacctgcgaagaatcaagcagttcctgcagagcaggtatcttgagaagccaatggaaattgcccggattgtggcccgatgcctgtgggaagagtctcgcctcctccagacggcagccacggcagcccagcaagggggccaggccaaccaccccacagctgccgtagtgacggagaagcagcagatgctggaacagcatcttcaggatgtccggaagcgtgtgcaggatctagaacagaaaatgaaagtggtggagaatctccaggatgactttgatttcaactataaaaccctcaagagtcaaggagacatgcaggatctgaatggaaacaaccagtctgtgaccagacagaagatgcagcagctggagcagatgctcacggccctggaccagatgcggaggagcatcgtgagcgagctggcagggctcttgtcagcaatggagtacgtgcagaagacactgaccgatgaagagctggctgactggaagaggcggcagcagatagcgtgcatcggaggccctcccaacatctgcctggaccgtctggaaaactggataacttcattagcagaatctcaacttcagacccgccaacaaattaagaaactggaggagctgcagcagaaagtgtcctacaagggggaccctattgtgcagcaccggccaatgctggaggagaggatcgtggatctgttcagaaacttaatgaagagtgccttcgtggtggagcggcagccctgtatgcccatgcacccggaccggcccttagtcatcaagactggtgtccagtttaccacaaaagtcaggttgctggtcaaatttcctgagttgaattatcagcttaaaattaaagtgtgcattgataaggactctggggatgttgctgccctcagagggtctcggaaatttaacattctgggcacgaacacaaaggtgatgaacatggaggagtccaacaacggcagcctgtctgcagagttcaagcacctgaccctgagggagcagagatgtgggaatgggggccgtgccaattgtgatgcctccttgattgtcactgaggagctgcacctgatcacctttgagacagaggtgtaccaccaaggtctcaagatcgacctagagacccactccttgccagtcgtggtgatctccaacatctgtcagatgcctaatgcttgggcatcaatcctgtggtataacatgctgaccaataaccccaagaacgtgaacttcttcactaagcctccgattggaacctgggaccaagtggccgaggtgctgagctggcagttctcgtccaccaccaagcgagggctgagcatcgagcagctgaccacgctggccgagaagctcttagggcctggtgtgaactactcagggtgtcagatcacatgggctaagttttgcaaagaaaacatggccggcaagggcttctcgttctgggtctggctagacaatatcatcgaccttgtgaaaaagtatatcttggccctttggaatgaagggtacatcatgggtttcatcagcaaggagcgggagagggccatcctaagcacaaagcccccgggcaccttcctgctgcggttcagtgagagcagcaaggaaggaggggtcactttcacttgggtggaaaaggacatcagtggcaagacccagatccagtctgtagaaccatataccaagcagcagctgaacaacatgtcatttgctgaaatcatcatgggctataagatcatggacgctaccaacatcctggtatccccactggtctacctctaccctgacattcccaaggaggaggcattcggaaagtattgtcgccccgagagccaggagcaccctgaagctgacccaggtagtgctgccccttacctgaagaccaagttcatctgtgtgacaccaacgacctgcagcaataccattgacctgccgatgtccccccgcactttagattcattgatgcagtttggaaataacggggaaggcgctgagccctcagcaggagggcagtttgagtcgctcacgtttgacatggatctgacctcggagtgtgctacctccccgatgtgaggagctgaaagcggaaactgcagagtatgactgagacacctgccccatgttccaccccttaagcagccaaactcccagatcatctgaaactcccaactttgtggttccagattttttctttaatctcctacttctgctatctttgggcaatctgggtactttttaaaatagagaactgagtgagcgtgggtgatgcgctcttatgtgaggaggagggcacctcttgagtccgtgatggctgtgaaagcagagggctgggggggagcgaaaggaaacacctatgttctgtccgcctgtcctcctggtttgttgttggacaagcgcctcctggtgcccatggcatcctgctgcccctttctgtgagctgataccccaggctggggactcccggctctgcactttcaacctggttaatgtccacatagaagctaggactaagcctataggcttctctttaaattaaaaaaaaaaatactaagaattaaagggcaaaacacactgaaacagcatagcctttccatatcaaagaaaactcggttaacagccttttcggtgctttaagctgtctttaggctgatcatttatataaactctgcaatggtttcaaatcaaatctgtaaaagacatctgagaactgtggttcaaacacaaagattagagagaacctagatatcccagactcttgtcatgaactgggttttgtttacccagtatgcttgtctgttggaggggtgaggtcggccaagggcactggaaaacctttgtcatacccccctccccagcccagactctggaccctgttccagggtcatcctgccctgtgggtgccttactgggcctagggtcaacctggcttcctttcccacttgaccttgctagtggtatgtctccttcccatgtccaaaggcccctgtcctgcttacattgggaatccctgtctcagaaccttgtgtcgagagggattgccttagaggtttgaacctaactcagactacaggtcctcagcaaagctcagggagtatggtccttattctatgtgcttggttcccaggggtacctgtaaccacaggacaaaagctgactgataaaatccaggtctgcccttcatgtgagtggcgtactcccgccccccccccccccgccccgagagtgccgattgtatgctccccgttggggctcctaggtgaggtgggacagagtgcctgcccctactgtggccttcctgtcacctgcttgcctcagtcactgagcatacttgaacactgagtggttcaaggcaagcctcccctgatacaggggcatggctagattcagtgactcaaagccaccttactcagctgatctgtctgtggaattgtttttctccagttaaccagtgtctgaattaagggcagtgaggacattgtctccaagacaaactgcttgccttgaccaccccagccttctgcttcgaggcagtcattgctctcccttccctggccaggttctttagttacacaataagctgaactcataaactgaaagggtatttaggaaggcaaggcgtgggcatgttgatggcttccaatcctggggacccaggaacaaggtgagggcttctctggggcaggtgttgtacctcaggggttctgggaagtctgtatctgggttaatcacccatagtgagcccttggaacggcccacttcccctccccttggccccacttgtccccaacctcacccagccatgcctgcagtttgcttggcctcccagtacagtggcttttattctgtcctggtcactgcactcaaattccaatgtatactttctagtgtaaaaatttatattattgtgggttgttttatgttgttgtttgtttttgtatattgctgtaactactttaacatccagaaataaagattatataggaaaaaaaaaaaaaaaaaaaaaaaaaaaaa

SEQ ID NO: 72 - DNA, 1257 bp, Rattus norvegicus
  misc_feature: >gi|1724074|gb|U66470.1|RNU66470 Rattus norvegicus cell growth regulator rCGR11 mRNA, complete cds
cacgaagccggagccccagacgcgcacaggccgccgcagcagctccggtgttcagcaggatgtcccggtggctaatgcaaatgttgatgctgcccctattgctgctccctttgggtcaagctgcccccaaggatggagttgcaaggctggaccctgaggcacaacagcagctcacaaccaaccccttccagccaggccctgagcagctccgacgtctgcgggattatctcaagggactggagaagatggaagaggaccctgaacaaatgaaccgggagcaagtcctgctatatctctttgctcttcacgactttgaccagaacggacaactggacggcctggaactcttgtccatgctgacagcagctctggcccctggagctgcacacttccccatcaacccggtgatcctggtagtagacatggtgcttgagactcaggacctggatggagacgggctcatgactcctgcagagctcatcaacttcccaggagaagcccccaagcgcgcagagtcccttcccccagctctccaggagccacaacctgccggaagtcagccgcttttagccaacagtccgctgcaatcagaaacccagcaatccctggggactaaagaagaaattacaagtcaggtagaggccaaaagggccttggagcctgaacaggaggctggacatcacatagagactaaagtagatgctctaagccctgaaggggaggctaggggtcaggcagagtctgaaggagatgccccaggtccccgagaggatgctgagagacaggtggagagcaaggacaacgaaggggaagccaaagacctgccagcggaaacactagagacccaaaacactccaaatgtggttgaggctcatagcatccaactggaaaacgacgagatatgagctagacacacaggctcatgcccctcaggatctcagtgcagaacagaagcatcaagtgtgaatacagtgggtctggagccacctctgacatgggtatggtggtccaagctcgtactcccagaagcactgagagctccaggctagcctaggccacttagcaagtttgaagccaggctgggctacttcgtgaggccctgtcttgaggaggggaaaagaaacgctctcccacgtctcctttctggccctcagtaggaagcaggacttttcttcgcagctcaggggagaccacaagctgagaggcagatttcaggccccaaacaatcaataaaaaacaaaatggatttgtctccat

SEQ ID NO: 73 - DNA, 1990 bp, Rattus norvegicus
  misc_feature: >gi|48675849|ref|NM_053727.2| Rattus norvegicus nuclear factor, interleukin 3 regulated (Nfil3), mRNA
cacgaatgtggtggaggctcagtgtggactgccccccgggaatgaagacaggcatcagatttcaaaattcagtcgaaagaacagcttcgtgggtcacagccatccgttcgttcctccgctggcaaccttggataacccataaaaggctcctgacaggttcaccctctgccggacagcagaaggaatattgatggattcgagaagctgaagcccttacaacctgaaagaagactggggaagtgattcgtctcctcctccatcgggtctgtaggacggggaggtcctttctgatgcagctgagaaaaatgcaggccatcaaaaaggagcctgcatccctggatcctaccggcagctcagacaagatgttgctgctgaactctgccttagctgaggtggctgaggacctagcctcaggggaagatctgctcctgaatgaagggagcatggggaaaaacaagtcctctgcctgtcggagaaaacgggaattcatcccggatgagaagaaggacgccatgtattgggagaaacggcggaaaaacaacgaggctgccaaaaggtctcgggagaagcgtcgcctcaatgacctggttctggagaacaagctgatcgccctgggagaagaaaatgccactttaaaagctgagctgctctccctgaaattaaagtttggcttaattagctccacggtgtacgcccaagaaatccagaaactcagtaattccacagctgtgtattttcaggactaccagacatccaaggctgccgtgagctcttacgtggacgagcatgagcctgcgatggtagccggaagttgcatctctgtcatcaagcactctcctcagagctcgctctcggatgtgtctgaggtgtcctctgtggagcacacacaggaaagccccgcacagggaggctgccggagccctgagaacaagttccctgtgatcaagcaggagcccgtggagttggagagctttgccagggagtccagagaggagcggggtgcctattccgcctccatctaccagagctacatgggtagctctttctccacctactcccactcccctcccctcctgcaggtccacggctccaccagcaactccccgagaacctcggaggccgacgagggtgtggtgggcaagtcttctgatggggaagacgagcagcaggtccccaaaggccccatccattctccagtggagctgcagcgggtgcacgccacggtggtgaaggttccggaagtgaacccttccgccttaccgcacaagctccggattaaagccaaggccatgcaggtcaaagtggaagctttggacagtgagttcgaaggcatgcagaaactctcttcaccggcagatgcgctagccaaaagacattttgacctggagaagcatggcgcttcgggtacggcccattcctccctccctcctttctcggtgcaggtgacgaacattcaagattggtccctcagatcggaacactggcatcacaaagaactgggcggcaaaactcagggtaccttcaaaacgggtgtggtggaagtcaaagacagtggctataaggtttccgaggctgagaatttgtatttgaagcagggggtggcaaacttatccgcagaggtggtctcgctcaagagattcatagccacacagcctatctcggcttcggactccaggtaaatggctactgactgagctatgcgtggaagaggatgctcttggtaccatactgaatttcccactggacctctaaagtcatttcactgtagtgtgcacaacggtgtctgtctggtgtccttgtgtacacaacgctgaagacttgatgccctcactctgcctggcgtgtagtcagatagcccttccctccccaccccccagaggctgtacataccgaacgttatttttgctctatgataaagtgtgtacgttgccagtttcaataaaggattggtggcaaacacaaaaaaaaaaaaaaaaaaaaaaaaaaaaaa

SEQ ID NO: 74 - DNA, 2285 bp, Homo sapiens
tcagcccacttccggggagggaggcagaggaacccctccccgccgctcacccctaagcccagccctcggctcccacccttgtgtacctgggccgaaccattcaccggagcgcgcagcgggtggagtgtggctcggaggaccgcggcgggtcaagcacctttctcccccatatctgaaagcatgccctttgtccacgtcgtttacgctcattaaaacttccagaatgcaacaggacggacttggagtagggacaaggaacggaagtgggaaggggaggagcgtgcacccctcctggccttggtgcgcgccgcgccccctaaggtactttggaagggacgcgcgggccagacgcgcccagacggccgcgatggcgctgttggccggcgggctctccagagggctgggctcccacccggccgccgcaggccgggacgcggtcgtcttcgtgtggcttctgcttagcacctggtgcacagctcctgccagggccatccaggtgaccgtgtccaacccctaccacgtggtgatcctcttccagcctgtgaccctgccctgtacctaccagatgacctcgacccccacgcaacccatcgtcatctggaagtacaagtctttctgccgggaccgcatcgccgatgccttctccccggccagcgtcgacaaccagctcaatgcccagctggcagccgggaacccaggctacaacccctacgttgagtgccaggacagcgtgcgcaccgtcagggtcgtggccaccaagcagggcaacgctgtgaccctgggagattactaccagggccggaggattaccatcaccggaaatgctgacctgacctttgaccagacggcgtggggggacagtggtgtgtattactgctccgtggtctcagcccaggacctccaggggaacaatgaggcctacgcagagctcatcgtccttgggaggacctcaggggtggctgagctcttacctggttttcaggcggggcccatagaagactggctcttcgtggttgtggtatgcctggctgccttcctcatcttcctcctcctgggcatctgctggtgccagtgctgcccgcacacttgctgctgctacgtcaggtgcccctgctgcccagacaagtgctgctgccccgaggccctgtatgccgccggcaaagcagccacctcaggtgttcccagcatttatgcccccagcacctatgcccacctgtctcccgccaagaccccacccccaccagctatgattcccatgggccctgcctacaacgggtaccctggaggataccctggagacgttgacaggagtagctcagctggtggccaaggctcctatgtacccctgcttcgggacacggacagcagtgtggcctctgaagtccgcagtggctacaggattcaggccagccagcaggacgactccatgcgggtcctgtactacatggagaaggagctggccaacttcgacccttctcgacctggcccccccagtggccgtgtggagcgggccatgagtgaagtcacctccctccacgaggacgactggcgatctcggccttcccggggccctgccctcaccccgatccgggatgaggagtggggtggccactccccccggagtcccaggggatgggaccaggagcccgccagggagcaggcaggcgggggctggcgggccaggcggccccgggcccgctccgtggacgccctggacgacctcaccccgccgagcaccgccgagtcagggagcaggtctcccacgagtaatggtgggagaagccgggcctacatgcccccgcggagccgcagccgggacgacctctatgaccaagacgactcgagggacttcccacgctcccgggacccccactacgacgacttcaggtctcgggagcgccctcctgccgaccccaggtcccaccaccaccgtacccgggaccctcgggacaacggctccaggtccggggacctcccctatgatgggcggctactggaggaggctgtgaggaagaaggggtcggaggagaggaggagaccccacaaggaggaggaggaagaggcctactacccgcccgcgccgcccccgtactcggagaccgactcgcaggcgtcccgagagcgcaggctcaagaagaacttggccctgagtcgggaaagtttagtcgtctgatctgacgttttctacgtagcttttgtatttttttttttaatttgaaggaacactgatgaagccctgccatacccctcccgagtctaataaaacgtataatcacaaaaaaaaa

SEQ ID NO: 75 - DNA, 2882 bp, Homo sapiens
gtccggccagggtggttggtggtaaggattcaggctccgtcctaacgaggccgtggcctgaggctcagggccccccgcccctccctcccagcccaccagcgtcacctcccagccccgagctggaccgcacaccttgggacacggttttccacttcctaaggacgagccccagactggaggagaggtccgaggaggtgggcgttggactctttgcgaggaccccggcggctggcccgggggaggcggccgaggcggcggcggcggcggccgggggcgacatggcggaggagcaggacctatcggaggtggagctgagccccgtgggctcggaggagccccgctgcctgtccccggggagcgcgccctcgctagggcccgacggcggcggcggcggatcgggcctgcgagccagcccggggccaggcgagctgggcaaggtcaagaaggagcagcaggacggcgaggcggacgatgacaagttccccgtgtgcatccgcgaggccgtcagccaggtgctcagcggctacgactggacgctggtgcccatgcccgtgcgcgtcaacggcgccagcaaaagcaagccgcacgtcaagcggcccatgaacgccttcatggtgtgggctcaggcagcgcgcaggaagctcgcggaccagtacccgcacctgcacaacgctgagctcagcaagacgctgggcaagctctggaggctgctgaacgaaagtgacaagcgccccttcatcgaggaggctgagcggctccgtatgcagcacaagaaagaccacccggactacaagtaccagcccaggcggcggaagaacgggaaggccgcccagggcgaggcggagtgccccggtggggaggccgagcaaggtgggaccgccgccatccaggcccactacaagagcgcccacttggaccaccggcacccaggagagggctcccccatgtcagatgggaaccccgagcacccctcaggccagagccatggcccacccacccctccaaccaccccgaagacagagctgcagtcgggcaaggcagacccgaagcgggacgggcgctccatgggggagggcgggaagcctcacatcgacttcggcaacgtggacattggtgagatcagccacgaggtaatgtccaacatggagacctttgatgtggctgagttggaccagtacctgccgcccaatgggcacccaggccatgtgagcagctactcagcagccggctatgggctgggcagtgccctggccgtggccagtggacactccgcctggatctccaagccaccaggcgtggctctgcccacggtctcaccacctggtgtggatgccaaagcccaggtgaagacagagaccgcggggccccaggggcccccacactacaccgaccagccatccacctcacagatcgcctacacctccctcagcctgccccactatggctcagccttcccctccatctcccgcccccagtttgactactctgaccatcagccctcaggaccctattatggccactcgggccaggcctctggcctctactcggccttctcctatatggggccctcgcagcggcccctctacacggccatctctgaccccagcccctcagggccccagtcccacagccccacacactgggagcagccagtatatacgacactgtcccggccctaaagggggccctgtcgccaccaccccccgcccagcccctgcccccagcctgtgtgccctgttccttgcccacctcaggcctggtggtggcagtggaggaggctgaggaggctgaagaggctgacaggtcggggggctttctgtctggctcactgccctgatgacccacccgccccatccaggctccagcagcaaagccccaggagaacaggctggacagaggagaaggaggttgactgttgcacccacactgaaagatgaggggctgcaccttcccccaggaatgaccctctatcccaggacctgagaagggcctgctcaccctcctcggggaggggaagcaccagggttggtggcatcggaggccttaccactcctatgactcctgttttctctctcacagatagtgagggtctgacatgcccatgccacctatgccacagtgcctaagggctaggccacccagagactgtgcccggagctggccgtgtctcccactcaggggctgagagtagctttgaggagcctcattggggagtggggggttcgagggacttagtggagttctcatcccttcaatgccccctccctttctgaaggcaggaaggagttggcacagaggccccctgatccaattctgtgccaataacctcattctttgtctgagaaacagcccccagtcctcctccactacaacctccatgaccttgagacgcatcccaggaggtgacgaggcaggggctccaggaaaggaatcagagacaattcacagagcctccctccctgggctccttgccagctccctcttcccttactaggctctatggcccctgctcagtcagccccactccctgggcttcccagagagtgacagctgctcaggccctaacccttggctccaggagacacagggcccagcacccaggttgctgtcggcaggctgaagacactagaatcctgacctgtacattctgcccttgcctcttaccccttgcctcccagtggtatttgaataaagtatgtagctatatctgcccctattttcctgttctgcagccccccaaatccacatgtaactcattactgtctcctgttatttatctcagtagtcccctctcctagccactctagcccctattaactctgcattaagcattccacataataaaattaaaggttccggttaaaaaaaaaaaaaaaaaaaaa

SEQ ID NO: 76 - DNA, 1661 bp, Homo sapiens
cggccgctgggtccgtgaaggggagtgaggcactcctcaccctgtggcagctcctagtcaggaggcctctcccctccacctgcacgtcatgtggctgtggctcccaggaccagcgcctggagcgggtgagtgaacgtccctctcctccgtgggcactgcctccgctgtgagccacccttcaggctacgtgcatttccgcctgttctcagggtttaaccgaaaccgaacattaaccgtagccctaacgcggctgagggatccaggccttgcctctgggccgggtgagagacagacactcgccaaccagtccgcagtggctccccgtgtgtgcgaggctcccgcctgcgtgtgtgcgtgtgcatgtgtgcctgcgcacaggggagggaaagttcttcagctgccgagggcgggagtaggggctggagcagcaccctgcggcgaggccccacaggagaatcgatggttctgatgctgtcagcctctgggtgcaaattctgagggcccgggagcgtctggcggccctgagcagcttgctggggaagctccgtggaggaggaagtccggaggcatgtgatgtgaatcagctgacatcatcttgacgggagccctgtgaggcggacacttaactccagtttcacaggagggagccatggatttgctgcgggagctgaaggccatgcctatcacgctgcacctgctccagtccacccgagtcgggatgtctgtcaacgcccttcggaagcagagctcggatgaggaggtcattgcactggccaagtctctcatcaagtcctggaagaagctcctggatgcttccgatgccaaagccagggagcgggggaggggcatgcctctgcccacgtcctcgagggatgcctcagaggccccggatcccagccgcaagaggccggagctgcccagggcaccgtcgactccgaggatcaccacatttcctccggtgcctgtcacctgtgatgccgtgcgcaacaagtgccgcgagatgctgaccgctgccctgcagacggaccatgaccacgtggccatcggtgcggactgcgagcgcctgtcggctcagatcgaggaatgcatcttccgggacgttggaaacacagacatgaagtataagaaccgtgtacggagtcgtatctccaacctgaaggatgccaagaaccctgacctgcggcggaatgtgctgtgtggggccataacaccccagcagatcgctgtgatgacctcagaggagatggccagtgatgagctgaaggagatccgtaaggccatgaccaaggaggccatccgagagcaccagatggcccgcactggcggcacgcagacagacctgttcacctgcggcaagtgcaggaaaaagaactgcacctacacacaggtgcagacccgcagctctgatgagcccatgaccacctttgttgtctgcaacgagtgtggaaaccgctggaagttctgctgacccctcgtgtagatgtgctgcagccttgggccctccccggcccacgtcctccgttgacacagcttctctggagaccctagaaggcggcatgtcctgccctcaacctgcctgcctggattgcacctttctgccctttccccctcattattaaatgtttctttttgccaaaaaaaacaaaaaaaaaaaaaaaaaa

SEQ ID NO: 77 - DNA, 1353 bp, Homo sapiens
cccttcccccagcaggccaaggctggtgacagccttcatatatttaaagaggacaagagcccctcagactcagttgagctgaacggagtccacacaggcagacttgaccttgaccagatagtcttcaagatgccaaactggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcgaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaatgaagaggtgcgccgtttctcagattttttgcgagcctaaaacacttgccaagtaatcctgcacagatcgatacctttccccaaatagcctctcctttgtagtcgtacattatgtgtttctcctcagaagtgatcaggtctttactgaatgttagaagaggcctttggaagaaaattatgtaaagtttaatctataacaaatgctttattatttataatgcttggaatgggagaggcaataaataaatgttttagtgctatcttgtatggctctagatcttttctttgagatagaaattttcaaaaacataaagctagttcaaaaaacgagttgcagagcatataataaatttggatgtcaactgagaaaggagtgagaaggaagaaacaatgcgcaaaggaaagcagtctttcagaatctgtcagccaagtgtctttctagttactgctaatggagaagaaaacagggggtctgggagaaaatagagaacatgatagcaaaatctaaaaggaaaatcaaaactaataaaattgctgaagagttgatccctttgtcctatcgtggggctttgtaatgttacacatctcgtgaaaactcagaaatgacaataaagcgtggcatttgcctctgtattataaatg

SEQ ID NO: 78 - DNA, 2602 bp, Homo sapiens
gaagtcggcgtcttgagtcataggagtgagccacgcccgggctgtgggaataagatggcggggaagaagaatgttctgtcgtctctcgcagtttacgcggaagattcagagcccgagtctgatggcgaggctggaatcgaggcggtgggcagcgcggctgaggagaaaggcggattggtatctgatgcctatggggaggatgacttttctcgtctagggggtgatgaagatggttatgaagaagaagaagatgagaacagtagacagtcggaagatgacgattcagagactgaaaaacctgaggctgatgacccaaaggataatacagaagcagaaaagcgagacccccaggaactcgtggcctccttttctgaaagagttcggaacatgtcgcctgatgaaatcaagatcccgccagaaccccctggcagatgttcaaatcacttgcaagacaagatccagaagctttatgaacgaaagataaaggagggaatggatatgaactacattatccaaaggaagaaagaatttcggaaccctagcatctacgagaagctgatccagttctgtgccattgacgagcttggcaccaactacccaaaggatatgtttgatccccatggctggtctgaggactcctactatgaggcattagccaaggcccagaaaattgagatggacaaattggaaaaggccaaaaaggagcgaacaaaaattgagtttgtgacgggcaccaaaaaaggcaccacgaccaacgccacgtccaccaccactaccactgccagcacagctgttgcagatgctcagaagagaaagagcaagtgggattcggctatcccagtgacaacgatagcccagcccaccatcctcaccaccacagccaccctgccagctgttgtcacggtcaccaccagcgccagcggctccaagaccaccgtcatctctgctgtgggcaccattgtgaagaaggccaagcagtgacctgaggggccaccctaggacttgaaaggaccgtgcagcccagtgaccactgcccagtgggaggcgccactttgtatatttcaggactgggacctactccccagatgccacctgagaggagcttctgtttggcattccagatggaaggacaggcagcacgggagccaggcgctgtggacagggtctgtccacgcaccacctggggtctgccgcctattaaaagtgccgtattcttacctcttggcatctcagatgcactggcctctcctgcattctgtttgcaggcaaatgcttcagctcacatgtcccccaagactcaatagtcttggttgggactattgcctcagggttgacaacagggtgatggaggcctgggacgctgttcagaggggtgacccaagaagtcaccgttgttatccgtgtatgcctctgggcatggacaggcgggagtccccgagccccacgtatgccccatctgccgccttgatcccaaaacccagccagttctcgggtgatggtggagctgtgctcccttgagtgcacttgagcactccccagaccagatcactttttggacacacccaggaaggcttttgaagtcaagccagtttccagaagggctggggtgagatcctgacctgtgcagagagtggggaggcgtcagcgcctggccagggctgccccagtcctttggtgcccggtgagcgcagccagcaggagctgagcagcgggggagatgaatcacctcctgccttggcgaggcggcagctcattgtttacaggcaagccctgctcctgggagggctcctgccaccccacccttcctctgtgtgttatctctgccccacagcagcccctgggcagcaccagtggccactgggctccccccggggttgaaacgggtttcccagaccaggggttcagaggagactatttacccttcatgatctttggcgattctccactggagcggaagggctgtgtgtcaaaagaaggaagccaggctgtgaagggccgtgttgctttcagtgggtgggcagaggtttagaaaggtggttctgaaatggcacctggtactcttgtgggacctggggaatataaggaactctgtgcatgaggtttcaaaaataaaaattgagtccactctccccgtacacacacactcgggcacttcaggaggtcagttctcaccccttcctcgatggggctttaaagctctggggagaagagctgctccccactcatgccccagagggactgtgaaaaggcttcctgtgacctccgatggcggcccctcattggccaagggcactgtggatgttttgtgggaggcagcatggcaccgtggaaagggcagtagacgtggagccggcacctctggtctggattccacttaaaaccagttgagtggtctcaagcccttaacctctgtaagcctgtttcttctctgaaatcacagtaataaagcgtcgtaagatggttgggaagaaccagaggtgacttatgcaaaagctttataaactgtaaagttcctgccaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 79 - DNA, 5319 bp, Homo sapiens
ctgatttcgtgcgcttcctcgtccttcatgttggatggccagtttttcgtttgtgcgtcatcctctacctgagaaatggtcgcttgcccctagtctagacacgcattaaagggcagtatttaaagtcagttggcaagcagtggaataagatttttgtaaagaaaccttgtgcagcatggattctctaccagatgaattttttgtgaggcatcctgctgtggaggatcagaggaaggaagaaactgagaataagctagaaaaatcatctggtcaactgaacaaacaggaaaatgacatacctactgatcttgtccctgttaacctactattagaagtgaagaagttattaaatgcaattaatactctaccaaaaggtgtggttcctcacattaagaagttcttacaagaagatttttccttccaaactatgcagagagaagttgcagctaacagccagaatggtgaggaaattgttcctgctttgactttacgtttcttgattacacagctagaagcagcacttaggaacattcaagctggcaattataccgcacaccagattaatattggttattatttgacattactgtttttatatggagtagcactcactgaaagaggaaagaaagaggattatacagaagctgagaataaatttctggtgatgaagatgatgatccaagaaaatgaaatttgtgaaaactttatgtctttagtttattttggacgtggtttactgcgatgtgctcaaaagagatataatggaggactgctagaatttcataaaagcttacaagaaattggagacaaaaatgaccattggtttgacatagatcctacagaagatgaagatttacctacaacttttaaagatcttcttaataattttatcaagacaactgaaagcaatataatgaagcagacgatttgtagttacctagattgtgaacgatcttgtgaagctgacattttgaagaacaccagttataagggattttttcagttaatgtgcagtaaaagttgctgtgtttatttccataaaatttgctggaaaaagttcaagaatttaaagtatccaggtgaaaatgatcagagttttagtgggaaaaaatgtttgaaggaaggatgtacaggtgacatggtaaggatgctgcaatgtgatgtacctggaattgttaaaattttgtttgaagttgtgagaaaggatgaatatatcaccattgaaaatttaggagcaagttatagaaagttgatatctctgaaaataactgatactgatataagaccgaagatcagtttaaaatttaatacaaaagatgaaatgcctatcttcaagcttgattataattatttctatcatctgcttcatataattattatttctggtactgacattgttcgacaaatatttgatgaggctatgccacctcctcttttgaaaaaagagcttcttatacacaagaatgtgctggaatcctactacaaccatctttggaccaatcatcctttgggtggatcatggcatctgctttatcctcctaacaaggagttaccgcaatccaaacagtttgacttatgcctcctgttagctcttataaaacatttaaatgtgttccctgcacccaaaaaaggatggaatatggaaccgccatcttctgacatctctaaatctgcagacatcctgagactgtgcaaatacagggatatcctccttagtgagattttgatgaatggtctcactgagtcacagttcaattcaatttggaaaaaagtttcagatattcttctgcgccttgggatgatgcaagaggatattgacaaagtgaaggagaatcccattgagaatatctcccttgattaccatcagctatctgtctacctaggcataccagtaccagaaatcatacagaggatgttatcctgctatcaacaaggaattgctctacagtcaataacaggcagtcagcgtattgaaatagaagagttacagaatgaggaagaagaactaagtccacctctcatggagtacaatataaatgtgaaatcacaccctgagatacagtttgcagaaattaataaagatgggacctcaatacccagtgaatcttcaacagaatctcttaaagatctccaggaagttaagagtaaacaaaggaaaaagaagaagactaagaataaaaagaataaagactcaaaagaagaccaagtcccatatgtggtagaaaaggaagagcagttgaggaaagaacaagcaaatccacactcagtcagtagacttataaaagatgatgcaagtgatgttcaagaggattctgcaatggaagacaagttctatagcctggatgaattgcatattctggacatgatagagcagggctcagctggcaaagtaactacagactatggagaaactgaaaaggaaaggcttgctcgtcaaaggcagctttataaattgcactatcagtgtgaagatttcaaaagacagttgagaacagtgacttttcggtggcaagaaaaccaaatgcagattaaaaagaaagacaaaattatcgcatctcttaatcaacaagttgcttttggaatcaataaggtttccaaattacagcgtcaaatccatgctaaagataatgaaatcaagaaccttaaagagcaactttctatgaaaagatctcagtgggaaatggaaaaacataatctggaaagcacaatgaaaacatacgtaagcaaactgaacgcagaaactagcagagctttaacagccgaggtgtatttcttacagtgtcgtagggattttggtttgcttcatctagagcagactgaaaaggaatgtctcaatcagcttgccagggtgactcacatggcagcaagcaacctagaatcacttcaattaaaggctgcggtagacagttggaatgccattgtggcagatgttagaaacaagattgcattcctcaggacacagtacaatgaacaaataaacaaagtcaagcaaggatttgccttgagtaccttgcctccagtccagcttcctcctccaccacccagtcctgagatactgatgcagcagttcttaggaagaccccttgtgaaagaatctttctttagacccatacttactgttcctcaaatgcctgcagtttgcccgggagtcgtctctgcaactggccaacctagagcccccctgatgactggcatagcctgggctctgccagcgcctgtgggagacgctgtgcctcccagtgcaggtctgcggagtgatccctccatcatgaattgggagagaattacagacaggctgaaaactgcctttccacagcaaaccagaaaggaacttacagatttcttacggaaattgaaggatgcttatggaaaatccttgtctgaactgacatttgatgaaattgtttgcaagatttcccagtttattgaccccaaaaagtctcagagtcaaggaaaatcagtgtcaaatgttaattgtgtttcacctagtcattctccatcacagcctgatgctgcccagcccccaaaaccagcctggaggccactcacttcacagggtcctgccacatgggaaggagccagtaatccagatgaggaagaggaagaagaagagccttgtgtgatctgtcatgagaatctgtctccagaaaatctttcagttttgccttgcgctcacaaattccatgctcagtgcattagaccatggttgatgcaacaggggacatgtccaacgtgcagactccacgttttgctaccagaagaattccctggtcaccccagccggcagttgcccaagatctgatacaaggtacccatgaagagattatgaactacttgaaggtggagactgtacggtggtgtgttggagctggcttgtaatgtcttatgagtgacaatcgttagtttgaggaattttgtgagacagttgtcaaattgttgctagcttgaaatctgcggcaattggagtatttacaccatagaaatgctataagtgaagacctacctttcccttaagagctagttgttaaacctttaccagcataccactggaccttgtctaaaatttctttgtgttcccagtgtcttgcccagtagatacaagataaatattgccagaatcagatcaggaagtagtaagaaaaggagttaatatgcaaactaaatcactcgctcaattgaataattgagatcttctgttcatttgttccttggaccttaatcatttgcattttggagaaaattttttctgctttaaaagtctgtaatttcagtttttgtgtcggggagagggaaaaactatttgtctgtagttgctttttgtgacaaagtgaatacccactgggctaagtttcatatctaaagcttgtcactaagaattttcatttttaggggtcaaaaacctattttgaaaatagtgttgtgtgaatgctgtaagtgttgtacatgtctctggtttcagaattaaaagaattcagagttaccttgtgagataaatgttctttgcttcatattcacagtttcacctccatttaaatttatggggaagaataaatgtttcctgactcagaaacaattttgcctccatatttcagtgcaaatatattttgaagttacttttaaatatttatttaaaacctctgtttctgtaactgtatgttaaacgtattcttctgtaatgagaatagactggaacatgtgagggaaaaatattaaaaaaaagatggcagtcatctagacatgtaaaatgccaagaatgtcttagaattgcacaatttagaatttatagagcctttaatcaccttgtctagcaccttgtattagtccattttcatgctgctgataaagacatacctgagattgggcaatttacaaaagaaagaggtttaatggacttacagttccacatgactaaggaggcctcacaatcatggtggaaggcaaagaggagcaagtcacatcttacatggatggcagcagacaaggagagagagcttgtgcagggaaactcccatttttaaaaccatcagatctcatgaggcttactatcacgagaacagcatgggaaagacctgcccccgtgattaaattatttcccaccaggcccctcccacaacatggaataatgggagatacaagatatttgggtggagacacagagccaaaccatatcacaccttaaggtcatggtcatatagctagttatcttgtaaaaccaatgtttcttagttcatacttacaactgcaatttcactttcatttagaaaagaataaatgtttcttaattgaacaactaaaaaaaaaaaaaaaaaaaaaaaaaaa

SEQ ID NO: 80 - DNA, 3900 bp, Homo sapiens
ccatgggctcaggatgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttagtggggctgcccagtgtggacacgtggctcacccagggatcaaggcgctgctgggccgctccccttggaggccctgctcaggaggccctgaccgtggaaggggagaggaactcgaaagcacagctcctcccccagcccttgggaccatcttctcctgcagtcccctgggccccagtgagaggggcaggggcagggccggcagtaggtggggcctgtggtccccccactgccctgagggcattagctggtctaactgcccggaggcacccggccctgggccttaggcacctcaaggacttttctgctatttactgctcttattgttaaggataataattaaggatcatatgaataattaatgaagatgctgatgactatgaataataaataattatcctgaggagaaaa

SEQ ID NO: 81 - DNA, 1994 bp, Homo sapiens
catattcagcagggttagcttgtcctcccctccctcttcagcttcccagacactgagtctggaatgaaaattcacctgcctctgagttggctcctaatgggggtgggagtgttacttcggttcccaggttggaagattatctcacccggccccagctatataagctgaccggtgtggaggggcccagcagggccaactccagggattccttccacgacagaaaaacatacaagactccttcagccaacatgatggtactgaaagtagaggaactggtcactggaaagaagaatggcaatggggaggcaggggaattccttcctgaggatttcagagatggagagtatgaagctgctgttactttagagaagcaggaggatctgaagacacttctagcccaccctgtgaccctgggggagcaacagtggaaaagcgagaaacaacgagaggcagagctcaaaaagaaaaaactagaacaaagatcaaagcttgaaaatttagaagaccttgaaataatcattcaactgaagaaaaggaaaaaatacaggaaaactaaagttccagttgtaaaggaaccagaacctgaaatcattacggaacctgtggatgtgcctacgtttctgaaggctgctctggagaataaactgccagtagtagaaaaattcttgtcagacaagaacaatccagatgtttgtgatgagtataaacggacagctcttcatagagcatgcttggaaggacatttggcaattgtggagaagttaatggaagctggagcccagatcgaattccgtgatatgcttgaatccacagccatccactgggcaagccgtggaggaaacctggatgttttaaaattgttgctgaataaaggagcaaaaattagcgcccgagataagttgctcagcacagcgctgcatgtggcggtgaggactggccactatgagtgcgcggagcatcttatcgcctgtgaggcagacctcaacgccaaagacagagaaggagataccccgttgcatgatgcggtgagactgaaccgctataagatgatccgactcctgattatgtatggcgcggatctcaacatcaagaactgtgctgggaagacgccgatggatctggtgctacactggcagaatggaaccaaagcaatattcgacagcctcagagagaactcctacaagacctctcgcatagctacattctgaggcaaacgacagactcttaatcagtaaatgttcactggcattttgaaggcatggcccaagagaagagacactagccataaaatctagtttctatttatcaacgtgttgtgaagatgtacctaatgaagttttgagaaagcacagggttataggtgtttaaatttcctttagtgaaactcttatttatttttatgtattcctgtttatttatttactgccacgctactgatattcagaccttcatgatcatccatctggtgagcagagcttcatttgtatataacactttcagagccttcccacccataggtagttcttaaaccaggtgaaagagcaaagttcaagtgcctacttatgtgtcattcgctcatgtaagagtttttaagagagggctgattatcacagccctcttttctcctgaatttttaatgcagaagtttgaatgaagcaagggaaggcatgtagggacaggaaaggaaacaatggaaggaaagtgattctgtgaaaaggacagtgaagccagctattttacccccaggctggattttttttttttttttttttttttttttttaccgagtacacagagtacccaagtgaagagaacgtcatgagtgtaagtgcaaatcagtggaaggagcggcaaactgggacatgcagaattgaatttgctcaaaaaagatgaaaggaaatgcaaactgtaaatgtataaatgtatattgtattgtatgtacattttatattcataataaaggcaatcaaactcaaaaaaaaaaaaaaaaaaaa

SEQ ID NO: 82 - DNA, 3447 bp, Homo sapiens
ggccagtccgcccggcggctcgcgcacggctccgcggtcccttttgcctgtccagccggccgcctgtccctgctccctccctccgtgaggtgtccgggttcccttcgcccagctctcccacccctacccgaccccggcgcccgggctcccagagggaactgcacttcggcagagttgaatgaatgaagagagacgcggagaactcctaaggaggagattggacaggctggactccccattgcttttctaaaaatcttggaaactttgtccttcattgaattacgacactgtccacctttaatttcctcgaaaacgcctgtaactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataatgtataatgggaaatgtttggccatgcttggttgttgcagttcagacaaatgtaacacacacacacatacacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaaaaaaaaaa

SEQ ID NO: 83 - DNA, 2122 bp, Homo sapiens
tgggagtccgtgctcctgctcctcggttggctcctaagtgccccgccaggtcccctctcctttcgctctcccggctccggctcccgactcttcggcccgctggcatctgcttccctcccctgcctcgtttctcgtcgcccctgctcgctccccccggcgctcgcccgggcgctgtgctcgctcctggatcgccagccgcgcagccgggctcggccggccgcccgcgcgccactgtgcagtggagtttggtggaatctctgctgacgtcacgtcactccccacacggagtaggagcagagggaagagagagggatgagagggagggagaggagagagagtgcgagaccgagcgagaaagctggagaggagcagaaagaaactgccagtggcggctagatttcggaggccccagtgcacccgtggactccttcggaacttggcaccctcaggagccctgcagtcctctcaggcccggctttcgggcgcttgccgtgcagccggaggctcggctcgctggaaatcgccccgggaagcagtgggacgcggagacagcagctctctcccggtagccgataacggggaatggagaccaactgccgcaaactggtgtcggcgtgtgtgcaattagagaaagataaaagccagcaggggaagaatgaggacgtgggcgccgaggacccgtctaagaagaagcggcaaaggcggcagcggactcactttaccagccagcagctccaggagctggaggccactttccagaggaaccgctacccggacatgtccacacgcgaagaaatcgctgtgtggaccaaccttacggaagcccgagtccgggtttggttcaagaatcgtcgggccaaatggagaaagagggagcgcaaccagcaggccgagctatgcaagaatggcttcgggccgcagttcaatgggctcatgcagccctacgacgacatgtacccaggctattcctacaacaactgggccgccaagggccttacatccgcctccctatccaccaagagcttccccttcttcaactctatgaacgtcaaccccctgtcatcacagagcatgttttccccacccaactctatctcgtccatgagcatgtcgtccagcatggtgccctcagcagtgacaggcgtcccgggctccagtctcaacagcctgaataacttgaacaacctgagtagcccgtcgctgaattccgcggtgccgacgcctgcctgtccttacgcgccgccgactcctccgtatgtttatagggacacgtgtaactcgagcctggccagcctgagactgaaagcaaagcagcactccagcttcggctacgccagcgtgcagaacccggcctccaacctgagtgcttgccagtatgcagtggaccggcccgtgtgagccgcacccacagcgccgggatcctaggaccttgccggatggggcaactccgcccttgaaagactgggaattatgctagaaggtcgtgggcactaaagaaagggagagaaagagaagctatatagagaaaaggaaaccactgaatcaaagagagagctcctttgatttcaaagggatgtcctcagtgtctgacatctttcactacaagtatttctaacagttgcaaggacacatacacaaacaaatgtttgactggatatgacattttaacattactataagcttgttattttttaagtttagcattgttaacatttaaatgactgaaaggatgtatatatatcgaaatgtcaaattaattttataaaagcagttgttagtaatatcacaacagtgtttttaaaggttaggctttaaaataaagcatgttatacagaagcgattaggatttttcgcttgcgagcaagggagtgtatatactaaatgccacactgtatgtttctaacatattattattattataaaaaatgtgtgaatatcagttttagaatagtttctctggtggatgcaatgatgtttctgaaactgctatgtacaacctaccctgtgtataacatttcgtacaatattattgttttacttttcagcaaatatgaaacaaatgtgttttatttcatgggagtaaaatatactgcatacaaaaaaaaaaaaaaaaaaaaaaaaa

SEQ ID NO: 84 - DNA, 658 bp, Homo sapiens
ggagcctcacgrgagcgkggtaacgttatagtatttgtcagaagttggggtctccgtgggcattgtgatccgtcccaggcagtggattaggaggccagaaggagatcccttccacggtgctaggctgagatggatcctctcagggcccaacagctggctgcggagctggaggtggagatgatggccgatatgtacaacagaatgaccagtgcctgccaccggaagtgtgtgcctcctcactacaaggaagcagagctctccaagggcgagtctgtgtgcctggaccgatgtgtctctaagtacctggacatccatgagcggatgggcaaaaagttgacagagttgtctatgcaggatgaagagctgatgaagagggtgcagcagagctctgggcctgcatgaggtccctgtcagtatacaccctggggtgtaccccaccccttcccactttaataaacgtgctccctgttgggtgtcatctgtgaagactgccaggcctaggctctctgtagagagtcttcaagatcccggagtggtagcgctgtctcctggtgaaggagtatttgtcacactggaatgtgactgtgtgtgtatgtatgtgtatatatatatatatatatatatataaacaagtttgttgacacctacaaaaaaaaaa

SEQ ID NO: 85 - DNA, 3892 bp, Homo sapiens
ggcgcagaagcgggacgtcggcttctaggggcccaggccggcggcggcgatggcctcggcagctgtggagagcttcgtgaccaagcaactggacctgctggagcttgagagagacgcggaggtggaggagcgcaggtcctggcaggagaacatctctctgaaagagctccagagccgaggcgtgtgtttgctgaagctgcaggtatccagccagcgcactgggctgtacggacggctgctggtcacctttgagcccaggcgatacgggtccgcggcagctcttcccagtaacagctttacttctggtgatatcgtgggcctgtacgatgctgctaatgagggcagtcagctggccactgggatcttgacccgggtcacccagaagtcggtcacggtggcctttgatgagtcccacgatttccagttgagcttggaccgagagaattcctacagactgttaaaacttgccaatgatgtcacttacaggcgactgaaaaaagccctgattgctctaaagaagtatcattctggcccagcctcctcactcatagaagtgctctttggcagatctgctcccagtcctgccagtgaaatacacccgctgacattcttcaacacctgcctggacacctcccagaaagaagcggtttcatttgcgctgtctcagaaagaacttgccatcatccatggacctcctggcactgggaaaaccacgactgtggttgagatcattcttcaagctgtgaaacaaggcttaaaggttctgtgctgcgccccctccaacatcgccgtggacaatctggtggagcgcctggctctgtgtaagcagcggattctgcgcctgggacaccctgcccgcctcctggagtccgttcagcagcactccctggatgcggttttagcgcggagcgacagtgcccagaatgttgcagatatcaggaaggacatcgaccaggtctttgtgaaaaacaaaaagacccaggataagagagagaaaagtaattttcgaaatgaaattaagctgttaagaaaagaactgaaggagagggaagaagcagctatgctcgagagcctcacttcggcaaacgtggtccttgcaacaaacacaggtgcgtctgccgatggccccctgaagttgctgcccgagagctacttcgacgtggtggtcattgacgagtgtgcccaggccctcgaggcgagctgctggatccccctgctgaaggccagaaagtgcatcctggcgggcgatcacaagcagctgccccccaccacagtctctcacaaggctgcgctggcaggactgtcactcagcctgatggaacgcctggctgaggagtacggcgcgagggtggtgcggacactgacggtgcagtaccgcatgcaccaggctatcatgcgctgggcctcagacaccatgtaccttgggcaggtcacagcccactcttccgtggcaaggcacctcctgagggacctcccaggtgtggctgccacagaagagacgggtgtgcccctgctcttggtggacaccgccggctgcgggctgtttgagctggaggaggaggacgaacagtcgaaagggaaccctggcgaagtccgcctcgtcagtttgcacatccaggctctggtggacgctggtgttccagcccgtgacattgctgtggtctcgccatacaacctccaggtggacctgctcagacagagccttgtgcacaggcaccctgagcttgaaatcaagtctgtcgatggcttccaaggccgagagaaggaggccgtgatactgtccttcgtcagatccaacaggaaaggtgaagttggttttcttgctgaggaccggaggatcaacgtggctgtcacccgtgcccgacgccacgtggcggtcatctgtgactcccgtactgtcaacaaccatgcatttttgaagaccctggtggagtatttcacacagcatggggaagtacgcacggcctttgagtatcttgacgatattgtcccagaaaactattcccatgagaactcccagggttccagccacgctgccaccaagccccagggacctgctacgtccaccaggaccggaagccagcggcaggagggaggccaggaggctgcagcacctgccagacagggccggaagaagccggctgggaagtctctggcctctgaagctccatctcagcccagcctcaacggaggcagcccagagggagtggagagccaagatggcgtggaccacttccgggccatgatagtggagttcatggccagcaagaagatgcagttggagtttcctccttccctcaattcccacgacaggctgcgggtccaccaaatagccgaggagcacgggctgaggcacgacagttccggggaagggaagaggaggttcatcactgtgagcaagagggccccgcgaccccgagcagccctgggacccccagcagggaccggtggcccagcccctctccagccagtgccccctacccctgcgcagacagagcagcctcccagggagcagcgtggcccagaccagcctgatctgaggacgctgcacctggagagactgcagagggtcaggagcgcgcaggggcagcccgccagcaaggagcagcaggcctcagggcagcagaaacttccagaaaagaaaaagaaaaaagccaaaggacatccggccacagatctgcccacggaggaggactttgaggccctggtttctgccgccgttaaggctgataacacctgcggctttgccaagtgcacagccggcgtcacaaccctgggccagttctgccagctctgcagccgccgctactgcctcagccaccacctgcccgagatccatggctgcggtgagagggctcgcgcccatgcccggcagagaatcagccgggaaggggtcctctatgccggcagcgggaccaagaacggatccctggacccagccaagagggcccagctgcagaggaggctggataagaagctgagtgagctcagcaaccagaggaccagccggaggaaggagagggggacgtgaccggccgcatccttgcacgccccgcggagctctctccatggtagcccagggcgctggcagaccatgctccgcctccaccagggccacagaggagcggaggggcctatgggggaggagcggagggccctgttggggaaggttgggtttttggaccccagggataagcttttccgatgtcacaatgtggaggaaagcacctgggggacaacagtgctcgtgcaggtggggcttgggaaatgcacgtcccttccccttactccccgccaaaacccacatcccagcctctggatcctggggaaggttccagtccctggagaatacccagggcctcaaacttgaagtcactcctccaatgtctgggacttgccagctcagcccgttaggatgagggtgctgagaggaaacaggaaacaagactgcgaatggcgctcaggcagggagcagggagtggcgtttggcttgcacgttcccatgtggccagatgctggggccactttccttctgtctgctggtgactgcagtgttccccctcctcctcaccacggggctcctgtgagtctggggggcacctctttctggcctgtgcacctctctctggcttataaaggtgcctggcctgtgccaacccctccttgttgcgcctcaccgtggggaccaggtgagccggctctcccacgtggttgtcccgggaaaagctgccccacagcctcagcatcttcagcacttaccgatccagagcctcccggccttctccggtgtcctgtaccaactcttctatttaagagaacctcagatgatgtacctgagcctcagggttttgtttcagagggatataaattatttaaaaattaaatgaaaacg

SEQ ID NO: 86 - DNA, 5288 bp, Homo sapiens
ggaccggaagtggggggcggaagtgcagtgggctcagcgccgactgcgcgcctctgcccgcgaaaactctgagctggctgacagctggggacgggtggcggccctcgactggagtcggttgagttcctgagggaccccggttctggaaggttcgccgcggagacaagtgagcagtctgtgccatagggattctcgaagagaacagcgttgtgtcccagtgcacatgctcgcatcgcttaccaggagtgcccgagaccctaagatgttcggagtggttttttcgcacagacccgaatagcctgcccctcagccacgctctgtgcccttctgagaacaggctgatatgcccaagatagtcctgaatggtgtgaccgtagacttccctttccagccctacaaatgccaacaggagtacatgaccaaggtcctggaatgtctgcagcagaaggtgaatggcatcctggagagccctacgggtacagggaagacgctgtgcctgctgtgcaccacgctggcctggcgagaacacctccgagacggcatctctgcccgcaagattgccgagagggcgcaaggagagcttttcccggatcgggccttgtcatcctggggcaacgctgctgctgctgctggagaccccatagcttgctacacggacatcccaaagattatttacgcctccaggacccactcgcaactcacacaggtcatcaacgagcttcggaacacctcctaccggcctaaggtgtgtgtgctgggctcccgggagcagctgtgcatccatcctgaggtgaagaaacaagagagtaaccatctacagatccacttgtgccgtaagaaggtggcaagtcgctcctgtcatttctacaacaacgtagaagaaaaaagcctggagcaggagctggccagccccatcctggacattgaggacttggtcaagagcggaagcaagcacagggtgtgcccttactacctgtcccggaacctgaagcagcaagccgacatcatattcatgccgtacaattacttgttggatgccaagagccgcagagcacacaacattgacctgaaggggacagtcgtgatctttgacgaagctcacaacgtggagaagatgtgtgaagaatcggcatcctttgacctgactccccatgacctggcttcaggactggacgtcatagaccaggtgctggaggagcagaccaaggcagcgcagcagggtgagccccacccggagttcagcgcggactcccccagcccagggctgaacatggagctggaagacattgcaaagctgaagatgatcctgctgcgcctggagggggccatcgatgctgttgagctgcctggagacgacagcggtgtcaccaagccagggagctacatctttgagctgtttgctgaagcccagatcacgtttcagaccaagggctgcatcctggactcgctggaccagatcatccagcacctggcaggacgtgctggagtgttcaccaacacggccggactgcagaagctggcggacattatccagattgtgttcagtgtggacccctccgagggcagccctggttccccagcagggctgggggccttacagtcctataaggtgcacatccatcctgatgctggtcaccggaggacggctcagcggtctgatgcctggagcaccactgcagccagaaagcgagggaaggtgctgagctactggtgcttcagtcccggccacagcatgcacgagctggtccgccagggcgtccgctccctcatccttaccagcggcacgctggccccggtgtcctcctttgctctggagatgcagatccctttcccagtctgcctggagaacccacacatcatcgacaagcaccagatctgggtgggggtcgtccccagaggccccgatggagcccagttgagctccgcgtttgacagacggttttccgaggagtgcttatcctccctggggaaggctctgggcaacatcgcccgcgtggtgccctatgggctcctgatcttcttcccttcctatcctgtcatggagaagagcctggagttctggcgggcccgcgacttggccaggaagatggaggcgctgaagccgctgtttgtggagcccaggagcaaaggcagcttctccgagaccatcagtgcttactatgcaagggttgccgcccctgggtccaccggcgccaccttcctggcggtctgccggggcaaggccagcgaggggctggacttctcagacacgaatggccgtggtgtgattgtcacgggcctcccgtaccccccacgcatggacccccgggttgtcctcaagatgcagttcctggatgagatgaagggccagggtggggctgggggccagttcctctctgggcaggagtggtaccggcagcaggcgtccagggctgtgaaccaggccatcgggcgagtgatccggcaccgccaggactacggagctgtcttcctctgtgaccacaggttcgcctttgccgacgcaagagcccaactgccctcctgggtgcgtccccacgtcagggtgtatgacaactttggccatgtcatccgagacgtggcccagttcttccgtgttgccgagcgaactatgccagcgccggccccccgggctacagcacccagtgtgcgtggagaagatgctgtcagcgaggccaagtcgcctggccccttcttctccaccaggaaagctaagagtctggacctgcatgtccccagcctgaagcagaggtcctcagggtcaccagctgccggggaccccgagagtagcctgtgtgtggagtatgagcaggagccagttcctgcccggcagaggcccagggggctgctggccgccctggagcacagcgaacagcgggcggggagccctggcgaggagcaggcccacagctgctccaccctgtccctcctgtctgagaagaggccggcagaagaaccgcgaggagggaggaagaagatccggctggtcagccacccggaggagcccgtggctggtgcacagacggacagggccaagctcttcatggtggccgtgaagcaggagttgagccaagccaactttgccaccttcacccaggccctgcaggactacaagggttccgatgacttcgccgccctggccgcctgtctcggccccctctttgctgaggaccccaagaagcacaacctgctccaaggcttctaccagtttgtgcggccccaccataagcagcagtttgaggaggtctgtatccagctgacaggacgaggctgtggctatcggcctgagcacagcattccccgaaggcagcgggcacagccggtcctggaccccactggaagaacggcgccggatcccaagctgaccgtgtccacggctgcagcccagcagctggacccccaagagcacctgaaccagggcaggccccacctgtcgcccaggccacccccaacaggagaccctggcagccaaccacagtgggggtctggagtgcccagagcagggaagcagggccagcacgccgtgagcgcctacctggctgatgcccgcagggccctggggtccgcgggctgtagccaactcttggcagcgctgacagcctataagcaagacgacgacctcgacaaggtgctggctgtgttggccgccctgaccactgcaaagccagaggacttccccctgctgcacaggttcagcatgtttgtgcgtccacaccacaagcagcgcttctcacagacgtgcacagacctgaccggccggccctacccgggcatggagccaccgggaccccaggaggagaggcttgccgtgcctcctgtgcttacccacagggctccccaaccaggcccctcacggtccgagaagaccgggaagacccagagcaagatctcgtccttccttagacagaggccagcagggactgtgggggcgggcggtgaggatgcaggtcccagccagtcctcaggacctccccacgggcctgcagcatctgagtggggtgagcctcatgggagagacatcgctgggcagcaggccacgggagctccgggcgggcccctctcagcaggctgtgtgtgccagggctgtggggcagaggacgtggtgcccttccagtgccctgcctgtgacttccagcgctgccaagcctgctggcaacggcaccttcaggcctctaggatgtgcccagcctgccacaccgcctccaggaagcagagcgtcatgcaggtcttctggccagagccccacaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgtgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccccctcttatttattctacatccttggcaccccacttgcactgaaagaggcttttttttaaatagaagaaatgaggtttcttaaagcttatttttataaagctttttcataaaactggttgtagttgc

SEQ ID NO: 87 - DNA, 1561 bp, Homo sapiens
gcggcgcggagggcgcgggcccgggagccagggagcgagcggggcgcccggcagcgcggagtcagcgccgcgggggccgcacccgactcgcgcctggacactcgcggggcgccgacctggcagggggccaaaccagtgctcctgccacctctctggctgccccctagagcctgcccatcccagcctgaccaatgtccacagccagggagcagccaatcttcagcacacgggcgcacgtgttccaaattgacccagccaccaagcgaaactggatcccagcgggcaagcacgcactcactgtctcctatttctacgatgccacccgcaatgtgtaccgcatcatcagcatcggaggcgccaaggccatcatcaacagcactgtcactcccaacatgaccttcaccaaaacttcccagaagttcgggcagtgggccgacagtcgcgccaacacagtctacggcttgggctttgcctctgaacagcatctgacacagtttgccgagaagttccaggaagtgaaggaagcagccaggctggccagggagaaatctcaggatggcggggagctcaccagtccagccctggggctcgcctcccaccaggtgcccccgagccctctcgtcagtgccaacggccccggcgaggaaaaactgttccgcagccagagcgctgatgcccccggccccacagagcgcgagcggctaaagaagatgttgtctgagggctccgtgggcgaggtacagtgggaggccgagtttttcgcactgcaggacagcaacaacaagctggcaggcgccctgcgagaggccaacgccgccgcagcccagtggaggcagcagctggaggctcagcgtgcagaggccgagcggctgcggcagcgggtggctgagctggaggctcaggcagcttcagaggtgacccccaccggtgagaaggaggggctgggccagggccagtcgctggaacagctggaagctctggtgcaaaccaaggaccaggagattcagaccctgaagagtcagactggggggccccgcgaggccctggaggctgccgagcgtgaggagactcagcagaaggtgcaggacctggagacccgcaatgcggagttggagcaccagctgcgggcgatggagcgcagcctggaggaggcacgggcagagcgggagcgggcgcgggctgaggtgggccgggcagcgcagctgctggacgtcaggctgtttgagctgagtgagctgcgtgagggcctggcccgcctggctgaggctgcgccctgagccggggctggttttctatgaacgattccggcctgggatgcgggccaggctgcaggcggcatagttgggcccattcgtcctggaaagggactggggggtcccaacttagccctgggtgggccgggccgggctgggctggggtgggccccagtcggctctggttgttggcagctttggggctgtttttgagcttctcattgtgtagaatttctagatcccccgattacatttctaagcgtgaaaaaaaaaaaaaaaaaaaaaaaaaaaaaaaaaaaaaaaaaaaa

SEQ ID NO: 88 - DNA, 1948 bp, Homo sapiens
gagcgccgggctgacgtgcggcggcgatggaagaacttacggcgttcgtctccaagtcttttgaccagaaagtgaaggagaagaaggaggcgatcacgtaccgggaggtgctggagagcgggccgctgcgcggggccaaggagccgaccggctgcaccgaggcgggccgcgacgaccgcagcagcccggcagtccgggcggccggcggaggcggcggcggaggaggcggaggcggcggcggaggaggcggaggaggtgtaggaggaggtggagcaggcggaggagctggaggagggcgctctcccgtccgggagctggacatgggcgccgccgagagaagcagggagcccggcagcccgcggctgacggaggtgtccccggagctgaaagatcgcaaagacgatgcgaaagggatggaggacgaaggccagaccaaaatcaagcagaggcgaagtcggaccaatttcaccctggaacaactcaatgagctggagaggctttttgacgagacccactatcccgacgccttcatgcgagaggaactgagccagcgactgggcctgtcggaggcccgagtgcaggtttggtttcaaaatcgaagagctaaatgtagaaaacaagaaaatcaactccataaaggtgttctcataggggccgccagccagtttgaagcttgtagagtcgcaccttatgtcaacgtaggtgctttaaggatgccatttcagcaggatagtcattgcaacgtgacgcccttgccctttcaggttcaggcgcagctgcagctggacagcgctgtggcgcacgcgcaccaccacctgcatccgcacctggccgcgcacgcgccctacatgatgttcccagcaccgcccttcggactgccgctcgccacgctggccgcggattcggcttccgccgcctcggtagtggcggccgcagcagccgccaagaccaccagcaaggactccagcatcgccgatctcagactgaaagccaaaaagcacgccgcagccctgggtctgtgacgccaacgccagcaccaatgtcgcgcctgtcccgcggcactcagcctgcacgccctccgcgccccgctgcttctccgttacccctttgagacctcgggagccggccctcttcccgcctcactgaccatccctcgtcccctatcgcatcttggactcggaaagccagactccacgcaggaccagggatctcacgaggcacgcaggctccgtggctcctgcccgttttcctactcgagggcctagaattgggttttgtaggagcgggtttgggggagtctggagagagactggacagggtagtgctggaaccgcggagtttggctcaccgcaaagctacaacgatggactcttgcatagaaaaaaaaaatcttgttaacaatgaaaaaatgagcaaacaaaaaaatcgaaagacaaacgggagagaaaaagaggaaggcaacttatttcttaactgctatttggcagaagctgaaattggagaaccaaggagcaaaaacaaattttaaaattaaagtattttatacatttaaaaatatggaaaaacaacccagacgattctcgagagactggggggagttaccaacttaaatgtgtgttttaaaaaatgcgctaagaaggcaaagcagaaagaagaggtatacttatttaaaaaactaagatgaaaaaagtgcgcaggtgggaagttcacaggttttgaaactgacctttttctgcgaagttcacgttaatacgagaaatttgatgagagaggcgggcctccttttacgttgaatcagatgctttgagtttaaacccaccatgtatggaagagcaagaaaagagaaaatattaaaacgaggagagagaaaaataatggcaaaactgtctggactgctgacagtaaattccggtttgcatggaaaaaaaaaaaaaaaaaaaaaaaaaaa

SEQ ID NO: 89 - DNA, 4703 bp, Homo sapiens
ataaatgatcttggcatagaagagaatttaaagagtgatggcttcattccagcgctccaatagtcatgacaaagtaaggagaatagttgcagaggagggtcgtacagcaagaaacctaatagcttggagtgttccactagaaagcaaagatgatgatggaaaacctaaatgtcaaactggtggaaaatctaagaggaccattcaaggcactcataaaactactaaacagagtactgcagtggactgtaaaataacatcgtctacgactggagataaacactttgataaaagtcccactaaaacaaggcaccctcggaaaattgatctaagagctcgatactgggcatttctttttgataatcttcgccgagcagtagatgaaatctatgtaacttgcgaatcagatcagagtgtggtcgaatgtaaggaggtgctaatgatgctggataactatgtaagagatttcaaagcattgattgactggattcagcttcaggaaaagctagagaagacagatgctcaaagcagaccaacatcattggcatgggaagtaaagaagatgtctccgggacgccatgtgattccaagtccatcaacagatagaataaatgtaacatcaaatgctcgacgaagcttaaattttggaggttcaactggcacagtgccagctcctcgtctggctcccacaggtgtcagttgggctgacaaggtaaaggctcatcatacaggctctactgcttcttcagaaataacacccgcccagtcttgcccaccaatgacagtgcagaaggcctcacgcaaaaatgaacggaaagatgctgaaggatgggagaccgttcagagaggaaggcctattcgttctcgatcaacagcagtgatgccaaaagtttcattggcaacagaagccacaagatcaaaggatgacagtgataaagaaaatgtatgtcttttacctgatgaaagcatacagaaaggtcaatttgttggagatggaacttctaatactatagaatctcatcccaaagactcattacactcttgtgaccatcctcttgccgaaaaaacccagttcacagtgagtacattggatgatgtgaagaattctggcagtattcgagacaattatgttcgaacttctgaaatatctgctgtccacattgatacagagtgtgtttcagttatgctgcaagctggtacacctcctttacaagtaaatgaagaaaaatttccagcagagaaagcaaggatagaaaatgaaatggacccttcagatatttcaaattccatggcagaagtccttgctaaaaaagaagagctagcagatcgtctagaaaaggccaatgaagaagccattgctagtgctattgctgaagaagaacagttaactagagaaattgaagctgaagaaaacaatgatattaacattgaaactgacaacgacagtgatttttctgccagcatgggcagtgggagtgtttctttctgtggtatgtccatggactggaacgatgtccttgcagattatgaagctcgtgagtcttggcgccaaaatacatcctggggggacattgtagaagaagaacctgctagacctccagggcatggaattcacatgcatgaaaaactttcttcaccctctcgtaaaagaacaattgcagaatctaagaagaaacatgaagaaaaacaaatgaaagcacagcagctaagggaaaagttacgcgaagagaaaacattgaagcttcagaaattgttagaaagggagaaggatgtccggaagtggaaggaagaattgctagatcaacgacgcaggatgatggaagaaaaattacttcatgctgagtttaagcgagaagtgcagttacaagcaattgtgaaaaaagcacaagaagaagaagctaaggtaaatgaaattgcctttataaatacccttgaagcccagaataaacgtcatgatgttttatcaaaattgaaggaatatgaacagaggcttaatgagctacaggaagagcgtcagagaagacaggaagaaaagcaagcacgtgatgaagctgtgcaggaacgcaagagagctctagaggcagagcggcaggcccgtgtagaagaattgttaatgaagaggaaagaacaagaagcccgaattgaacaacagaggcaagaaaaggaaaaagcccgtgaggatgcagcccgggaaagagctagagacagggaagaacgattggcagcactcacagctgctcaacaagaagctatggaagagttacagaaaaaaattcagctcaagcatgatgaaagtattcgaaggcacatggaacagattgaacaaagaaaagaaaaagctgctgagctaagcagtgggcgacatgcaaatactgattatgcccccaaactgaccccttatgaaagaaagaagcagtgttctctctgcaatgtcctgatctcttcagaggtatatctttttagccatgttaaagggagaaaacaccagcaagccgtgagagagaataccagcatccaggggcgtgaactgtcagatgaagaagtggagcatctttccttgaagaagtacattattgacattgtggttgaaagtacagctccagcagaagctttgaaagatggagaagagcggcaaaaaaataaaaaaaaagccaaaaagataaaagcccggatgaacttcagggctaaggaatatgagagtttaatggaaaccaaaaattctggctctgattcaccttataaagcaaagcttcagcgattagccaaagatcttctaaaacaagtacaagttcaagacagtggctcatgggcaaacaataaagtgtctgctttggatcggaccctaggagagatcactagaatactggaaaaagagaatgtggcagatcagattgcatttcaagctgctggtggattaacagcccttgaacacatccttcaagcagtagtcccagccacaaatgtgaacacagttttaagaattcctcctaagtctctctgcaatgcaatcaatgtttacaacctcacctgcaataactgttcagaaaactgcagtgatgttctgtttagtaacaagattaccttcttaatggacctcctgatacaccagttgacggtttatgttccagatgaaaataatactattttggggagaaatacaaataaacaagtttttgaaggcttgacaactggacttctcaaagtcagtgctgtggttttgggctgcctgattgccaatcgaccagatggaaactgccagccagctaccccaaaaataccaacacaggaaatgaaaaacaaaccctcacaaggtgatccttttaacaatcgagttcaggaccttatcagctacgtggtgaacatgggtctgattgacaaactgtgtgcctgcttcctctcggtgcaaggcccagtggatgagaatcccaagatggccatatttctgcagcatgccgcaggactcttacatgcaatgtgtacactgtgctttgctgtcactggaaggtcatacagcatatttgacaataatcgccaggatcccacagggctgacagctgctcttcaggcaaccgacctggctggagttcttcatatgctctactgtgtcctcttccatggcaccatcttggaccccagcactgccagtcccaaggagaattacactcaaaataccatccaagtggccattcagagtttacgtttcttcaacagctttgcagctcttcatctgcctgcttttcagtctattgtaggggcagagggcttgtcccttgcattccggcacatggccagctccctgctgggccactgcagccaagtctcctgtgaaagcctccttcatgaggtcatcgtctgtgtgggctacttcactgtcaaccacccagataaccaggtgatcgtgcagtccggccgccaccccacagtgctgcagaagctctgccagttgcccttccagtatttcagtgacccacggctgatcaaagtactgttcccttcacttatcgctgcttgttacaacaaccatcagaacaagatcattctggagcaagagatgagctgtgttttactggccactttcattcaggatttggcacagactccaggtcaagcggaaaaccagccttaccaacccaaagggaaatgccttggttcccaagactatcttgagctggctaacagatttcctcagcaggcctgggaagaagctcgacagtttttcttgaaaaaagagaaaaaataaatgttttggttgattctgtatttgagtacccttgttaatattttaaattgtccaaacaaacattctaattgttccttaagaactcattttcccatgtttatactcttcccacactgtagatatggcatgtactttacactatttataatgactgtagatacttgaatgttctacttgctaattttgcaagttgagtttatttcatttatgcagagtatcttggagtttggtaatttccatcttatgataatatatactttgcatttgtgatatgggtgaaaggagacataaaattagcaagtctgttttgttcttgtaataaaagtaacttattctgttttcattgttgacttttcatgttaaggaaatacgaatctgaaagaaaaatgttaactccagctcttgaagtatcttaaataaagacttaattaaagtttaaaaaaaaaaaaaaaaaaaaaaaaaaaaa

SEQ ID NO: 90 - DNA, 3641 bp, Homo sapiens
ctgcactgaagagggagagcgagagagagactggagacgcacagatccccccaaggtctcccaagcctaccgtcccacagattattgtacagagccccaaaaatcgaaacagaggaaacgaacagcagttgaacatggacgaaggaattcctcatttgcaagagagacagttactggaacatagagattttataggactggactattcctctttgtatatgtgtaaacccaaaaggagcatgaaacgagacgacaccaaggatacctacaaattaccgcacagattaatagaaaagaaaagaagagaccgaattaatgaatgcattgctcagctgaaagatttactgcctgaacatctgaaattgacaactctgggacatctggagaaagctgtagtcttggaattaactttgaaacacttaaaagctttaaccgccttaaccgagcaacagcatcagaagataattgctttacagaatggggagcgatctctgaaatcgcccattcagtccgacttggatgcgttccactcgggatttcaaacatgcgccaaagaagtcttgcaatacctctcccggtttgagagctggacacccagggagccgcggtgtgtccagctgatcaaccacttgcacgccgtggccacccagttcttgcccaccccgcagctgttgactcaacaggtccctctgagcaaaggcaccggcgctccctcggccgccgggtccgcggccgccccctgcctggagcgcgcggggcagaagctggagcccctcgcctactgcgtgcccgtcatccagcggactcagcccagcgccgagctcgccgccgagaacgacacggacaccgacagcggctacggcggcgaagccgaggcccggccggaccgcgagaaaggcaaaggcgcgggggcgagccgcgtcaccatcaagcaggagcctcccggggaggactcgccggcgcccaagaggatgaagctggattcccgcggcggcggcagcggcggcggcccggggggcggcgcggcggcggcggcagccgcgcttctggggcccgaccctgccgccgcggccgcgctgctgagacccgacgccgccctgctcagctcgctggtggcgttcggcggaggcggaggcgcgcccttcccgcagcccgcggccgccgcggcccccttctgcctgcccttctgcttcctctcgccttctgcagctgccgcctacgtgcagcccttcctggacaagagcggcctggagaagtatctgtacccggcggcggctgccgccccgttcccgctgctataccccggcatccccgccccggcggcagccgcggcagccgccgccgccgctgccgccgccgccgccgcgttcccctgcctgtcctcggtgttgtcgccccctcccgagaaggcgggcgccgccgccgcgaccctcctgccgcacgaggtggcgccccttggggcgccgcacccccagcacccgcacggccgcacccacctgcccttcgccgggccccgcgagccggggaacccggagagctctgctcaggaagatccctcgcagccaggaaaggaagctccctgaatccttgcgtcccgaaggacggaggttcaagcagagtgagaagttaaaatacccttaaggaggttcaagcagagtgagaagttaaaatacccttaaggtctttaagggaggaagtgtaatagatgcacgacaggcataaacaagaacaacaaaacaggtgttatgtgtacattcggagttcctgttttgctcatcccgcaccaccccaccctccacacactaacatccctttcttccccccaccagctgtaaaagatcctatgcgaaagacactggctcttttttttaatcccccaaataaattttgcccccttttaggccatgttccattatctcttaaaattggaacctaattcgagaggaagtaagaagggtctgttctgtggctgagctaggtgaaccccggggtaggggaaagatgttaacacctttgacgtctttggagttgacatggaacagcaggtagttgttatgtagagctagttctcaaagctgccctgcctgttttaggaggcgttccacaaacagattgaggctctttttagaattgaatttactcttcagtattttctaatgttcagctttctaaaaggcatatatttttcaaagaagtgaggatgcagtttctcacgttgcaacctattctgaagtggtttaaatggtatctcttagtaacttgcactcgttaaagaaacacggagctgggccatcgtcagaactaagtcagggaaggagatggatgagaaggccagaatcattcctagtacatttgctaacactttattgagaaattgaccatgaattaatggactcatcttaatttcttctaagtccatatatagatagatatctatctgtacagatttctatttatccatagataggtatctatacatacacatctcaagtgcatctattcccactctcattaatccatcatgttcctaaatttttgtaatcttactgtaaaaaaaagtgcactgaacttcaaaacaaaacaaaaaacaacaacaacaaaaaacaagtccaaactgatatatcctatattctgttaaaattcaaaagtgaacgaaagcatttaactggccagttttgattgcaaatgctgtaaagatatagaatgaagtcctgtgaggccttcctatctccaagtctatgtattttctggagaccaaaccagataccagataatcacaaagaaagcttttttaataaggcttaaaccaagaccttgtctagatatttttagtttgttgccaaggtagcactgtgagaaatctcacttggatgttatgtaaggggtgagacacaacagtctgactatgagtgaggaaaatatctgggtcttttcgtcagtttggtgcatttgctgctgctgttgctactgtttgcctcaaacgctgtgtttaaacaacgttaaactcttagcctacaaggtggctcttatgtacatagttgttaatacatccaattaatgatgtctgacatgctatttttgtagggagaaaatatgtgctaatgatattttgagttaaaatatcttttggggaggatttgctgaaaagttgcacttttgttacaatgcttatgcttggtacaagcttatgctgtcttaaattattttaaaaaaattaaatactgtctgtgagaaaccagctggtttagaaaagtttagtatgtgacgataaactagaaattacctttatattctagtattttcagcactccataaattctattacctaaatattgccacactattttgtgatttaaaaattcttactaaggaataaaaactttaatatacgatatgatattgtctaataattaaaaaagacataatggatgctcaattagttttaagatatctataactatagggatacaaatcactacagttctcagatttacaccttttttttgtcattggcttgatgtcacacatttccaatctcttgcaagcctccaggctctggctttgtctacctgctcgttcccaatgtatcttaatgaaaagtgcaaaagaaaaacctaccaattaaaaaaaaaaaaaaaaaa

SEQ ID NO: 91 - DNA, 2772 bp, Homo sapiens
ggtgtccggagccgcggacctagagatcccagaagccacagcgcagcggcccggcccgccactatttccaggctctgcggggcaggggccgccgcccaccgcccgccgccatggggctgaaggccgcccagaagacgctgttcccgctgcgctccatcgacgacgtggtgcgcctgtttgctgccgagctgggccgagaggagccggacctggtgctcctttccttggtgctgggcttcgtggagcattttctggctgtcaaccgcgtcatccctaccaacgttcccgagctcaccttccagcccagccccgcccccgacccgcctggcggcctcacctactttcccgtggccgacctgtctatcatcgccgccctctatgcccgcttcaccgcccagatccgaggcgccgtcgacctgtccctctatcctcgagaagggggtgtctccagccgtgagctggtgaagaaggtctccgatgtcatatggaacagcctcagccgctcctacttcaaggatcgggcccacatccagtccctcttcagcttcatcacaggcaccaaattggacagctccggtgtggcctttgctgtggttggggcctgccaggccctgggtctccgggatgtccacctcgccctgtctgaggatcatgcctgggtagtgtttgggcccaatggggagcagacagctgaggtcacctggcacggcaagggcaacgaggaccgcaggggccagacagtcaatgccggtgtggctgagcggagctggctgtacctgaaaggatcatacatgcgctgtgaccgcaagatggaggtggcgttcatggtgtgtgccatcaacccttccattgacctgcacaccgactcgctggagcttctgcagctgcagcagaagctgctctggctgctctatgacctgggacatctggaaaggtaccccatggccttagggaacctggcagatctagaggagctggagcccacccctggccggccagacccactcaccctctaccacaagggcattgcctcagccaagacctactatcgggatgaacacatctacccctacatgtacctggctggctaccactgtcgcaaccgcaatgtgcgggaagccctgcaggcctgggcggacacggccactgtcatccaggactacaactactgccgggaagacgaggagatctacaaggagttctttgaagtagccaatgatgtcatccccaacctgctgaaggaggcagccagcttgctggaggcgggcgaggagcggccgggggagcaaagccagggcacccagagccaaggttccgccctccaggaccctgagtgcttcgcccacctgctgcgattctacgacggcatctgcaaatgggaggagggcagtcccacgcctgtgctgcacgtgggctgggccacctttcttgtgcagtccctaggccgttttgagggacaggtgcggcagaaggtgcgcatagtgagccgagaggccgaggcggccgaggccgaggagccgtggggcgaggaagcccgggaaggccggcggcggggcccacggcgggagtccaagccagaggagcccccgccgcccaagaagccagcactggacaagggcctgggcaccggccagggtgcagtgtcaggacccccccggaagcctcctgggactgtcgctggcacagcccgaggccctgaaggtggcagcacggctcaggtgccagcacccgcagcatcaccaccgccggagggtccagtgctcactttccagagtgagaagatgaagggcatgaaggagctgctggtggccaccaagatcaactcgagcgccatcaagctgcaactcacggcacagtcgcaagtgcagatgaagaagcagaaagtgtccacccctagtgactacactctgtctttcctcaagcggcagcgcaaaggcctctgaactactggggacttcggaccgcttgtggggacccaggctccgccttagtcccccaactctgagcccatgttctgcccccagcccaaaggggacaggcctcacctctacccaaaccctaggttcccggtcccgagtacagtctgtatcaaacccacgattttctccagctcagaacccagggctctgccccagtcgttagaatataggtctcttctcccagaatcccagccggccaatggaaacctcacgctgggtcctaattaccagtctttaaaggcccagcccctagaaacccaagctcctcctcggaaccgctcacctagagccagaccaacgttactcagggctcctcccagcttgtaggagctgaggtttcacccttaacccaagggagcacaggtcccacctccagcccggggagcctaggaccactcagcccctaggagtatatttccgcacttcagaattccatatcttgcgaatccaagctccctgccccaaataacttcagtcctgcttccagaatttggaaatcctagtttcctctccttcgtatcccgagtctgggacacaaaactccgcccccagcctatgagcatcctgagccccgccctcttcctgacgaaactggccccggatcagagcaggacctcccttccgaccctctgggaacctcccagaggtccagcccatctcggagcatcccggaggaaatctgcagaggggttaggagtgggtgacaagagcctgatctcttcctgttttgtacatagatttatttttcagttccaagaaagatgaatacattttgttaaaaaaaaaaaaaaaa

SEQ ID NO: 92 - DNA, 5171 bp, Homo sapiens
gtgagccgcaggcttcgggaggcgagggggcggggggagcagcgccgaggccgccgcctccgcctccgccgcctaggactagggggtgggggacggacaagccccgatgccgggggagacggaagagccgagacccccggagcagcaggaccaggaagggggagaggcggccaaggcggctccggaggagccccaacaacggccccctgaggcggtcgcggcggcgcctgcagggaccactagcagccgcgtgctgaggggaggtcgggaccgaggccgggccgctgcggccgccgccgccgcagctgtgtcccgccggaggaaggccgagtatccccgccggcggaggagcagccccagcgccaggcctcccgacgtccccgggcagcagccccaggccgcgaagtccccgtctccagttcagggcaagaagagtccgcgactcctatgcatagaaaaagtaacaactgataaagatcccaaggaagaaaaagaggaagaagacgattctgccctccctcaggaagtttccattgctgcatctagacctagccggggctggcgtagtagtaggacatctgtttctcgccatcgtgatacagagaacacccgaagctctcggtccaagaccggttcattgcagctcatttgcaagtcagaaccaaatacagaccaacttgattatgatgttggagaagagcatcagtctccaggtggcattagtagtgaagaggaagaggaggaggaagaagagatgttaatcagtgaagaggagataccattcaaagatgatccaagagatgagacctacaaaccccacttagaaagggaaaccccaaagccacggagaaaatcagggaaggtaaaagaagagaaggagaagaaggaaattaaagtggaagtagaggtggaggtgaaagaagaggagaatgaaattagagaggatgaggaacctccaaggaagagaggaagaagacgaaaagatgacaaaagtccacgtttacccaaaaggagaaaaaagcctccaatccagtatgtccgttgtgagatggaaggatgtggaactgtccttgcccatcctcgctatttgcagcaccacattaaataccagcatttgctgaagaagaaatatgtatgtccccatccctcctgtggacgactcttcaggcttcagaagcaacttctgcgacatgccaaacatcatacagatcaaagggattatatctgtgaatattgtgctcgggccttcaagagttcccacaatctggcagtgcaccggatgattcacactggcgagaagccattacaatgtgagatctgtggatttacttgtcgacaaaaggcatctcttaattggcacatgaagaaacatgatgcagactccttctaccagttttcttgcaatatctgtggcaaaaaatttgagaagaaggacagcgtagtggcacacaaggcaaaaagccaccctgaggtgctgattgcagaagctctggctgccaatgcaggcgccctcatcaccagcacagatatcttgggcactaacccagagtccctgacgcagccttcagatggtcagggtcttcctcttcttcctgagcccttgggaaactcaacctctggagagtgcctactgttagaagctgaagggatgtcaaagtcatactgcagtgggacggaacgggtgagcctgatggctgatgggaagatctttgtgggaagcggcagcagtggaggcactgaagggctggttatgaactcagatatactcggtgctaccacagaggttctgattgaagattcagactctgccggaccttagtggacaggaagacttggggcatgggacagctcagactttgtatttaaaagttaaaaaggacaaaaaaaaaatctaaagcatttaaaatctagtgaaataactgaagggcctgctctttccattgtggatcacagcacacacatacatacaccctccacctccccatcccctgttctccctctgttgctccccttataaaattgatgttgtctttaccagaaaggtagacaaaaaagaagcagcagcagctcttaaagtgagggttattctcatactcggttccagccatcagcagacttcctgctcatcggcagatccccctttccaacctgtaactctgatgtgctctggatcagcttttaacttttaatcatatattactgtcttctaaatcccttctcctcctctactgctgccctatggttctggctcctaccccctgcggcacacttatcttcaaataccatagaattctaatctctggaggctggcagcttgacttggcactttagggccccttagcagggtgagctgttaaaacagcacacatctctcatcccctcttcctttattcccccctgggtttcagaaaggaaggatatatggggaccacctcccccttctttgatcccagcatctcagtccccctcccaaccctccatatggctctcaatggtgctcacttgcttggaagcaggctcccaatagggagggggctgccctctacagtctctttgactgtaagacagggctctgtatcagtgagacgatgagaaaagtcccaggctaatggcagaaatttgcactttgaacatgtgtgtttttgtgttgtggaacctgagattccttatttattaacaggaagtctgattttttttttttggagtctttgttgctatattttgtggggctgggagagagagattagattattttgacatgggatcccttccataacaggtactttgaaggcaagacatagggttgaagaagcacagccagcctctgaaatcatagctctccagtggcttttaaagaaagctggtcctcagcactaacaaaatcactacaatagcctagtgcttttttggaagcctttttagggaagaatgttaggttcatggtaactagtatgctctttgagatttttacagtgttgaaacttaagaattttgagagggtgaggagggttgttcagaatctaaattacagatagatgattgtttcttgtgaatttgtttcttttcctttttttttgtccctaccatttccttacatttcccttggggcccatctctggctccttgctttttgtttcttgctttgctttatcagttcattccagctccctgttagtgaaggacactgctgttagtgaaggaacaaagtctatgagtcctaaaattttaagtcaaagaaaactgctctgtttcccctttagtaacacttctgaagaggaaaaacttcaatagccaaagttaataatcctatataataattgctttggctttcacctaaaattctgggcatcacaatttccttgggatagaggttgtgttggggaatagattgcttattgctgttcactggagagaaaaggtagtgtttttgtacaaggtcataccgccagaagccccaaatcctattttggctcatcttcaggtaaagagtaattcctatcctgtgtgcctcagaagctagaatcgaaggcttaccctattcattgtttattgtcagaaatgcatgatggctcttggaaagaatgacgttttgctggaaaaaaaaaaaagaacagtttgtgtttcacaaacatggcttatcaattttttcaaagaattcttttttcccaaaaagaggagtaacaaaatgtcatttctgaaagaggcttactttataccaactagtgtcagcatttgggatgccagggaacagagagtgagacacctacaatcaccagtctcaaatgcgctattgtttcttttcagagtgttgcagatttgccatttctccataatatggggatagaaaatggaataaagatagaagggatgtagaatatgctttcctgccaacatggtttggagtcgactttggtatattgactagatttgaaaatacaagattgattagatgaatctacaaaaaagttgtcctcctctcaggtcccttttacactttttgactaactagcatctatattccacacttagcttttttgtcacacttatcctttgtctccgtaaatttcatttgcagtggttagtcatcagatattttagccacctacacaaaagcaaactgcatttttaaaaatctttctgagatgggagaaaatgtattctcctttcctataccgctctcccaacaaaaaaacaactagttagttctactaattagaaacttgctgtactttttcttttcttttaggggtcaaggaccctctttatagctaccatttgcctacaataaattattgcagcagtttgcaatactaaaatattttttatagactttatatttttccttttgataaagggatgctgcatagtagagttggtgtaattaaactatctcagccgtttccctgctttcccttctgctccatatgcctcattgtccttccagggagctcttttaatcttaaagttctacatttcatgctcttagtcaaattctgttacctttttaataactcttcccactgcatatttccatcttgaattggtggttctaaattctgaaactgtagttgagatacagctatttaatatttctgggagatgtgcatccctcttctttgtggttgcccaaggttgttttgcgtaactgagactccttgatatgcttcagagaatttaggcaaacactggccatggccgtgggagtactgggagtaaaataaaaatatcgaggtatagactagcatccacatagagcacttgaacctcctttgtacctgtttggggaaaaagtataatgagtgtactaccaatctaactaagattattatagtctggttgtttgaaataccatttttttctccttttgtgtttttcccactttccaatgtactcaagaaaattgaacaaatgtaatggatcaatttaaaatattttatttcttaaaagccttttttgcctgttgtaatgtgcaggacccttctcctttcatgggagagacaggtagttacctgaatataggttgaaaaggttatgtaaaaagaaattataataaaagggatactttgcttttcaaatctttgttttctcttattctaggtaaggcatattaaaaataaatatgtaaagaaaaaaaataaaagttgtcttcatggaaaaaaaaaaaaaaaaaa

SEQ ID NO: 93 - DNA, 1687 bp, Homo sapiens
cgggcgcgcggggagggggcgcaggaagcgggcgaggccgggcgcgggaggcggcggcggccccgggagctgccgatcggcgccgggacaaaggcgcggccgcccggcgcccccagcagcccgagccggggcgcacagccggggcgcagcccgcgccccccgccgcgattgacatgatgtttccacaaagcaggcattcgggctcctcgcacctaccccagcaactcaaattcaccacctcggactcctgcgaccgcatcaaagacgaatttcagctactgcaagctcagtaccacagcctcaagctcgaatgtgacaagttggccagtgagaagtcagagatgcagcgtcactatgtgatgtactacgagatgtcctacggcttgaacatcgagatgcacaaacaggctgagatcgtcaaaaggctgaacgggatttgtgcccaggtcctgccctacctctcccaagagcaccagcagcaggtcttgggagccattgagagggccaagcaggtcaccgctcccgagctgaactctatcatccgacagcagctccaagcccaccagctgtcccagctgcaggccctggccctgcccttgaccccactacccgtggggctgcagccgccttcgctgccggcggtcagcgcaggcaccggcctcctctcgctgtccgcgctgggttcccaggcccacctctccaaggaagacaagaacgggcacgatggtgacacccaccaggaggatgatggcgagaagtcggattagcagggggccgggacagggaggttgggaggggggacagaggggagacagaggcacggagagaaaggaatgtttagcacaagacacagcggagctcgggattggctaaactcccatagtatttatggtggccgccggcgggggccccagcccagcttgcaggccacctctagctttcttcctaccccattcccggcttccctcctcctcccctgcagcctggttaggtggatacctgccctgacatgtgaggcaagctaaggcctggagggtcagatgggagaccaggtcccaagggagcaagacctcgcgaagcgcagcagccccggcccttcccccgttttgaacatgtgtaaccgacagtctgcctgggccacagccctctcaccctggtactgcatgcacgcaatgctagctgcccctttcccgtcctgggcaccccgagtctcccccgaccccgggtcccaggtatgctcccacctccacctgccccactcaccacctctgctagttccagacacctccacgcccacctggtcctctcccatcgcccacaaaagggggggcacgagggacgagcttagctgagctgggaggagcagggtgagggtgggcgacccaggattccccctccccttcccaaataaagatgagggtactaaagttgtcttggtttttattttattattatttttttctttttccagtatactagcttgtcttttaagaaaggggatattaaaaaaaaaaaaagacaaaagtgtttttaaaaaaaagcaacacccacacctgtgtctgtatatagtcagcttatctcgtgttcaatcgtctgatctctacagagagaagtggaaaatgctgtatcaagggtgggcttagctgtgcctttccaataaagatgtgagaagcttcaaaaaaaaaaa

SEQ ID NO: 94 - DNA, 3600 bp, Homo sapiens
cccagactccagccctggaccgcgcatcccgagcccagcgcccagacagagtgtccccacaccctcctctgagacgccatgttcaactcgatgaccccaccaccaatcagtagctatggcgagccctgctgtctccggcccctccccagtcagggggcccccagtgtggggacagaaggactgtctggcccgcccttctgccaccaagctaacctcatgtccggcccccacagttatgggccagccagagagaccaacagctgcaccgagggcccactcttttcttctccccggagtgcagtcaagttgaccaagaagcgggcactgtccatctcacctctgtcggatgccagcctggacctgcagacggttatccgcacctcacccagctccctcgtagctttcatcaactcgcgatgcacatctccaggaggctcctacggtcatctctccattggcaccatgagcccatctctgggattcccagcccagatgaatcaccaaaaagggccctcgccttcctttggggtccagccttgtggtccccatgactctgcccggggtgggatgatcccacatcctcagtcccggggacccttcccaacttgccagctgaagtctgagctggacatgctggttggcaagtgccgggaggaacccttggaaggtgatatgtccagccccaactccacaggcatacaggatcccctgttggggatgctggatgggcgggaggacctcgagagagaggagaagcgtgagcctgaatctgtgtatgaaactgactgccgttgggatggctgcagccaggaatttgactcccaagagcagctggtgcaccacatcaacagcgagcacatccacggggagcggaaggagttcgtgtgccactgggggggctgctccagggagctgaggcccttcaaagcccagtacatgctggtggttcacatgcgcagacacactggcgagaagccacacaagtgcacgtttgaagggtgccggaagtcatactcacgcctcgaaaacctgaagacgcacctgcggtcacacacgggtgagaagccatacatgtgtgagcacgagggctgcagtaaagccttcagcaatgccagtgaccgagccaagcaccagaatcggacccattccaatgagaagccgtatgtatgtaagctccctggctgcaccaaacgctatacagatcctagctcgctgcgaaaacatgtcaagacagtgcatggtcctgacgcccatgtgaccaaacggcaccgtggggatggccccctgcctcgggcaccatccatttctacagtggagcccaagagggagcgggaaggaggtcccatcagggaggaaagcagactgactgtgccagagggtgccatgaagccacagccaagccctggggcccagtcatcctgcagcagtgaccactccccggcagggagtgcagccaatacagacagtggtgtggaaatgactggcaatgcagggggcagcactgaagacctctccagcttggacgagggaccttgcattgctggcactggtctgtccactcttcgccgccttgagaacctcaggctggaccagctacatcaactccggccaatagggacccggggtctcaaactgcccagcttgtcccacaccggtaccactgtgtcccgccgcgtgggccccccagtctctcttgaacgccgcagcagcagctccagcagcatcagctctgcctatactgtcagccgccgctcctccctggcctctcctttcccccctggctccccaccagagaatggagcatcctccctgcctggccttatgcctgcccagcactacctgcttcgggcaagatatgcttcagccagagggggtggtacttcgcccactgcagcatccagcctggatcggataggtggtcttcccatgcctccttggagaagccgagccgagtatccaggatacaaccccaatgcaggggtcacccggagggccagtgacccagcccaggctgctgaccgtcctgctccagctagagtccagaggttcaagagcctgggctgtgtccataccccacccactgtggcagggggaggacagaactttgatccttacctcccaacctctgtctactcaccacagccccccagcatcactgagaatgctgccatggatgctagagggctacaggaagagccagaagttgggacctccatggtgggcagtggtctgaacccctatatggacttcccacctactgatactctgggatatgggggacctgaaggggcagcagctgagccttatggagcgaggggtccaggctctctgcctcttgggcctggtccacccaccaactatggccccaacccctgtccccagcaggcctcatatcctgaccccacccaagaaacatggggtgagttcccttcccactctgggctgtacccaggccccaaggctctaggtggaacctacagccagtgtcctcgacttgaacattatggacaagtgcaagtcaagccagaacaggggtgcccagtggggtctgactccacaggactggcaccctgcctcaatgcccaccccagtgaggggcccccacatccacagcctctcttttcccattacccccagccctctcctccccaatatctccagtcaggcccctatacccagccaccccctgattatcttccttcagaacccaggccttgcctggactttgattcccccacccattccacagggcagctcaaggctcagcttgtgtgtaattatgttcaatctcaacaggagctactgtgggagggtgggggcagggaagatgcccccgcccaggaaccttcctaccagagtcccaagtttctggggggttcccaggttagcccaagccgtgctaaagctccagtgaacacatatggacctggctttggacccaacttgcccaatcacaagtcaggttcctatcccaccccttcaccatgccatgaaaattttgtagtgggggcaaatagggcttcacatagggcagcagcaccacctcgacttctgcccccattgcccacttgctatgggcctctcaaagtgggaggcacaaaccccagctgtggtcatcctgaggtgggcaggctaggagggggtcctgccttgtaccctcctcccgaaggacaggtatgtaaccccctggactctcttgatcttgacaacactcagctggactttgtggctattctggatgagccccaggggctgagtcctcctccttcccatgatcagcggggcagctctggacataccccacctccctctgggccccccaacatggctgtgggcaacatgagtgtcttactgagatccctacctggggaaacagaattcctcaactctagtgcctaaagagtagggaatctcatccatcacagatcgcatttcctaaggggtttctatccttccagaaaaattgggggagctgcagtcccctgcacaagatgccccagggatgggaggtatgggctgggggctatgtatagtctgtatacgttttgaggagaaatttgataatgacactgtttcctgataataaaggaactgcatcag

SEQ ID NO: 95 - DNA, 1586 bp, Homo sapiens
gcacagccacagcctcctcgcctccccaaactcccagccaaggcgcgcggtggcgtcctcgcgccctcgctcgcgtccccgcccgccgcctgcgcaagccaggcatgaatgctgagacttgcgtctcttactgcgagtcgccggccgctgccatggacgcctactacagcccggtgtcgcagagtcgggagggctcgtcgccttttagggcatttcccggaggcgacaagttcggcacaactttcctgtcggccgccgccaaagcacagggattcggggacgccaagagccgggcccgttacggcgctgggcagcaggacctggcgacacccctggagagtggagctggggcgcggggctcctttaacaagttccagccccagccgtcgaccccgcagccccagccgccgccgcagccgcagccgcagcagcagcagccgcagccccagccgcccgcgcaaccgcatctttacttgcagcgaggcgcctgcaagacgcccccggacggcagcctcaaactccaggaaggcagcagcggccacagcgcggccttgcaggttccctgctacgctaaagagagctccctgggtgagccagagttaccccctgactctgacactgtggggatggacagcagctacctgagtgtcaaggaggctggggtgaaggggccccaggaccgggccagctcagacctccccagcccattggagaaggccgactcagagagcaacaagggcaagaagcggcggaaccggaccaccttcaccagctaccagctggaggagctggagaaggtcttccagaagacccactacccagacgtgtatgcgcgggaacagctggccatgaggacagacctcactgaggcccgcgtgcaggtctggttccagaaccgaagggccaagtggaggaagcgggagcgttttgggcagatgcagcaggttcgaacccacttctccactgcatatgagctgcccctcctcacccgagctgagaactacgcccagattcagaacccgtcctggctcggcaacaacggggctgcctcaccagtgccagcctgcgtggtcccctgcgacccggtgcctgcctgcatgtcccctcatgcccacccccctggctctggggccagcagcgtcaccgacttcctgagtgtgtctggggctggcagtcacgtgggccagacgcacatgggcagcctgtttggagcagccagcctcagcccaggcctcaatggctacgagctcaacggcgagccggaccgcaagacctcgagcatcgcggccctccgcatgaaggccaaggagcacagtgcggccatttcctgggccacatgacagggcacccctgccccgtccccacctcgggacaccatgggccacgcccatgttttccaggcccccagcctcccactcgactttcctcttaggaacctggcctgggccaggggcctgaccctcagcactttcagccgccccaagtctgaggccccgtggactgctgggagggagggggcagcaggcccctggcccctccctggcactgaggccctgacccctgctcccggccacaggcagtggaga

SEQ ID NO: 96 - DNA, 1378 bp, Homo sapiens
cattgctgctgctacctgctttccagagcctttcatcaatgaaggaaagcggctgggctatatccatcgaaattttgctggaaacagattttctgatcacgtagcccttttatcagtattccaagcctgggatgatgctagaatgggtggagaagaagcagagatacgtttttgtgagcacaaaagacttaatatggctacactaagaatgacctgggaagccaaagttcagctcaaagagattttgattaattctgggtttccagaagattgtttgttgacacaagtgtttactaacactggaccagataataatttggatgttgttatctccctcctggcctttgtagccaagacatgaagtacccatctcccttctttgtatttggtgaaaagattcgaactcgagccatctctgctaaaggcatgactttagtcacccccctgcagttgcttctctttgcctccaagaaagtccaatctgatgggcagattgtgcttgtagatgactggattaaactgcaaatatctcatgaagctgctgcctgtatcactggtctccgggcagccatggaggctttggttgttgaagtaaccaaacaacctgctatcatcagccagttggaccccgtaaatgaacgtatgctgaacatgatccgtcagatctctagaccctcagctgctggtatcaaccttatgattggcagtacacggtatggagatggtccacgtcctcccaagatggcccgatacgacaatggaagcggatatagaaggggaggttctagttacagtggtggaggctatggcggtggctatagcagtggaggctatggtagcggaggctatggtggcagcgccaactcctttcgggcaggatatggtgcaggtgttggtggaggctatagaggagtttcccgaggtggctttagaggcaactctggaggagactacagagggcctagtggaggctacagaggatctgggggattccagcgaggaggtggtaggggggcctatggaactggctactttggacagggaagaggaggtggcggctattaaaacttggttatgtcagttcctgtgtgtagacagtaaggaaaaaaaggcatgctatgtgttacgtgttttttccagtatgtttatttgccaccaaaaagtaaatgcattttcacccattctgtggttcattgtagtttaaggaaaccaagcatatagatgcattagtgattttgtttatattatgtaaaatataacgatctcttaaaaataccacagtttgtattttttctttaaggagtaaagatttgcctttaaataacttggtattttcctggctttcgtttaatacaatagaaaataaagtattacaccg

SEQ ID NO: 97 - DNA, 3138 bp, Homo sapiens
ctcttgacagcggcggcggcgcagccggttccgggttcggcgcggggcggggatgtgaatcccgatggagcggcccgaggaaggcaagcagtcgccgccgccgcagccctggggacggctcctgcgtctgggcgcggaggagggcgagccgcacgtcctcctgaggaagcgggagtggaccatcgggcggagacgaggttgcgacctttccttccccagcaataaactggtctctggagatcactgtagaattgtagtggatgaaaaatcaggtcaggtgacactggaagataccagcaccagtggaacagtgattaacaagctgaaggttgttaagaagcagacatgccctttacagactggggatgtcatctacttggtgtacaggaagaatgaaccggaacacaacgtggcatacctctatgaatctttaagtgaaaagcaaggcatgacacaagaatcctttgagatggtgccttgctgtgttgcccaggctggtctaaaactcctgggatcaagtgatcctcccaccttggcctcccaaagtattgtgattacagggtctgggggtggtggcatctcccctaaaggaagtggtccctctgtggcaagtgatgaagtctccagctttgcctcagctctcccagacagaaagactgcgtccttttcgtcgttggaaccccaggatcaggaggatttggagcccgagaagaagaaaatgagaggagatggggaccttgacctgaacgggcagttgttggtcgcacaaccgcgtagaaatgcccaaaccgtccacgaggacgtcagagcagcggctgggaagccagacaagatggaggagacgctgacatgcatcatctgccaggacctgctgcacgactgcgtgagtttgcagccctgcatgcacacgttctgcgcggcttgctactcgggctggatggagcgctcgtccctgtgtcctacctgccgctgtcccgtggagcggatctgtaaaaaccacatcctcaacaacctcgtggaagcatacctcatccagcatccagacaagagtcgcagtgaagaagatgtgcaaagtatggatgccaggaataaaatcactcaagacatgctgcagcccaaagtcaggcggtctttttctgatgaagaagggagttcagaggacctgctggagctgtcagacgttgacagtgagtcctcagacattagccagccatacgtcgtgtgccggcagtgtcctgagtacagaaggcaggcggcgcagcctccccactgcccagcacccgagggcgagccaggagccccacaggccctgggggatgcaccccccacgtccgtcagcctgacgacagcagtccaggattacgtgtgccctctgcaaggaagccacgccctgtgcacctgctgcttccagcccatgcccgaccggagagcggagcgcgagcaggacccgcgtgtcgcccctcagcagtgtgcggtctgcctgcagcctttctgccacctgtactggggctgcacccggaccggctgctacggctgcctggccccgttttgtgagctcaacctgggtgacaagtgtctggacggcgtgctgaacaacaacagctacgagtcagacatcctgaagaattacctggcaaccagaggtttgacatggaaaaacatgttgaccgagagcctcgtggctctccagcggggagtgtttctgctgtctgattacagagtcacgggagacaccgttctgtgttactgctgtggcctgcgcagcttccgtgagctgacctatcagtatcagcagaacattcctgcttccgagttgccagtggccgtaacatcccgtcctgactgctactggggccgtaactgccgcactcaggtgaaagctcaccacgccatgaaattcaatcatatctgtgaacagacaaggttcaaaaactaagcatccagaggccctgagcagctttcagcactggaggtgaagagagcgtgtttttaaaatacagaggcaagcacgtcaaggtgttttcacagccccctgagggaagggacgcagggtctccgacaggtgctctggggtgactcttctgtggagctttaccctctgagtgagaccctccccagagccccgggggccgcagcccgccctcctggtgagcgctgggcagggctcgtggtggcatcagcagcagagacgaagcctttctgtaacatgcggccgtcctgccgagaggggcagttttgctcttttgtacattttccgaaactacagttaaagcggaagtctgttttcaggaaaagtttcaagggagaagggcaagtttatcaaaaacattgtttcaggagaagggagcataagtttacagcctacaggacgtacacaatatcctgctgctgggaaaaccacagcattttatctattttttattttaataggtttggtgcttatcttctaataagatttaaatgtcacaaactgtagcacaaataatataatttataatttacaaattgactaaaattgggtatagtatggtatttgaaagaataagcatatgcttctgtttattaaaaaaagaaaccttccaatgtccaaaactgctaaccctcgacgtggccgccaagttagtcgctccttgctaaccggtgagtgaccgcggccccgagcctggggctggacgcaggtcccaggacatgctgctcccttgtgtgagtgaccgcggccccgagcctggggctggacgcaggtcccaggacgtgctgctcccttgtgtgagtgaccacggccccaagcccagggctggaggcaggtcccaggacgcgccgctccctcatgctgcccgggcccttcctccaagaccctacagagcctgaggggcaccttggcttccgcctgtgctagctttgccatgtcatctggaataatacttgaaattttgattcttggaaaaaaaagtttcttatcttttgttgaaatcacctgttatccttgtttgtaaactgataacttttttgcttcttctcaggaatacagttttcaactgttgtcttgctcttgatagaaactgagaagcagcaatctgtatttgtggaggaaagtcctctcttttgcatattctaataaatgagccgcgtttgctcctc

SEQ ID NO: 98 - DNA, 1640 bp, Homo sapiens
ggatccacgagctggagtgattagagccctggaagggaattgttactcccgtggagaagtccccttttcctggcagtcgtctgcactgtacacgctggatgcctctctccatccaccccactcactcgctcctctctcacctcctctctccctctcctgcattgatttttttttttcctttttagttgactgaaacaaaacaaaacaaaagggccactggatgtctgccttcttggggggtgagccagacagactgacaaacaaacagccccaactgtgttcgggggagggtttcgcctcccgttttgcccggcagcagcagcatggacgtgttggctagttatagtatattccaggagctacaacttgtccacgacaccggctacttctcagctttaccatccctggaggagacctggcagcagacatgccttgaattggaacgctacctacagacggagccccggaggatctcagagacctttggtgaggacttggactgtttcctccacgcttcccctcccccgtgcattgaggaaagcttccgtcgcttagaccccctgctgctccccgtggaagcggccatctgtgagaagagctcggcagtggacatcttgctctctcgggacaagttgctatctgagacctgcctcagcctccagccggccagctcttctctagacagctacacagccgtcaaccaggcccagctcaacgcagtgacctcattaacgcccccatcgtcccctgagctcagccgccatctggtcaaaacctcacaaactctctctgccgtggatggcacggtgacgttgaaactggtggccaagaaggctgctctcagctccgtaaaggtgggaggggtcgcaacagctgcagcagccgtgacggctgcgggggccgttaagagtggacagagcgacagtgaccaaggagggctaggggctgaagcatgtcccgaaaacaagaagagggttcaccgctgtcagtttaacgggtgccggaaagtttatacaaaaagctcccacttaaaggcccaccagaggactcacacaggtgagaagccttataagtgctcatgggagggatgtgagtggcgttttgcacgaagcgatgagctcacgaggcactacaggaaacacacaggtgcaaagcccttcaaatgcaaccactgcgacaggtgtttttccaggtctgaccatcttgccctccacatgaagagacatatctaaaaaaccgaaaggccagagttgccatggcatcggctagtgtctaaaggaaacgccatgaggcagggggctggacttcaggcggggacccattgcctcgcagaagaaagctctcacttataaacctctgtacacacacacacacacacacacatatacacacactcacagacccacacacatacacactgtcatgcactcaactatatttaaaatatatacgtctattctttatgccttgccctagccagatggaagaagatgaagaaggaaaccaggtgaactcagcaaggcagactggctgcttacttcagcactattggaattatttcccgctgttgccaatggaaatcaaagaaaatggatgtgacgtctgtgcaggtggacggcagtccgaggggcttatttcac

SEQ ID NO: 99 - DNA, 2906 bp, Homo sapiens
acctcgcgcccccgccctgcgggtgtctcgcgcgcgttccgtgcgtgtgagtgtgtgggtctgtctcgctccagaagtgcgtgcccgcgcgctgcgccttgcgctttttcccctccctcgccccttcctggtcctcccaccctcctcggctccctcctttcccagcaaacgccgcccctcccgcgccctggctcaggctctggcgccgccgcagccgtcgccgcccgaaagttcaggagccctggaaaggagaaggaataagacggcaggaggaagagagagagagggtagaatggaagaatctcacttcaattctaacccgtacttctggccttctatccccacagtctcaggtcagatcgagaacacaatgttcatcaacaagatgaaggatcagctgttgccagagaagggctgtggtctggccccacctcactaccccaccttgctgacagtgcctgcctcagtgtccctgccctcaggcatcagtatggacacagagtccaagtcagaccagctgaccccacacagccaagcgtccgttacccagaatatcacggtggtccctgtgccgtctacaggactgatgactgctggagtctcctgttctcagaggtggagaagagaagggagtcaatcaaggggtccgggtttggtaatcacgtccccctcaggctctcttgtgaccacagcatcatcagctcagaccttccccatttcggctcccatgattgtctcagctcttccccctggctcacaagccctgcaggttgtccctgacctctccaagaaggtagcatcgaccctaaccgaggaaggaggcggaggtggtggtggaggtggcagtgtggctcctaagccaccccggggccggaagaagaagcggatgctggaatcagggctgcccgagatgaatgacccttatgtcctctcccctgaggatgatgatgaccatcagaaagacggcaagacctacaggtgccggatgtgctcactgacattctactccaagtcggagatgcagatccactccaagtcacacaccgagaccaagccccacaagtgcccacattgctccaagaccttcgccaacagctcctacctggcccagcacatccgtatacactcaggggctaagccctacagttgtaacttctgtgagaaatccttccgccagctctcccaccttcagcagcacacccgaatccacactggtgatagaccatacaaatgtgcacacccaggctgtgagaaagccttcacacaactctccaatctgcagtcccacagacggcaacacaacaaagataaacccttcaagtgccacaactgtcatcgggcgtacacggatgcagcctcactagaggtgcacctgtctacgcacacagtgaagcatgccaaggtgtacacctgcactatctgcagtcgggcatacacatcagaaacataccttatgaaacatatgcgcaaacacaacccgcctgatcttcagcaacaggtgcaggcagcagcagcagcggcagcagtggcccaggcccaggctcaagctcaagcccaggctcaggctcaggctcaagcccaggcccaggcccaggcctcccaggcatcacagcagcagcagcagcagcagcagcagcagcagcagcagcagcaacagccaccaccacacttccagtctcctggggcagccccccagggtgggggtggtggggacagcaatcccaaccctccaccccagtgttcctttgacctgaccccgtataagacggcggagcatcataaggacatctgcctcactgtcaccaccagcaccatccaggtggagcacctggccagctcttagagatccgtgctgccacccactgggaagaggaagaagtagtcctggtgtcttctttctccaactcttggtgggaaaagtccttttcttccttgacaggccttggctccatctccttgggcctctgtcacggctttccttcacaggataccatcctttttctgaactcttcttcaaaaggaacatcagccctcctgattgcaaaggaatactgagctgatggtgtcatccagcagcctcccctcccaagcaaagcttctaaaactgggggtcggtgctcaagggaaggatttgctatgacctcatagaaccttgtccagtgtggccacttaccctatccttaccctccttatcctcaaagtttgggctgatgtaagactagaggctggccctcccagataacagagaaaagggagccccaaatgcaaccagcctcttgttctattcttgcctgcaaaagaacagaggtttctcaaatgcctcagtccctgagagccatttcttcccctacatcgtctcactttgcttcctattgactgctggtagaaggagatttggggtaggggctagacctccttttatttgaagggggcaagggctgagatgtggtccccaaggggccagaaattcccaagttggtcacaggtggcttagaagtgtgtgttatggttttacggatttccttgaagcctctctccttctctgcctacaaagaccctatactctcagtctccccaacccacccccaaggagctgtgggaggctttgtgttatctgtgaaactccaaaacaggggtgttgcggagaagggagagttcaaggcaaacgcaaggactggacttagctccctaggtgccacagtcagatgccggacacggatttatatataaatatatatatataaatatattatactcactcatcacggccatctttgttgtaaccatttctgtgtttataaatgcattatctctgagaattttcatatttgatgttttgtttatttttgtcctttttttccctctctccacccctgtcctctagccacagcatttttctttttgtc

SEQ ID NO: 100 - DNA, 3338 bp, Homo sapiens
gacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaaaaaaaaaaaaaaaaa

SEQ ID NO: 101 - DNA, 1980 bp, Homo sapiens
gagaggcggaagtaaaagcgaactacagtggatgggtgcagttcaggagatagctgttctccagcctttctggagggatgcagagcctttccttaggacaaacaagcatttcaaaaggtttgaactacctaaccattatggcaccaggaaacctctggcatatgagaaataactttctctttggttcaagatgttggatgactcgattttcagcagaaaacatcttcaaatcagtttcatttaggctttttggtgtgaagtgtcataatacagacagtgagcctttgaaaaatgaggacctactgaaaaacttacttactatgggagtagatattgacatggcaaggaaacgacagcctggagtttttcataggatgattaccaatgagcaggacctgaagatgttccttctttccaaaggagctagcaaagaagtgatcgctagcatcatatcaagatatccacgagcaataacacgtactcccgagaatctttcaaaacggtgggatctgtggagaaagattgtgacatcagaccttgaaattgtaaatattttggaacgttctcctgaatccttttttcggtccaataacaacctaaacttagagaataatataaagttcctctactcagttggattgacccgtaaatgcctttgtcgattgttgaccaatgcccctcgtaccttctccaatagtcttgatctgaataaacagatggttgaatttttgcaggcagccggtttgtcattgggtcacaatgatcccgcagattttgtcagaaagataatttttaaaaacccttttatcttaattcagagcaccaagcgggtgaaagctaacattgaattcttacggtcaactttcaatttgaacagtgaggaactgctggttctgatatgtggtccaggagctgaaatcctagacctttccaatgactatgccagaagaagctacgcaaacatcaaagagaagctgttttctcttggatgtactgaagaagaggtacagaagtttgtcttaagctatccagatgtgatcttcttggcagagaaaaagtttaatgataaaatagactgcctcatggaagaaaacattagcatttcacaaataatcgaaaatcctcgggttctggattcaagcataagtactttaaaaagtcgaatcaaagaattggtaaatgctggctgtaacttgagtactttaaacatcactcttctatcttggagtaaaaaaagatatgaagctaaattgaaaaagttaagcagatttgcctaaggatgccaatgtttttaattctcaggaactgtgaatatgttatgccacattccaaaagaattttgcagaagtgattaatttaaaatgttattaagggccttgagatggggagattatcctgaattactttggagggcctaatataattgtagggatctttaaaagttgaagagggagccaaaagaggaggtcagagtgatcctgtataagaaagacaactacttttgaaggaagttaaaaggtggctttgaaggaggaagacaccttgagccaaggaatgaagggggcctgtaaaaaaagattgaaaaggcaaggaaacagattctccattagagtccccagaaaggaatgcagccttgctggcacctttttagcccagtgagatcaatgtccaacttctgacccacagaaccataagatggtaaatttgtgttgttttaaaccactgagtcagtgataatttgctgaagaagcaaatagattaacatagcaactaagtagatgattagttgcattataggtgaagtccaaaatacagtcctttagtaaagtctctatttcttccttttataagagtaattatatttaaaaaatctacctacccttctggaattgctaatgttagtgtttataaagtcttaaatgtgtgcataaaaaactaaatgtgaatataaaatctaaataaaggaatatgtttgcctaa

SEQ ID NO: 102 - DNA, 1203 bp, Homo sapiens
gatctggggtgctgccaggaaaaagcaaattctggaagttaatggttttgagtgatttttaaatccttgctggcggagaggcccgcctctccccggtatcagcgcttcctcattctttgaatccgcggctccgcggtcttcggcgtcagaccagccggaggaagcctgtttgcaatttaagcgggctgtgaacgcccagggccggcgggggcggggccgaggcgggccattttgaataaagaggcgtgccttccaggcaggctctataagtgaccgccgcggcgagcgtgcgcgcgttgcaggtcactgtagcggacttcttttggttttctttctctttggggcacctctggactcactccccagcatgaaggcgctgagcccggtgcgcggctgctacgaggcggtgtgctgcctgtcggaacgcagtctggccatcgcccggggccgagggaagggcccggcagctgaggagccgctgagcttgctggacgacatgaaccactgctactcccgcctgcgggaactggtacccggagtcccgagaggcactcagcttagccaggtggaaatcctacagcgcgtcatcgactacattctcgacctgcaggtagtcctggccgagccagcccctggaccccctgatggcccccaccttcccatccagacagccgagctcgctccggaacttgtcatctccaacgacaaaaggagcttttgccactgactcggccgtgtcctgacacctccagaacgcaggtgctggcgcccgttctgcctgggaccccgggaacctctcctgccggaagccggacggcagggatgggccccaacttcgccctgcccacttgacttcaccaaatcccttcctggagactaaacctggtgctcaggagcgaaggactgtgaacttgtagcctgaagagccagagctagctctggccaccagctgggcgacgtcaccctgctcccaccccacccccaagttctaaggtcttttcagagcgtggaggtgtggaaggagtggctgctctccaaactatgccaaggcggcggcagagctggtcttctggtctccttggagaaaggttctgttgccctgatttatgaactctataatagagtatataggttttgtaccttttttacaggaaggtgactttctgtaacaatgcgatgtatattaaactttttataaaagtt

SEQ ID NO: 103 - DNA, 4746 bp, Homo sapiens
gttctccgctgaggaggagcggggcagaggagggaggcagcgggtgagagttcagagttcagcagcagcagcccgagcccatgattcccatatgccctgtagtttctttcacctatgtgcccagccggctgggggaagatgccaaaatggcgaccggcaactactttggattcacccacagcggggcggcggcggcggcggctgcggcccaatatagccagcagccagcttcgggtgtagcctattctcatccaactacagttgctagctacactgtccatcaggctccagtagctgctcacacagttactgctgcctatgcaccagcagccgccacagttgcagttgccaggcctgctccagtagctgttgcagctgctgcaacagctgctgcttatggaggctaccccactgcacacacagcaactgactatggttatactcagaggcaacaagaagcaccaccaccaccacccccagctactacacaaaactaccaggattcatactcatatgtaaggtccacagctcctgctgtagcttatgatagtaagcaatactaccaacaaccaacagcaactgctgctgctgtagctgccgctgcccaacctcagccttctgttgctgaaacttactatcagacagcccccaaagcaggttacagccaaggtgcaactcagtatactcaagcccagcaaactcgacaagtgacagccataaaaccagccacaccaagtccagctaccactactttctccatctatcctgtatcctccaccgtacagccagtagcagctgcggctactgtggtgccatcctatactcagagtgctacttacagtaccacagcagttacatattctggtacgtcttattcaggttatgaagcagcagtgtattcagctgcatcttcctactatcaacagcagcagcagcaacagaagcaggcagcagcagcagctgctgctgctgctgcaacagctgcctggacagggaccacctttactaaaaaagcaccattccaaaataaacaactgaaaccaaaacagcctcccaaaccaccacagattcactattgtgatgtttgtaagatcagctgtgctggaccacagacttataaagaacatttagaaggacagaaacataaaaaaaaagaagctgcattgaaagcctcacaaaataccagcagcagcaacagttctactcgtgggactcaaaatcagctacgttgtgagctctgcgatgtgtcttgtacaggagcagatgcgtatgctgcccacattcgtggtgctaagcatcagaaagtggttaagttacacacaaaacttggtaaacccattccatcaacagaaccaaatgttgttagccaagctacttcttcaacagctgtatctgcttcaaagccgactgcctctccttcaagcattgcagcaaacaattgtactgtgaatacgtcatcagttgcaacgtcttcaatgaagggtcttacgactacaggaaactcgtctcttaatagcacatctaacactaaagtatcagcagtgcctacaaatatggctgccaagaaaacatctacccccaaaataaattttgttggtggtaataagctgcagtcaacaggaaataaagcagaagacataaaaggaaccgaatgtgttaaaagtactcctgtcacttctgctgtgcagattcctgaagtaaagcaagacacagtgtcagaaccagtcacacctgcatctcttgctgctttacagagtgatgtgcagccagtgggccatgattatgtggaagaggtacgaaatgatgaaggaaaagtaattcggttccattgtaaattatgcgagtgcagctttaatgatcccaatgctaaggagatgcacttaaaagggcgaagacacagacttcaatataaaaaaaaagtaaatccagatttgcaagtagaagtaaagcctagtattcgagcaagaaagattcaagaagagaaaatgaggaagcaaatgcagaaggaggagtactggcgaagacgagaagaagaggagcgttggagaatggaaatgagacgttatgaagaggacatgtactggaggagaatggaggaagaacaacatcattgggatgatcgccgccgaatgccagatggaggttatcctcatggtcctccaggcccattaggccttctgggagtccgaccaggcatgcctcctcagcctcaggggcctgcacccttacgtcgtcctgactcatctgatgaccgttatgtaatgacaaaacatgccaccatttatccaactgaagaggagttacaggcagttcagaaaattgtttctattactgaacgtgctttaaaactcgtttcagacagtttgtctgaacatgagaagaacaagaacaaagagggagatgataagaaagagggaggtaaagacagagctttgaaaggagttttgcgagtgggagtattggcaaaaggattacttctccgaggagatagaaatgtcaaccttgttttgctgtgctcagagaaaccttcaaagacattattaagccgtattgcagaaaacctacccaaacagcttgctgttataagccctgagaagtatgacataaaatgtgctgtatctgaagcggcaataattttgaattcatgtgtggaacccaaaatgcaagtcactatcacactgacatctccaattattcgagaagagaacatgagggaaggagatgtaacctcgggtatggtgaaagacccaccggacgtcttggacaggcaaaaatgccttgacgctctggctgctctacgccacgctaagtggttccaggctagagctaatggtctgcagtcctgtgtgattatcatacgcattcttcgagacctctgtcagcgagttccaacttggtctgattttccaagctgggctatggagttactagtagagaaagcaatcagcagtgcttctagccctcagagccctggggatgcactgagaagagtttttgaatgcatttcttcagggattattcttaaaggtagtcctggacttctggatccttgtgaaaaggatccctttgataccttggcaacaatgactgaccagcagcgtgaagacatcacatccagtgcacagtttgcattgagactccttgcattccgccagatacacaaagttctaggcatggatccattaccgcaaatgagccaacgttttaacatccacaacaacaggaaacgaagaagagatagtgatggagttgatggatttgaagctgaggggaaaaaagacaaaaaagattatgataacttttaaaaagtgtctgtaaatcttcagtgttaaaaaaacagatgcccatttgttggctgtttttcattcataataatgtctacattgaaaaatttatcaagaatttaaaggatttcatggaagaaccaagtttttctatgatattaaaaaatgtacagtgttaggtattatttgaatggaaagacacccaaaaaaaaaaatgtgctccgactagggggaaaacagtagttccgattttttcccattatttttattttattttctggttgccctagcttccccccctatttttgtgtcttttattaactagtgcattgtcttattaaatcttcactgtatttaatgcaggatgtgtgcttcagttgctctgtgtattttgatattttaatttagaggttttgtttgctttttgacactagttgtaagttactttgttatagatggtatcctttaccccttcttaatattttacagcagtacgtttttttgtaacgtgagactgcagagtttgtttttctatatgtgaaggattacaacacaaaaagttatcctgccattcgagtgctcagaactgaatgtttctgcagatcttgtggcatttgtctctagtgtgatatataaaggtgtaattaagacagagttctgttaatctaatcaagtttgctgttagttgtgcattagcagtataaaagctaatatatactatatggtcttgcaacagttttaaagcctctgcataattgataataaaaatgcatgacattcttgtttttaatagacttttaaaatcataattttaggtttaacacgtagatctttgtacagttgactttttgacatagcaaggccaaaaataactttctgaatatttttttcttgtgtataagtggaaagggcatttttcacatataagtgggctaaccaatattttcaaaagaacttcatcattgtacaactaacaacagtaactagcccttaattatggtgacagttccttattggtgtgtgtgagattactctagcaactattacagtataacacagatgatcttctccacacaccccatcacccagataatttacagttctgttaacagtgaggttgataaagtattactgataaaaaattatctaaggaaaaaaacagaaaattatttggtgtggccatcttacctgcttatgtctcctacacaaagctaaatattctagcagtgatgtaatgaaaaattacatcttactgttgatatatgtatgctctggtacacagatgtcattttgttgtcacagcactacagtgaaatacacaaaaaatgaaattcatataatgacttaaatgtattatatgttagaattgacaacataaactacttttgctttgaaatgatgtatgcttcagtaaaatcatattcaaatttaaaaaaaaaaaaaaaaaaaaaaaaaaaaaaaa

SEQ ID NO: 104 - DNA, 4284 bp, Homo sapiens
agcagagctgcggccgggggaacccagtttccgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaaggtgaagtcggcgcggggaggggtagggccaacggcctggacgccccaagggcgggcgcagatcgcggagccatggattgcactttcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataatctggaaaaaa

SEQ ID NO: 105 - DNA, 3407 bp, Homo sapiens
gaagaactggaactggtctggccatagggggaagcggcgcgttaaatccgccccctctccataccctactttcaaggggtgggcggtagaggggaggttggggttggagcagcggcgagggggcccctcccttgcacctccccccaccggacttggtcgcgcccgaagtgtaacactttctctttgtcggaggagctcctctgtttcctgtgcagtagctcccgttgcggcggcacccgtggcagccctggcggacgcaggagcgatggcagcgaccgatatagctcgccaggtgggtgaaggttgccgaactgtccccctggctggacatgtggggtttgacagcttgcctgaccagctggtgaataagtccgtcagccagggcttctgcttcaacatcctgtgcgtgggagagacaggtttgggcaagtccaccctcatggacaccctgttcaacaccaaattcgaaggggagccagccacccacacacagccgggtgtccagctccagtctaatacctatgacctccaagagagcaacgtgaggctaaagctcacgatcgttagcacagttggctttggggaccagatcaacaaagaggacagctacaagcctatcgtggaattcatcgatgcacaattcgaggcctacctgcaggaagagctaaagatccgaagagtgctacacacctaccatgactcccgaatccatgtctgcttgtatttcattgcccccacgggtcattccctgaagtctctggacctagtgactatgaagaagctggacagtaaggtgaacatcatccccatcattgccaaagcagatgccatttcgaagagtgagctaacaaagttcaaaatcaaaatcaccagcgagcttgtcagcaacggagtccagatctatcagtttcctacagatgatgagtcggtggcagagatcaatggaaccatgaacgcccacctgccgtttgctgtcattggcagcacagaagaactgaagataggcaacaagatgatgagggcgcggcagtatccttggggcactgtgcaggttgaaaacgaggcccactgcgactttgtgaagctgcgggagatgctgattcgggtcaacatggaggatctgcgggagcagacccacacccggcactatgagctgtatcgccgctgtaagctggaggagatgggcttcaaggacaccgaccctgacagcaaacccttcagtttacaggagacatatgaggccaaaaggaacgagttcctaggggaactccagaaaaaagaagaggagatgagacagatgttcgtccagcgagtcaaagagaaagaagcggagctcaaagaggcagagaaagagctgcacgagaagtttgaccgtctgaagaaactgcaccaggacgagaagaagaaactggaggataagaagaaatccctggatgatgaagtgaatgctttcaagcaaagaaagacggcggctgagctgctccagtcccagggctcccaggctggaggctcacagactctgaagagagacaaagagaagaaaaataatccatggctgtgtactgaatagtattccccgctacagctggactggactccatttagccttttaagccgaggttcctattttaactgacagctttcctttggggtgccaggcagcgaggccccccacccctatcctgccatgtacttcaagctcacttcttctttttgagttccgcaacttgctcctgcctcccagccccactggcactgaccatgaccacctacttctattttttttttagagtttctttttttgatcacttactttcaaagcacacagtcaaacaaggttatgccaaatttccaggcctttttgaagtattgagaaggggaaggggatttctcacttcaattatagatcataataggaagcaaaaagaaaaaaatgaaaagcaaacatatgcacgcacttttcttgttgacaaagcaagaatataggtttgctgtgtaggtttggtgctctattgattggtgagtgaccagagcaagtatgaaggtgatgctgccaaagcacaagccagtttcttgggaaaattcaagttacagtggagtatttttttgaagaccatatgcttggaggtagaaacaaaccaacgaccaaaaaaaaaaaaaaaaaaatctgctcagatactcagccagtagctcagagagatgctgagttaggcctgtcaggtctccttgggaaaggcttcatatttgcaactttgatgattctatgtccagcttcagagctgctttcccagaaattcacgcttaaacaaccaaccggtaaccaccacttccccacaccgccgccctggtaattatttgcattacaaaccggaggcgccctcatttgcatttgtgtacagattaactagttaaggcttgagaagctctgaataattcaaaagtattagacccacacagccttggagagaccttcagaaactaaggaggagttttatattaagggagacattttagtcagtaagacgatataacctacttactccgtaaggggaaatgaaggcccagagaagggaagggacttgaccgaggtcccacttctgtttcgaggcagaagccagactaattttcatgcctcctgactcccaatcagtttcacaaagggattcaatctgtttatatacgttacattcctggatacgaggtcttttgatgttcagagtaactgactagttagtattagaagaccctcgaggtttttttccacagaaaaacatctgaagatggattgggtgagggctggcaaaacgaaggcatgccgggccagctccttaacccaatgacccagtgatgctgcaaggctggaacggggtccaggagactgtgtgtaacaggtgccctaggtgacccttataatcagggaagtttggtgaacaaaaatcgaacccatgagtgaacataaattaaaaagttgatcaacctattaaaatgtgtatttcattgggtagcttttctcactgtagacagattttttccttcttcaatgaaaaggcttttaaattagtacaactgttactatttaaaaaaaaaataccctaagtactctgtttacttctggtgaaacaaaaccagtcattagaaatggtctgtgcttttattttcccagactggagtggcttttctgaaacacacacacacacacacacacacacacacacacacacacacacacacacgtacacacatccctcacttctcttaagccaagaagtttgctttccctagctgcagtgtagatggctcttgtttttgtttttttgttttaatcatttggcattcacatgtggctgttaatatgtgcttgtttttaattaaaacaagaagctttaaaaaaaaaaaaaaaaaaaaaa

SEQ ID NO: 106 - DNA, 1726 bp, Homo sapiens
agaacactggcggccgatcccaacgaggctccctggagcccgacgcagagcagcgccctggccgggccaagcaggagccggcatcatggattccttcaaagtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcagcagaatgggtgcagacccctgacaaacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgctgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctggggctccctgggccctgccccacccaccttatcctcccaccccactccctccaccaccacagcacaccggtgctggccacaccagccccctttcacctccagtgccacaataaacctgtacccagctgtg

SEQ ID NO: 107 - DNA, 3866 bp, Homo sapiens
gcgagtgttgaaagtcggtggcgtaggtcgtcgtcctggatgctggcgagatagatgttatcttccagaggaagaggaggaggcggcgaagcgttttcccagcctcagtctctctttcgttttccttttcccttcccccaaccctccgcccttctctaaatcagccggccttccttgacctcagtgacccgtctggccccgcccaccctcgtcgacgtgattcccgccgtgaggtaagcgccggtggaacctagagccccgcggaagagcagaacgtttgggagtgtgcagctcctgggcccggctcaggcccgtcgcggaggcgcggcgcaggggagcgctcgggtgggagtctcgctcctccaccagtttattgcgacgtagcatccaggcctaggcctccccgtccatccccgccggactcgggcctctggcagcagcgggtgacgcagacggaacatcatgtcgtccgcggccgagcctccgccacccccgcctcccgagagcgcgccttccaagcccgcagcctcgatcgccagcggcgggagcaacagcagcaacaaaggcggccccgaaggcgtcgcggcgcaggcggttgcgtctgcggtcagcgctggtcccgcagacgccgagatggaggaaatatttgatgatgcgtcacctggaaagcaaaaggaaatccaagaaccagatcctacctatgaagaaaaaatgcaaactgaccgggcaaatagattcgagtatttattaaagcagacagaactttttgcacatttcattcaacctgctgctcagaagactccaacttcacctttgaagatgaaaccagggcgcccacgaataaaaaaagatgagaagcagaacttactatccgttggcgattaccgacaccgtagaacagagcaagaggaggatgaagagctattaacagaaagctccaaagcaaccaatgtttgcactcgatttgaagactctccatcgtatgtaaaatggggtaaactgagagattatcaggtccgaggattaaactggctcatttctttgtatgagaatggcatcaatggtatccttgcagatgaaatgggcctaggaaagactcttcaaacaatttctcttcttgggtacatgaaacattatagaaacattcctgggcctcatatggttttggttcctaagtctacattacacaactggatgagtgaattcaagagatgggtaccaacacttagatctgtttgtttgataggagataaagaacaaagagctgcttttgtcagagacgttttattaccgggagaatgggatgtatgtgtaacatcttatgaaatgcttattaaagagaagtctgtgttcaaaaaatttaattggagatacttagtaatagatgaagctcacaggatcaaaaatgaaaaatctaagttgtcagaaatagtgagggaattcaagactacaaatagactattattaactggaacacctcttcagaacaacttgcatgagctgtggtcacttcttaactttctgttgccagatgtgtttaattcagcagatgactttgattcctggtttgatacaaacaactgccttggggatcaaaaactagttgagaggcttcatatggttttgcgtccattcctccttcgtcgaattaaggctgatgttgaaaagagtttgcctccaaagaaggaagtaaaaatctatgtgggcctcagcaaaatgcaaagggaatggtatactcggatattaatgaaggatatagatatactcaactcagcaggcaagatggacaaaatgaggttattgaacatcctaatgcagttgagaaaatgttgtaatcatccatatctctttgatggagcagaacctggtccaccttatacaacagatatgcatctagtaaccaacagtggcaaaatggtggttttagacaagctgctccctaagttaaaagaacaaggttcacgagtactaatcttcagtcaaatgacaagggtattggacattttggaagattattgcatgtggagaaattatgagtactgcaggttggatggtcagacaccccatgatgagagacaagactccatcaatgcatacaatgaaccaaacagcacaaagtttgttttcatgttaagcacgcgtgctggtggtcttggcatcaatcttgcgactgctgatgtagtaattttgtatgattctgattggaatccccaagtagatcttcaggctatggaccgagcacatagaattgggcagactaagacagtcagagtgttccgctttataactgataacactgtagaagaaagaatagtagaacgtgctgagatgaaactcagactggattcaatagtcattcaacaagggaggcttgtggatcagaatctgaacaaaattgggaaagatgaaatgcttcaaatgattagacatggagcaacacatgtgtttgcttcaaaggaaagtgagatcactgatgaagatatcgatggtattttggaaagaggtgcaaagaagactgcagagatgaatgaaaagctctccaagatgggcgaaagttcacttagaaactttacaatggatacagagtcaagtgtttataacttcgaaggagaagactatagagaaaaacaaaagattgcattcacagagtggattgaaccacctaaacgagaaagaaaagccaactatgccgttgatgcatatttcagggaagctcttcgtgttagtgaacctaaagcacccaaggctcctcgacctccaaaacaacccaatgttcaggatttccagttctttcctccacgtttatttgaattactggaaaaagaaattctgttttacagaaaaactattgggtacaaggtacctcgaaatcctgagctgcctaacgcagcacaggcacaaaaagaagaacagcttaaaattgatgaagctgaatcccttaatgatgaagagttagaggaaaaagagaagcttctaacacagggatttaccaattggaataagagagattttaaccagtttatcaaagctaatgagaagtggggtcgtgatgatattgaaaatatagcaagagaagtagaaggcaaaactccagaagaagtcattgaatattcagctgtgttttgggaaaggtgcaacgagctccaggacatagagaagattatggctcagattgaaaggggagaggcgagaattcaaagaagaataagcatcaagaaagcacttgacacaaagattggacggtacaaagcaccttttcatcagctgagaatatcatatggtactaacaaaggaaaaaactatactgaagaagaagatcgttttctgatttgtatgcttcacaaacttggatttgacaaagaaaatgtttatgatgaattgcgacagtgtattcgcaactctcctcagttcagatttgactggtttcttaagtccagaactgcaatggagctccagaggagatgtaataccttaattactttgattgaaagagaaaacatggaactagaagaaaaggagaaggcagagaaaaagaaacgaggaccaaagccttcaacacagaaacgtaaaatggatggcgcacctgatggtcgaggaagaaaaaagaagctgaaactatgaatatgtttttgtttcataatcactaactttaaaccagtagttctttaatttacgggtcttcataagatgtactgtacaatgctcaattgttatgtcatttaaagacatcaggttcatctgtttactgagctagaaacatagtatgtagtttcacttttttaaatgcaacagctgtgctgaaatttttttatcattaacacttgaagtaataaaataggcttcatttattaaaaaaaaaaaaaaaa

SEQ ID NO: 108 - DNA, 927 bp, Homo sapiens
gaggtcagagacttaagtctaaggcactgagcgtatcatgttaaagatgagcgggtggcagcgacagagccaaaatcagagctggaacctgaggagagagtgttcaagaaggaagtgtatcttcatacatcaccacacctgaaagcagatgtgcttttccagactgatccaactgcagagatggcagctgagtcattgcctttctccttcgggacactgtccagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagaaccaggaaacggaaacagagtggtcattccccagcccgggctggaaagcagcgcatgaaggagaaagaacaggagaatgaaaggaaagtggcacagctagctgaagagaatgaacggctcaagcaggaaatcgagcgcctgaccagggaagtagaggcgactcgccgagctctgattgaccgaatggtgaatctgcaccaagcatgaacaattgggagcatcagtcccccacttgggccacactacccacctttcccagaagtggctactgactaccctctcactagtgccaatgatgtgaccctcaatcccacatacgcagggggaaggcttggagtagacaaaaggaaaggtctcagcttgtatatagagattgtacatttatttattactgtccctatctattaaagtgactttctatgagccaaaaaaaaaaaaaaaaaa

SEQ ID NO: 109 - DNA, 2185 bp, Homo sapiens
agcggcgcagggcaccatgggaaacggacggaagctccattgagccaaataagttggccacgtggggcggaacggaaacctcgcagggtcagaccgtagcgacgcgggaagtccggacgcagtagctccctgaagcggaggcgaaggggagtttaagccccagcggcggcaatggcggagaggcccgaggacctaaacctgcccaatgccgtgatcaccaggatcatcaaggaggcgctcccggacggtgtcaacatctccaaggaggcccggagcgccatctcccgcgccgccagcgtcttcgtgctgtacgccacatcctgtgctaacaactttgcaatgaaaggaaagcggaagacgctgaatgccagtgatgtgctctcagccatggaagagatggagttccagcggttcgttatcccattgaaagaagctctggaagcatataggcgggagcagaaaggcaagaaggaggcctcagagcaaaagaagaaggacaaagacaaaaaaacagactcggaagagcaagacaagagcagggatgaggacaatgatgaagacgaagaaaggctggaagaagaagaacagaatgaagaggaagaagtagacaactgaaaagggcgggagactgtggcaccttggaaggtaccacttgaaatgagacgtgctacgtgtttctgtgaagcttttccatgctgggcagagtagtctcaggcaaaagtgcctgaaaagatcagaataaaaactgactagaaataccgtcaaaaccagcacttaccaaactcagagcatctgagtagcagaatcctgttttgcttcatcaatctgaaggttatggccttttgacaagagggattctgaacgactaaattagtcagtttgctcgcttttgtttacttaaatgtatggttagatggtttttgatctgcagtcttccctagtcccccaaataagtaataataacttccatgctgcctgctgtgttccagatcacagaggcagccaaggctcaggaaaaactggggctttgcttatggtcagcctatgtttgattgcacagtctttgacaatagtagcttactaagactgctcagaatgagtaagctttaggaaactactaaaatgctgtgggagaataacaaaatccgttctctctcttcacttgacagaagcattaactgaattttgtttttcatagacccacttggcccaaaacctttctatattgcctgtagatgatcggcctgctggtgtggtagtaatctgatcacagatattcttaaatcagcagtgtggaatggtattagtcaaacatggtgccagtgtcttttacaaaaacagatggcatagtgtcagatgtaccttactctctgctactgtacagctttaggatttgtcatcacctcccggaaggtgtaaaaccccaggcttcctcttcttgtagtccatctcccagacttgtgactatggcttttcattcttcaattcatgacgagtgccaaacttgattttctctaggcacagctttctccctctgtgcctcctgcttcctttctctctcctgcctctcctctgctccccatcccactttctcatctgcctccttttctcacttctgtcagtctgtaagctttgataacctgcttaatactccaaagtgtgagttcctctgatctcttgattccttagttctaatctcacgttttgtttttaagagatggagtctctcactctgtggcccaggctggagtgcagtggcatgatcatagctcattgcatccttgaaatcctgggctcaggtgatcctcccgcctgagcctcctgagtatctgggactacagatgcgtgccaccaagcctggctaattttgtctcatgtcttctaaaaattattttgtgaagccccttcacaaaaaaccttaagggaaatctgatggtgctcaggaatctaactctccctaaaccatcctctttaactgcttctaaaatatctctgttggcctttcttagcctttttctgtttccattcagtgctccaagcgctttttgtttctaagttgagtgcttggggtttgacaggtagtgacgtgtagtttgacactgttaacttgttaaatacagtgaaaagtttgtgagtgaagaatgctgagaagattgtaatgctttgtacaaataaaagtcttgtt

SEQ ID NO: 110 - DNA, 2397 bp, Homo sapiens
cccccgagccgcgccgagtctgccgccgccgcagcgcctccgctccgccaactccgccggcttaaattggactcctagatccgcgagggcgcggcgcagccgagcagcggctctttcagcattggcaaccccaggggccaatatttcccacttagccacagctccagcatcctctctgtgggctgttcaccaactgtacaaccaccatttcactgtggacattactccctcttacagatatgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactataaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaggaaactttttttgtttgctcttgcattgcaaaaattataaagtaatttattatttattgtcggaagacttgccacttttcatgtcatttgacattttttgtttgctgaagtgaaaaaaaaagataaaggttgtacggtggtctttgaattatatgtctaattctatgtgttttgtctttttcttaaatattatgtgaaatcaaagcgccatatgtagaattatatcttcaggactatttcactaataaacatttggcatagat

SEQ ID NO: 111 - DNA, 1402 bp, Homo sapiens
ggggacgaagggaagctccagcgtgtggccccggcgagtgcggataaaagccgccccgccgggctcgggcttcattctgagccgagcccggtgccaagcgcagctagctcagcaggcggcagcggcggcctgagcttcagggcagccagctccctcccggtctcgccttccctcgcggtcagcatgaaagccttcagtcccgtgaggtccgttaggaaaaacagcctgtcggaccacagcctgggcatctcccggagcaaaacccctgtggacgacccgatgagcctgctatacaacatgaacgactgctactccaagctcaaggagctggtgcccagcatcccccagaacaagaaggtgagcaagatggaaatcctgcagcacgtcatcgactacatcttggacctgcagatcgccctggactcgcatcccactattgtcagcctgcatcaccagagacccgggcagaaccaggcgtccaggacgccgctgaccaccctcaacacggatatcagcatcctgtccttgcaggcttctgaattcccttctgagttaatgtcaaatgacagcaaagcactgtgtggctgaataagcggtgttcatgatttcttttattctttgcacaacaacaacaacaacaaattcacggaatcttttaagtgctgaacttatttttcaaccatttcacaaggaggacaagttgaatggacctttttaaaaagaaaaaaaaaatggaaggaaaactaagaatgatcatcttcccagggtgttctcttacttggactgtgatattcgttatttatgaaaaagacttttaaatgccctttctgcagttggaaggttttctttatatactattcccaccatggggagcgaaaacgttaaaatcacaaggaattgcccaatctaagcagactttgccttttttcaaaggtggagcgtgaataccagaaggatccagtattcagtcacttaaatgaagtcttttggtcagaaattacctttttgacacaagcctactgaatgctgtgtatatatttatatataaatatatctatttgagtgaaaccttgtgaactctttaattagagttttcttgtatagtggcagagatgtctatttctgcattcaaaagtgtaatgatgtacttattcatgctaaactttttataaaagtttagttgtaaacttaacccttttatacaaaataaatcaagtgtgtttattgaatggtgattgcctgctttatttcagaggaccagtgctttgatttttattatgctatgttataactgaacccaaataaatacaagttcaaatttatgtagactgtataagattataataaaacatgtctgaagtcaaaaaaaaaaaaaaaaaaaaaaaaaaaaaaaaaaaaaaaa

SEQ ID NO: 112 - DNA, 4221 bp, Homo sapiens
cagcggcagtggagttcgctgcgcgctgttgggggccacctgtcttttcgcttgtgtccctctttctagtgtcgcgctcgagtcccgacgggccgctccaagcctcgacatgtcgtacaactacgtggtaacggcccagaagcccaccgccgtgaacggctgcgtgaccggacactttacttcggccgaagacttaaacctgttgattgccaaaaacacgagattagagatctatgtggtcaccgccgaggggcttcggcccgtcaaagaggtgggcatgtatgggaagattgcggtcatggagcttttcaggcccaagggggagagcaaggacctgctgtttatcttgacagcgaagtacaatgcctgcatcctggagtataaacagagtggcgagagcattgacatcattacgcgagcccatggcaatgtccaggaccgcattggccgcccctcagagaccggcattattggcatcattgaccctgagtgccggatgattggcctgcgtctctatgatggccttttcaaggttattccactagatcgcgataataaagaactcaaggccttcaacatccgcctggaggagctgcatgtcattgatgtcaagttcctatatggttgccaagcacctactatttgctttgtctaccaggaccctcaggggcggcacgtaaaaacctatgaggtgtctctccgagaaaaggaattcaataagggcccttggaaacaggaaaatgtcgaagctgaagcttccatggtgatcgcagtcccagagccctttgggggggccatcatcattggacaggagtcaatcacctatcacaatggtgacaaatacctggctattgcccctcctatcatcaagcaaagcacgattgtgtgccacaatcgagtggaccctaatggctcaagatacctgctgggagacatggaaggccggctcttcatgctgcttttggagaaggaggaacagatggatggcaccgtcactctcaaggatctccgtgtagaactccttggagagacctctattgctgagtgcttgacataccttgataatggtgttgtgtttgtcgggtctcgcctgggtgactcccagcttgtgaagctcaacgttgacagtaatgaacaaggctcctatgtagtggccatggaaacctttaccaacttaggacccattgtcgatatgtgcgtggtggacctggagaggcaggggcaggggcagctggtcacttgctctggggctttcaaggaaggttctttgcggatcatccggaatggaattggaatccacgagcatgccagcattgacttaccaggcatcaaaggattatggccactgcggtctgaccctaatcgtgagacttatgacactttggtgctctcttttgtgggccagacaagagttctcatgttaaatggagaggaggtagaagaaaccgaactgatgggtttcgtggatgatcagcagactttcttctgtggcaacgtggctcatcagcagcttatccagatcacttcagcatcggtgaggttggtctctcaagaacccaaagctctggtcagtgaatggaaggagcctcaggccaagaacatcagtgtggcctcctgcaatagcagccaggtggtggtggctgtaggcagggccctctactatctgcagatccatcctcaggagctccggcagatcagccacacagagatggaacatgaagtggcttgcttggacatcaccccattaggagacagcaatggactgtcccctctttgtgccattggcctctggacggacatctcggctcgtatcttgaagttgccctcttttgaactactgcacaaggagatgctgggtggagagatcattcctcgctccatcctgatgaccacctttgagagtagccattacctcctttgtgccttgggagatggagcgcttttctactttgggctcaacattgagacaggtctgttgagcgaccgtaagaaggtgactttgggcacccagcccaccgtattgaggacttttcgttctctttctaccaccaacgtctttgcttgttctgaccgccccactgtcatctatagcagcaaccacaaattggtcttctcaaatgtcaacctcaaggaagtgaactacatgtgtcccctcaattcagatggctatcctgacagcctggcgctggccaacaatagcaccctcaccattggcaccatcgatgagatccagaagctgcacattcgcacagttcccctctatgagtctccaaggaagatctgctaccaggaagtgtcccagtgtttcggggtcctctccagccgcattgaagtccaagacacgagtgggggcacgacagccttgaggcccagcgctagcacccaggctctgtccagcagtgtaagctccagcaagctgttctccagcagcactgctcctcatgagacctcctttggagaagaggtggaggtgcataacctacttatcattgaccaacacacctttgaagtgcttcatgcccaccagtttctgcagaatgaatatgccctcagtctggtttcctgcaagctgggcaaagaccccaacacttacttcattgtgggcacagcaatggtgtatcctgaagaggcagagcccaagcagggtcgcattgtggtctttcagtattcggatggaaaactacagactgtggctgaaaaggaagtgaaaggggccgtgtactctatggtggaatttaacgggaagctgttagccagcatcaatagcacggtgcggctctatgagtggacaacagagaaggacgtgcgcactgagtgcaaccactacaacaacatcatggccctctacctgaagaccaagggcgacttcatcctggtgggcgaccttatgcgctcagtgctgctgcttgcctacaagcccatggaaggaaactttgaagagattgctcgagactttaatcccaactggatgagtgctgtggaaatcttggatgatgacaattttctgggggctgaaaatgcctttaacttgtttgtgtgtcaaaaggatagcgctgccaccactgacgaggagcggcagcacctccaggaggttggtcttttccacctgggcgagtttgtcaatgtcttttgccacggctctctggtaatgcagaatctgggtgagacttccacccccacacaaggctcggtgctcttcggcacggtcaacggcatgatagggctggtgacctcactgtcagagagctggtacaacctcctgctggacatgcagaatcgactcaataaagtcatcaaaagtgtggggaagatcgagcactccttctggagatcctttcacaccgagcggaagacagaaccagccacaggtttcatcgacggtgacttgattgagagtttcctggatattagccgccccaagatgcaggaggtggtggcaaacctacagtatgacgatggcagcggtatgaagcgagaggccactgcagacgacctcatcaaggttgtggaggagctaactcggatccattagccaagggcagggggcccctttgctgaccctccccaaaggctttgccctgctgccctccccctcctctccaccatcgtcttcttggccatgggaggcctttccctaagccagctgcccccagagccacagttcccctatgtggaagtggggcgggcttcatagagacttgggaatgagctgaaggtgaaacattttctccctggatttttaccagtctcacatgattccagccatcaccttagaccaccaagccttgattggtgttgccagttgtcctccttccggggaaggattttgcagttctttggctgaaaggaagctgtgcgtgtgtgtgtgtgtatgtgtgtgtgtgtatgtgtatctcacactcatgcattgtcctctttttatttagattggcagtgtagggagttgtgggtagtggggaagagggttaggagggtttcattgtctgtgaagtgagaccttccttttacttttcttctattgcctctgagagcatcaggcctagaggcctgactgccaagccatgggtagcctgggtgtaaaacctggagatggtggatgatccccacgccacagcccttttgtctctgcaaactgccttcttcggaaagaagaaggtgggaggatgtgaattgttagtttctgagttttaccaaataaagtagaatataagaagaaaaaaa

SEQ ID NO: 113 - DNA, 2880 bp, Homo sapiens
cactgcatgtgtattcgtgagttcgcggttgaacaactgttcctttactctgctccctgtctttgtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 114 - DNA, 5228 bp, Homo sapiens
cgggcgagggccgggcaggaggagcgggcgcggcgcgggcgaggctgggacccgagcgcgctcacttcgccgcaaagtgccaacttcccctggagtgccgggcgcgcaccgtccgggcgcgggggaaagaaaggcagcgggaatttgagatttttgggaagaaagtcggatttcccccgtccccttccccctgttactaatcctcattaaaaagaaaaacaacagtaactgcaaacttgctaccatcccgtacgtcccccactcctggcaccatgaaggcggccgtcgatctcaagccgactctcaccatcatcaagacggaaaaagtcgatctggagcttttcccctccccggatatggaatgtgcagatgtcccactattaactccaagcagcaaagaaatgatgtctcaagcattaaaagctactttcagtggtttcactaaagaacagcaacgactggggatcccaaaagacccccggcagtggacagaaacccatgttcgggactgggtgatgtgggctgtgaatgaattcagcctgaaaggtgtagacttccagaagttctgtatgaatggagcagccctctgcgccctgggtaaagactgctttctcgagctggccccagactttgttggggacatcttatgggaacatctagagatcctgcagaaagaggatgtgaaaccatatcaagttaatggagtcaacccagcctatccagaatcccgctatacctcggattacttcattagctatggtattgagcatgcccagtgtgttccaccatcggagttctcagagcccagcttcatcacagagtcctatcagacgctccatcccatcagctcggaagagctcctctccctcaagtatgagaatgactacccctcggtcattctccgagaccctctccagacagacaccttgcagaatgactactttgctatcaaacaagaagtcgtcaccccagacaacatgtgcatggggaggaccagtcgtggtaaactcgggggccaggactcttttgaaagcatagagagctacgatagttgtgatcgcctcacccagtcctggagcagccagtcatctttcaacagcctgcagcgtgttccctcctatgacagcttcgactcagaggactatccggctgccctgcccaaccacaagcccaagggcaccttcaaggactatgtgcgggaccgtgctgacctcaataaggacaagcctgtcattcctgctgctgccctagctggctacacaggcagtggaccaatccagctatggcagtttcttctggaattactcactgataaatcctgtcagtcttttatcagctggacaggagatggctgggaattcaaactttctgacccagatgaggtggccaggagatggggaaagaggaaaaacaaacctaagatgaattatgagaaactgagccgtggcctacgctactattacgacaaaaacatcatccacaagacagcggggaaacgctacgtgtaccgctttgtgtgtgacctgcagagcctgctggggtacacccctgaggagctgcacgccatgctggacgtcaagccagatgccgacgagtgatggcactgaaggggctggggaaaccctgctgagaccttccaaggacagccgtgttggttggactctgaattttgaattgttattctattttttattttccagaactcattttttaccttcaggggtgggagctaagtcagttgcagctgtaatcaattgtgcgcagttgggaaaggaaagccaggacttgtggggtgggtgggaccagaaattcttgagcaaattttcaggagagggagaagggccttctcagaagcttgaaggctctggcttaacagagaaagagactaatgtgtccaatcatttttaaaaatcatccatgaaaaagtgtcttgagttgtggacccattagcaagtgacattgtcacatcagaactcatgaaactgatgtaaggcaattaatttgcttctgtttttaggtctgggagggcaaaaaagaggtgggtgggatgaaacatgttttggggggggatgcactgaaaatctgagaactatttacctatcactctagttttgaagcaaagatggacttcagtggggaggggccaaaaccgttgttgtgttaaaatttattttattaaattttgtgccagtattttttttcttaaaaatcgtcttaagctctaaggtggtctcagtattgcaatatcatgtaagtttgtttttatttgccggctgaggattctgtcacaatgaaagaaaactgtttatatagaccccattggaaaagcaaaacgctctcactgagatcagggatcccaaattcatgggacttatataagaaggacaattaatgctgatttgggtacaggggaattatgtgtgtgaatgtcatctacaattaaaaaaaattagcacatccctttacttacttgttatcagtggattctcggggtttggacttaatgttgagctaagaagcattaagtctttgaactgaatgtattttgcatccctggttttggacgacagtaaacgtaggagcactgttgaagtcctggaagggagatcgaaggaggaagattgacttggttctttcttagtcctatatctgtagcatagatgacttggaataaaagctgtatgcatgggcattacccctcaggtcctaagaaataagtcctgaatgcatgtcgttccaaactaacactctgtaatttttcttttatgtcttattttccaagagtcctccattttttgcaccccctcaccgccaactctgttattcagtagagagaagtgtacggctttctgattggtgagtgaaaaagtaacttgagacacgacctaagttgaagagtttagacttgctgagttttagaagtgatggaaattaagagagcatttcaataaaatgtgacttggctgtctttggaagagaagtgcaaggctttcctttgaagaatttaaattagtccggtaggatgtcaggtgagactgtgtatgcaaaatgaatggcacaggtgatgccagggcctcttgcttgggtctgatgtcttggcacagggtaagtgaaggttaattccagaagagaggaatgacttgaaggcaaaggaaactaaggaaggaggttcagtgaggaaaataaggttgtccatgagatttgaatagatttttagttcccccaaggtttaaatacaaacatagtcaagcaaggtagtcatctttctgctggttgtgagggggaatctgaaaatggagttttagaggaaaagtcaacatctaactagtgaggaaaagtgcctaatacaattagaatctccctcactctatagttgcccagttgaaaggataaggaggaggggtggcttttatggacttccatgagagaaggaaagaaatatttcaggtaagcttctcagggctggccctttttgggatttggatgagaaattggaagtactaactactttctagcatatctttaagaaaattgattgttatttactcccagatcctcttgcagacccagaattatcaggaacatagctctgtgattcatgagtgtccccatactgatgaattggagcatccatatggaaagcaaaggcagaattatcccagctgtattattttgatcttttggatgcaggtgccttaatgaagctctcaaaatattttaggagctgctcagggagtgttgggtggaactgtttggactacattgttttctcttagattatgtgatttttgttgggcactggcaaaaggtgtgtgtgtgaatgtgtgcatgtgtgtgaatgttgtgtgtgtgtgtgtgtgtgtgtgtgtgtgtgtgtgtgtttgcagacatgcaaaactgcagctgaaataataccttagatttctaggtaagtctttccacatttcaataatgggtaagagtagaaccagggccgggtatcaattattgcttgctgtttgcaaccaggcataaaatcactttctcaaatcatccaccgttcctattaaatttatgccggaaactctccttctgtgagtataactcctgcagttcctatagcagataagatataagaaagtgcctcctagtgctcctccgcccgcttgtttgctaaaattccctttctctctaagtccaccattttcaagatttgtagatagtgtattagttaagacagctttgtcgatctggccagatgttttttctcctttgtccaaaggccagagaccatcccaggaagagtggtgggtggtttatacactggaaatgttgcgtttatgctttttaaaaacacacgttaacttcagaggaaggatgggcaaatctggtctagctgggtgaaacccttattttcccagagatgccttaacctttgttggttttggctttagggttcagagtcacttttgttcccttctccattctggagagggacttcccctacatagagccctgatttttgtggctgtggggattggaggtagcattcaaagatcagatgtgcttttcctcactttggagatgaacactctgggttttacagcattaacctgcctaaccttcatggtgagaaatacaccatctctcttctagtcatgctgtgcatgccgcttactctgttggggtctatataaatttgttgaactcttacctacattccaaagaagtttcaaggaaccataaatatatgtatacatatacatatataaaatatatatattaaaataaaattatcaggaatactgcctcagttattgaacttttttttttaagaatactttttttttaagctgagaagtatagggatgaaaaagatgttatattgtgtttgactattttccaacttgtattttcatataatttatattttttaaaagctgaaaatttagaagcaagatgaaaaaaaggaaaagcaggtgctttttaaaaatcagaactgaggtagcttagagatgtagcgatgtaagtgtcgatgtttttttaaaaaaaaatgcaaaaaaattcttatggcggagttttttgtttgtttattttagtagctgatgctggcacatcattttgctggagagttttttatatactgtagcctgatttcatattgtattttaaactgtgtgaaattaaaaacaaagaatttcattcataaaaaaaaaaaaaaaaaaaaaaaaaaaaaaaaaaaaaaaaaa

SEQ ID NO: 115 - DNA, 1398 bp, Homo sapiens
agcggaggggcgccctaacgaacccgggatacggtctgttcctagtccgctccggaaatgcaactgcgtacgggctggccgcgtaatcgtgacgacagcgcgccagcgccggctagccggacgccctaggcttccgcgagatcttcggtgggggtacgggtgttttacgccaggacgctgatgcgtttgggttctcgtctgcagaccctctggacctggtcacgattccataatgtaccacaacagtagtcagaagcggcactggaccttctccagcgaggagcagctggcaagactgcgggctgacgccaaccgcaaattcagatgcaaagccgtggccaacgggaaggttcttccgaatgatccagtctttcttgagcctcatgaagaaatgacactctgcaaatactatgagaaaaggttattggaattctgttcggtgtttaagccagcaatgccaagatctgttgtgggtacggcttgtatgtatttcaaacgtttttatcttaataactcagtaatggaatatcaccccaggataataatgctcacttgtgcatttttggcctgcaaagtagatgaattcaatgtatctagtcctcagtttgttggaaacctccgggagagtcctcttggacaggagaaggcacttgaacagatactggaatatgaactacttcttatacagcaacttaatttccaccttattgtccacaatccttacagaccatttgagggcttcctcatcgacttaaagacccgctatcccatattggagaatccagagattttgaggaaaacagctgatgactttcttaatagaattgcattgacggatgcttaccttttatacacaccttcccaaattgccctgactgccattttatctagtgcctccagggctggaattactatggaaagttatttatcagagagtctgatgctgaaagagaacagaacttgcctgtcacagttactagatataatgaaaagcatgagaaacttagtaaagaagtatgaaccacccagatctgaagaagttgctgttctgaaacagaagttggagcgatgtcattctgctgagcttgcacttaacgtaatcacgaagaagaggaaaggctatgaagatgatgattacgtctcaaagaaatccaaacatgaggaggaagaatggactgatgacgacctggtagaatctctctaaccatttgaagttgatttctcaatgctaactaatcaagagaagtaggaagcatatcaaacgtttaactttatttaaaaagtataatgtgaaaacataaaatatattaaaacttttctattgttttctttccctttcacagtaactttatgtaaaataaaccatcttcaaaagagctagaataaaaaaaaaaaaaa

SEQ ID NO: 116 - DNA, 3663 bp, Homo sapiens
ggcggcggcggcggcgcggactgggtgcgcggcgcagcgtcctgtgttggaatgtgcggctgccgcgagctcgcggcgcagcagcggagcgagcgccgccgaggcccggggccccagaccctggcggcggctgcggcagccgagacggcagggcgaggcccggaggcctgagcaccctctgcagccccactcctgggccttcttggtccacgacggccccagcacccaactttaccaccctcccccacctctcccccgaaactccagcaacaaagaaaagtagtcggagaaggagcggcgactcagggtcgcccgcccctcctcaccgaggaaggccgaatacagttatggccacccaggtaatggggcagtcttctggaggaggagggctgtttaccagcagtggcaacattggaatggccctgcctaacgacatgtatgacttgcatgacctttccaaagctgaactggccgcacctcagcttattatgctggcaaatgtggccttaactggggaagtaaatggcagctgctgtgattacctggtcggtgaagaaagacagatggcagaactgatgccggttggggataacaacttttcagatagtgaagaaggagaaggacttgaagagtctgctgatataaaaggtgaacctcatggactggaaaacatggaactgagaagtttggaactcagcgtcgtagaacctcagcctgtatttgaggcatcaggtgctccagatatttacagttcaaataaagatcttccccctgaaacacctggagcggaggacaaaggcaagagctcgaagaccaaaccctttcgctgtaagccatgccaatatgaagcagaatctgaagaacagtttgtgcatcacatcagagttcacagtgctaagaaattttttgtggaagagagtgcagagaagcaggcaaaagccagggaatctggctcttccactgcagaagagggagatttctccaagggccccattcgctgtgaccgctgcggctacaatactaatcgatatgatcactatacagcacacctgaaacaccacaccagagctggggataatgagcgagtctacaagtgtatcatttgcacatacacaacagtgagcgagtatcactggaggaaacatttaagaaaccattttccaaggaaagtatacacatgtggaaaatgcaactatttttcagacagaaaaaacaattatgttcagcatgttagaactcatacaggagaacgcccatataaatgtgaactttgtccttactcaagttctcagaagactcatctaactagacatatgcgtactcattcaggtgagaagccatttaaatgtgatcagtgcagttatgtggcctctaatcaacatgaagtaacccgccatgcaagacaggttcacaatgggcctaaacctcttaattgcccacactgtgattacaaaacagcagatagaagcaacttcaaaaaacatgtagagctacatgtgaacccacggcagttcaattgccctgtatgtgactatgcagcttccaagaagtgtaatctacagtatcacttcaaatctaagcatcctacttgtcctaataaaacaatggatgtctcaaaagtgaaactaaagaaaaccaaaaaacgagaggctgacttgcctgataatattaccaatgaaaaaacagaaatagaacaaacaaaaataaaaggggatgtggctggaaagaaaaatgaaaagtccgtcaaagcagagaaaagagatgtctcaaaagagaaaaagccttctaataatgtgtcagtgatccaggtgactaccagaactcgaaaatcagtaacagaggtgaaagagatggatgtgcatacaggaagcaattcagaaaaattcagtaaaactaagaaaagcaaaaggaagctggaagttgacagccattctttacatggtcctgtgaatgatgaggaatcttcaacaaaaaagaaaaagaaggtagaaagcaaatccaaaaataatagtcaggaagtgccaaagggtgacagcaaagtggaggagaataaaaagcaaaatacttgcatgaaaaaaagtacaaagaagaaaactctgaaaaataaatcaagtaagaaaagcagtaagcctcctcagaaggaacctgttgagaagggatctgctcagatggaccctcctcagatggggcctgctcccacagaggcggttcagaaggggcccgttcaggtggagctgccacctcccatggagcatgctcagatggagggtgcccagatacggcctgctcctgacgagcctgttcagatggaggtggttcaggaggggcctgctcagaaggagctgctgcctcccgtggagcctgctcagatggtgggtgcccaaattgtacttgctcacatggagctgcctcctcccatggagactgctcagacggaggttgcccaaatggggcctgctcccatggaacctgctcagatggaggttgcccaggtagaatctgctcccatgcaggtggtccagaaggagcctgttcagatggagctgtctcctcccatggaggtggtccagaaggagcctgttcagatagagctgtctcctcccatggaggtggtccagaaggaacctgttaagatagagctgtctcctcccatagaggtggtccagaaggagcctgttcagatggagttgtctcctcccatgggggtggttcagaaggagcctgctcagagggagccacctcctcccagagagcctccccttcacatggagccaatttccaaaaagcctcctctccgaaaagataaaaaggaaaagtctaacatgcagagtgaaagggcacggaaggagcaagtccttattgaagttggcttagtgcctgttaaagatagctggcttctaaaggaaagtgtaagcacagaggatctctcaccaccatcaccaccactgccaaaggaaaatttaagagaagaggcatcaggagaccaaaaattactcaacacaggtgaaggaaataaagaagcccctcttcagaaagtaggagcagaagaggcagatgagagcctacctggtcttgctgctaatatcaacgaatctacccatatttcatcctctggacaaaacttgaatacgccagagggtgaaactttaaatggtaaacatcagactgacagtatagtttgtgaaatgaaaatggacactgatcagaacacaagagagaatctcactggtataaattcaacagttgaagaaccagtttcaccaatgcttcccccttcagcagtagaagaacgtgaagcagtgtccaaaactgcactggcatcacctcctgctacaatggcagcaaatgagtctcaggaaattgatgaagatgaaggcatccacagccatgaaggaagtgacctaagtgacaacatgtcagagggtagtgatgattctggattgcatggggctcggccagttccacaagaatctagcagaaaaaatgcaaaggaagccttggcagtcaaagcggctaagggagattttgtttgtatcttctgtgatcgttctttcagaaagggaaaagattacagcaaacacctcaatcgccatttggttaatgtgtactatcttgaagaagcagctcaagggcaggagtaatgaaactttgaacaaggtttca

SEQ ID NO: 117 - DNA, 3812 bp, Homo sapiens
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 118 - DNA, 668 bp, Homo sapiens
cggggacccggctgcggtggctgcgggactgacgagaaactactaaagttcctggggaagcaaagtagaatttcataagaacaaaatggatggagaggagaaaacctatggtggctgtgaaggacctgatgccatgtatgtcaaattgatatcatctgatggccatgaatttattgtaaaaagagaacatgcattaacatcaggcacgataaaagccatgttgagtggcccaggtcagtttgctgagaacgaaaccaatgaggtcaattttagagagataccttcacatgtgctatcgaaagtatgcatgtattttacgtacaaggttcgctacactaacagctccaccgagattcctgaattcccaattgcacctgaaattgcactggaactgctgatggctgcgaacttcttagattgttaaataaaataaattataataaactgttaactcttttcagtatttaatacctgtagttcagttagtaactttttcatatatagcatgttgcctgtatgcagttgaactatataaagttcattgcaaagcagattatcttgtttttttgcatagcaatcaaagttgaaatttgtttgctacatcaacaaattaaggacattttcacaaactgagaaataaacaaatatgccaattaaaaaaaaaaaaa

SEQ ID NO: 119 - DNA, 4396 bp, Homo sapiens
gcctgaggtgcccgccctggccccaggagaatgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggcgacatgcacacgctgctgcctggccacggggcgctggcctcaggtttcaccggccccatgtcgctgggtgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcccagacgaggacgaggacgaccttctccccccagagcagaaggccgagcgggagaaggagcgccgggtggccaataacgcccgggagcggctgcgggtccgtgacatcaacgaggcctttaaggagctggggcgcatgtgccaactgcacctcaacagcgagaagccccagaccaaactgctcatcctgcaccaggctgtctcggtcatcctgaacttggagcagcaagtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatgtgaaaggtatgcctccgtgggacgagccacccgctttcagccctgtgctctggccccagaagccggactcgagaccccgggcttcatccacatccacacctcacacacctgttgtcagcatcgagccaacaccaacctgacaaggttcggagtgatgggggcggccaaggtgacactgggtccaggagctccctggggccctggcctaccactcactggcctcgctccccctgtccccgaatctcagccaccgtgtcactctgtgacctgtcccatggatcctgaaactgcatcttggccctgttgcctgggctgacaggagcattttttttttttccagtaaacaaaacctgaaagcaagcaacaaaacatacactttgtcagagaagaaaaaaatgccttaactataaaaagcggagaaatggaaacatatcactcaagggggatgctgtggaaacctggcttattcttctaaagccaccagcaaattgtgcctaagcgaaatattttttttaaggaaaataaaaacattagttacaagattttttttttcttaaggtagatgaaaattagcaaggatgctgcctttggtctctggtttttttaagctttttttgcatatgttttgtaagcaacaaatttttttgtataaaagtcccgtgtctctcgctatttctgctgctgttcctagactgagcattgcatttcttgatcaaccagatgattaaacgttgtattaaaaagaccccgtgtaaacctgagccccccccgtccccccccccggaagccactgcacacagacagacggggacaggcggcgggtcttttgtttttttgatgttgggggttctcttggttttgtcatgtggaaagtgatgcgtgggcgttccctgatgaaggcaccttggggcttccctgccgcatcctctcccctcaggaaggggactgacctgggcttgggggaagggacgtcagcaaggtggctctgaccctcccaggtgactctgccaagcagctgtggccccagcggtaccctacacaacgccctccccaggcccccctaagctgctctcccttggaacctgcacagctctctgaaatggggcattttgttgggaccagtgacccctggcatggggaccacaccctggagcccggtgctggggacctcctggacaccctgtccttcactccttgccccagggacccaggctcatgctctgaactctggctgagaggagtctgctcaggagccagcacaggacaccccccaccccaccccaccatgtccccattacaccagagggccatcgtgacgtagacaggatgccaggggcctgaccagcctccccaatgctggggagcatccctggcctggggccacacctgctgccctccctctgtgtggtccaagggcaagagtggctggagccgggggactgtgctggtctgagccccacgaaggccttgggctgtggctccgaccctgctgcagaaccagcagggtgtcccctcgggcccatctgtgtcccatgtcccagcacccaggcctctctccaggtctccttttctggtcttttgccatgagggtaaccagctcttcccagctggctgggactgtcttgggtttaaaactgcaagtctcctaccctgggatcccatccagttccacacgaactagggcagtggtcactgtggcacccaggtgtgggcctggctagctgggggccttcatgtgcccttcatgcccctccctgcattgaggccttgtggacccctgggctggctgtgttcatccccgctgcaggtcgggcgtctccccccgtgccactcctgagactccaccgttacccccaggagatcctggactgcctgactcccctccccagactggcttgggagcctgggccccatggtagatgcaagggaaacctcaaggccagctcaatgcctggtatctgcccccagtccaggccaggcggaggggaggggctgtccggctgcctctcccttctcggtggcttcccctgcgccctgggagtttgatctcttaagggaacttgcctctccctcttgttttgctcctgccctgcccctaggtctgggtggcagtggccccatagcctctggaactgtgcgttctgcatagaattcaaacgagattcacccagcgcgaggaggaagaaacagcagttcctgggaaccacaattatggggggtggggggtgtgatctgagtgcctcaagatggttttcaaaaaattttttttaaagaaaataattgtatacgtgtcaacacagctggctggatgattgggactttaaaacgaccctctttcaggtggattcagagacctgtcctgtatataacagcactgtagcaataaacgtgacattttataaag

SEQ ID NO: 120 - DNA, 2402 bp, Homo sapiens
ggtgctgcggtgctagagcgcggcgcgaccggacgctgcgggcggggaagaggatggagactgtggcgtccgctgcaacggttggggctgcgcgtgagaaggtggcggtgtaggcacctgcgctcggggaaggctggcggcggcggccgagccatggcgggagacccccttctctgggctccctgaagtctcggggagccgtgacccatgggatcgttgagcagccgggtgctgcgccagccaagaccagcccttgcccagcaggcgcagggtgccagggcggggggctcggcccggaggccggacactggagacgatgcggcgggccacggattctgttactgtgcgggcagccacaagcgcaagcggagcagcgggtccttctgctactgtcaccctgactcggagacggacgaggatgaggaggagggggacgagcagcagcggctcctcaacacccctcgaaggaaaaaattaaagagtacatctaaatatatttatcaaacattatttttgaatggtgaaaacagtgacattaagatttgtgctctaggagaagaatggagcttacacaaaatatatttatgtcaatctggctacttttctagtatgttcagtggttcttggaaagaatccagcatgaatattattgaactggagattcctgaccagaacattgatgtagaagcactgcaggttgcatttggttcactgtatcgagatgatgtcttgataaagcccagtcgagttgttgccattttggcagcagcttgtttgctgcagttggacggtttaatacagcagtgtggtgagacaatgaaggaaacagttaatgtgaaaactgtatgtggctattacacatcagcagggacctatggattagattctgtaaagaaaaagtgccttgaatggcttctaaacaatttgatgactcaccagaatgttgaactttttaaagaactcagtataaatgtcatgaaacagctcattggttcatctaacttatttgtgatgcaagtggagatggatatatacactgctctaaaaaagtggatgttccttcaacttgtgccttcttggaatggatctttaaaacagcttttgacagaaacagatgtctggttttctaaacagaggaaagattttgaaggtatggcctttcttgaaactgaacaaggaaaaccatttgtgtcagtattcagacatttaaggttacaatatattatcagtgatctggcttctgcaagaattattgaacaagatgctgtagtaccttcagaatggctctcttctgtgtataaacagcagtggtttgctatgctgcgggcagaacaggacagtgaggtggggcctcaagaaatcaataaagaagaactagagggaaacagcatgaggtgtggtagaaagcttgccaaagatggtgaatactgctggcgttggacaggttttaacttcggcttcgacctacttgtaacttacaccaatcgatacatcattttcaaacgcaatacactgaatcagccatgtagcggatctgtcagtttacagcctcgaaggagcatagcatttagattacgtttggcttcttttgatagtagtggaaaactaatatgtagtagaacaactggctatcaaatacttacacttgaaaaggatcaggaacaagtggtgatgaacttggacagcaggcttctgatcttccctttatatatctgctgtaacttcttgtatatatcaccagaaaaaaagaattgaaaataatcgtcacccagaaaatccagaaaactgaagatttcatcagttggaaacagtagcactttgaaaactttttaggccagctttaatttaatggccctactgatattcacatcgaaggtgactaacaatgacaaaggccttatgaactgtacagacaatacagaagattattcttatcctcattgcatttctatgcatatgcgtaagaacattttaaagccaagaaaatatctgtcaaaccatttctgttagaacgatgtcaattcatgcttttaatttagcatcaatagaaaattgctgtaggtaaatctcacatttatctgcaacaaaatatagatttaatttttagcttaaactttgtttctaccttatgttagtggacctcagttatccatctgtaaatttctttttatttggctaaaataatctaaaagaataatttggttggccaattagaaatgcctttttcagttggtgtattgaaagctttcctttaacattttcacctgctcattgtgattcctccttttagtctaatatctttccaggtcatacttgtttttaatcattaaatattttcttcctggtaaaaaaaaaaaaaaaaaaaaaaaaaaaaaaaaaaaaaaaaaaaaaaaaaaaaaaaa

SEQ ID NO: 121 - DNA, 4172 bp, Homo sapiens
agtaatagcaggagcagcaacagaaggcgtcggagcgggcgtcggagctgcccgctgtgggagagagaggagacagaaagagcgagcgaggagagggagcccgaggcgaaaaagtaactgtcaaatgcgcggctcctttaaccggagcgctcagtccggctccgagagtcatggcgaccgcagcgtctaaccactacagcctgctcacctccagcgcctccatcgtgcacgccgagccgcccggcggcatgcagcagggcgcggggggctaccgcgaagcgcagagcctggtgcagggcgactacggcgctctgcagagcaacggacacccgctcagccacgctcaccagtggatcaccgcgctgtcccacggcggcggcggcgggggcggtggcggcggcggggggggcgggggcggcggcgggggcggcggcgacggctccccgtggtccaccagccccctgggccagccggacatcaagccctcggtggtggtgcagcagggcggccgcggagacgagctgcacgggccaggcgccctgcagcagcagcatcagcagcagcaacagcaacagcagcagcaacagcagcaacagcagcagcagcagcagcaacagcggccgccgcatctggtgcaccacgccgctaaccaccacccgggacccggggcatggcggagcgcggcggctgcagcgcacctcccaccctccatgggagcgtccaacggcggcttgctctactcgcagcccagcttcacggtgaacggcatgctgggcgccggcgggcagccggccgggctgcaccaccacggcctgcgggacgcgcacgacgagccacaccatgccgaccaccacccgcacccgcactcgcacccacaccagcagccgccgcccccgccgcccccgcagggtccgcctggccacccaggcgcgcaccacgacccgcactcggacgaggacacgccgacctcggacgacctggagcagttcgccaagcagttcaagcagcggcggatcaaactgggatttacccaagcggacgtggggctggctctgggcaccctgtatggcaacgtgttctcgcagaccaccatctgcaggtttgaggccctgcagctgagcttcaagaacatgtgcaagctgaagcctttgttgaacaagtggttggaggaggcggactcgtcctcgggcagccccacgagcatagacaagatcgcagcgcaagggcgcaagcggaaaaagcggacctccatcgaggtgagcgtcaagggggctctggagagccatttcctcaaatgccccaagccctcggcccaggagatcacctccctcgcggacagcttacagctggagaaggaggtggtgagagtttggttttgtaacaggagacagaaagagaaaaggatgacccctcccggagggactctgccgggcgccgaggatgtgtacggggggagtagggacactccaccacaccacggggtgcagacgcccgtccagtgaactcgagctgggggaggggcagagcgcggggctccccctccccttcggtccttggccctttcccggccctcttgttccctctctaacttctgattgttcttttatttttaattattatttccccgtcccttaaaaagacaaaaaaaataaggcaaaaggaaagcaactaagacactggactatcctttaaaggtagcaggtgtaatgatgtgttttgacctttgcaggcgagtaaccaggcaatggagtggagtgtctcctggagagagtgaggagagtgtgtgatagctagaaagagagagagacagagagatggcaagcactgagataaatacctggcaaaactaaataaattaccaaaaaggaaaaaaaatccaccaaaccatgataaacacaaaatgcagcttcctgatgcttagagttggcacatgctgctgtgtttatttattgtggattcccatcaggaaagaggaaaaaatacacatgttctttcatataggcaaaatttaaccacataaatttgcactgcaagaaaattgaagtttacgtgaacaaattcatgagcatattttctctttctccccaccgttaatttgggagttgccgttttgggggattttgttttgctttgctttattcatcggagagagttgaagccagctcttggccactctccatttctaatgttcttgtgttgccccttcttcgtactgtttgtgaactttggttaccttcacattccccttacgagggtgtaacatctatttgttcctcttaccaaagcaaaaggattggcttcatacaaaatagacaattctctgatttcaggaaatgtgcatggtctacccgctttatcgaaggcaagaatccggtttggaatataaaaataagcattggttgttcttaccagccacaaagtaaacttcattttcaggcagtgtttctgggggaggttatggagggaagaaaaaagaaaaatcgatagtgagtgactgattgcttcattttatcaggcgggcccattgtgaaagagctcaggggaaatgtggaggttaaatatatttccagagttgtccagcagaaagaaagtggcactttgaagagaactagggaagtacatatcttcagatatccctatatagttctctaccttcagttttagtaacaattatgaagaattatttgtgctgacagcagcagttaaactttgtttctctaatagctttttttttacataaaaaaagacccaggaacttaatagtgtatgcataagactgtgttttttagcacacagatacccacagcatacactgacgatctccacgcagtagacaggttttgtcttcactagctcatttgtttatcaagtcatatttagggtcccacaccctcttttcctgtaatttattgcagaatacaccactttgacttggacagctttctgccccctctttcactaaggaaggcaaatgaagtgaaaaaaaaaaatgccattttcaatccttcctttctcccctttgttaatagttttaagtgaatttttgaccttatcttaatggaaaacggttaactccaaacacaaaagactctactggaaagtgtaggtgaaaaaacttgtaactgtattgaaaataaataccattaaactgtgatcagttaaaatttaaaagaaaaatcagcacaaaagggcgctaaaagggaaaacactttttattaatcttaaaagtttgggggtttttttccagttaggtattagataaatttttattttaaaaaatgaaagtctcactaccataaaattatggttcagcatcagattagcattgcactcagtagtctttaaggttttaggaaatatgctttatattgtcttttcaaacacctgtgattgtttcattttccatgtttttgcaagataaatggtgacttataatgggcatatttatttgcctgtatttcatttcccccaatgaatgtcacaaggagatgggcacggagctgcttcgggtgcatcacgctgctcgttcctgaggtatgggaactggcctttagtgaagctatccagagcagggcaaatagccactggtaaagggaggaaatgaatttccagatacttattaccaagtaggtaaggtcagaagctggagttcagagaatgtgtctacagcttctctgactcttataggtttactaagatgaaagttaccactgaaccttaccactatgtatatatgtttaatatctgtcttttgaaatgcagaaatagtttaaatgtttctttgtctatttttctttttttttaatgctacccagggaaatattttcatatcatttttaagtggcctgcctcaatgtatatttatttcttttgaaacaaaaaggttctggaaactgtttttctgtagctttaaatgaataggtgagcaaaatctatatgggatgtaatttttttgttcagtctcttaaaaaatactttgttttggtacatttggttgtgcttgtggggaaaataaaaacgcagagatccttatatatttatgttaaagtaatattttattatctacataaaacagaaatgcacaataaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 122 - DNA, 3344 bp, Homo sapiens
ggcctctggccgcctggctccaacatcaagcaccgggctccgagtggccgggatcagcgccccgaggcagaggccggagggcgcgcgcactgctaggaagtgctggtcccccgcgccgctctgccagcttggtcccccggcagacgcccctgtacgatcgccgctcgccccgcgggcgaggctgcggtggacagcgcggggctccggctggctcgccttcccgcctgccgtgtcctgctgagcgaccctggagtacacatccagatgccagcccagctaccacaggggatccctctgggagactgaaagtacaggttctggggcccaggttgaagccgaccaaccctgagcctcaggccaggggaatggcagcccccttggaggcccaggaccaggcccctggggagggagaagggcttctgattgtgaaagtggaagattcctcctgggaacaggaatctgcccagcatgaggatggcagggattccgaagcctgccgccagcgcttccggcaattctgctacggggatgtgcatgggcctcatgaggccttcagccagctctgggagctctgctgccgctggctgcggcccgagctgcgtaccaaggagcagatcctggagctgctggtgctggagcagttcctgacagtgctgccaggggagatccagggctgggtgcgtgagcagcacccgggaagcggtgaggaggctgtcgccttggtggaggacctacagaagcagccagtgaaagcctggcgacaggatgtgccctcggaggaggcggaacccgaggctgcaggccggggatcccaggccacggggcctcccccgacggtgggggcacggaggcggccgtctgttccccaggagcagcacagccatagcgcccagcctcctgctcttcttaaagagggtcgtcccggagagacgacggacacctgctttgtctctggggtccatggacctgtggcattgggagacatcccattctatttctcccgggaagaatggggcaccctggaccctgctcagcgggatctcttctgggacataaagcgggagaactcccggaacaccaccctgggttttgggctcaaaggccaaagtgagaagtccctgctgcaggagatggtgccggtggtgccaggccagacaggcagcgacgtgactgtgtcctggagccccgaggaggctgaggcctgggagagcgagaaccggccgagggcggccctgggcccagtggtgggcgcgcgacgggggcggccacccactcgccggcgccagttccgggacctggcagccgagaagccgcacagctgcgggcagtgtggaaagcgcttccgctggggctcggacctggcgcggcaccagcgcacgcacacgggcgagaagccacacaagtgccctgagtgcgacaagagcttccgcagctcctcggacctggtgcgccaccaaggcgtgcacacgggcgagaagcccttctcctgttccgagtgcggcaagagcttcagccgcagcgcctacctggccgaccaccagcgcatacacacgggcgagaagcctttcggctgcagcgactgcggcaagagcttctcgctgcgctcctacctgctggaccatcggcgtgtgcacaccggtgagcggcccttcggctgcggagagtgcgacaagagcttcaagcagcgcgcgcacctcatcgcgcatcagagcctgcacgccaagatggcccagcccgtggggtgagcagctgacttggccggaaacccgggggaggcccagccacggcacatcctgctttgttcaccactgggactctccttccatctgtggccacctcccgggctgtccgagggaccccagggtacctcacactcggagctcgcctgccctgcttggctctgaggacctgcccagcgctcaaagggaacggaagccttcccctcccgcccccgatcttgtcctctttcccccttctgcgcctagcgttcctcttcccctctagtttcctggagccccaacacattcctggcagggacagcagggtggcaaggactcaggtctaggtcccttcccagaagcccccgagcctcatttgactgtgtggctctttggcccccaccctgtggggtgggtccatgggtcaggcctctgccctaccaacctgtgcctttcagtgggcgtggaggactggccttggccccccagggggctgctggactttgggagagacagcccacacctgtgggaccgcgggtcttagtcacggcggcaggggctttctggccccctcccactcccgtttccaggccatgaccactctgccctgtcctggccatacggactcggcctgcctttgccctcggcctacttgccctagcatgaggctctgagagccacctgcccaccaatctggtgaggataatggtggctccagcgacaggaggccaaccctggagaccaagaacagggcgcctggctgccatcttttcctccagaggtggggctgcaccagactcagcactagcactccatcagcactagcacctcactccatcagcactagcacctcactccatcggccccggcaccctgctccatcggcactggcgccctgctccatcggcactaatgctccactcggcgccccactccatcggccccgctccatcggcactaatgccccactcggcgccccactccatcagcactaatgctccactccattggcactaacgccccaactccagcggcactaatgccccactcggcgccccactccatcagcactaatgctccactccattggcactaacgccccaactccagcggcactaatgacccgctcctttgacattggtgccccactccatcagcactaacgccctgctccatcggcactggtgtcccactccattgtcactaacgtccggctccatcggcactaccaccccgctccatcatcactatgtccagctccgtcggcactaccaccctgctccatcatcactatgtccagctccgtcggcactaccaccctgctccatcatcactacgtccagctccaacggcactggtgccccattccatcggcactaacgccccgctccaccggcaccagtgcctcgctccattggcaccaacgcccagctccaccggtactggctccccgctccatcggcactaacgccccgcttcattggcactttgctgctgcctcctgagcactgccttccatgaacagggacagaccagaggccgtgcaaggactccccctcagacctccaaagggcaacagaagagtattaataaacgtgaaaacttaaaaaaaaaaaaaaaaaaaaa

SEQ ID NO: 123 - DNA, 1306 bp, Homo sapiens
cgcgcttggccttgcccgcgcccgctcgcctcgtctcgcccggcctccccgcgtcgcctcgtcgcctgttccgcgccaggcatggcccccagcactgtggccgtggagctgctcagccccaaagagaaaaaccgactgcggaagccggtggtggagaagatgcgccgcgaccgcatcaacagcagcatcgagcagctgaagctgctgctggagcaggagttcgcgcggcaccagcccaactccaagctggagaaggccgacatcctggagatggctgtcagctacctgaagcacagcaaagccttcgtcgccgccgccggccccaagagcctgcaccaggactacagcgaaggctactcgtggtgcctgcaggaggccgtgcagttcctgacgctccacgccgccagcgacacgcagatgaagctgctgtaccacttccagcggcccccggccgcgcccgccgcgcccgccaaggagcccaaggcgccgggcgccgcgcccccgcccgcgctctccgccaaggccaccgccgccgccgccgccgcgcaccagcccgcctgcggcctctggcggccctggtgacccggcgggacctgcgggcgcgcggcccgacgaccagagggcgagcctgctcctctcgcctgtagggaagcgccttcccgccgtcgtccgccccgggcttggacgcgcccttctccggaaggctctggccccaagctggccggcccgcaggagccccattctcagagaatgtgtgtgcagagtccctgccgttttaggacaatcagggcccatcttctgccaagtgtctgaccccatggggttgttctgtgtttgcatttaagcaagtgacttctgggaagtccccggccgcccggggttctatgatatttgtagtgccggggctcgcacactgctgcccccagcctgtagaggactttcttcagggcccgtagctgctgggcgtacccctggcaggcgggctgtgccgcgggcacatttgccttttgtgaaggccgaactcgagctgtatcctcataggaaacagtgatcaccccggacgggcgtccaggaccctgagggccatggccaaaaggctcctgagtgtgcctggtggtctggctggggctcacggtgggctgtctggggagggtgggtgcctccactatgatccttaaaggattcctctgtgtgggtggatgcgtgtgggcacgactttgtactcagaaattgaactctcagtcacgtggaagccacgggactgctccgaagccgccataataaaatctgattgttcagcccccaa

SEQ ID NO: 124 - DNA, 1914 bp, Homo sapiens
gcagccaggcgcgcactgcacagctctcttctctcgccgccgcccgagcgcacccttcagcccgcgcgccggccgtgagtcctcggtgctcgcccgccggccagacaaacagcccgcccgaccccgtcccgaccctggccgccccgagcggagcctggagcaaaatgatgcttcaacacccaggccaggtctctgcctcggaagtgagtgcttctgccatcgtcccctgcctgtcccctcctgggtcactggtgtttgaggattttgctaacctgacgccctttgtcaaggaagagctgaggtttgccatccagaacaagcacctctgccaccggatgtcctctgcgctggaatcagtcactgtcagcgacagacccctcggggtgtccatcacaaaagccgaggtagcccctgaagaagatgaaaggaaaaagaggcgacgagaaagaaataagattgcagctgcaaagtgccgaaacaagaagaaggagaagacggagtgcctgcagaaagagtcggagaagctggaaagtgtgaatgctgaactgaaggctcagattgaggagctcaagaacgagaagcagcatttgatatacatgctcaaccttcatcggcccacgtgtattgtccgggctcagaatgggaggactccagaagatgagagaaacctctttatccaacagataaaagaaggaacattgcagagctaagcagtcgtggtatgggggcgactggggagtcctcattgaatcctcattttatacccaaaaccctgaagccattggagagctgtcttcctgtgtacctctagaatcccagcagcagagaaccatcaaggcgggagggcctgcagtgattcagcaggcccttcccattctgccccagagtgggtcttggaccagggcaagtgcatctttgcctcaactccaggatttaggccttaacacactggccattcttatgttccagatggcccccagctggtgtcctgcccgcctttcatctggattctacaaaaaaccaggatgcccaccgttagattcaggcagcagtgtctgtacctcgggtgggagggatggggccatctccttcaccgtggctaccattgtcactcgtaggggatgtggagtgagaacagcatttagtgaagttgtgcaacggccagggttgtgctttctagcaaatatgctgttatgtccagaaattgtgtgtgcaagaaaactaggcaatgtactcttccgatgtttgtgtcacacaacactgatgtgacttttatatgctttttctcagatctggtttctaagagttttggggggcggggctgtcaccacgtgcagtatctcaagatattcaggtggccagaagagcttgtcagcaagaggaggaacagaattctcccagcgttaacacaaaatccatgggcagcatgatggcaggtcctctgttgcaaactcagttccaaagtcacaggaagaaagcagaaagttcaacttccaaagggttaggactctccactcaatgtcttaggtcaggagttgtgtctaggctggaagagccaaagaaatattccattttcctttccttgtggttgaaaccacagtcagtggagagatgtttggaacacagtcagtggagctggtggtaccaggtttagcattattggatgtcaaaagcattttttttgtcatgtagctgttttaagaaatctggcccagggtgtttgcagctgtgagaagtcactcacactggccacaaggacgctggctactgtctattaaaattctgatgtttctgtgaaattctcagagtgtttaattgtactcaatggtatcattacaattttctgtaagagaaaatattacttatttatcctagtattcctaacctgtcagaataataaatattgtggtaaaa

SEQ ID NO: 125 - DNA, 1534 bp, Homo sapiens
ggaagcgcagcaactcgtgtctgagcgcccggcggaaaaccgaagttggaagtgtctcttagcagcgcgcggagaagaacggggagccagcatcatggcagaacaggatgtggaaaacgatcttttggattacgatgaagaggaagagccccaggctcctcaagagagcacaccagctccccctaagaaagacatcaagggatcctacgtttccatccacagctctggcttccgggactttctgctgaagccggagctcctgcgggccatcgtggactgtggctttgagcatccttctgaggtccagcatgagtgcattccccaggccatcctgggcatggacgtcctgtgccaggccaagtccgggatgggcaagacagcggtcttcgtgctggccaccctacagcagattgagcctgtcaacggacaggtgacggtcctggtcatgtgccacacgagggagctggccttccagatcagcaaggaatatgagcgcttttccaagtacatgcccagcgtcaaggtgtctgtgttcttcggtggtctctccatcaagaaggatgaagaagtgttgaagaagaactgtccccatgtcgtggtggggaccccgggccgcatcctggcgctcgtgcggaataggagcttcagcctaaagaatgtgaagcactttgtgctggacgagtgtgacaagatgctggagcagctggacatgcggcgggatgtgcaggagatcttccgcctgacaccacacgagaagcagtgcatgatgttcagcgccaccctgagcaaggacatccggcctgtgtgcaggaagttcatgcaggatcccatggaggtgtttgtggacgacgagaccaagctcacgctgcacggcctgcagcagtactacgtcaaactcaaagacagtgagaagaaccgcaagctctttgatctcttggatgtgctggagtttaaccaggtgataatcttcgtcaagtcagtgcagcgctgcatggccctggcccagctcctcgtggagcagaacttcccggccatcgccatccaccggggcatggcccaggaggagcgcctgtcacgctatcagcagttcaaggatttccagcggcggatcctggtggccaccaatctgtttggccgggggatggacatcgagcgagtcaacatcgtctttaactacgacatgcctgaggactcggacacctacctgcaccgggtggcccgggcgggtcgctttggcaccaaaggcctagccatcacttttgtgtctgacgagaatgatgccaaaatcctcaatgacgtccaggaccggtttgaagttaatgtggcagaacttccagaggaaatcgacatctccacatacatcgagcagagccggtaaccaccacgtgccagagccgcccacccggagccgcccgcatgcagcttcacctcccctttccaggcgccactgttgagaagctagagattgtatgagaataaacttgttattatggaaaaaaaaaaaaaaaaaaaaaaaaaaaaaaaaaaaaaaaaa

SEQ ID NO: 126 - DNA, 4071 bp, Homo sapiens
gagtccagccgctggtgcgcggagcggttcaccgtcttcggagcggttcggcccagcctttcgcccaggcgcccaggcccgctgcgcgcgtgcgtgagcgcgcctgcgccgccagggccgctgcaaggggaggagagcggccgcctcaggaggatcccttttcccccagaaattactcaatgctgaaacctctcaaagtggtattagagacgctgaaagcaccatggacgggttttatgatcagcaagtcccttttatggtcccagggaaatctcgatctgaggaatgcagagggcggcctgtgattgacagaaagaggaagtttttggacacagatctggctcacgattctgaagagctatttcaggatctcagtcaacttcaagaggcttggttagctgaagcacaagttcctgatgatgaacagtttgtcccagattttcagtctgataacctggtgcttcatgccccacctccaaccaagatcaaacgggagctgcacagcccctcctctgagctgtcgtcttgtagccatgagcaggctcttggtgctaactatggagaaaagtgcctctacaactattgtgcctatgataggaagcctccctctgggttcaagccattaacccctcctacaacccccctctcacccacccatcagaatcccctatttcccccacctcaggcaactctgcccacctcagggcatgcccctgcagctggcccagttcaaggtgtgggccccgcccccgccccccattcgcttccagagcctggaccacagcagcaaacatttgcggtcccccgaccaccacatcagcccctgcagatgccaaagatgatgcctgaaaaccagtatccatcagaacagagatttcagagacaactgtctgaaccctgccaccccttccctcctcagccaggagttcctggagataatcgccccagttaccatcggcaaatgtcagaacctattgtccctgcagctcccccgccccctcagggattcaaacaagaataccatgacccactctatgaacatggggtcccgggcatgccagggcccccagcacacgggttccagtcaccaatgggaatcaagcaggagcctcgggattactgcgtcgattcagaagtgcctaactgccagtcatcctacatgagagggggttatttctccagcagccatgaaggtttttcatatgaaaaagatccccgattatactttgacgacacttgtgttgtgcctgagagactggaaggcaaagtcaaacaggagcctaccatgtatcgagaggggcccccttaccagaggcgaggttcccttcagctgtggcagttcctggtcacccttcttgatgacccagccaatgcccacttcattgcctggacaggtcgaggcatggagttcaagctgatagaaccggaagaggttgctcggcgctggggcatccagaagaaccggccagccatgaactatgacaagctgagccgctctctccgctattactatgaaaagggcatcatgcagaaggtggctggagagcgatacgtctacaaatttgtctgtgacccagatgccctcttctccatggctttcccggataaccagcgtccgttcctgaaggcagagtccgagtgccacctcagcgaggaggacaccctgccgctgacccactttgaagacagccccgcttacctcctggacatggaccgctgcagcagcctcccctatgccgaaggctttgcttactaagtttctgagtggcggagtggccaaaccctagagctagcagttcccattcaggcaaacaagggcagtggttttgtttgtgtttttggttgttcctaaagcttgccctttgagtattatctggagaacccaagctgtctctggattggcacccttaaagacagatacattggctggggagtgggaacagggaggggcagaaaaccaccaaaaggccagtgcctcaactcttgattctgatgaggtttctgggaagagatcaaaatggagtctccttaccatggacaatacatgcaaagcaatatcttgttcaggttagtacccgcaaaacgggacatgatgtgacaatctcgatcgatcatggactactaaatggcctttacatagaagggctctgatttgcacaatttgttgaaaaatcacaaacccatagaaaagtgagtaggctaagttggggaggctcaaaccattaagggttaaaaatacatcttaaacattggaaagctcttctagctgaatctgaaatattaccccttgtctagaaaaaggggggcagtcagaacagctgttccccactccgtgttctcaaaatcataaaccatggctactcttgggaaccacccggccatgtggtcgccaagtagagcaagccccctttctcttcccaatcacgtggctgagtgtggatgacttttattttaggagaagggcgattaacacttttgacagtattttgttttgccctgatttgggggattgttttgttttggtggttgttttggaaaaacagtttataaactgatttttgtagttttggtatttaaagcaaaaaaacgaaaaacaaaaaacaaaaacaaaccttttggtaatgtgcactgtgtctttagccagggccgtgcaacttatgaagacactgcagcttgagaggggctttgctgaggcttccccttggccatgtgaaagcccgccttgttgcctgctttgtgctttctgcaccagacaacctgatggaacatttgcacctgagttgtacatttttgaagtgtgcagggcagcctggacacaagcttagattctctatgtatagttccccgtgttcactaacatgccctctctggaaagcatatgtatataacatgtgtcatgtcctttggaaacctggtcacctggtgaaaacccttgggattcttccctgggcatgactgatgacaatttccatttcatcagtttgttttgttttcctttttctttaaatcttggactttaaaccctacctgtgtgattcagtagggtttgagacttagctgtgatactgacaggtaagcaacagtgctagcattctagattcctgcctttttttaaaaagaaattattctcattgctgtattatattggaaaagttttaaacaaccaagctaaagctatgtgaaagttgagctcaaagtagaggaaaagttactggtggtaccttgctgcctgctctgctggtagaattctgtgctccccgtgacacttagtacattaagaatgactacactgttcctcgtatgtgaaggaggcagtgctgactccgtgagtgtgagacacgtgctttgaactgcttttctattcatggagcactccatagtctcaaactgtcccccttatgaccaacagcacatttgtgaagaggttcgcagggataaggggtgcactttatagctatggaaacatgagattctcctctattggaagctaattagcccacaaaggtggtaaacctgtagattgggccttaattagcattgtactctaatcaaaggactctttctaaaccatatttatagctttcttaacctacacatagtctatacatagatgcatattttacccccagctggctagagatttatttgttgtaaatgctgtatagatttggttttcctttctttacttaccctggtttggattttttttttttttttttttgaatggatttatgctgtcttagcaatatgacaataatcctctgtagcttgagctacccctcccctgctgtaacttacgtgacctgtgctgtcactgggcataggacagcggcatcacggttgcattcccattggactcatgcacctcccggatggtttttgtttttttcgggggttctttggggtttgtttgtttgcttcttttccagagtgtggaaagtctacagtgcagaaaggcttgaacctgccagctgatttgaaatactttcaccctgcgcagggccgtatgcatcctgccaagctgcgttatattctgtactgtgtacaataaagaagtttgcttttcgttta

SEQ ID NO: 127 - DNA, 2269 bp, Homo sapiens
cagtacattgagctccatagagacagcgccggggcaagtgagagccggacgggcactgggcgactctgtgcctcgctgaggaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagttttttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaagttgcagctgttttgttgacattctgaatgcttctaagtaaatacaattttttttattagtattgttgtccttttcataggtctgaaatttttcttcttgaggggaagctagtcttttgcttttgcccattttgaatcacatgaattattacagtgtctatcctttcatatagttagctaataaaaagcttttgtctacacaccctgcatatcataatgggggtaaagttaagttgagatagttttcatccataactgaacatccaaaatcttgatcagttaagaaatttcacatagcccacttacatttacaaactgaagagtaatcaatctactcaaagcatgggattattagaatcaaacattttgaaagtctgtccttgaaggactaatagaaaagtatgttctaacctttacatgaggactctattctttaactcccattaccatgtaatggcagttatattttgcagttcccacattaaagaagacctgagaatgtatccccaaaagcgtgagcttaaaatacaagactgccatattaaattttttgttgacattagtctcagtgaagactatgaaaatgctggctatagatgtcttttcccatttatctaaatatggactgctcaggaaacgagactttcattacaagtatttttaattaattgggccagcttttcaaacaaagatgccacattcaaaatagggtatattttcctatattacggtttgcccctttataaatccaagtagataggaagaaagaagacaaactttgcatctcagtatgaattattcaatttatttgaatgatttttctttacaaaacaaactcattcattagtcatgtttatctgcttaggagtttagggaacaatttggcaattttgtggttttcgagattatcgttttcttaaagtgccagtattttaaaatagcgttcttgtaattttacacgcttttgtgatggagtgctgttttgttatataatttagacttggattctttccatttgcatttgtttatgtaatttcaggaggaatactgaacatctgagtcctggatgatactaataaactaataattgcagtaaaaaaaaaaaaaaaaaaaaaaaaaaaaaa

SEQ ID NO: 128 - DNA, 2357 bp, Homo sapiens
gaatcacgacccctccctgccatgtatccgcagggcagacatccggctccccatcaacccgggcagccgggatttaaattcacggtggctgagtcttgtgacaggatcaaagacgaattccagttcctgcaagctcagtatcacagcctcaaagtggagtacgacaagctggcaaacgagaagacggagatgcagcgccattatgtgatgtactatgagatgtcctatggcttgaacattgaaatgcacaagcagacagagattgcgaagagactgaacacaattttagcacagatcatgcctttcctgtcacaagagcaccagcagcaggtggcgcaggcagtggagcgcgccaagcaggtcaccatgacggagctgaacgccatcatcgggcagcagcagctccaggcgcagcacctctcccatgccacacacggccccccggtccagttgccaccccacccgtcaggtctccagcctccaggaatccccccagtgacagggagcagctccgggctgctggcactgggcgccctgggcagccaggcccatctgacggtgaaggatgagaagaaccaccatgaactcgatcacagagagagagaatccagtgcgaataactctgtgtcaccctcggaaagcctccgggccagtgagaagcaccggggctctgcggactacagcatggaagccaagaagcggaaggtggaggagaaggacagcttgagccgatacgacagtgatggagacaagagtgatgatctggtggtggatgtttccaatgaggaccccgcaacgccccgggtcagcccggcacactcccctcctgaaaatgggctggacaaggcccgtagcctgaaaaaagatgcccccaccagccctgcctcggtggcctcttccagtagcacaccttcctccaagaccaaagaccttggtcataacgacaaatcctccacccctgggctcaagtccaacacaccaaccccaaggaacgacgccccaactccaggcaccagcacgaccccagggctcaggtcgatgccgggtaaacctccgggcatggacccgataggtataatggcctcggctctgcgcacgcccatctccatcaccagctcctatgcggcgcccttcgccatgatgagccaccatgagatgaacggctccctcaccagtcctggcgcctacgccggcctccacaacatcccaccccagatgagcgccgccgccgctgctgcagccgctgcctatggccgatcgccaatggtgagctttggagctgttggttttgaccctcaccccccgatgcgggccacaggcctcccctcaagcctggcctccattcctggaggaaaaccagcgtactcattccatgtgagtgctgatgggcagatgcagcccgtgcccttcccccacgacgccctggcaggccccggcatcccgaggcacgcccggcagatcaacacactcagccacgggggggtggtgtgtgccgtgaccatcagcaaccccagcaggcacgtctacacaggtggcaagggctgcgtgaagatctgggacatcagccagccaggcagcaagagccccatctcccagctggactgcctgaacagggacaattacatgcgctcctgcaagctgcaccctgatgggcgcacgctcatcgtgggcggcgagggcagcacgctcaccatctgggacctggcctcgcccacgccccgcatcaaggccgagctgacgtcctcggctcccgcctgttatgccctggccattagccctgacgccaaagtctgcttctcctgctgcagcgatgggaacattgctgtctgggacctgcacaaccagaccctggtcaggcagttccagggccacacagatggggccagctgcatagacatctcccatgatggcaccaaactgtggacagggggcctggacaacacggtgcgctcctgggacctgcgggagggccgacagctacagcagcatgacttcacttcccagatcttctcgctgggctactgccccactggggagtggctggctgtgggcatggagagcagcaacgtggaggtgctgcaccacaccaagcctcacaagtaccagctgcacctgcacgagagctgcgtgctctccctcaagttcgcctactgcggcaagtggttcgtgagcactgggaaagataaccttctcaacgcctggaggacgccttatggagccagcatatcccagtctaaagaatcctcgtctgtcttgagttgtgacatttcagcggatgacaaatacattgtaacaggctctggtgacaagaaggccacagtttatgaggtcatctactaaacaagaactccagcagg

SEQ ID NO: 129 - DNA, 3824 bp, Homo sapiens
gcggggctagagctgtcggagaagcgggaccgcgaggccggcgcgcggcgctctgcgcggtcagagggagcgcctggcagcagcaggagcagcagcagcagcccgcggcggggccgccgccagccgccgcgaccgccgcggctgcagcctccgaagggaggccgggtgagccggcgtacgcactttcccgcggactttcggagtgtttgtggatatacatgccaagccgccacgatgatgtccatgaacagcaagcagcctcactttgccatgcatcccaccctccctgagcacaagtacccgtcgctgcactccagctccgaggccatccggcgggcctgcctgcccacgccgccgctgcagagcaacctcttcgccagcctggacgagacgctgctggcgcgggccgaggcgctggcggccgtggacatcgccgtgtcccagggcaagagccatcctttcaagccggacgccacgtaccacacgatgaacagcgtgccgtgcacgtccacttccacggtgcctctggcgcaccaccaccaccaccaccaccaccaccaggcgctcgaacccggcgatctgctggaccacatctcctcgccgtcgctcgcgctcatggccggcgcgggcggcgcgggcgcggcggccggcggcggcggcgcccacgacggcccggggggcggtggcggcccgggcggcggcggcggcccgggcggcggcggccccgggggaggcggcggtggcggcccggggggcggcggcggcggcccgggcggcgggctcctgggcggctccgcgcaccctcacccgcatatgcacagcctgggccacctgtcgcaccccgcggcggcggccgccatgaacatgccgtccgggctgccgcaccccgggctggtggcggcggcggcgcaccacggcgcggcagcggcagcggcggcggcgtcggccgggcaggtggcagcggcatcggcggcggcggccgtggtgggcgcagcgggcctggcgtccatctgcgactcggacacggacccgcgcgagctcgaggcgttcgcggagcgcttcaagcagcggcgcatcaagctgggcgtgacgcaggccgacgtgggctcggcgctggccaacctcaagatcccgggcgtgggctcactcagccagagcaccatctgcaggttcgagtcgctcacgctctcgcacaacaacatgatcgcgctcaagcccatcctgcaggcgtggctcgaggaggccgagggcgcccagcgcgagaaaatgaacaagcctgagctcttcaacggcggcgagaagaagcgcaagcggacttccatcgccgcgcccgagaagcgctccctcgaggcctacttcgccgtgcagccccggccctcgtccgagaagatcgccgccatcgccgagaaactggacctcaaaaagaacgtggtgcgggtgtggttttgcaaccagagacagaagcagaagcggatgaaattctctgccacttactgagggggctgggaggtgtcgggcgggacagaatggggagctgaggaggcatttttggggggctttcctctgcttgcctcccctcggatttggagtgtccgttatcctgcctgcatttggggagtcccttctcgctctctttcctccacccattctctgattttcctgcctttgctgtcccctagccttgaggactggggtgctgggtgtggggattggagtatagggtaggggagaagggggggagcattcgggggagtggggagtggggggaaggaaagcggagacccgagcaggggttttaaggagcaggatggttctggggtttgggtggggggagacgcgggaagggtaggaaaatggactgtttctgaccagagacacttacctaaatatcctggggaccaaggaactatgtacaaaaacaaacctaccaaccaccaaaaactagacaaataaagacaaactaaaacaaaacagaacaaaagcaaaggaaaatgctttagaaattttaactccggggagccataatctgcaacttcattttcccccatagaagagaaaaaagagcaccaccattattaccacctccccaaccctacacgcacgaactgagtcgaaaaacgaaaaccaaacgagcgagaagttgaagttctgggtatcaaagctagttgttctgtctgcgtgtttaatttttccctctctcacctccaccccatccatatcctctttatttcctccgttccaatgagaggcctatggctgctctccaatcccgggaagtgagtgggagcacagctgaaaagagagggtcagggggaggctggctgcttgcttaggtggaatccaacttttcccgtggccctgcctatactctggtggcctggtcctgttggggtgggggtctttggagagaagggcatagtctttgagctactaaaaagcagaattccggagcttcgagatatcttattctaggaaaatgaaacaattttaacaacagttttttttcctcttatgtcgaagatctagttttagacaatttcaaaataagcttttcccactcatagaactttaacttgccctttcagttttatcttttttttagagagaggtttaaactactgatttttcctgttgattcaaatagactaatggggtgaaagttattaggagagatactctctcctgttttctccactgaacgagactcatcttgctcttctaggtcccgtttcttcctctcttggaggacatgaaattatagaaatgttgagaagttcctgctttcttttgcggtaggacttggctgtgagaaaatcacctaaatcccagaaaagaggaagacagatttaaagtgcccccacccccatttgtttcaaagaggtctgcatgttgggcgaaaacagaacaactgtgtttccttttacttgttcttattattcaagagtcatttattacaggggataaatgttgggtagcaagaactttaatttgcactaccagtctcccaaatagaaaatcatgtatagtatttcatagtaataatcaggtaccttacaagctgctggtggattttaaaaaattaagatagttgaaggtggttaggtaaaatgcctgctttgtgtacaagatactctttggatctctcgtagagatggtttgttaccatcctttaatcataactaaaacattgaaaacagaacaaatgagaaaagaaaaaaaacctgccgattaacaagactgaaatcatgcatgatctgaaaggtgtggaaagaaacacaattaggtctcactctggttaggcattatttatttaattatgttgtatatcattgtttgcagggcaaacattctatgcatttgaaactgagcactaaactgggctagctttctggtagaccgttttgtggctagtgcgatttcacagtctactgcctgtttccactgaaaacatttttgtcatattcttgtattcaaagaaaacaggaaaaaagttattgtaaatattttatttaatgcacacattcacacagtggtaacagactgccagtgttcatcctgaaatgtctcacggattgatctacctgtctatgtatgtctgctgagctttctccttggttatgttttttctcttttacctttctcctcccttacttctatcagaaccaattctatgcgccaaatacaacagggggatgtgtcccagtacacttacaaaataaaacataactgaaagaagagcagttttatgatttgggtgcgtttttgtgtttatactgggccaggtcctggtagaacctttcaacaaacaaccaaacaaaaaaaaa

SEQ ID NO: 130 - DNA, 4789 bp, Homo sapiens
ctctcgctccggagccgctgcacatttcggaatcttctgcggcttgtccatagtgtgaataaaaactaaatcacatctataattctactgaactggtcatacagacgctgccatatatgtcacagtacactgaaaaggagccagcagcaatggaccaagaatctggtaaggctgtctggcccaaaccagcaggagggtatcagacaattacaggcaggagatatggaagaagacatgcttatgtcagttttaaaccatgtatgaccagacatgaaagaagcttaggtcgggctggtgatgactatgaagtgttggaactagatgatgttccaaaggaaaattcctcaggttccagtcctttggatcaagttgattcttctttaccaagtgaacctatatttgaaaaaagtgaaacagaaattcccacttgtggttcagcattgaatcaaaccactgagagcagtcaatcctttgttgcagtacatcacagtgaggaaggcagggataccttaggaagcagtacaaatcttcataatcactctgagggagagtatattccaggagcttgtagtgcttcaagtgtccaaaatggaattgcattggttcatacagactcttatgatccagatggcaaacatggagaagataatgaccatcttcaactttctgcagaagtcgtggaaggtagtagataccaggaatcattaggcaatacagtatttgagttggaaaacagagaggcagaggcatacactggtctttcaccaccagttccctcatttaactgtgaagtaagagatgagtttgaagagttagattctgtaccattagtgaaaagttctgctggtgatactgagtttgtccatcagaatagccaggaaattcagaggtcttctcaagatgaaatggttagtacgaaacaacaaaataatactagccaggaaagacagacagaacattcacctgaagatgcagcctgtggtccagggcatatttgtagtgaacgaaataccaatgatagggaaaagaaccatggaagttctcctgaacaggtagtgaggccaaaagttagaaaactgataagttcaagccaggtggaccaagaaacaggttttaataggcatgaggcgaaacaaagaagtgttcaaagatggagagaggctttggaagttgaggaaagtggctcagatgacctcttaataaaatgtgaagaatatgatggagagcatgactgtatgttcttggatccaccatactcaagagttattacacaaagggaaacagaaaataaccaaatgacatcagaaagtggagccacagcgggaaggcaagaagtggataacaccttttggaatggctgtggagattattaccaactctatgacaaagatgaagatagttctgaatgcagtgatggggaatggtctgcttctttgcctcatcgattttctggtacagaaaaagatcaatcctcaagtgatgaaagctgggagactctgccaggaaaagatgagaatgaacctgagctacaaagtgatagcagtggccctgaagaagaaaaccaagaattatctcttcaggaaggggaacagacatccttggaagagggagaaattccttggttacagtacaatgaagtcaatgaaagcagcagtgatgagggaaatgaacctgccaatgaatttgcacagccagctttcatgttggatggtaacaataacctggaggatgactccagtgtgagtgaagacttagatgtggattggagcatatttgatggctttgcagatggactaggagttgctgaagctatttcatatgtggatcctcagttccttacatacatggcactagaagaacgcttagcccaggctatggagactgctctggcccatttagagtctcttgcagtggatgttgaggtggccaatccaccagctagtaaggaaagcattgatggtcttccagagacccttgttcttgaagatcacactgctattggtcaggaacaatgctgtccaatctgttgcagtgagtatattaaggatgatatagcaacagagttgccctgtcaccatttctttcacaaaccttgtgtctcaatttggctacaaaagtcgggaacatgccctgtgtgccgccgtcatttcccacctgcggttattgaagcatctgcagctccttcctctgagcctgatcctgatgccccaccttcgaatgacagtattgcagaagcaccctaaaccttgacagttgaaatgagatcagtgtatcaaagtaaatctgcaaattccttctaaatttcatgtgcaaataattatatataaatatatttaaaaatgctatatatagtatatgccatagtttagaaagaatattaacctttctaaactaaatttaggtttgcagaaagtattaaacatttttaagctgaatgttaagacagtgcatccattttctttagttgaatatgtttgtattaattgtaaagccaagcttatcagttgactctctccagaataaataatcatctgtgtggcatacgttattggctttgtctgtaatactgccactaagtgattataattaagctgtcatgtttgcatcaaatgtaaagactgtgttcacaaccgtagtaaaatttggtttcattggaaatgaaacaaattctaaagtatgctttttcactagtccctttgattttgctatatcataacctcttggttcatatgctgagaaatttttcagaaagtccatttttgtttaaaattagaacttttcagaatgccaaatgaaggctaaaattttggccagaacattacaaaagttttaaatcgtagacgtaactccccctgaaataaagttaggtagtaaaatccttaatgaaaccagtggatgtgcttaacgtaaggttagtaaagcatacaaagaatctagtgtgctcagggcttggtacaatgagctgaattagatggccttatgaaactctttctaacctcttacccaacctgtttctccttggttaaaattatacttgaaggcccagaacactcatggcacatttgtttaatattgcttatagttagtttaaggtaattttgcttctacagtattttggaaggtctgaaaacttgcacagggtcatctttgtaattatataaccccaaactaagatgcacaatgtctccttcaggtgatcacacacagtggacgagtatgtgcaaacatggacataatagttcacttacaaatgtgatttgatgttaacactagagaatgatgactgtagaacatttgagcaagtaaaatagtaaagcacatagtgagtgtatgtccgtctaactggtacattgataatttagtttgggcacataaaaggaatatttatatggcttcccaaatgcagagttacatcttattcgtgtatttctctgagtatttatatcccgtctccttttttcattcttaaaaataaatgaattttcactgttggcacatatgaggcttaaatataaggaacataacacttgcattctaatttttgcatatattgtaaatgtgtctggtatttacagcaaaatactgtgtatccttttatgggtaaaacaaaagtgaacattgcatgcatgtaatgtgatgaatttgtaatttaggagttctttggggcttctgtgacttgggtaatgcttacattcaggccttaatgttgcattagctagcatgttttccctctgatgtatatagtcactgttgtataaactaatctttgcttgttttctactctgtgatctttccatatcatatttcattaatgatcagttagtgtcaaggagtcaaaacagattaaaattaatttcatgtgtatatggtggaaatttgtggctagtgtgatttttgtttgtttccttttaagtactgttgatcagttgtgacacttactggttaaacttacgttgctaaagatttctctataataagccacacattatatttagactatattaagggaccttggttttcttctagatagcagctgtcccaaagaaaatatttcttctttgtctgttaagatttagctattatctgccagttgttaagaggttttggttccaaactcaaccagcaatgttgagagctgaacttaagatagctgttgtactttttgctttccatctgttactgtccttcattcttggctccctactatctataaacagctgctgtgaagaagaaaagttgaataagagttggcttaaattttaaaaaagaaaaagaaaattgaggttttaggattttcatggtaacaagctctggtataagctaaggctggcaagttcagatactaaaatattatttgatcatatcttggatccttttgaaaaagttaagactatatgaaggtaaattagaaataagtatgaatattaataaaatagcatttatcttatttctctattttatgttgtgacttaacctaattttatttttttaacattttcttatttcttataatatgaatgctgatatttaaaggtagatctatgtggtattctttgtgtttcttaattgtttaactcttaagattatttgtgatctggatttatgtatttgttagatacatacgaattgttaaaatggaatgcaagtttttcaaaagcccaggtctaaatgtaatggttggtttattgttctataaccccagcccatcattttctgtgtaaatcataaacaataaacagaatatactcggtggtcaaaaaaaaaaaaaaaaagaaaaaaaaaaaaaaaa

SEQ ID NO: 131 - DNA, 2256 bp, Homo sapiens
tcagagtccggctcaggctccggctgcggctccagcccgcgatgccccattccgtgaccctgcgcgggccttcgccctggggcttccgcctggtgggcggccgggacttcagcgcgcccctcaccatctcacgggtccatgctggcagcaaggctgcattggctgccctgtgcccaggagacctgatccaggccatcaatggtgagagcacagagctcatgacacacctggaggcacagaaccgcatcaagggctgccacgatcacctcacactgtctgtgagcaggcctgagggcaggagctggcccagtgcccctgatgacagcaaggctcaggcacacaggatccacatcgatcctgagatccaggacggcagcccaacaaccagcaggcggccctcaggcaccgggactgggccagaagatggcagaccaagcctgggatctccatatggacaaccccctcgctttccagtccctcacaatggcagcagcgaggccaccctgccagcccagatgagcaccctgcatgtgtctccaccccccagcgctgacccagccagaggcctcccgcggagccgggactgcagagtcgacctgggctccgaggtgtacaggatgctgcgggagccagccgagcccgtggccgcggagcccaagcagtcaggctccttccgctacttgcagggcatgctagaggccggcgagggcggggattggcccgggcctggcggcccccggaacctcaagcccacggccagcaagctgggcgctccgctgagcggcctgcaggggctgcccgagtgcacgcgctgcggccacggcatcgtgggcaccatcgtcaaggcacgggacaagctctaccatcccgagtgcttcatgtgcagtgactgcggcctgaacctcaagcagcgtggttacttctttctggacgagcggctctactgtgagagccacgccaaggcgcgcgtgaagccgcccgagggctacgacgtggtggcggtgtaccccaatgccaaggtggaactcgtctgagctgggaccctgctcccacgcctgcttcttaaggtccctgctcggccggtgtaaatatgtttcaccctgtccctctaataaagctcctctgctccaccttgaacctgtcacctggcctgccaccctcctgcgcaggccatgcatggctcccgagtacagtagtatctgcttaggtgccaggcatgtcctaggctctgggtgcagtagtgagcaggacgggtaccatgctgccctgaaggggccacagcctgggtggaaggcagacctgaatcacaacgggccagctctagtaatacgaaggtgaggtctgggatgctgcgtgcggcgcgacgacaggaggtatgacctgggtggggtcagggaaagtcttagctgagaactaagagatgagggaggcacaaactctgcacgggggaactgtgtgtgcaaaggtgagctgggggcgagaaaggcctctgtggccgtcatcggcactagcgggttgggggtggtggtggggtgctggcagcctcaggagcgatcgctcccctggagagggtgattgagtgtggagatcaccggggccgccgcgggccgtctgcacttctgttccaggtttttctggcattttctgttccaggtttcttccacccacgagcactttattctcctccgagacccccttttcttccccttcctctctcccaccagggggcgcgcttaaatttccagacagagaccagaaggaaggtttagaaagaaaagaacacttgcccagggcagctttgccctccaagaggctccctcctgtctctaagtcaattccacccctcctcgaagtcccggcttccacccctctcctcagagtcccggcgtttacctcgtggtgtgtttgccttgggcctttcatgccccggctgcacacatcgtctgtgaattgtggctgttcactcccgaggatgtgtcctagactccgggtcgcgtggatctaccctctagtttacttgctcgggagaagaaactgactcgttttatttagtgcctatttagcgagcccagagtaacgtacatttgtgctgttttcaattttgtgctatcgcaaatcacaaaaaaactgttatcaattcacattcatcatcagcacgagacccatttccttgtgctcctgccagctcaggatattatgtttcttttttcatttttgccagtctgttaagtaaaatgtcatatgcgtttgttttaa

SEQ ID NO: 132 - DNA, 2180 bp, Homo sapiens
gggcggaaaagcctgtttacacagactgcacaccgcctggggaataatgcagtaaaggaagtgagccggctcggcctgactgctccaacttcctgctctcacacacaccagaggggaaaaaaaaagaggagcgagagaaagaaaaaaagggggaaaaatcaggatctcattacaagagccacagaccgtctgcagacgcctgtcagcatggaaagtcgggggctttcgcccgggtcctcctagaaattccccccgaagaagactcccccacatctgggtatggagagtgcaatcacgctgtggcagttcctgttgcagttgctgctggatcagaaacatgagcatttgatctgctggacctcgaacgatggtgaattcaagctcctcaaagcagaagaagtggccaagctgtggggactccgaaaaaacaaaacaaatatgaactatgataagctgagcagagccctgcgatactattatgacaagaacatcatcaagaaggtgatcgggcagaagtttgtgtacaagtttgtctctttcccggagatcctgaagatggatcctcacgcggtggagatcagccgggagagccttctgctgcaggacagcgactgcaaggcgtctccggagggccgcgaggcccacaaacacggcctggccgccctcagaagcacgagccgcaacgaatacatccactcaggcctgtactcgtccttcaccattaattccctgcagaacccaccagacgccttcaaggccatcaagacggagaagctggaggagccgcccgaagacagcccccccgtggaagaagtcaggactgtgatcaggtttgtgaccaataaaaccgacaagcacgtcaccaggccggtggtgtccctgccttccacgtcagaggctgcggcggcgtccgccttcctggcctcgtccgtctcggccaagatctcctctttaatgttgccaaacgctgccagtatttcatccgcctcacccttctcatctcggtccccgtccctgtcccccaactcacccctcccttctgaacacagaagcctcttcctggaggccgcctgccatgactccgattccctggagcccttgaacctgtcatcgggctccaagaccaagtctccatctcttcccccaaaggccaaaaaacccaaaggcttggaaatctcagcgcccccgctggtgctctccggcaccgacatcggctccatcgccctcaacagcccagccctcccctcgggatccctcaccccagccttcttcaccgcacagacaccaaatggattgcttctgactccgagtccactgctctccagcatacatttctggagcagccttagtccagttgctccgctgagtcctgccaggctgcaagggccaagcacgctgttccagttccccacactgcttaatggccacatgccagtgccaatccccagtctggacagagctgcttctccagtactgctttcttcaaactctcagaaatcctgatgacgtctggccacaattaaggactcattaactgatgaaacaaatttgtccccacgggctagtttacctgtgtcgtgagaaggacattgtgaaactcttgttaatttggtttgcacttttcataacatggatagtctagatttatgttagcattttaaaaactgtttttgatatattcaagtatatatgaaaatctgtttggcattaagtgaattttaatgtttttgtttttatatccttttagctcttaagtgttgaacactgttgacagtgaagaacttttcttaatggttttcagtataactaataaggatgtgaagcttttttctctttagttctgagtatgctaaactgtgtgcttatatagactataaccagttgtgccttcctttgcatttaatgtaaatgaatgatttatatattttttagtattaagaggaaatgtttgaaagatgaaaattagtatcaaacagctctctagtagaatttcattatttttcaccagtgggcaatatgaaagcatatatcacgttttgttttactttcaattgtataagaattgccttagaacctcttttgaactgaaattcagtaaatgtccaagtaatgtttttataataaactaagccatatttagacaataaacatcgaaaaaaaaaaaaaaaaaaaaaaaaaaa

SEQ ID NO: 133 - DNA, 4181 bp, Homo sapiens
atgaacgcggccctctgtaatggcggagcgtggcggggacgggggcgagagtgaacgattcaacccgggggagctcaggatggcccaacagcaggccttgaggttccgaggtccggctcccccaccaaatgcagtgatgcgaggcccaccacctctgatgcgacctcctccaccttttggtatgatgcgaggccctcctccaccaccacggccgccctttggacgtcctccttttgatcctaatatgccgccaatgcctcctccaggagggatacctccacctatgggccctccacacctccagagaccacctttcatgcctcctcccatgagttccatgcctcctcctccgggtatgatgtttccaccaggaatgcctcctgtgactgctcctggtactccagcactacctcctacggaggagatatgggttgaaaataaaactccagatgggaaggtttattattataatgctcggacacgtgaatctgcatggaccaagccagatggagttaaggttattcagcaatcagaactgacacctatgcttgcagcccaggcacaggttcaggctcaggcccaggcgcaggctcaggcccaggcgcaggctcaggcccaggcacaagctcaggcccaggctcaggctcaggcccaggcccaggcccaggcccaggcccaggcccaagcccaagcccaggcccaggctcaggctcaggcacaagctcaggcccaggcccaggctcaggtccaggcccaggtccaggcacaagtgcaagcacaagcagttggagcttccacccctacgaccagtagcccagcacctgcagtatccacttcaacatcatcatccaccccttcctctaccacttctaccacaacaactgctacttcagttgcgcagacagtatcaacacccacaacacaagatcagaccccaagttctgctgtttcagttgccacgcctacagttagtgtttcaactcctgctcctacagccacacctgtgcaaaccgttccccagccgcaccctcagacgttacctcctgctgttcctcattcagtacctcagccaacaacagcaatacctgcttttccaccagtaatggtacctccgtttcgtgttccccttcctggcatgccaattccacttccaggtgtagcaatgatgcaaatagtcagctgcccgtatgtaaagacagtcgctaccaccaagaccggtgtattgccaggaatggcccctcctatcgtacccatgatacatccccaggttgctattgcagcttcacctgctaccttagctggagcaacagcagtttctgaatggactgaatataaaacagcagatgggaagacatattattataataatagaacattagaatcaacctgggaaaaaccccaagaactaaaggaaaaagaaaagttagaagagaagattaaagagccaattaaagaaccctctgaagagcctctgccaatggagacggaggaggaggatcctaaagaagagcctataaaggagataaaggaggagcccaaagaagaggagatgactgaagaagaaaaggctgcccagaaggcaaagccagttgctactgctcctattcctggtactccatggtgtgtcgtttggactggtgatgagcgggtcttcttttataatcccaccactcgtctttctatgtgggaccgacctgatgatctgattggcagggcagatgttgacaaaattattcaggagccccctcataaaaaaggaatggaggaattgaagaaactaaggcacccaactccgacaatgctgtcgatccaaaagtggcaattctctatgagtgcaattaaagaggaacaagaattaatggaagaaattaatgaagatgagcctgttaaagcaaaaaaacggaagagagacgataataaagacattgactcagagaaagaagctgccatggaagctgaaattaaagctgcccgagaaagggccattgtccctctggaggctcgaatgaagcagttcaaggacatgctgctagagagaggggtgtctgctttttcaacgtgggagaaggagttgcacaagatagtttttgatccccggtacttacttctcaatcctaaagagagaaaacaggtgtttgatcagtatgtaaagaccagggcagaggaagaacgcagggaaaagaaaaataaaataatgcaagccaaggaagatttcaaaaaaatgatggaagaagcaaaatttaatccaagagcaacttttagtgaatttgcagccaagcatgctaaagattcaagattcaaagcaattgaaaagatgaaagaccgagaagccttgtttaatgagtttgtggccgctgctaggaagaaagagaaagaagattcgaagaccagaggtgagaagattaaatcggatttctttgaactattatctaatcatcacttggacagtcagtctcgatggagcaaagtaaaagacaaagtagaaagtgatccacgttacaaagcagtagatagttcatcaatgagagaagaccttttcaaacagtacattgaaaaaatagccaagaatttagactcagaaaaagaaaaggagcttgaaaggcaagcccgcattgaggcaagccttcgagaacgagaaagggaggttcaaaaggcccgttcagaacaaacaaaagaaatagatcgagagagagagcagcacaaacgagaagaagctatccagaatttcaaagctcttctgtctgacatggtacgttcttcagatgtgtcatggtctgatactcgtaggaccctccgaaaagatcaccgctgggaatctggatccttattggaaagagaggagaaagagaagctttttaatgaacacattgaagcacttaccaaaaaaaagagagagcactttaggcaacttctggatgaaacttctgcaattaccttaacatccacgtggaaagaagtaaaaaaaatcattaaggaagatcctcgatgtattaagttctcctccagtgacaggaaaaaacaaagagaatttgaagaatatatcagagacaaatatatcacagccaaagctgacttcaggacgcttttgaaagagaccaaatttataacatatagatccaaaaaattaatccaagaatcagatcagcacctgaaagatgtagaaaaaattttacagaatgacaaacggtatctagtactggactgtgtgccagaggagaggcgtaaactgattgtggcatatgttgatgacctggatcgccggggtccacccccacctcccacagcatcggagcccacgagacgatcaacaaaataattctaaatactcttccataggggcatctattcaaaatgcttgcatgagccaattttcaggtttttacatatatgtgcattagtcaacctattgcgaaaccatctgacaaacagaaggagaagcatttgtgaacagtttctgaacagaacactttggaaatatttatgcttttctttgtgtggcatgactgacatacatactcaaatataggctgtctctagtaaatcttaaaatcttgaagctaaaattcatccttttatgaggtgtggaagtcagtgacttggtgacgttcttcctagcagtgttaatacatgcaagaagtaagagcatttgtggcttgaacttgccagatgcaaataccacagactccaagaaaacccgagttggggtttgttttgttttgattttttttttttaaagcgggtaaaagagaaaacactgaaaattgaattcttatcttccagaggctacaattattataatggacaatacttttacctttgtctctaaagatcagattagttttatttgttcacttacgtgctttgattatcccctctgaattatagaccgagtcttgttgtttagcctaagagaagatttatgtagtaatttcttctcaggtatggaaccacggtcataactaacatgttggccagaatagaaccactggttaaacatattttattcaccattaagtgatctttatcaatattctggattagacaacaaattacctttctgggtgtttcttgtaaactatactcctgtttgaatgttaaactttgttgctaaagtttaattttaagatgtttgaatgttcagtttatgtatttgaactacaataaaccaaccctttttatat

SEQ ID NO: 134 - DNA, 3952 bp, Homo sapiens
attttagacacaagcgagaggatcatggcggatggccccaggtgtaagcgcagaaagcaggcgaacccgcggcgcaataacgttacaaattataatactgtggtagaaacaaattcagattcagatgatgaagacaaactgcatattgtggaagaagaaagtgttacagatgcagctgactgtgaaggtgtaccagaggatgacctgccaacagaccagacagtgttaccagggaggagcagtgaaagagaagggaatgctaagaactgctgggaggatgacagaaaggaagggcaagaaatcctggggcctgaagctcaggcagatgaagcaggatgtacagtaaaagatgatgaatgcgagtcagatgcagaaaatgagcaaaaccatgatcctaatgttgaagagtttctacaacaacaagacactgctgtcatttttcctgaggcacctgaagaggaccagaggcagggcacaccagaagccagtggtcatgatgaaaatggaacaccagatgcattttcacaattactcacctgtccatattgtgatagaggctataaacgctttacctctctgaaagaacacattaaatatcgtcatgaaaagaatgaagataactttagttgctccctgtgcagttacacctttgcatacagaacccaacttgaacgtcacatgacatcacataaatcaggaagagatcaaagacatgtgacgcagtctgggtgtaatcgtaaattcaaatgcactgagtgtggaaaagctttcaaatacaaacatcacctaaaagagcacttaagaattcacagtggagagaagccatatgaatgcccaaactgcaagaaacgcttttcccattctggctcctatagctcacacataagcagtaagaaatgtatcagcttgatacctgtgaatgggcgaccaagaacaggactcaagacatctcagtgttcttcaccgtctctttcagcatcaccaggcagtcccacacgaccacagatacggcaaaagatagagaataaaccccttcaagaacaactttctgttaaccaaattaaaactgaacctgtggattatgaattcaaacccatagtggttgcttcaggaatcaactgttcaacccctttacaaaatggggttttcactggtggtggcccattacaggcaaccagttctcctcagggcatggtgcaagctgttgttctgccaacagttggtttggtgtctcccataagtatcaatttaagtgatattcagaatgtacttaaagtggcgatagatggtaatgtaataaggcaagtgttggagaataatcaagccaatcttgcatccaaagaacaagaaacaatcaatgcttcacccatacaacaaggtggccattctgttatttcagccatcagtcttcctttggttgatcaagatggaacaaccaaaattatcatcaactacagtcttgagcagcctagccaacttcaagttgttcctcaaaatttaaaaaaagaaaatccagtcgctacaaacagttgtaaaagtgaaaagttaccagaagatcttactgttaagtctgagaaggacaaaagctttgaagggggggtgaatgatagcacttgtcttctgtgtgatgattgtccaggagatattaatgcacttccagaattaaagcactatgacctaaagcagcctactcagcctcctccactccctgcagcagaagctgagaagcctgagtcctctgtttcatcagctactggagatggcaatttgtctcctagtcagccacctttaaagaacctcttgtctctcctaaaagcatattatgctttgaatgcacaaccaagtgcagaagagctctcaaaaattgctgattcagtaaacctaccactggatgtagtaaaaaagtggtttgaaaagatgcaagctggacagatttcagtgcagtcttctgaaccatcttctcctgaaccaggcaaagtaaatacccctgccaagaacaatgatcagcctcaatctgcaaatgcaaatgaaccccaggacagcacagtaaatctacaaagtcctttgaagatgactaactccccagttttaccagtgggatcaaccaccaatggttccagaagtagtacaccatccccatcacctctaaacctttcctcatccagaaatacacagggttacttgtacacagctgagggtgcacaagaagagccacaagtagaacctcttgatctttcactaccaaagcaacagggagaattattagaaaggtcaactatcactagtgtttaccagaacagtgtttattctgtccaggaagaacccttgaacttgtcttgcgcaaaaaaggagccacaaaaggacagttgtgttacagactcagaaccagttgtaaatgtaatcccaccaagtgccaaccccataaatatcgctatacctacagtcactgcccagttacccacaatcgtggccattgctgaccagaacagtgttccatgcttaagagcgctagctgccaataagcaaacgattctgattccccaggtggcatacacctactcaactacggtcagccctgcagtccaagaaccacccttgaaagtgatccagccaaatggaaatcaggatgaaagacaagatactagctcagaaggagtatcaaatgtagaggatcagaatgactctgattctacaccgcccaaaaagaaaatgcggaagacagaaaatggaatgtatgcttgtgatttgtgtgacaagatattccaaaagagtagttcattattgagacataaatatgaacacacaggtaaaagacctcatgagtgtggaatctgtaaaaaggcatttaaacacaaacatcatttgattgaacacatgcgattacattctggagaaaagccctatcaatgtgacaaatgtggaaagcgcttctcacactctgggtcttattctcaacacatgaatcatcgctactcctactgtaagagagaagcggaagaacgtgacagcacagagcaggaagaggcagggcctgaaatcctctcgaatgagcacgtgggtgccagggcgtctccctcacagggcgactcggacgagagagagagtttgacaagggaagaggatgaagacagtgaaaaagaggaagaggaggaggataaagagatggaagaattgcaggaagaaaaagaatgtgaaaaaccacaaggggatgaggaagaggaggaggaggaggaagaagtggaagaagaagaggtagaagaggcagagaatgagggagaagaagcaaaaactgaaggtctgatgaaggatgacagggctgaaagtcaagcaagcagcttaggacaaaaagtaggcgagagtagtgagcaagtgtctgaagaaaagacaaatgaagcctaatcgtttttctagaaggaaaataaattctaattgataatgaatttcgttcaatattatccttgcttttcatggaaacacagtaacctgtatgctgtgattcctgttcactactgtgtaaagtaaaaactaaaaaaatacaaaatacaaaacacacacacacacacacacacacacacacacacacacacacacaaaataaatccgggtgtgcctgaacctcagacctagtaatttttcatgcagttttcaaagttaggaacaagtttgtaacatgcagcagattagaaaaccttaatgactcagagagcaacaatacaagaggttaaaggaagctgattaattagatatgcatctggcattgttttatcttatcagtattatcactcttatgttggtttattcttaagctgtacaattgggagaaattttataattttttattggtaaacatatgctaaatccgcttcagtattttattatgttttttaaaatgtgagaacttctgcactacaaaattcccttcacagagaagtataatgtagttccggaattc

SEQ ID NO: 135 - DNA, 3448 bp, Homo sapiens
attaaccttctcttagtcttcaacctaagtacttgaatgtcaagtaccctccaaccctcaatgtcccaagacttttaagagcggaaggtaccgatgagttccatcctttactagggtcaccaaggaaggcatgggtatatggaaatttttattattattccatctgaatatcattttctagagaataggagcttttgttctgaagggctgccggcttccttctgggatctagcagccagggttagatcacaggtgtcactttcaggcgagtagttagcaacggtatcgctagcaactgagccgacccctgcagccagaggtttgcagtgggtagtgtgtattccagaaagggccctgacatgtgaaaggaaggaatgtgccctaatattctacagttgttttatcgttgctactgattaggtccatggagggaagcccatccctgagacgcatgacagtgatgcgggagaagggccggcgccaggctgtcaggggcccggccttcatgttcaatgaccgcggcaccagcctcaccgccgaggaggagcgcttcctcgacgccgccgagtacggcaacatcccagtggtgcgcaagatgctggaggagtccaagacgctgaacgtcaactgcgtggactacatgggccagaacgcgctgcagctggctgtgggcaacgagcacctggaggtgaccgagctgctgctcaagaaggagaacctggcgcgcattggcgacgccctgctgctcgccatcagcaagggctacgtgcgcatcgtagaggccatcctcaaccaccctggcttcgcggccagcaagcgtctcactctgagcccctgtgagcaggagctgcaggacgacgacttctacgcttacgacgaggacggcacgcgcttctcgccggacatcacccccatcatcctggcggcgcactgccagaaatacgaagtggtgcacatgctgctgatgaagggtgccaggatcgagcggccgcacgactatttctgcaagtgcggggactgcatggagaagcagaggcacgactccttcagccactcacgctcgaggatcaatgcctacaaggggctggccagcccggcttacctctcattgtccagcgaggacccggtgcttacggccctagagctcagcaacgagctggccaagctggccaacatagagaaggagttcaagaatgactatcggaagctctccatgcaatgcaaagactttgtagtgggtgtgctggatctctgccgagactcagaagaggtagaagccattctgaatggagatctggaatcagcagagcctctggaggtacacaggcacaaagcttcattaagtcgtgtcaaacttgccattaagtatgaagtcaaaaagtttgtggctcatcccaactgccagcagcagctcttgacgatctggtatgagaacctctcaggcctaagggagcagaccatagctatcaagtgtctcgttgtgctggtcgtggccctgggccttccattcctggccattggctactggatcgcaccttgcagcaggctggggaaaattctgcgaagcccttttatgaagtttgtagcacatgcagcttctttcatcatcttcctgggtctgcttgtgttcaatgcctcagacaggttcgaaggcatcaccacgctgcccaatatcacagttactgactatcccaaacagatcttcagggtgaaaaccacccagtttacatggactgaaatgctaattatggtctgggttcttggaatgatgtggtctgaatgtaaagagctctggctggaaggacctagggaatacattttgcagttgtggaatgtgcttgactttgggatgctgtccatcttcattgctgctttcacagccagattcctagctttccttcaggcaacgaaggcacaacagtatgtggacagttacgtccaagagagtgacctcagtgaagtgacactcccaccagagatacagtatttcacttatgctagagataaatggctcccttctgaccctcagattatatctgaaggcctttatgccatagctgttgtgctcagcttctctcggattgcgtacatcctccctgcaaatgagagctttggccccctgcagatctctcttggaaggactgtaaaggacatattcaagttcatggtcctctttattatggtgttttttgcctttatgattggcatgttcatactttattcttactaccttggggctaaagttaatgctgcttttaccactgtagaagaaagtttcaagactttattttggtcaatatttgggttgtctgaagtgacttccgttgtgctcaaatatgatcacaaattcatagaaaatattggatacgttctttatggaatatacaatgtaactatggtggtcgttttactcaacatgctaattgctatgattaatagctcatatcaagaaattgaggatgacagtgatgtagaatggaagtttgctcgttcaaaactttggttatcctattttgatgatggaaaaacattacctccacctttcagtctagttcctagtccaaaatcatttgtttatttcatcatgcgaattgttaactttcccaaatgcagaaggagaaggcttcagaaggatatagaaatgggaatgggtaactcaaagtccaggttaaacctcttcactcagtctaactcaagagtttttgaatcacacagttttaacagcattctcaatcagccaacacgttatcagcagataatgaaaagacttataaagcggtatgttttgaaagcacaagtagacaaagaaaatgatgaagttaatgaaggtgaattaaaagaaatcaagcaagatatctccagccttcgttatgaacttttggaagacaagagccaagcaactgaggaattagccattctaattcataaacttagtgagaaactgaatcccagcatgctgagatgtgaatgatgcagcaacctggatttggctttgactatagcacaaatgtgggcaataatatttctaagtatgaaatacttgaaaaactatgatgtaaatttttagtattaactacctttatcatgtgaacctttaaaagttagctcttaatggttttattgttttatcacatgaaaatgcattttatttgtctgctttgacattacagtggcataccattgtgttgaaaagcccaatattactatattattgaaacttttattcattttagagtaaactccacatctttgcactacctgtttgcctccaagagactatcagttccttggggacagggaccatgtcttattcatctttgtgtctccagcatctagtacagtgcctggtatatagtaggtgctcaataaatgttgaaaccaactgaactgccaacaaaataaaaataaaaagtcttcactatgtagcataaaaaaaaaaaaaaaaaaa

SEQ ID NO: 136 - DNA, 1836 bp, Homo sapiens
gcaaagagccgaggccgggcgcgcgaccctcgtccttctgcccctggccgcacactttgcgcacatctctttttctgcatggtggatattatttttcattatccttttctgggtgctatgggtgatcattccaagaagaagcccgggacggccatgtgcgtgggctgcgggagtcagatccacgaccagtttatcctgcgggtgtcgcccgacctcgagtggcacgcggcctgcctcaagtgtgccgagtgcagccagtacctggacgagacgtgcacgtgcttcgtgagagacgggaagacctactgcaagcgggactacgtcaggctgttcggcatcaagtgcgccaagtgccaggtgggcttcagcagcagcgacctggtgatgagggcgcgggacagcgtgtaccacatcgagtgcttccgctgctccgtgtgcagccgccagctgctgcctggggacgagttctcgctgcgggagcacgagctgctctgccgcgccgaccacggcctcctgctcgagcgcgccgcggccggcagcccgcgcagccccggcccgcttcccggcgcccgcggcctgcatctgcccgacgctgggtcgggccggcagcccgcgttgcgcccgcacgtgcacaagcagacggagaagacgacccgcgtgcggactgtgctgaacgagaagcagctgcacactctgcggacctgctacgccgccaacccgcggcccgacgctctcatgaaggagcagctggtggagatgaccggcctgagcccgcgggtcatccgcgtctggttccagaacaagcgctgcaaggacaagaagaaatccattctcatgaagcagctgcagcagcagcagcacagcgacaagacgagccttcagggactgactgggacgcccctggtggcgggcagtcccatccgccatgagaacgccgtgcagggcagcgcagtggaggtgcagacgtaccagccgccgtggaaggcgctcagcgagtttgccctccagagcgacctggaccaacccgccttccaacagctggtctccttctccgagtccggctccctaggcaactcctccggcagcgacgtgacctccctgtcctcgcagctcccggacacccccaacagtatggtgccgagtcccgtggagacgtgagggggacccctccctgccagcccgcggacctcgcatgctccctgcatgagactcacccatgctcaggccattccagttccgaaagctctctcgccttcgtaattattctattgttatttatgagagagtaccgagagacacggtctggacagcccaaggcgccaggatgcaacctgctttcaccagactgcagacccctgctccgaggactcttagtttttcaaaaccagaatctgggacttaccagggttagctctgccctctcctctcctctctacgtggccgccgctctgtctctccacgccccacctgtgtccccatctcggccggcccggagctcgcccacgcggacccccgccctgccccagctcagcgctccctggcggcttcgcccgggctcctagcggggaaaaggaaggggataactcagaggaacagacactcaaactcccaaagcgcatgattgctgggaaacagtagaaaccagacttgccttgaaagtgtttaagttattcgacggaggacagagtatgtgagcctttgccgaacaaacaaacgtaagttattgttatttattgtgagaacagccagttcatagtgggacttgtattttgatcttaataaaaaataataacccgggaaaaaaaaaaaaaaaaaaaaaaaaaaaaaaaaaaaaaa

SEQ ID NO: 137 - DNA, 2639 bp, Homo sapiens
tcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctg

SEQ ID NO: 138 - DNA, 2630 bp, Homo sapiens
tgaaatcagactttggagccacaaagaaccattccaattctgggtcgtccgcttacacacggtgaacggagcacatttcttctctgagccttggttcctttcctgtaaatggagtataaccactcccatcttgcaggagctttttggtgataattaaggtgacttcgtgaaaagtaccagggtctcaaaaatctcaaattttgtggaaagtatgcagtttctaggttggagaggctagagtttggtttcaaaagcctcgaccacacacaggggcggttacagctcttgaggtcccgggggtcacaggacgccttccgagttggggggaggagaaaacaaaaccacaggaagaaacgaacaacgctttcgggtgggcccgggaccatagagggtcgctccgccactttcctagagcgaccgcggcggcgccagagcgcggcgaggccctcacttccggcggcgcgggaggcgcccagcgagccagagtggtggctggtcccgcgcgaaaattctgagctgtacacctctaggaaatgaaacactagttcagaagaagcctgtaaactctcttacaaatacatttggttattcaccatgaggttagcaaagcctaaagcgggtatttctcggagctcaagccaaggaaaggcctatgagaacaagcgcaaaacaggccggcagcggcagaagtggggcatgactattcgatttgactcaagcttcagtagactcagaagaagcttggatgacaaaccctataaatgtactgaatgtgaaaagagtttcagtcagagttcaactctttttcaacaccagaagatccatactggaaagaaatcccataaatgtgctgattgtgggaaaagtttctttcagagttctaatctcattcagcatcgacggatccatacgggggaaaagccctacaaatgtgatgagtgtggagaaagcttcaaacagagctcaaatctcattcagcaccagagaattcatactggagaaaaaccctatcagtgtgatgagtgtggccggtgtttcagccagagctcccaccttattcaacatcagagaacccacactggggagaaaccctaccagtgcagtgaatgtggcaaatgtttcagtcagagctctcatctgaggcagcacatgaaggtgcataaagaagagaagcctcgtaaaacccggggcaaaaatatcagggtgaagactcacttaccctcttggaaagctggtacaggaaggaagtctgtggctggtctccgttaagtatagggctttttgacagctttttgagacctcttaagaaaaaataaaaagtaaaaaatgaaaggaatcttttttagaaatagagatgctttatagtagatcacttaaatactggatcttttgctagtgtgaaaacattgggaatttaatgacattattgagctgaaagaaattacatgagtccagctaccctcatttcttttttatgtgacatggagcacaaagaactgaaaatatgttttgagggagcatgacatccttgacctcatttgaaattacttccattttcaaaaaccatcatgttttgcagatacattttgagaaaaaccatcatgttttgaggatacatttgtgaaagtgcaggcatgccaatgactactcagttttaggacttttgtggaagaaaaattaaaagggagaagaaatcaattccactacttttgaagttctgcaacagatgagattttgtttgtaaagttttgtagtatattaaccattggtttatcactctagattcaacatattaaaatgtattcaagctcacagatttttaatcagtaaggcctatctatattagttgtcttttatttcttctccttgtggacagttgttaatgaaaggagtaaggatttccctttttttgttttgtttgttttgttttttttaaccaaattcttagagatactatagaatccaaatgagaactgaattggacctcaagtcttctattcctactaatagagttctttgtgatggtaactgctgtgtcgtttgttttccacaagttgggatggattcatgtcgatacatccccatgcccttgacctcttctggcattctcctgtgctctgacaaactgagccagccttttagatctacatgaataaacaaactattttaccaagaaaaatctcagcttgcttactgcttaattaaaaacctacaatttacacacctccctgccttcaagactgtgagctttggatagccctgcttaaatgtttgtcaacaacaagagtcgtaatgtgtcagtacattgtagctcctttgaaatttaactctttctgtgttacatgaattgttttaaggagtccggcaaacatgtttcttcacttccatgagaatggtgccaagtgtcagactctaatgagccctcagctcaggttttaatttctattgaatgctaacattcttctgattttttggtatcttttataatcatatcggtttctgctgtattaatgacaattattcatgaaaataaaaaatgaaatattttgtatactaaaaaaaaaaaaaaaaaaaaaaaaaaaaaaaaaaaaaaaaaaaaaaaaaaaaaaaaaaaaaaaaaaaaaaaaaaa

SEQ ID NO: 139 - DNA, 1866 bp, Homo sapiens
cgttcctcggcgccgccggggccccagagggcagcggcagcaacagcagcagcagcagcagcgggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcagcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggagctcaggtggccccagttcaatctcccgctgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaa

SEQ ID NO: 140 - DNA, 1986 bp, Homo sapiens
agtctgggttggactggcggccgtggagtttgtgacatacgaggtgacacccctcgagtcacttcccttcaactccagctggagcgcctgcttggctttgggttcgttctgcagccttcgccccgctcctagcctcagggccggactccagcgcagagcccagcccagcgcagcctgccagcagccacccagccgcccagccgcccagccccgcacgaaacccggccagagcttcctagcagcccgagccatgaacaccgaaatgtatcagacccccatggaggtggcggtctaccagctgcacaatttctccatctccttcttctcttctctgcttggaggggatgtggtttccgttaagctggacaacagtgcctccggagccagcgtggtggccatagacaacaagatcgaacaggccatggatctggtgaagaatcatctgatgtatgctgtgagagaggaggtggagatcctgaaggagcagatccgagagctggtggagaagaactcccagctagagcgtgagaacaccctgttgaagaccctggcaagcccagagcagctggagaagttccagtcctgtctgagccctgaagagccagctcccgaatccccacaagtgcccgaggcccctggtggttctgcggtgtaagtggctctgtcctcagggtgggcagagccactaaacttgttttacctagttctttccagtttgtttttggctccccaagcatcatctcacgaggagaactttacacctagcacagctggtgccaagagatgtcctaaggacatggccacctgggtccactccagcgacagacccctgacaagagcaggtctctggaggctgagttgcatggggcctagtaacaccaagccagtgagcctctaatgctactgcgccctgggggctcccagggcctgggcaacttagctgcaactggcaaaggagaagggtagtttgaggtgtgacaccagtttgctccagaaagtttaaggggtctgtttctcatctccatggacatcttcaacagcttcacctgacaacgactgttcctatgaagaagccacttgtgttttaagcagaggcaacctctctcttctcctctgtttcgtgaaggcaggggacacagatgggagagattgagccaagtcagccttctgttggttaatatggtataatgcatggctttgtgcacagcccagtgtgggattacagctttgggatgaccgcttacaaagttctgtttggttagtattggcatagtttttctatatagccataaatgcgtatatatacccatagggctagatctgtatcttagtgtagcgatgtatacatatacacatccacctacatgttgaagggcctaaccagccttgggagtattgactggtcccttacctcttatggctaagtctttgactgtgttcatttaccaagttgacccagtttgtcttttaggttaagtaagactcgagagtaaaggcaaggaggggggccagcctctgaatgcggccacggatgccttgctgctgcaaccctttccccagctgtccactgaaacgtgaagtcctgttttgaatgccaaacccaccattcactggtgctgactacatagaatggggttgagagaagatcagtttgggcttcacagtgtcatttgaaaacgttttttgttttgttttgtaattattgtggaaaactttcaagtgaacagaaggatggtgtcctactgtggatgagggatgaacaaggggatggctttgatccaatggagcctgggaggtgtgcccagaaagcttgtctgtagcgggttttgtgagagtgaacactttccactttttgacaccttatcctgatgtatggttccaggatttggattttgattttccaaatgtagcttgaaatttcaataaactttgctctgtttttctaaaaataaaaaaaaaaaaaaaaaaaaaaaaaaaaaaaaaaaa

SEQ ID NO: 141 - DNA, 4740 bp, Homo sapiens
ttccggtttttctcaggggacgttgaaattatttttgtaacgggagtcgggagaggacggggcgtgccccgcgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcacgagttgacctagatgagatgtcgttcactttactgagctacagaaaaacatacgaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatcgaagattatttttggatcatgataaaactcttcagactgattctatagacagttttgaaacacagagaacaccacgaaaaagtaaccttgatgaagaggtgaatataattcctccacacactccagttaggactgttatgaacactatccaacaattaatgatgattttaaattctgcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgagtcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtc

SEQ ID NO: 142 - DNA, 4518 bp, Homo sapiens
acaaaatggtcctttagaaaatacatctgaattgctggctaatttcttgatttgcgactcaacgtaggacatcgcttgttcgtagctatcagaaccctcctgaattttccccaccatgctatctttattggcttgaactcctttcctaaaatggtccttctgttgatcctgtcagtcttacttttgaaagaagatgtccgtgggagtgcacagtccagtgagaggagggtggtggctcacatgccgggtgacatcattattggagctctcttttctgttcatcaccagcctactgtggacaaagttcatgagaggaagtgtggggcggtccgtgaacagtatggcattcagagagtggaggccatgctgcataccctggaaaggatcaattcagaccccacactcttgcccaacatcacactgggctgtgagataagggactcctgctggcattcggctgtggccctagagcagagcattgagttcataagagattccctcatttcttcagaagaggaagaaggcttggtacgctgtgtggatggctcctcctcttccttccgctccaagaagcccatagtaggggtcattgggcctggctccagttctgtagccattcaggtccagaatttgctccagcttttcaacatacctcagattgcttactcagcaaccagcatggatctgagtgacaagactctgttcaaatatttcatgagggttgtgccttcagatgctcagcaggcaagggccatggtggacatagtgaagaggtacaactggacctatgtatcagccgtgcacacagaaggcaactatggagaaagtgggatggaagccttcaaagatatgtcagcgaaggaagggatttgcatcgcccactcttacaaaatctacagtaatgcaggggagcagagctttgataagctgctgaagaagctcacaagtcacttgcccaaggcccgggtggtggcctgcttctgtgagggcatgacggtgagaggtctgctgatggccatgaggcgcctgggtctagcgggagaatttctgcttctgggcagtgatggctgggctgacaggtatgatgtgacagatggatatcagcgagaagctgttggtggcatcacaatcaagctccaatctcccgatgtcaagtggtttgatgattattatctgaagctccggccagaaacaaaccaccgaaacccttggtttcaagaattttggcagcatcgttttcagtgccgactggaagggtttccacaggagaacagcaaatacaacaagacttgcaatagttctctgactctgaaaacacatcatgttcaggattccaaaatgggatttgtgatcaacgccatctattcgatggcctatgggctccacaacatgcagatgtccctctgcccaggctatgcaggactctgtgatgccatgaagccaattgatggacggaaacttttggagtccctgatgaaaaccaattttactggggtttctggagatacgatcctattcgatgagaatggagactctccaggaaggtatgaaataatgaatttcaaggaaatgggaaaagattactttgattatatcaacgttggaagttgggacaatggagaattaaaaatggatgatgatgaagtatggtccaagaaaagcaacatcatcagatctgtgtgcagtgaaccatgtgagaaaggccagatcaaggtgatccgaaagggagaagtcagctgttgttggacctgtacaccttgtaaggagaatgagtatgtctttgatgagtacacatgcaaggcatgccaactggggtcttggcccactgatgatctcacaggttgtgacttgatcccagtacagtatcttcgatggggtgaccctgaacccattgcagctgtggtgtttgcctgccttggcctcctggccaccctgtttgttactgtagtcttcatcatttaccgtgatacaccagtagtcaagtcctcaagcagggaactctgctacattatccttgctggcatctgcctgggctacttatgtaccttctgcctcattgcgaagcccaaacagatttactgctaccttcagagaattggcattggtctctccccagccatgagctactcagcccttgtaacaaagaccaaccgtattgcaaggatcctggctggcagcaagaagaagatctgtaccaaaaagcccagattcatgagtgcctgtgcccagctagtgattgctttcattctcatatgcatccagttgggcatcatcgttgccctctttataatggagcctcctgacataatgcatgactacccaagcattcgagaagtctacctgatctgtaacaccaccaacctaggagttgtcactccacttggatacaatggattgttgattttgagctgcaccttctatgcgttcaagaccagaaatgttccagctaacttcaacgaggccaagtatatcgccttcacaatgtacacgacctgcattatatggctagcttttgtgccaatctactttggcagcaactacaaaatcatcaccatgtgtttctcggtcagcctcagtgccacagtggccctaggctgcatgtttgtgccgaaggtgtacatcatcctggccaaaccagagagaaacgtgcgcagcgccttcaccacatctaccgtggtgcgcatgcatgtaggggatggcaagtcatcctccgcagccagcagatccagcagcctagtcaacctgtggaagagaaggggctcctctggggaaaccttaagttccaatggaaaatccgtcacgtgggcccagaatgagaagagcagccgggggcagcacctgtggcagcgcctgtccatccacatcaacaagaaagaaaaccccaaccaaacggccgtcatcaagcccttccccaagagcacggagagccgtggcctgggcgctggcgctggcgcaggcgggagcgctgggggcgtgggggccacgggcggtgcgggctgcgcaggcgccggcccaggcgggcccgagtccccagacgccggccccaaggcgctgtatgatgtggccgaggctgaggagcacttcccggcgcccgcgcggccgcgctcaccgtcgcccatcagcacgctgagccaccgcgcgggctcggccagccgcacggacgacgatgtgccgtcgctgcactcggagcctgtggcgcgcagcagctcctcgcagggctccctcatggagcagatcagcagtgtggtcacccgcttcacggccaacatcagcgagctcaactccatgatgctgtccaccgcggcccccagccccggcgtcggcgccccgctctgctcgtcctacctgatccccaaagagatccagttgcccacgaccatgacgacctttgccgaaatccagcctctgccggccatcgaagtcacgggcggcgcgcagcccgcggcaggggcgcaggcggctggggacgcggcccgggagagccccgcggccggtcccgaggctgcggccgccaagccagacctggaggagctggtggctctcaccccgccgtcccccttcagagactcggtggactcggggagcacaacccccaactcgccagtgtccgagtcggccctctgtatcccgtcgtctcccaaatatgacactcttatcataagagattacactcagagctcctcgtcgttgtgaatgtccctggaaagcacgccggcctgcgcgtgcggagcggagccccccgtgttcacacacacacaatggcaagcatagtcgcctggttacggcccagggggaatatgccaagggaccccttaatggaaacacagatcagtagtgctatctcatgacaaccacaagaaaccgacgacaaatcttttgcgagattttcttctagtggcttagaaacatggcttttaagaaacacggtgatatctttgagggtgacaaggcgtctcttcaaacagttccataccaactgctttgctctagggaagcagtgcgtgtgaaacagcgtaacggagggtgaagagcatagttaataagcaactgtaaaaagttttatttgtttactttaattcttttcccctgtaaaaagttttatttgtttactttaattcttttcccagaaaagagtctttgattcaccaaacatgaatgtacattttctaacaaactcaaaatctgggaccaaaacatcaacttttttctttcttttttctttctttttgttttttctttcctgtaaagaccttgaaaagaccttgaaaagcagtaacttgggtccagtatttacggaggcgttgtgaatgtgtcccatgcataacacactactggatagtgagtcgtgcgctaatgtactacgtagggcttctaccagagattttcctctccaattgggttgtgaaatactcttccaaaagcctgcatcggggattccacctacttatttcagattcacctccattaaccaagaaaaccagtggaagatttcttgactatttcaccatgttgccaatc

SEQ ID NO: 143 - DNA, 3302 bp, Homo sapiens
cggtgctgccgccgttgccgggagccgcggagacaagtcattacgttttcatttctcacaactgggctgagcacaactgaaccatgggggaacacagtccagacaacaacatcatctactttgaggcagaggaagatgagctgacccccgatgataaaatgctcaggtttgtggataaaaacggactggtgccttcctcatctggaactgtttatgataggaccactgttcttattgagcaggaccctggcactttggaggatgaagatgacgacggacagtgcggagaacacttgccttttctagtagggggtgaagagggctttcacctgatagatcatgaagcaatgtcccagggttatgtgcagcacattatctcaccagatcagattcatttgacaataaaccctggttccacacccatgccaagaaatattgaaggtgcaaccctcactctgcagtcggaatgtccggaaacaaaacgtaaagaagtaaagcggtaccaatgtacctttgagggctgtccccgcacctacagcacagcaggcaacctgcgaacccaccagaagactcaccgaggagagtacacctttgtctgtaatcaggagggctgtggcaaagccttccttacctctcacagcctcaggatccacgtgcgagtgcacacgaaggagaagccatttgagtgtgacgtgcagggctgtgagaaggcattcaacacactgtacaggctgaaagcacatcagaggcttcacacagggaaaacgtttaactgtgaatctgaaggctgcagcaaatacttcaccacactcagtgatctgaggaagcacattcgaactcatacaggggaaaagccatttcggtgcgatcacgatggctgtggaaaagcatttgcagcaagccaccaccttaaaactcacgttcgtacacatactggtgaaagacccttcttctgccccagtaatggctgtgagaaaacattcagcactcaatacagtctcaaaagtcacatgaaaggtcatgataacaaaggacactcatacaatgcacttccacaacacaatggatcagaggatacaaatcactcactttgtctaagtgacttgagccttctgtccacagattctgaattgcgagaaaattccagtacgacccagggccaggacctcagcacaatttcaccagcaatcatctttgaatcaatgttccagaattcagatgatacggcaattcaggaagatcctcaacagacagcttccttgactgaaagttttaatggtgatgcagagtcagtcagtgatgttccgccatccacaggaaattcagcatctttatctcttccacttgtactgcaacctggcctctccgagccaccccagcctctactacctgcctcagctccgtctgctcctccgcctgctccctccctaggacctggctcccagcaagctgcatttggcaacccccctgctctcttacaacctccagaagtgcctgttccccacagcacacagtttgctgctaatcatcaagagtttcttccgcacccccaggcaccgcagcccattgtaccaggactttctgttgttgctggggcttctgcatcagcagcggcagtggcatcagctgtggcagcaccagccccaccacaaagtactactgagcccctgccagccatggtccagactctgcccctgggtgccaactctgtcctaactaataatcccacaataaccatcaccccaactcccaacacagctatcctgcagtccagcctagtcatgggagaacagaacttacaatggatattaaatggtgccaccagttctccacaaaaccaagaacaaattcagcaagcatctaaagttgagaaggtgttttttaccactgcagtaccagtagccagtagcccagggagctctgtccagcagattggcctcagtgttcctgtgatcatcatcaaacaagaagaggcatgtcagtgtcagtgtgcatgccgggactctgcaaaggagcgggcatccagcaggagaaagggctgctcctccccaccccctccagagccgagcccccaggctcctgatgggcccagcctgcagctcccagcgcagactttctcttcagcccctgttcccgggtcatcatcctctaccttgccctcctcctgtgagcaaagccgacaagcagagactccttcagaccctcagacagaaacattaagtgccatggatgtgtcagagtttctatccctccagagcctggacaccccgtccaatctgattcccattgaagcactactgcagggggaggaggagatgggcctcaccagcagcttctccaagtgaagggcccatgtgtgctcacctctgggaaaagcgggtgagcaggaggcatgaggtacaatgcctgccatcatgggtcagaaatttgaaggatgaagaaatctactgtttgaaatcctcacctttcagacgtattttctttattcacatcccaggagcatccattttaaggaactattctttggaaaaaaacaaaaaacaaaaaaaacaacaaaaaaagctaagttataagtgaactgtttggctgcactgtatgtcacttttgcttgttgtcatgtgaacttggaaactaaggttactcgtgtgcataaaaattctaaatgaaagggtgtggtttccatcaatctgatgctgcccatcgcttgcactggggtctttgtggatcgggcaggagttttcagtgtgttgggtgttgctccttcctatgtgtcttttgaatctgaggctgacatttgcttggaaggccagacccttgctccatcagagagggcagtggcaaaggccagtgaggcagctgtgagttggacagggttcaggtgagatggtgttgtcatttgtgcttagtgttggtggtgctcagggtggataacacgggtcgttctgcagcccgcttcagcacaaataggcagcttaaggcctggctcacaggctgtggggttgatctggctctgcagaggccctaggcagcttgttgactgctgtctgttgatgacgtgtgtgcaaagcaggctctagcaacatgatcactgtccttgccttcctggttctttctctcggttggttgccagggcttgcagatcgcagtgaattttccttggggaacatcgctgttttgtcctagagtgaacttgtggcttatggccagtgctgtttggtggtctgccttctttttaatggtattttcttcctcagagcagaagggctgcattttgcttatcagaagaaggtgcagatttaagg

SEQ ID NO: 144 - DNA, 4978 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 145 - DNA, 1887 bp, Homo sapiens
cgcgcagcccctccggccgcgggcgcagcgggggcgctggtggagctgcgaagggccaggtccggcgggcggggcggcggctggcactggctccggactctgcccggccagggcggcggctccagccgggagggcgacgtggagcggccacgtggagcggcccgggggaggctggcggcgggaggcgaggcgcgggcggcgcagcagccaggagcgcccacggagctggacccccagagccgcgcggcgccgcagcagttccaggaaggatgttacctttgacgatgacagtgttaatcctgctgctgctccccacgggtcaggctgccccaaaggatggagtcacaaggccagactctgaagtgcagcatcagctcctgcccaaccccttccagccaggccaggagcagctcggacttctgcagagctacctaaagggactaggaaggacagaagtgcaactggagcatctgagccgggagcaggttctcctctacctctttgccctccatgactatgaccagagtggacagctggatggcctggagctgctgtccatgttgacagctgctctggcccctggagctgccaactctcctaccaccaacccggtgatattgatagtggacaaagtgctcgagacgcaggacctgaatggggatgggctcatgacccctgctgagctcatcaacttcccgggagtagccctcaggcacgtggagcccggagagccccttgctccatctcctcaggagccacaagctgttggaaggcagtccctattagctaaaagcccattaagacaagaaacacaggaagcccctggtcccagagaagaagcaaagggccaggtagaggccagaagggagtctttggatcctgtccaggagcctgggggccaggcagaggctgatggagatgttccagggcccagaggggaagctgagggccaggcagaggctaaaggagatgcccctgggcccagaggggaagctgggggccaggcagaggctgaaggagatgcccccgggcccagaggggaagctgggggccaggcagaggccagggagaatggagaggaggccaaggaacttccaggggaaacactggagtctaagaacacccaaaatgactttgaggtgcacattgttcaagtggagaatgatgagatctagatcttgaagatacaggtaccccacgaagtctcagtgccagaacataagccctgaagtgggcaggggaaatgtacgctgggacaaggaccatctctgtgccccctgtctggtcccagtaggtatcaggtctttctgtgcagctcagggagaccctaagttaaggggcagattaccaataaagaactgaatgaattcatccccccgggccacctctctacccgtccagcctgcccagaccctctcagaggaacggggttggggaccgaaaggacagggatgccgcctgcccagtgtttctgggcctcacggtgctccggcagcagagcgcatggtgctagccatggccggctgcagaggacccagtgaggaaagctcagtctatccctgggccccaaaccctcaccggttccccctcacctggtgttcagacaccccatgctctcctgcagctcagggcaggtgaccccatccccagtaatattaatcatcactagaactttttgagagccttgtacacatcaggcatcatgctgggcattttatatatgattttatcctcacaataattctgtagccaagcagaattggttccatttgacagatgaagaaattgaggcagattgcgttaagtgctgtaccctaaggtgatatgcagctaattaaatggcagatttgaaaaaaaaaaaaaaaaaaaaaaaaaaaaaaaaaaaaa

SEQ ID NO: 146 - DNA, 2104 bp, Homo sapiens
acgtagcgcggcgctcggaactgacctactaacacacatctctccgcgcggccacggcgcccgcggacccggcgcgcccgcccgcctcccgcgccgcgccctcgccgccgcccgcctcccgccgcggccccggaggcccggcccggcccgagccccgagcgccggcggcccgactcccggccgcccctttctttctcctcgccggcccgagagcaggaacacgataacgaaggaggcccaacttcattcaataaggagcctgacggatttatcccagacggtagaacaaaaggaagaatattgatggattttaaaccagagtttttaaagagcttgagaatacggggaaattaatttgttctcctacacacatagatagggtaaggttgtttctgatgcagctgagaaaaatgcagaccgtcaaaaaggagcaggcgtctcttgatgccagtagcaatgtggacaagatgatggtccttaattctgctttaacggaagtgtcagaagactccacaacaggtgaggagctgcttctcagtgaaggaagtgtggggaagaacaaatcttctgcatgtcggaggaaacgggaattcattcctgatgaaaagaaagatgctatgtattgggaaaaaaggcggaaaaataatgaagctgccaaaagatctcgtgagaagcgtcgactgaatgacctggttttagagaacaaactaattgcactgggagaagaaaacgccactttaaaagctgagctgctttcactaaaattaaagtttggtttaattagctccacagcatatgctcaagagattcagaaactcagtaattctacagctgtgtactttcaagattaccagacttccaaatccaatgtgagttcatttgtggacgagcacgaaccctcgatggtgtcaagtagttgtatttctgtcattaaacactctccacaaagctcgctgtccgatgtttcagaagtgtcctcagtagaacacacgcaggagagctctgtgcagggaagctgcagaagtcctgaaaacaagttccagattatcaagcaagagccgatggaattagagagctacacaagggagccaagagatgaccgaggctcttacacagcgtccatctatcaaaactatatggggaattctttctctgggtactcacactctcccccactactgcaagtcaaccgatcctccagcaactccccgagaacgtcggaaactgatgatggtgtggtaggaaagtcatctgatggagaagacgagcaacaggtccccaagggccccatccattctccagttgaactcaagcatgtgcatgcaactgtggttaaagttccagaagtgaattcctctgccttgccacacaagctccggatcaaagccaaagccatgcagatcaaagtagaagcctttgataatgaatttgaggccacgcaaaaactttcctcacctattgacatgacatctaaaagacatttcgaactcgaaaagcatagtgccccaagtatggtacattcttctcttactcctttctcagtgcaagtgactaacattcaagattggtctctcaaatcggagcactggcatcaaaaagaactgagtggcaaaactcagaatagtttcaaaactggagttgttgaaatgaaagacagtggctacaaagtttctgacccagagaacttgtatttgaagcaggggatagcaaacttatctgcagaggttgtctcactcaagagacttatagccacacaaccaatctctgcttcagactctgggtaaattactactgagtaagagctgggcatttagaaagatgtcatttgcaatagagcagtccattttgtattatgctgaattttcactggacctgtgatgtcatttcactgtgatgtgcacatgttgtctgtttggtgtctttttgtgcacagattatgatgaagattagattgtgttatcactctgcctgtgtatagtcagatagtccatgcgaaggctgtatatattgaacattatttttgttgttctattataaagtgtgtaagttaccagtttcaataaaggattggtgacaaacacagaaaaaaaaaaaaaaaaaaaaa